SEQUENCE LISTING (139 sequences)

SEQ ID NO: 1 - DNA, 2418 bp, Mus musculus
  sig_peptide 1..51
  misc_feature 52..2220: note = Extracellular_domain
  misc_feature 2221..2283: note = Transmembrane Domain
  misc_feature 2284..2418: note = Cytoplasmic Domain
  source 1..2418: mol_type = other DNA
atgtccagctcctcctggctccttctcagccttgttgctgttactactgctcagtccctcaccgaggaaaatgccaagacatttttaaacaactttaatcaggaagctgaagacctgtcttatcaaagttcacttgcttcttggaattataatactaacattactgaagaaaatgcccaaaagatgagtgaggctgcagccaaatggtctgccttttatgaagaacagtctaagactgcccaaagtttctcactacaagaaatccagactccgatcatcaagcgtcaactacaggcccttcagcaaagtgggtcttcagcactctcagcagacaagaacaaacagttgaacacaattctgaacaccatgagcaccatttacagtactggaaaagtttgcaacccaaagaacccacaagaatgcttattacttgagccaggattggatgaaataatggcgacaagcacagactacaactctaggctctgggcatgggagggctggagggctgaggttggcaagcagctgaggccgttgtatgaagagtatgtggtcctgaaaaacgagatggcaagagcaaacaattataacgactatggggattattggagaggggactatgaagcagagggagcagatggctacaactataaccgtaaccagttgattgaagatgtagaacgtaccttcgcagagatcaagccattgtatgagcatcttcatgcctatgtgaggaggaagttgatggatacctacccttcctacatcagccccactggatgcctccctgcccatttgcttggtgatatgtggggtagattttggacaaatctgtaccctttgactgttccctttgcacagaaaccaaacatagatgttactgatgcaatgatgaatcagggctgggatgcagaaaggatatttcaagaggcagagaaattctttgtttctgttggccttcctcatatgactcaaggattctgggcaaactctatgctgactgagccagcagatggccggaaagttgtctgccaccccacagcttgggatctgggacacggagacttcagaatcaagatgtgtacaaaggtcacaatggacaacttcttgacagcccatcacgagatgggacacatccaatatgacatggcatatgccaggcaacctttcctgctaagaaacggagccaatgaagggttccatgaagctgttggagaaatcatgtcactttctgcagctacccccaagcatctgaaatccattggtcttctgccatccgattttcaagaagatagcgaaacagagataaacttcctactgaaacaggcattgacaattgttggaacactaccgtttacttacatgttagagaagtggaggtggatggtctttcggggtgaaattcccaaagagcagtggatgaaaaagtggtgggagatgaagcgggagatcgttggtgtggtggagcctctgcctcatgatgaaacatactgtgaccctgcatctctgttccatgtttctaatgattactcattcattcgatattacacaaggaccatttaccaattccagtttcaagaagctctttgtcaagcagctaagtataatggttctctgcacaaatgtgacatctcaaattccactgaagctgggcagaagttgctcaagatgctgagtcttggaaattcagagccctggaccaaagccttggaaaatgtggtaggagcaaggaatatggatgtaaaaccactgctcaattacttccaaccgttgtttgactggctgaaagagcagaacagaaattcttttgtggggtggaacactgaatggagcccatatgccgaccaaagcattaaagtgaggataagcctaaaatcagctcttggagctaatgcatatgaatggaccaacaacgaaatgttcctgttccgatcatctgttgcatatgccatgagaaagtatttttcaataatcaaaaaccagacagttccttttctagaggaagatgtacgagtgagtgatttgaaaccaagagtctccttctacttctttgtcacctcaccccaaaatgtgtctgatgtcattcctagaagtgaagttgaagatgccatcaggatgtctcggggccgcatcaatgatgtctttggcctgaatgataacagcctggagtttctggggattcacccaacacttgagccaccttaccagcctcctgtcaccatatggctgattatttttggtgttgtgatggcactggtagtggttggcatcatcatcctgattgtcactgggatcaaaggtcgaaagaagaaaaatgaaacaaaaagagaagagaacccttatgactcgatggacattggaaaaggagaaagcaatgcaggattccaaaacagtgatgatgctcagacttccttttag

SEQ ID NO: 2 - protein, 805 aa, Mus musculus
  SIGNAL 1..17
  REGION 18..740: note = MISC_FEATURE - Extracellular Domain
  REGION 741..761: note = MISC_FEATURE - Transmembrane Domain
  REGION 742..805: note = MISC_FEATURE - Cytoplasmic Domain
MSSSSWLLLSLVAVTTAQSLTEENAKTFLNNFNQEAEDLSYQSSLASWNYNTNITEENAQKMSEAAAKWSAFYEEQSKTAQSFSLQEIQTPIIKRQLQALQQSGSSALSADKNKQLNTILNTMSTIYSTGKVCNPKNPQECLLLEPGLDEIMATSTDYNSRLWAWEGWRAEVGKQLRPLYEEYVVLKNEMARANNYNDYGDYWRGDYEAEGADGYNYNRNQLIEDVERTFAEIKPLYEHLHAYVRRKLMDTYPSYISPTGCLPAHLLGDMWGRFWTNLYPLTVPFAQKPNIDVTDAMMNQGWDAERIFQEAEKFFVSVGLPHMTQGFWANSMLTEPADGRKVVCHPTAWDLGHGDFRIKMCTKVTMDNFLTAHHEMGHIQYDMAYARQPFLLRNGANEGFHEAVGEIMSLSAATPKHLKSIGLLPSDFQEDSETEINFLLKQALTIVGTLPFTYMLEKWRWMVFRGEIPKEQWMKKWWEMKREIVGVVEPLPHDETYCDPASLFHVSNDYSFIRYYTRTIYQFQFQEALCQAAKYNGSLHKCDISNSTEAGQKLLKMLSLGNSEPWTKALENVVGARNMDVKPLLNYFQPLFDWLKEQNRNSFVGWNTEWSPYADQSIKVRISLKSALGANAYEWTNNEMFLFRSSVAYAMRKYFSIIKNQTVPFLEEDVRVSDLKPRVSFYFFVTSPQNVSDVIPRSEVEDAIRMSRGRINDVFGLNDNSLEFLGIHPTLEPPYQPPVTIWLIIFGVVMALVVVGIIILIVTGIKGRKKKNETKREENPYDSMDIGKGESNAGFQNSDDAQTSF

SEQ ID NO: 3 - DNA, 2418 bp, Homo sapiens
  sig_peptide 1..51
  misc_feature 52..2220: note = Extracellular Domain
  misc_feature 2221..2283: note = Transmembrane Domain
  misc_feature 2284..2418: note = Cytoplasmic Domain
  source 1..2418: mol_type = other DNA
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 4 - protein, 805 aa, Homo sapiens [sequence omitted]

SEQ ID NO: 5 - DNA, 45619 bp, synthetic construct
  misc_feature 1..45619: note = 7878 allele
  misc_feature 1..1377: note = 5' Mouse Sequence
  misc_feature 1378..37630: note = Human 36253 bp from CTD-3010D6
  misc_feature 37631..37636: note = Xho1 restriction site
  misc_feature 37637..37670: note = LoxP sequence
  misc_feature 37671..38357: note = Prm
  misc_feature 38358..39498: note = Crei
  misc_feature 39511..39740: note = SV40 p(A)
  misc_feature 39795..41007: note = hUbi
  misc_feature 41008..41074: note = EM7
  misc_feature 41075..41878: note = Neo
  misc_feature 41879..42363: note = PGK p(A)
  misc_feature 42369..42402: note = LoxP site
  misc_feature 42409..42434: note = IceuI
  misc_feature 42435..42440: note = NheI
  misc_feature 42441..42929: note = Human equence 3'
  misc_feature 42930..45610: note = Mouse sequence 3'
  source 1..45619: mol_type = other DNA
aggcccatgagccctgccatttaaagtggctcctctcttacactctgggaatgaggacacggagccagctgctgaacttcaccaggataaccattaaaattgctttggagttcatatttccacgatcccatgcctatggatgccaaggacttgtcatggatgcgctttggatttcataatgcagagtcattattacttccttgagttctcagctgagttgtaagcaggtaagtgaaagggaaagaggcacctgataaagtcagctgtaggactagagtttagcatgtctcctgagaaatagaaaatgactgcttgaaactttaccaaagccacacaggaagcatcaaactccctatggagtggagaagagtcttataattttttaaatgggcagagaaatgaatttatttttaatttttagagacagggtttctttgtatagctctagctgtctttgattggtagacaaagctgtcctcaaactcagagatcttccttcctttgtctcctgagtgctgggattaaaggcatggaccaccactgccctgccccattctctccattaattttaagtgaatgcttgcaaaagctcacttctttggtgaacagcttcctttacaaataagtacctttgccttcgtttttataggattcttaaaaagaaaaaaaagattcagccaggtggttgtggtgcacacctttaatcccagcagtcaggaggcagaggaaagcagatctcttgagtttgaggctagcctagtctacagagggagttccaggacagccaaggctacagagaggaactgtctaaaaacaccaagaaagagagaaaggagagagggagaggatggatagcttattgatagaattgtcagaaaaggctataagttccaatatgtgtcccatgatttctaagtctagccctttctgttatagtaaaatcatagtacaccctcctcctccagtgtatctttaacagcttttaaggaacatattaactaaatgtccaggttttgatttggccataaaatgttagcaaagctaaggttttctaggattaatgaataacatgtctttatttagtttacttaaaaaaatcattctaaaatatctgtttacatatctgtcctctccaggattaacttcatattggtccagcagcttgtttactgttctcttctgtttcttcttctgctttttttttcttctcttctcagtgcccaacccaagttcaaaggctgatgagagagaaaaactcatgaagagattttactctagggaaagttgctcagtggatgggatcttggcgcacggggaaagatgtccagctcctcctggctccttctcagccttgttgctgttactactgctcagtccaccattgaggaacaggccaagacatttttggacaagtttaaccacgaagccgaagacctgttctatcaaagttcacttgcttcttggaattataacaccaatattactgaagagaatgtccaaaacatggtgagttctcatggctctattgggctatttgttgcctcttaaagattagattactgcccgtgaaactgaaaagggaaatcaaaaaaaatgctctgagttgtgagattggatgtttgcctccatatccctgattttggcgtcgacgataactaaacttaattgaccctggggttcattcagaaaattgttacaaaataattcactggtctcaatccagatgcttggaaacaagagaatattctttataaagttaccaccataattctcatcacagtcaggatttcaaagcatttcatggaaatgccataagttattgagttaataatttcctttagcctacaatatcaataggaatatctaaagattctctacaaatgatatattaactgaaaaatgcaactggaggcttagtgaaagaactagtaattaagctaatcagggcttgggtgaggctggacttgggaattcctttcctctttgtcacagacctccaagggactccaaaaggtcacagaatccaggcagagccctccaaaacaaaaacaaaaacaaacaacaaaaaaactatgattaaaccagttctgacaaatatcagaatgctctgggaaagccagatgctttaacaagtgcaaggatttaggaatttcttctctcacagatcccaaaacagtatccttaagtggtgatccttatgtcaagctgtgagaatatacctcaaaataatagaaggcatccaaacttgatcagtatagtgtcataaagctgctgatgtagaagtgtggagaggtccatcagatagagatatggaaaaaaaagcttggttaaaacaggtttccaaagttctctccctttctctgccattgtggtatgtgtggttgaccatttctcatcatgttttcttacaagactttcaggatcaagcaattcctcgccaaaaaacaaaatcaaggacacgttgtagctatttcttggtgagttaccagaagctgtgaagagagagcatgaggcccatacttttttttttttttgaagggtctcgctctgttgcccaggctggaatggagtggcatgatcatgattcattgcagcttgacctcctgggctcaaatgattctctcgcatcatttaacatttttttttgtagagatgagatctcactttgtctcccaggctggtcttgaactcctgggctcaagtgatcctcttgcctcaggctcccaaagtgctgggatgacaggcatgagccactgcacctagcctaccacacttactctttgcgtggtctctagagcttcttcagcccccctgctgccagggtccctagggtcttgggcatgccccatttctgaacctattcctgtctccagggatagagtcacagtgaactcagaccagggtaattctaaactgctattctgtatatacccaaggaaatttcatccctgtctctaatgagtctgtcccatgccccttaatacacatgaacacctacacacaggtacaagtcggggaagatcactgtgcagcacataaaataaaattcaggggcgagcactttttttcccatgagataaagttaggaaaaaataaaaatgttcatacagattaaaaattttaaatgttaaaatattaaacccccaatctaaaataaagaaaaaataatttcttaaatcctaagcttgagttatggaattcacattcttataatttacattacctattttgttaaatggaagacttttttttttaatatgcaagtacctgggcatatttagaaagtaaccaaggcagcttttgtctttgaacctgatgcggttttgacagctctatctggaaattgagccagcatatgggagccaagggtgaaagttttcctgggagatttgggctccaaggtactggatagatgctaaagattccattcagtttttcattttctgtcttcatggaactctcacacctacaggggaagaaatggtttagaggttactaaaagtatatagcctggtttaggtgatagtgagctatagaaaaagagaaaataagttgtcagtactttaaatctcaaacaacttcttacaaaaggattttaatagtttaaatacaattatcacaagttgggtaaagacaaccagacctatggcctgataaatcatgtatttaatttatgcatatagttagggccaaaaagagcagagtatctttttgaatgttttaaaatataatctaggattttatgcctacagtccttgaaattacaaaggatctctctcaagttgggaatatgctatccattgcagttacggatgggatttagagactgttttaatttcttgtgattatctcaaacaacgtgttccttactcccaaaactcccttgtatggctacagaaactgttgcagttttagttgcttctcccctgcctttgcccagaggtagtgtggaatgccaccaattttatctgtaatggttcctttcaagcctggggctatgtaagctgctatatatgcttcattccattcatcattcagtctaaatatagctatcaaccactaaattatctgataggtatgcatgaactgactgattctttgcataactactaagctatctgttttatttaggcttgggagccctagggtaaggatacggaataaattgtaaatgatattaaaagaatagcttggcaaggaataatacctcctcaggggccaaaatgcaaggcaacacaaagtgatttttttggtgatataattataacacatgtagtatggtagtgataactaaaataaaattggatagcctcaaaattcctacatgtagtatgataaacaatgttctaagactgaggcctttgggaggtagtgagttatttcctatagatagagagtaaaattattcctataatttggtatgaaagttttaccagaaattttgcttcacagtaaatcatctggattgggtgaagccaggtagggattgagaagtgagtcaggtgggttgcatagatgagtttgtgctgcagaccaaactcctcttcccatagctcacttcttcccactcctgcatgttagagagaagactagaatgtgtcaaatacaacctataggataatagttcagagcattagcttcggaagtaaacactaggttgatgccctggttcttctaatttctagctgtgtgacctagggaaagttacctgctgtgtgcctcatttcccttatttgtaaagttgagacaataacaactccctcacaaggttgttgtgaagattaatgaaaaagcagtttctgagaggttgtgagtgttcagtaaagaaagaaattgctttcctttttttttttttttgagacagattctcattctgtcattcaggctggagtgcagtggcgcgatcatggctcactgcgcctcgacttccccaagctcaggcaatcttcccacctcagcctcctgagtagctgggactacaggtgcgagccaccacacctggctagtttttgtattatttgcagagatggggtctcgccttgttgcccaggcttgtctcaaacttctgggctcaagcgatcttcccacctcaacctcccaaagtgctgggattacaggcatgaaccactacacacagcctattttccattttcactgccattattttctctgtctcttgtgtggctcattactgccacttctgtgcaccatttccatcatgtcacatgcgtagtcaaaaccctcagtgactgcctaattctcagagggtaaaagtgaaattccttggcctggaaagtgcaatcatgcctgaagttctacctgtgggagctgtactagttatctattgtgtaaaaagttaccacaagcttagcagattaaaacaacacatatttaatatcccacagtatctgtgggctaggagtccaggcacagttcagctggatcttctgcttctaagtctcatatggttgcattcaaggtgctggctagagctatggtctcttctgaggcttgactggggatggatatacttccaagccaatgcaattactcagaggccgtctttaattatttgccatgtggccttttccatagggtagctcacaacgtggcatcttgctgcaacaagtcagcaaaggagagagtcttctatcaagattaaagttaaaatcttatgtaacgtaaacatgacatcctgtcacctgtgttgtatgctattggttgaaagtaaatcacgggtctgctcatacttatggggaggggatcacacaagggtgtgaccaccaaaggcagagatcatgggagcattcttagagtctgtctgccacagggccaggatcaaatgctggtggctgattgccagctccactgtttatttctctcagccgctttctgtatgtacaaaaaaggagagagagtgattttctacctcatagtgttgttaagaaaatcaaatgaggtaaattattttgcaaagtattctctactcatctgactcctacctactttcaggtctgtcttaagttgcaacttctgcatgagggcttggccaagtatggtcacctgcatgaatttcctctttcttgtaacagaaactgcttggcccactcataatttatacattattatatactcttatgtttcattaatgaaacatttataatgcatatcttatctgtctgataaaatggaaagcaatctaaggacaatgacttttcccctatacataacaaaatataaaataaatcataacataataaataataaataaataataaaataactaacatttgttgaatgattaggatgtgttaggcactcttgtaagcatttcatttttttgttacttcttcaaatccttacaacagtccttggaggtaagtcctattattactttcattttgcagataagacaactgtggctcagagaggctaaggtacttgagtccaggagcagaattcttactttcctcaaacgttatctcttttgtgtggtctagggcagtacaatataccaagtagttgctcattttggtctttggtaaatggaaatgaatttgtaagttgagaaaagactacactacagtagtttggagaagtccactttccaactgatgttcaagattcaggagggggggaaatgtgtcctggcatcatcacttgcttgatgatttcttttgctggtagaaaatcaccatggctaccaatggccattttctgggaagccttttttgggaatcctaacttaagatcaaaatccctgggctaagtcacggcagctaggaggttactgtgaaccggttctcatttcttatggtttccattgttattagggggctgtattctcattaagtccactctgagctctactggattctctttttagagcactcgtctttgaattcttggccaactcatctatgtcacagcactaaggctataaattccagggccccacaatgtggattatctgtgagaattcatttccagttctggggacaagacccttgggaaaaccaggcaataggccagagagagacagagagagagagagtggtcaaaagtggcctggtcactcttaacctaaaccttgaccttcagcggagtagaggactaaatcactcaggaaatattaaatgtttcaccagatattatttggactatatcttctgttctatctcttcaagcaatgccattccaacttcaattttcttttctgtcatttcagaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacgggtacgtttgtgaacattttagcattgatcccaagagttgaaatatttgggaaatattaaacattaatatcacccacagcagctgcctttatttgaaaaaatagtaatgctgatgttagaaaaatctctctctatgcctaggtaaggtgtccatataatttattattcaaactggaatctttgagagtgaaagacactggtaattattatattcaaacacagagttaaactgagattgtccctggcactgtggtgtgtatggacaccccctagtgttaggtcaccttcctgaaggatcaggcagcctgaagaatacagttagtttatgtcagtggttctcaactgggggtgatttagccctccggggatatttgacaatgtctggagacgtttttggttgccacaaatggggatactgttgacatctagtgggtagggatgctgcagaacatttcacaatgcatgggatggcccccaacaacaaagaatgatcttgtccacaatgtcaacagcaaacttttttttctgtaaaggaacagatgacagtaaatattttaggttttgtgtatttaggtattgtctgtgttgcaactagtcaactgtgctgttgtatcaggaaatcagctgtagctaatatataaatgaacaagtatggctgtgttcaaataaaactttacaaaaacaggcagcagacaggatttggtctgcagaacacagtttgcagacctctgatatagcctattgaaatatttttaacctaaaatatttagctatttaaaaagaaagtcttctttctcagttgcttgtcctctggaactttgtatgttctaatctgtgaatataaaaataaaaatgcatcaaaaaggcaaagataaaaccatgtgaaatgtatctttctacaagttaacatttatttttctgcattctttgggtccaattatccaactaattatcaatgcactttactttatccattctatgtttctccattttttcccccaacaaggacactataagaaaccttgcccattcgcatatagacaatctggtctagcctaggtggaacctagaaattctattttttaaaaaatccccagataattctaatgtgtagccaggactgaaaaccactgaaatgcatgtaagagcttaaagtgtttgggaagaggagggactcaaggttgcttagatttgatggagaggaagccgtggagattgaagaagagctggagagagaccatgaggggttttgtgccatggtaaggaatttaggtttgttcctttggactatgggatcgtgtggaagttttaaacataggatctatttcatgttttaggatgactctcaaggcagaaccaaagggaaataggagaaggagaaacagatcctttaatccagtgcttcttaaatcaatctgagggagagaaccatttccttccttccttccttcctttcttctcttcttccttccctccttctctcctccctcccgtctttcctttctccctcctttattcctttatttccaatacattgcagatcaatacttgcttaagttcattgattacatgcttagatgtcatggaaatatcaaattgttctccaaagtttataaacttttattctcttaattcctgtacttatcacagaatggaaacaaactgtcacttgcactggtccacggattacacctggagtagcactgcttcactatatcaccttcactttatctgcagggtaaagtttaaaaactttggtatggcatagacggtccctcgtgatctggcccctgcttgtctctccattctcatgtctttcatttcccctaatgaacaccattgctccagtttcattcaacatctcactgtcccctgagaatgccttgtcttttccaacctccttccatcttttagactcttccctctacctcccttctctacatgaccagtgcctacttcttctccaagacacagctcttatcttcccttctctttggacccattcttgaatctcctggttattagttattaatttctccctgctcctatactaccgcatcactttttggtgacagttatagcacttgtcattctgttatgtattaatagttattgatttcatggctgcctcactgtcctatgactttatggtctttgacttgttcacttcagaattgtcagagtgtaggacatattccaggttcaataaatgctgaatgaaacaaaaaatgcacctctctgtgtatgtgtggataagaaaacttctttgggttttggtagatctggttatttcaattactccatatatatttattatgtgcttatagtactttgtgctagtcgacagtggggaaacttaactgggctattccttctccacatacagaaagatacatggcaaagaagtaaattgctgtattttgagtaatcaaaaaatcatgtggtcaaaaggatatctttatattagcattctcttcagcaaaatttccattgttaacattgtttattatctttaatttgcagttgaacacaattctaaatacaatgagcaccatctacagtactggaaaagtttgtaacccagataatccacaagaatgcttattacttgaaccaggtaggctactaatttttagtagtgattatgaaatttacttttctctcagattttaaaaatgattgcagatatgtgtgtttcaacacatagatgctcttttaacttttagtaaaactttcttgtaaattgtatcacatttacttaatttgatcaagtcagtaagtaagtcatttcagtggtttattttctctgcagacatattttggaactataagggcaaggacatgaatgaatcatgagattttatttaaccactttatgacctgatgtctgccaaccttacttttaattctttaaagttcatgtagttctttacaaataaaatttttacaaaggcatcttttcttgtctttgtgtgctttgggataacaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcgtaagtttgctgatctgtagaggtcctgaagctctctgtgtggccacggggctatttcttctgcattaccaagcctgccctttgatatcagggatacccctcaattaacaatcatgtcctcaacaattcagattacctgttttcaaaagaaattactggggaaaaagaaaaggtgcactggcattctacaattcaagaagctcaagaatgagactcaactacaaggcctaaagtaaattatccaggaattatgaattataagtaatctaggaagatctcaggcccctaaaagttcccgagggagtctggggattaagggggggaatcccatttctcaggatgaaagacagagtcactgccttttgtctaaagcccccacagttcactttcaaaggtcatttgtgtgtctaccttagggtgataccccagaaagaaaaaatatcagattagtttaggctggaaatgtattattttctgcagttgtaattatatctcagattatgtggtcataaaaaatcttaacataggaaggaatttccatatctcttaacttattctcttccctgaatagtgagctgaagtcatggaatcatgattcacattttgctttgatgatttactgcccactgaggttgagtaaggaagaatttttttcccgcatttgttgttttaacatttctttcactgccacttaaattcagtattcttaagccttaggcaagctggagacaatagcttatcaccatttttaaaacaatatcctgcatgtcctttcatccaaatatttatttctctcctcatcttcctgatcattaattgccaatgtgaatgtaaaaataaaaattattaaattatgtttacaaactttattagctaatatcctaacacaagcttgtgttttctgatggtgagatactaaaggaagcacatagaaacatgctattgatacatataatttgcaaacaaacacaggttcactgtggcaaagtctaagatgagttgggtctgatctaaaaataaaaggccatgaacaaaagaagaatttaaaaacttagagatccagtcacatattttaaagctaaagatacaaactgatgtatcaagagagtacacacccagggttaagagactaggaatgtgcttctttcgcttaccagctgtgtgatcttggacaagtgatttaaccactctaagcacaggttgttggtgggaatataaactggtgaagctgttatggaaaaatagtatgaggattcctaaataaattaaaaatagaattaccgtatgatccagcaatccctcttctgggtatatacctaaattagatgaaattaccccttcataaagatatctacactgccatgttcactgcagcattattcgcaatagccaagatagggaaacaacctaagtgtttgtcaagggatgaatgaataaagaaaccgtggtaggtatatggaatgggatattattcagccttaaaaaagaggagatcctgtcatttgcctcaacatggatagacctggaggacattatgctaagtgaaataatccagacataaaaagaaaaatatggcatcatctcactcaaaagtggattctacaaaaaaatcaaatatacagagatagagtaatagtgattaccaggggtgaggtggagcggggtgggatatggggagaagtgggttaaaggatacaaagtagcagatatgcaggatgaacaagtctaaagatctaatgtacaacataaggactgtagttagtaataatgttttgtattcaggatttttgcaaaatgagtagattttagctgctcttccatgggggggataatgcttaactatgtgagatgatggatatgttaatttgttttactatagtaaccattttattatataagtatctcataacatcatgttgtacaccttaaatatacacaataaaatttatttaaaaataaacataggctgttactctgcacagtgggagttataataatgacaatcttgtatggttcttgtgcttattaaataaaataatacatttaaactcctggcacagagcaagtgcttaaacagagattacttaatgttattattcatttgcatccctttgagttgatgtttatcacaataaaaatgtgataagcctttaaatttcttttaacctcccagcaaggctaatctgtgtattttgtgctttctctctttctttcccttatgttcttccctttctctttaaaaaaaaaaacaaacagattatgaggactatggggattattggagaggagactatgaagtaaatggggtagatggctatgactacagccgcggccagttgattgaagatgtggaacatacctttgaagaggtaagcaaggaactgtacacacaaacagttgtggggcagtgtatgggaaagagggacagtgactaaaggcattctggctcattcttaacacattggtcagccattcccaggcctcataaagttatggaatattagagtttgaaagattttagagaataataggacagcccctacagatgatcagaattgcgctgctttcctccttctttcagtcactgaccattaattaccaatagaaagactataagaatgaaatattataagaacaaatggtcctttagattataagtaggaatgatcttcttgagctacctggggctggcactgtcttcctgtgttagcagagctatggactttgctttcttcctcaggacaattgatactattactttgagtaagctagtcttcatagggaagagagagttttgtgaacagaaatgtcccatggatgtttttccttcattttggccccatgcctcccctttacgtgaaccttcaaacttctgatgtgagttaccttgtcacagccatagtttggcagagctatcaaaaatggaagtaagacaaacaactccccaaaaaggaaatgggaagaagacttgaataagtacatcatcaaagaaattacacaaatggcaaataggcaaataaaaagttattagggaaatgcaaattaaagctatgatgaaaatccactccacacccaccaggatagctatgattaaaaagatggacaataccaagtattggggagaatgtggagacactggagccctcttacagtgcaggaagaaatgtaaaatggggcagtcactttggaaaacagtttggcgatttcttaaaaagttaaacacacacttaccatgactcttaggtttctatatgaaataaatgaaaacatgtgtccttatccatagaaagacatacacaaatgttcatagcagcaatattcataatagccccaaactggaaaacccagctgtccttcaactagtgaatgaataaacagactctgatatactcaaagtagccggagataggagtggaactgactacaagaaaccttttgtggttatagaaatattaaaaaactagatagtggtgatggtttcccggtagttttaatttattgaaactcatggaactacacacttgaaaatgggtggcttttatagtatgtaaattatacctcaatgtaaaaacaaaggaagtaggtaacaatcttaaaaatgaatatatattttttgcatctgcttttattttgggacctgtgttctcccaagtatgtttattggtttatgcagtggagttatcatcaagttctaggaagaaggaatacttttaaattttccagaaatttgagagaagaagaattataatttggggaaattctacttgtttaaagaatctcctatttctagtgttgtggaatggaaattagaattggttactctttgtcttaaactttcttcctgggcttttcagattaaaccattatatgaacatcttcatgcctatgtgagggcaaagttgatgaatgcctatccttcctatatcagtccaattggatgcctccctgctcatttgcttggtaagaagccccatgaattcttcgtgtacttggctctttgttatttctaacaataaaattgcctttttgagtgagccttaacataaaacttttgaaaaattctgtaagtggttagtaacattgtagaaatcgcattttgaaagcattttcttcaaccaaaagtctattttaaacaattctctattgtattactttttttgtacataaagagttattattttggctgcaattcaaatgataaaggggttatttaagataactagagaaagaattacaggctcaaatgagagtggaaatggggaaaataatttttagtattcaagttgattggctaagctcttaaggtggaatttgagattttagtatcatattgtcagagcttggttggctgggggtggtcaggggtaacagaaggttatagcttgagcgaaagcttcctaagttcattctagacttcaccaattactcttgctgaaaagtgcttggcttggaatcgtataagtgaagaatgaatgaaagtgaccatcttcccctctcccaccgcagcagctgaggccaaataaaatataaatatttgaaaatttcatgccatgcatatatagcatttctactgtagtcaaactaagttgctggaagctaatctcctccacagccatacctcgtgtctgtcagcaggagagaatgcggagaaaggacctgtggaggcttttaactctaaagtaatatggaaatccttcctacaatgtcaatggcaagcagtatttagtaattcagtttctgcttaaacctgtctggtgtgatgggataatttcttcattccaatatagactaatttactgctacatggaaccaatgtaatacaattctaccttatgtggaacttaagcaacacccctctgatgccttccattcattagccctatttccatgctatgaggttacccagaatgtcaattcctattctacaagatgaccttccagatattcataaagaaccatcatataagatatagcatagtacctggtgcaatacattttagtgactattattgttattgtcatgtttaccctattttcttttgtcaagttaaatatcttatacattatcaaccattccttaaaacctcacttttctataattgttctcctttcaatgagctgtactttgtcaatgtccctcctcaattatcatgcttagagatgaacagttactccagatgaggtttgaccaacataaagaaagacatggtaccatgactacctttgttctagacactatacttctattgatttagctcaagttcacatttatgctttagaagccaggtcatactgtttgttggctcatttttggtcttttacatgtggactgtgctttagcctgatgtcacatatatgtttttatgatggataggtttttctttaaaatttaagcataggacttcacatgtatcactactaaattttatgttttaaaatataagtttagggagtacttttatttttttcagagtctcgctctgtcgcctaggctggagtgcagtggctgatctcagctcaccgcaacctctgcctcctgggttcaagcgattctcctgcttcagcctcctgagtagctgggattacaggctcaagccgccatgcccagctaatttttgtatttttagtagagacagggcttcaccatgttggccaggctggtctcgaactcctgatctcaagtgatctgcctgcttcagctctgcaaagtgctgggattacaggagtactcttcatgaagtttgttgccccatttttttggcaccaggattctggtcctgctcttggattgactttttctagatacatcttcttttttatggtccctggccttggaatacaacttaacatttgtttgagatgtttacaaagtcaccaagttaagtacacgaatataggaataaaagtggaatttagttaccagttagtagaagttcctgaatgatgatcttatatgatctcttttgaagccaacactaggaattactaacagcttaatttgtaatattttgtaccatggatggtagattatagaaatctctttctaatttacagtgtcaatgatggtccatatatttaataaattatgtctacatttctgctgtgttgtcatatactaacagattcttttttaaatataggtgatatgtggggtagattttggacaaatctgtactctttgacagttccctttggacagaaaccaaacatagatgttactgatgcaatggtggaccaggtaggaaaaagagcccttaaaaactaaatctaaattctcaacttcatttatttttatgtcatccttctatttttatttttatgtgaaaccatgtaaataaaaaatttaagctaattgaaatttatttcttatgtagctatagtttcatggaaaagcagtttacaatggcctcagataacttgaattgcaggtgtagtattgatctcattgaaaggtatttgaaatggagattacctgagttttccattaagcggtaagatgtagaattgtcaactggttggtatcactcattccaaagtttgtcattgctaagtaaccagccagtaatctctatggatagtaaaaacaagaatattatgaggggatttttgcatatttaaaaaattattagtgttaaaaatattacaagaatctttaaaacaattttaagtaacatctttattactactttggtttaattatttaaatgtgctaggttcctcaaaacacaagtaagtacatagcccatggacccaataaagcaactacacaatcgagactacaaagcaaccagctaacaacagcatgacaggaacgcttcacctaacagacagagagtggcaaattggagagaaaaacaaaacctaaccttctactgtcttcgagattcatctcacgtgtaatgacacccatgggctcaaagtaaagtgatgcagaaagatctatcatgcaaatagaaaacaaaaaagagcagggggctgctattcttgtatcagataaaacagactttaaaccaacaacagtaaaaaaggacaaagagggtcattacataatgataaagggttcaatttaacaagaagacttaattatcctaaatatatatgcacccaacattggagtatccagattcataaacatagcacttttagacctaggaaaagacttagacatccagaaaataatagtgggggacttcatcaccccactgacagtgttatacagatcattgaggcagaaaactaacaaagaaattctggaaattctggacttaaacttgacacttgaccaattgaacctaagagacatctatagaatattccacccaacagctatggaatatacattcttctcatctgtatacgaaacatactctaagactggcaacatgctcagtcataaagcaagtcttaataaattcaaaaaattaaaattatgccaaacatattctcagaccacagtagaataaaaatagaaatcaataccaggaggaactctcaaaaccatgaaataacctggaaactgaacaacttattgctgaatgacttttgagtaaacaatgaaattaaggcagatatcaaaaaattctttgaaacaaatgaaaacagagacacagcataccaacaacatatctgggatgtggcaaaagcaatgttaagaggaatgtttatagtgctaaatgcctacatcaagaacctagaaagatatcaaattaataatctaacaccacacttaaaggaactagaaaaacaagaacaaactagttccaaagctagaaaaaaagagataactaaaatcagggcagaattaatgaaattgagacctaaaaaatcatacaaaggatcaacaaaatgaaaagtcagttttttaaaaggataaacaagatggaaagacctctagctagattaacagagaaagagagaagatccaaataagcacaatcagaaatgacaaaggtgacattgcaactgatcccacagaagtacaaaccttcctattgagtactatgttcatatctggttgataggaccaatagaaacccaacgtcagtatcacacaatatacccttgtaacaaacctgcacatgtactccctgaatttaaaattaaaattaaaattaaagttaaaaaattatcaggccgggcacggtggctcacagctgtaatcccagcactttgggaggccgaggtgggtggatcacgaggtcaggagatcgagaccagcctggccaacatggtgaaaccccgtctctactaaaaatacaaaaaaaaaaaaaaaattagctgggcatagtggtgtgcgcctgtagtcccagctatttaggaggctgaggcaggagaatcacttgaactcgggaggcagaggttgcagtgagccgagatcatgccactgcactccagcctgggtgacaaagtgagactctgtctcaaaaaaaaaaattctctgtgttcccttctgttgatgaaatggtattatagttataattatgtatatttgttcattcaactaaaattctacgtttctgagtagaatatctaatcatatttctatttgccttataaaatttattcagataattgcaccataatttactatttgtgcattgctgaacctttgcttgttaatagttttacatctgtaagaagcaatgcaatgaacatcttcacatacataacatactcctgggaaaagtccactgcagaataaaaagtgaaactgcaagggcctctaaactcttgtagctgttgtagctccactttgaagttgagcagcagtatagggccatcttgatgtagatttttagtctttctaaagggtttttacacccgtctcttctattgtcatctctgactcaggataggtattgtgacagctccatgctataggtgaggaaactgaggctcaggtaaatgatttgcccaaagtcacaaatcacacagcctcattctttctgttttttttgtaccaattaaccatcctcacctgcccctgccaccttcccactactctttccagcctctggtaaccatccttccactctgtatgtccatgagttcaactcttttgatttttagatgccataaataagtgagaacatgtaatgtttgtctttctgtgcctggcttatttcacttaatataatgaccttcagttccatctacgttgttgcaaatggcaggatttcgtttttttatggctgaatagtattccaatttgtatgtgtaccacattttctttatccattcatctactgatggacacttaggttgtttccaaatcttggttattgtgaatagtgctgcaacaaacatgggagtgcagatatctcttcaaaatactgatttcctttcttttgggtatatacccagcagtggaattgttggatcatatggtagctctatttttacttttttgaggaacctccaaactgttttccatagtggttgtactaacttacattccttccaacagtgtaggagggttcccttttctccacaccctcatcagcatttgttattgcctgtcttttggatataaaccaaaaaatttttcataaatataatttgggtggctgcacattattctatttttcaaaggtatcattatttatttaaccattctgatggtcgtttcaactttttctctattaaaaataattctcaatgtaattgttgaatctaatttctgataattttttaggttagttccttagagacagcattactgagacaaagagaagaatttcaaatgctatccgtgtgtgttatcaaattaccttttggaaaggttatgccaatgtatatatccaacagaaataatgctgctattttaaaaatgacattagcacttttttccattttaaaattttattgtaaattgacatttattattgtatatatttatggggtacaaggtgatttcatgatttatgaatacaatgaaaaataattaaataaagctaattaacatatcaattacttcacatactttttttgcagtgagaacatttgaaatttactctcttagcaatttcaaaatgtacaatgttctattattaactatatttactatgctgtgcagtagatctcaaaaaaataaaaccaacttattcatcctgtttgagactttgtacgctttgaccatcatctccccgttgcccccaccccatagcctctgtaaccaccattctactctctgcttctaccagttccattgttttagattccacacataagtgagaacatgtggtatttgtttttcggtgcctggcttatttaatttaagaatgctgttgttttgatgctggaattaatactgttctcttttcccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggtagtggggctgatacttacacaactgatactaaatgggagacaacagaggcaggctgaagtttaacttgaattctttctctgcttttctgagcacagaaaagataagttaattctccttgaactaaggctgggagtccaggagagcacatttgggttttcccaggaaaagaggattgccagcaggctctaacttgaacctgtggacaatctgctttcttggaaatatcatgcatccattggcgttgtatggtaatggggatggttgcacccatatcaggaaagatttggtgctaagatttttggaagatggacatatgaatcacacattgatcttcccctccatgtttcactcttgtgaatgagacagaataggtgaataaatacgcttggatcgaagggagaaagtggtacatgtcgtaagagagacacagggcatgctctatggagaactggaagaaaactgaccacatttgcaataggagataggatcagaccgtgctttacaagtgggatttgaattaggtttggaaagacaagaaggattcagatacacagagtcgggaggaggacccaagctgtgagaacagcaggatcaaatacagagaggcaggacctgacctgcatactgaagtcggcaagttaggctagaatgagaaataagtgaaggagagtttgtgtaatgtggcagaatgagcacaggcttcagaatcctaggtgtgtcacttaatgactatgcaaccttggacaaggtatttaactttctttggtttcagtttccttattttataaagtagaatagtaattcccaggttgcaggcttgtgagagccttaggttggattccctagcttgaaaaggagatcgttttacaagtgcttcattgaggagagctctgaggcagaggggaatgagggaagcaggctgggacaaaggagggaggtaagtaagaatgtgatctttactggaaacttggcctcagccttatcccatgaagctctctggagcataacttgagccacagactaggccacacctcaagacaagggggctagccttttatatcaggcagtctttggctgtggactgccccagagtatgggggagcataacccccatttagccaaaggcagtgaagggagcagctgtaggcccttatcagctgtgactcaggcagctaggggatggtggataaagtcttggcaggcccgcaccagcattgactagggtagggattggatctacagtccttttctgtaaagggccagatagtaaatagtttaagctttgcagatctctgttacaactactcagctctgcccttgtgacatgaaaacagccatagacaatacacaaaccaatggatgtggctgtgtgccaataaaaatttatttataaaaacaatggctggccagcaggccatagtttactgatgccagtactagagtgaggcttaaaaatgagaaagtataagtaagttttcaagcacagaatcttacatacagtaggcagtaaatatatagtagttatttttataattatttcaaaagagaatagagagaaaccaaattagaaaagtaggttggggttgtgaaaggaaaatagatttactgagcatgagatgaatacataactgaatatttccttaccttccccctttctcttgcaatgtgtgaattaccatgccctcccactatcctctccagctcacttttctcctataaatgttgaagccctcaaaattatttttggagaaaggcacaaaccacagactgtttctataaattctgtgttcttttcttctgggcatgttcttaaccttggcaaaataaacttgtgaattgattgagacctatctcaaatagtttttggtttacagggtcatattggtgtataaaaggtggctgagtttattcttgagcaatgaggaggcctcgcaggatattggagggggtggtgacatgatccaagtggtatgaaatacatttaggaatctactgatgtatgacccctacacccatctgaatgaaaatgctcttgctaagttaaaaaggaccaccatctcattgccaaatgcagttcttatcccactgggctatagcagcatttgacaagatttacaagttgtctttcaaatgctttttttttttaaccgacttccacattgtcattcttttccagatttccttgacctctctgattgtactttctccgtctcccctgaataatcttcctctccccactccttaggtacttggaccttctaaaggtttatctttggtcctcttttcttgcattctatacctgttccatggcccatcatatctatttcccaacttacatgctaatgattcccaagtttcaatttgcagccccttctactatttctctatcctggcaataccaaacagcgtgcagtgtcctgaatgccacatttccacccccctgcccctaagcctttctcattgagcactagaacgtcaacttccatgtcatctaccgggcaaactcctgcacattgattaaggtccaaattgctcattgtatcctcagtcatgcccttcttggtgcttctagggaactgtgcctacattcactcatttttacgctcaaggctgtgtgtactgatcacttatagagtacttgcctcagagttcttgtagcggagaactctgcttatctgtcttcctgttagtctatgagcaagagaacaggattcagctgtgcccattgctttatattgagcaactagtattgtctctaggacataagaggtgggtactcaagattcactggtgaataaatcttggaaagatgattcatgctaccaaaaccccatacaactccactgtaatggttaaatgaaagtcagtgagattcattttcatcttgtccattttcatgcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatggtatggacattttctcatggcttgttttggaggttctttaatctgatatgggaaaaggtatttattatcttatgtaggaaatatttactttttgccacataaggtatgaggtatttatgggtaactggaaattactactggtgaaaaaatttaagtatatatcagaacatgtcaacttttggttttgtattattgaaaagggaataagagaggtaactgaatgaatggacttgggatcctgtgaaatatctaacagaagtttttttcttaaagatgatgaaacagtctcacagaactgaaatggcttgtccaattagtgtcagcattctaactagacccctctggttctaggtaaaaacttcccaaaactttagagttggaatatattctaaaaaccattttatagaggagtaaactgaagacaagagagggaaagtagcccgaagtcaaaacgcaatgtagtggcggaaccagaacaagagccctaatctaaagtcttttccataatattatgcaaagcaaagtgtagctttaaattttggaattcttaaagaacaggtgtttaatatcaagattagaagggattgaaacattttgttcatcctgctgaagcgtgaatttcccctgtaacatccccttagatctgtgctatccttcaacatttctgcctgaggcagcccatttccttttagatgccttggacttgatggaacatgacaaatgttcaggagatccaatttcttcagtgaggatgccatggcttttgattcttcctcataaatggaggacaaatagagcctaataccttcttctcttctgtatattctctcccctatttttagttatttgtgacataaagatcagctcctctgtctggagcatttggtcgatgaggtcaaggtcatgagctccatccttgagtagacaattcactttgccttgatctctggttccagatacttggtgatcccacaagggcctgggagtgagccagtgtaaagcctatcatcatgtttgaaatagttactgtaagcacagtcctagtggattacagaccagtagcagcatcctcccatgaagacagtaacaaacaaaggtagttaacagtaaccatacctgcttcctaattttaaaatatcattaattcaagtcaatgaacagatattttattatttttaaatattccctgacagcaggtaaaaatggttgatgactatgtttcttaagatgatattagatcagtgatgaaataatctgtacaacaaaccccatgacacgagtttacctatataataaacctggacacatacccctaaacctaaaataaaagctttttttgtttttttttataaaaaagatagtattagacatggttaaaccaccggtttggaaaactcctgctttttttcttgactctgcccctgatttgtgaataatgttaaataaattcctttattccttccattcctccctccctctttctctttctttcttcttccaaacatatttatcgagcacatactgtgtgcaagacatttttctagacactggagacacagtgatgtgaaaagtagataacatcccctgcttacatttcaatgcatttccttcatacactagagatgaactccactgcctctttatgtcactaataaaggataaaatgaaataacatgatttgttaaagcattttaaattcttggtaaggaagatatccttacatttaaagtacattgtacttttcaccatccagtggatagtgccctccaggagggctcttttgtttcatttcccctccctggtttacaggcttttcactgaatctgtttagtatttgtttatttaacaaagactcacagaaggcatcacgtagcagacatgggactaagttatacagtcttaaatgagacattaacagtcactgacttcatggagctttccagaataaacagtgtgactgggaaagtggatgaaatagctctaatgaactctgataagaggtatttaagggaggaaactgaaactaatgaagaaaatgaggattagaagtaactttgggttttatttacctgttctgttccagggcttcaacctactccaaatcccttagctgagaaaataaatatatacatatcattatgaatactgtcaatttttctcatgtatccttatagtaagtacatcttgaggaataatattttgaggttaggaaaattctctttaacacaaaacatccactgtcatcttcatcgtaatatttatccttttctattttactttcagaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagtaagtcaatgaatatgcaatcagtaaaatgttttctaatgagtttgctgtgtatttaaaagcatttgactggcgtgtacacacacacacagaggcacacattaactaactactttttgtatttttagtttgttatattttatattttagtctccatttggataccatctcctcagataaaatttccttgggtcaggtgacactacttcatactctatattcttaagtattggcttactcattaaactatgatattatgttgaaagtgatcaatttattcattttacatattcttttatccagccttttaaacaaaacggtgaccccacattttaggcaaagcactggggatccaatgttaataatttcaggtatggtcctcaaccccaaaaaacgtttagtttatggcaggatgtggaaaattaaaccactgcaatttgtagtgttgtgatgggagggtacagggtccgttagcgtccaggggagatccaaagcccaactgggggtgtcagatgaggagaggcatttaagccctgagaaaggtgtgggtgtagaggggaacacagagaattccaagctagagagaacctgacacatttgggcaacagtgaccatttccatggttgcagcactacgttacttattgctgtgtcacaagtgcctcacactgtgcctgtcacatgcaagtatgaaataaataggtttttggaggaaggaattataagagaaaaagaaaaagaaggaaaaaaagaaaggagagagggagggagagaatggaaacaagaaagaaatctatcttctttttaccatcatagaccctggtcaattatttctttctttatttttcttatattttctttttgaagtgaagaactcatgtgtaacaagcagttccacatgcacaggtttaaaacagtacacttcttctgtcaagattctctctgttaacataaatcgtacttgggaggaaagacgcagcacctataagaactgttgccagtgtttaaatattactgaagttatgtctacaatgatcctgcaattttaaaggaaaaatgtattccatttaatcaaagccaaatacatgattttgctacctttttttctggttggattaatcttttgacccctcttagcttattttaaaggcacatggaatcagatccttagagtgattcattaatgcatcaattgttcctttgttcagtaaatataaattaattctttctgtagtgtcaggcaccatgtttgacactggaattagagagaagaccaagacacagccccaccctctaggagctcaagtctctagggcaggaagttactatagttgaataactacaaaacggggagataaatggcaaaggaggcacaactagagaaggctaaaggagattgaggggttaattctgccagaaggtggatgggatcagggccaggcttcacagaggagggaacacttgagctaggtaggcgtggagaataagtaagatttcaattttattaaattctctctgccaggagtacatgaagtgtggtgcctgagaggaattattagggctttcactgtacatttttggccatgcaatgcactttatgaatgttattaaattagtcttttgaagacatagctattttccaagggtatattctggctgaattaattaaaactaagagataactacataatgagctttactgtctgattttttaaaaatttttatttattttttatttttgtaggtgatttttttttacattgtttctatttcacccaacaaccatttctaatgtaaataccattgattggctggacacggtggcacctgtaattccagcattttgggaggccgaggtgggcagatcacttgaggccaggagttcgagaccagcctggccaacatggtgaaaccctgtctctattaaaaatacacaaattagttagatgtggtggtgcacacctataaaccaagctacttgggaggctgagatgggagaatcgcttgaacctgggaggcggaggttgcagtgagccaagatcgtgccactgcactacagcctgggtgacggagtgagaccctgtctcaaaaaaaaaaaaaaaaaatttgattgatgaaactgcactagttatgcccacctgcttgtgatgcttgtgatgggtgatccacagctaatgtattgttttcttttctccccaaaggcgagagatagttggggtggtggaacctgtgccccatgatgaaacatactgtgaccccgcatctctgttccatgtttctaatgattactcattcattcggtaaattacagttttcttgtttctgtttaacttctagggtttgtggacagctgtgctaataacgacaaaaagtagggataaatgaagtgataattataatttttctatttccacccataactaccaagctcaatacagatgctccttgctttacaatggggttacatcccaataaactcatcataagttgaaaataccttaagaatgcgctgaatacacctaacctatggtacaccaaagcatagcctagtctaccctaaacatgctcagaacactcacattagcctactgttgggcaaaatcatctaacacaaaccttattttataataaagtgttcaatatctcatgtaattatttaatactgtgctgaaagtgaaaaactttgggtacttaccattaacatatacagctgaaagcgctattgtaaagtcgaaaaatcaaaagtccaaccgttgtaagttgggaactgtctgtatagatgatcaatttcaattcaatttaaactaacacaatttattgggttaaattaaacttgtgtaagatcttgtccccgtcatcagcaatcaatagtatcattgggagaaataaaaagtagttttgtcttcttgttactggcagtttattgtacattgtgatagagaatttttaacttgaagtccaaaaacatatgttcttcacctactaaccccagtccttgaatttgctggagctcagtttatttgtaaaatgaaaataatattatcgacctgttataaggatggatttgcatcatttatgtgaaagggctattaatctgtaaaacacaaaacaaatgttagctaacatttaacagagtaatattgccatgtttatcaacagatacataaaacacaaataggttatgatgagtgtagagttcatgtctggagaaagagatttcagagatttgatcagtatctctttggttacttgggctccagatttaaatatatgccctaatctataactctaagacagtaagtaaacacgggactgcttttttgttgctttgtctcctgtgcagatattacacaaggaccctttaccaattccagtttcaagaagcactttgtcaagcagctaaacatgaaggccctctgcacaaatgtgacatctcaaactctacagaagctggacagaaactgttgtaagaaatacctcaaaatgttgaacctctcctagtattcagtattactcatttccatgcctaggtttgtatttgatttctttgttctaaaaagaaaattttatggcctcaaaatgtcctcatttacaaaccaaacatttaatttgtggtcagacaggaacctagaccatacaacaattgggtgggccacctcttttctccctatcataactacagccctctcttcctggtaattggaaggaaagagcggtttagggtggaatatatctgttaatatgcattcttttcttatctgccagaagcaaatttagccaagtcaaagagaagaaaccatagatcatagatgtaaatatatgtacatctggaacccctcaaaaggccctgaaccccctttttttgtgtagcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatgtgagtacacccagttgacaagtttcacacccattccttgtctacttcgtctagtctcctttggccccagtctcatgaggacttgtgacacagctgagaatgtttctttctctatagtacctgcttctttctctgctatggtgacagctgccattttatgggagaaaaatcacaactgttgagcacctatgtatgagacatagtgaaagcaactgtacagacaaacacacacagatgcatgcacatgtgtgcatacacatacaagagtgtgtactctttcaagtctgttttattagtctcatctttaaaatacatattataactgtctccaaccaactgtactcgaatcctgctttccaaagacaaatactttttttaaaacaattttaatgctattatctcttctttctggcagtcactttcatatttctaaataattttcttatattgctattttgttattgctcagttttaaaccttcgttttggtctactcttccacttccatttcttgctcttccaataaggccttataatgatttttagtaaaacagttatcaatgtttacatcactatgcctgaagagccaagtaatgtattattttgtgtcctttcttatgtggcttgttgtttgtctcaaagtttctacttgcctgaatttttcggcttacctaatttgctatctacgttgtatttgtttttctcccaaatatttcaacatatctattcttttcagtctcattttttttccctagagacttccctcttggagtcttatattctcctttcccaacatttgttgttctcctgggttgaatccactgcttcctagattccatgtcttctttcttggattacctcttgtttttctggagcatagcctcccacagactgtccaagcaggggcacaaggggtaggaggatcaactgttctccaaagtggttttaaccacaccctgacttccattcctcttcttttcagcccagtattattttcccaccctcacctgtgcttgaattccaaacctcttttggattttttggctctttgtgggtctgtgcctcctctgtgtacaaattaggtcatggcttcctgtccctgagaaagcagttacccctgtctcatcatttctcgttttccaaaagcctgttgatacctttaaaagatccctttactatcactttagtgggatctttggaggaaaaggaaataagcacatgtgggcaatctgttgtctgtaaccagcagtctgctatttctctttaatcagatgcagaccaaagcatcaaagtgaggataagcctaaaatcagctcttggagataaagcagtgagtattctggacagtgaattgattatttttgagtgcacagtcttcacttaaataggacacaataaacccatttcagtgtgaactgaaagaagagaggccatgtggttcttttcaaaactataagttccagattctggcctttgcctttgggtttttaaagtaacctattccttattggataagctattcctacttcttattgccttgaaggtgttatgaacttcaatgagaaaatatctgtatcaatccttaaatatcagataccaatcctttcaattgactctcgtcaccattacttttattctagaaagagaatatggttatattttgaaaggtagtttgtttttagaagcggtacagttaggtttttttagtttgttttgttttagtcaatcacctgtatcctggcttcactcagagtgtggcccatggtccagcagcatttgaatcacctggaagcttgttaaaaatgcgtaatttcaagcctagccctggaccacttaatcagaatctgcattttagcaagcctccaggtgatttgtacgtacaatcaagactgggaagctctaatttagaacactgcttctcaaacttggctgcacagtggaatcacatgggaagtttaaaaaatattaatgcctaggttatacacctccagagattctgatttaattgttctggggtatggagtcaagagttgggcatcaagagtgtttaaagctccccaggtgattctagtcacagcaaagtttgagaaccacttatttagggaaatgcattttgggggtaacaaaatattttacaggaaattatgttatttacaccttaatgtgaattatttacttagctattgctctgtcgttaacacctgtctcagatgatccagtctgaggaagtggtagctttgattgggaaagaggagaggaaagagtagtctcgggtagatggggctcaaaagcctgagagtgggtatgacaccctgaaggctttgcagggttagaagtcaatctgggactattggataaaatggtccaagatgctacagcaaattgttgtctgcattggatagatccaaagattgtgtttctacccccttacctttcctgaaaagttaaacattgtccaagattcaataacacttttagtataattttcatagttaagacatcaaaacataaataactcaggagtaaaaagatgaacattcaagaaagaaaaaatgaaaatggaagtgcatttctttccaatcattaagagttcaagttcctatttcataataatcattgggtgattgtgggactatttctctgtcatatttttattagctcttccttggcttaatacattttaattagtctcggcagatcaggatattttcaattatcttttttcttgacatttatactcatcctctttccatgcagtgagggttggtaaatagtgttcagggagtttgattctgtaatgttctgaggccaattcactgagcaacaatctgttttgatgaaacgagatcatatatttagtgcatctacacaacagggtcagaatatctgctcagttcaaactatttcatggtgaattccaatggatctatggcattattttgagaactcagcctttaaaatcttttatattttatatttatagagatagtagccagactgttattagtaacaatattaataatattattactattaaaggaactcctctaaacctcggtgaaaccaagctgaagtgaatgtaacaattcttttgtgaaatattataattttttaaagggaaaggagaaacatacaactagtgccattcattgattcctactgccagctactggaggaaagagtgaatgctttcttttaagtctttaaaagctatgcaaacataagctatttgtttagcactggaaaacaagaacaaacagattagcttgctgtttacaaagtgttatttttcatttgaacgtcaagtttttcttttacacttatagataagtacatttctgagaacgtaccataaaaatagtaaaaactgattttgtccacaaggtacagacattttgctacaaaacccacaacttttgtttgactcccatcggtaaacctacactcaaggtgttaatttcagtttacatcaaactaaagtaaaaggacccgtgaacccaatggttcctaaagcatggttgcctgaccagcagcattaccaccacctgggagtttgttagagatgtaaattattgggtttcatctcgggcttcctgaatcagacgctctaggggtggacccagcactcagtgttgtaactaacccttcaggtgatacggatgcacacttacattgaaggtcactgacttaatgaatagcaagaatctctggaatattaagaataattcttgaggagtatcagattataaatgtgtcttacgagtccctctgagcagtgtctttcttctgaatttgcagtatgaatggaacgacaatgaaatgtacctgttccgatcatctgttgcatatgctatgaggcagtactttttaaaagtaaaaaatcagatgattctttttgggtgagttgatttgctgggttctcaaattactccaactaggatttctcttactcttgagtctgaggagatactctgcctaaacttttcttcaagtgaaattgataaaaatctcctagtaacttatacttctttggtattgtggaatttgggtgactccatagagggctgatcgtgccttatttgactggtaaattgcaagagtattgttcttactaaacagctgtcactctcttctcagaatgctctcatacaccctcagcactgaaagaaaaaaaaggcagaacttcctgccttcattgacctatattctagtatgtatgttgtgtatgtgtctatgtgtgtaatatgtaagtgaaatatagagttacaaggtactaagtgctaggagaaaaatataaaagggtctggggagggctggtgtgtgtgtgtgtgtgttgggcagggggtaggtcaattttaaccaaaatggtcagaaaagacgtcactaatacttgaacaagtatatgaagcaggagagagrgagmgagagagcgagagagcgagagagcgagagcgagagccagcaatgcagacaatggggaaagtgtctcagctgggggaaacagcaggtgcagaccctgaggaaggaatatcctcagcctgttaaaggaacagcaaggaggccagtgtggctggagcagattagaagacaagtggagacatggccaggacgatgttagggcatagaggaaggagggagagacaaaaggtcatgtgggccttgaaggccattctaaggactttggcttttattctgagttagatgagtagtcactcaagggctatgagccacatgatctaccttattttgtgctaggatcgcttgacagctgtgagagcacagtcctgtcacatcatatggtagaccccactcattaccttcatccatgggtgcaacacgacgttttctgctgggtaggaccaggttgtgggtgtaaatccgtgtgtggtactcagccatttctgcccaccacattgtttgccacaatcctgggccaccgattattccttgttgattctcctgctgttttcagtttggaaattttctataattgaaaaagatgtgggttttatataaaggcagaacaaatagtgccaaaacttgatatcacaatttttatgtagttgatttcatgtgctttgggcagcattaatgttttttttgacaattacatcctctcattgtttgccctcccccatgtctctctatcctgtgattctctctctaatctccttttcaccagcaggggaagtcttcattctcttgattgtgtcctctgtgccacaagtgaagatgtttgttttgtttctctacagggaggaggatgtgcgagtggctaatttgaaaccaagaatctcctttaatttctttgtcactgcacctaaaaatgtgtctgatatcattcctagaactgaagttgaaaaggccatcaggtgacattttactttcatctaagggtgagggggttaccaaatacaacaacaataaccagtattttgtgtgttcttcctgtgtgccaagcattattctgagtcatttacatgtgttacctcatgctacaaactccacgtaattccagaaggatcccacagcaggggaataatcttgatttgagttccgccagatgtaggctcttacaaaagggaagaaaaactatagattacatgttatcaagcaagttaccactatgggcaactagagctcagtcctgctgggaaatgtggagagactgtgtagaatgtgcacctgaattgtcccatcagagggctgaggaagctggggtatttatttaccacctcccatcagttattggctgagaactgcttccaggtggcattaattccccagcatttcagccctgcaggcagggcagatcttgtggtctgagaatgctctctggtaaaggggagcaggtgctggttgaagaaagtctggctagaactcatggaaaatagtaagtgcagaagggacgtggacaggtattgccatcatttgctctcaggactttgatgtaggattcatttaactgcccaaatcctgaaacaaggtaggcaagtacaggattccttacatattgcttggcctctatgctaggtgagggacttgcaatgaatgtttagaagaaacagtttgtccaatttgcctcaattgtttcccacaacagacagcaggagcgaagaatggattgaccttgcagatggtttcctgtcattataaaaaggcttaatatacctactgcttctgggcagctcactaggtggccctctcgagctgcgtcagagtcctgtttaacttcggaagcagatctaagacctgctgggcagggtgtgcctagaaaatctggcttggttggagtgtatgacccaactgtgcccagagactcactcacttcctccagaagacttgtgtctgtcttgggaaaaacttctggctgagatctttatgaaaaagctcagccttcctggatactcagctgcaagctacaggggacctcagactctctgtttccaggatgactcttttcagatcttttctggctgaggttgccaggggttacctgactaattctgcgttgatctctctctctctctctggtggactctgacctcctgacctgggctatacccaagggtaggaagatgagatgccagacactttggtctttgatccagaatgttctccatatcattgtttttttttttctttaaacaatctttatccttttgcctccataaaagtaagccatttcccatcccagtgctgaagaaactggcaaaggtccctttcttatgtgcctccccagtgctacctccaaatgccaataccttttatttggaaaatattactatagagacttggtcataggacctgattcatttgtataatagaagaggtgtatccaattatccgtttccattatttgatataaagtcattgtagatgtactctgacagaagggaaattcttgccaaatatgataactttgcccttaaacacagcagtcacaaatgaataaatgccaaccatttatacatttccacacttacaactcaattttccaatggagctgttgatgaacctaatctcgagataacttcgtataatgtatgctatacgaagttatatgcatgccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccatgggaacccccaagaagaagaggaaggtgcgtaccgatttaaattccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggtaaatataaaatttttaagtgtataatgatgttaaactactgattctaattgtttgtgtattttaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggcggccggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcccccggctagagtttaaacactagaactagtggatcccccgggatcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagataacttcgtataatgtatgctatacgaagttatgctaggtaactataacggtcctaaggtagcgagctagcctaggttgcaaggcatgaaagatgcataattgtcaaagacttatatctttaattagacctatttactttgctcttatgattttaggtgtatattccttttttttttttttgaaacagagtcttgctctgtcacctaggctagagggcagtggcacaatctcggctcactgcaacctctgcctcccgggttcaagcgattctcctgcctcagcctcccaagtagctgggactacagacatgcgccaccatgcctggctaggtgtatattcttatgataagctctattatatcctttcaggaacaatgatataaacagtaaataactacacacaattttattttgtctaagtgtcccctttgctgttttttgcttttgcaaataggatgtcccggagccgtatcaatgatgctttccgtctgaatgacaacagcctagagtttctggggatacagccaacacttggacctcctaaccagccccctgtttccatatggctgattatttttggtgttgtgatggcactggtagtggttggcatcatcatcctgattgtcactgggatcaaaggtcgaaagaagtgagtatcctttcctagatttatctgtctttttagaatgcatgaattggaaattttgcaacaaataacactgtatccacacaggataattacttacttggacatttatctccctaaatattctgatatatcccatagaatcttgtcagatgctagcagtaccttaccttactagcaagctcacatattatagatgtctagcctgaggtttattgggatgctgtagtcaagtgcatcctatccacagtggcatagccagactgcaaactcagtaaggcattgttctcactcatgactcttgcttcatccacagagactgacatggagcacaaagtgtaatgtcatctaagaatgaggtgcttcaaactactactctttagttactttcattcctccttgggaaactcaaatttatcatttgagttcatgggcaggcaccctttaatttgttctagagctgagttgattgagggctttaatcaccagggactgacttctagctcctgtcttgagaaagtattaactcttgttggtctgttcaaataacttagtattttgtatcactctgacaacattatctgcatgttttcagttgtttaaacatactctcagttgaatctttacacaaattagtttattttatttaattttctttactatagagatctctcgattctaccaaataacttcatttgaaaaagcaaggctgtggagatgggtaaaatcacctgccccacaagcatgaggacctgactttgaatccccagagcccatatataaagcagatacaggaggcaagcatctataatcccagcactcctccagggagatggaaggcagaggcaggaaagtccccagaagtctgtaggccactcagcctggtgtacacagcagaggcaacagaccccatctcaatcacaatgggtgttgagaatcaacactggatattgctgtctgatccccacgtgtgcaatgtagcatacacacttacacacatgcacatgtgtacgcatacacagatgtgcacaaagcactttctaggtagtgcagtcaacatggttacaatgttcaaaaatacaagggggtcatagagttccgttaccattctttgtttctgagtttccttttaaccaaagttatcagtttttagtaaaatgaactatgcactacatattcttatttaccaactagtatgttttagaggtatttccagatttttcaaaaaattcccaagcaatatattttagagattctttcagattggctatgtaaagcacttccatttgcctggtaatgtagttgcccactttgtagcaaatgggtactcccatggttaccaaatgtttgtgtctatttaaaaaatacaacaatttcaaataggcaatttcctgtgcatgctaaagtaggtgcaagatatgtcccagcaggtaaaaggctaggttaaaatcatcatgaattctaattttaattaatacttctgaactgttctcataggggcagaattaatgtacaaacagcaccagcttatgaaagctctccttttctccaactcagcatccttagcacagggtgccatcagactttttgacagttatcctttgggtcagtgtaagtaagtgtctttctaccatcactttaataaggagactttgttttatttgacaggaaaaatgaaacaaaaagagaagagaacccttatgactcgatggacattggaaaaggagaaagcaatgcaggattccaaaacagtgatgatgctcagacttccttttagcaaagcacttgtcatcttcctgtatgtaaatgctaacttcatagtacacaaaatatgagagtatacacatgtcattagctatcaaaactatgatctgttcagtaaacgttgtccaaagagcatcagacttgagtggacatcttcactgacattgctttcagtatttatttctgcctaaggatttgacatctcttctgtttattaatagagatgtttatcttagcataaaagagggaaatgtgcctttggcctcacagtctatccagggtgatatggttgggtaactggagttagaagatgagatgatgtctcttgggggcaagtgttggcttcggtgtggcatctgggctgtgaactggtgggactgttgaggttgagaatggtgctcgctggtcacttgaatccaagtgtgacgtcatgctctgtggcttctgccttcacacttctcacttcaagtactgtaggaatttgttcacagtaatacttgaaatggactgtcccttctttggaggtgcagttcaacggagaaagaagccagacatcaggtagagaccatgaccttttctcttccaaacttgatcaacatctctctaacaagacacagctagcacaggaaactccacgaacccagagcatgcctgtcagaaactacttccattattctcccattgtggagtaagggaaaattccagatgaatgctcgatctgtgagatgggtgcccagtctctgaaattgtttgtattttctcacagggtctgagcaatggtgaacacaaagccgacctcaataaatacttattagatttagacactcctct

SEQ ID NO: 6 - DNA, 23 bp, synthetic construct
  misc_feature 1..23: note = 7878mTU Fwd
  source 1..23: mol_type = other DNA
cccagatggctaaattcaattga

SEQ ID NO: 7 - DNA, 16 bp, synthetic construct
  misc_feature 1..16: note = 7878mTU Probe (MBG)
  source 1..16: mol_type = other DNA
ttcatctggaaaattg

SEQ ID NO: 8 - DNA, 24 bp, synthetic construct
  misc_feature 1..24: note = 7878mTU Rev
  source 1..24: mol_type = other DNA
ggaatctgggcaaataattcattc

SEQ ID NO: 9 - DNA, 19 bp, synthetic construct
  misc_feature 1..19: note = 7878mTD Fwd
  source 1..19: mol_type = other DNA
gggccgcatcaatgatgtc

SEQ ID NO: 10 - DNA, 24 bp, synthetic construct
  misc_feature 1..24: note = 7878mTD Probe (BHQ)
  source 1..24: mol_type = other DNA
tggcctgaatgataacagcctgga

SEQ ID NO: 11 - DNA, 23 bp, synthetic construct
  misc_feature 1..23: note = 7878mTD Rev
  source 1..23: mol_type = other DNA
gtggctcaagtgttgggtgaatc

SEQ ID NO: 12 - DNA, 20 bp, synthetic construct
  misc_feature 1..20: note = 7878hTU Fwd
  source 1..20: mol_type = other DNA
gtgaggctggacttgggaat

SEQ ID NO: 13 - DNA, 26 bp, synthetic construct
  misc_feature 1..26: note = 7878hTU Probe BHQ
  source 1..26: mol_type = other DNA
cctttcctctttgtcacagacctcca

SEQ ID NO: 14 - DNA, 19 bp, synthetic construct
  misc_feature 1..19: note = 7878hTU Rev
  source 1..19: mol_type = other DNA
ggagggctctgcctggatt

SEQ ID NO: 15 - DNA, 23 bp, synthetic construct
  misc_feature 1..23: note = 7878hTD Fwd
  source 1..23: mol_type = other DNA
gcaaaggtccctttcttatgtgc

SEQ ID NO: 16 - DNA, 23 bp, synthetic construct
  misc_feature 1..23: note = 7878hTD Probe (BHQ)
  source 1..23: mol_type = other DNA
cccagtgctacctccaaatgcca

SEQ ID NO: 17 - DNA, 23 bp, synthetic construct
  misc_feature 1..23: note = 7878hTD Rev
  source 1..23: mol_type = other DNA
caggtcctatgaccaagtctcta

SEQ ID NO: 18 - DNA, 120 bp, synthetic construct
  misc_feature 1..120: note = 7878 5'mouse/5'human
  source 1..120: mol_type = other DNA
aagatgtccagctcctcctggctccttctcagccttgttgctgttactactgctcagtccaccattgaggaacaggccaagacatttttggacaagtttaaccacgaagccgaagacctg

SEQ ID NO: 19 - DNA, 160 bp, synthetic construct
  misc_feature 1..160: note = 7878 human//xho//loxP
  source 1..160: mol_type = other DNA
catttatacatttccacacttacaactcaattttccaatggagctgttgatgaacctaatctcgagataacttcgtataatgtatgctatacgaagttatatgcatgccagtagcagcacccacgtccaccttctgtctagtaatgtccaacacctccct

SEQ ID NO: 20 - DNA, 192 bp, synthetic construct
  misc_feature 1..192: note = 7878 (loxP)/ICEUI//NheI//human
  source 1..192: mol_type = other DNA
cattctcagtattgttttgccaagttctaattccatcagacctcgacctgcagcccctagataacttcgtataatgtatgctatacgaagttatgctaggtaactataacggtcctaaggtagcgagctagcctaggttgcaaggcatgaaagatgcataattgtcaaagacttatatctttaattagacct

SEQ ID NO: 21 - DNA, 120 bp, synthetic construct
  misc_feature 1..120: note = 7878 3'mouse/3'human
  source 1..120: mol_type = other DNA
agcctagagtttctggggatacagccaacacttggacctcctaaccagccccctgtttccatatggctgattatttttggtgttgtgatggcactggtagtggttggcatcatcatcctg

SEQ ID NO: 22 - DNA, 40887 bp, synthetic construct
  misc_feature 1..40887: note = 7679 allele
  source 1..40887: mol_type = other DNA
aggcccatgagccctgccatttaaagtggctcctctcttacactctgggaatgaggacacggagccagctgctgaacttcaccaggataaccattaaaattgctttggagttcatatttccacgatcccatgcctatggatgccaaggacttgtcatggatgcgctttggatttcataatgcagagtcattattacttccttgagttctcagctgagttgtaagcaggtaagtgaaagggaaagaggcacctgataaagtcagctgtaggactagagtttagcatgtctcctgagaaatagaaaatgactgcttgaaactttaccaaagccacacaggaagcatcaaactccctatggagtggagaagagtcttataattttttaaatgggcagagaaatgaatttatttttaatttttagagacagggtttctttgtatagctctagctgtctttgattggtagacaaagctgtcctcaaactcagagatcttccttcctttgtctcctgagtgctgggattaaaggcatggaccaccactgccctgccccattctctccattaattttaagtgaatgcttgcaaaagctcacttctttggtgaacagcttcctttacaaataagtacctttgccttcgtttttataggattcttaaaaagaaaaaaaagattcagccaggtggttgtggtgcacacctttaatcccagcagtcaggaggcagaggaaagcagatctcttgagtttgaggctagcctagtctacagagggagttccaggacagccaaggctacagagaggaactgtctaaaaacaccaagaaagagagaaaggagagagggagaggatggatagcttattgatagaattgtcagaaaaggctataagttccaatatgtgtcccatgatttctaagtctagccctttctgttatagtaaaatcatagtacaccctcctcctccagtgtatctttaacagcttttaaggaacatattaactaaatgtccaggttttgatttggccataaaatgttagcaaagctaaggttttctaggattaatgaataacatgtctttatttagtttacttaaaaaaatcattctaaaatatctgtttacatatctgtcctctccaggattaacttcatattggtccagcagcttgtttactgttctcttctgtttcttcttctgctttttttttcttctcttctcagtgcccaacccaagttcaaaggctgatgagagagaaaaactcatgaagagattttactctagggaaagttgctcagtggatgggatcttggcgcacggggaaagatgtccagctcctcctggctccttctcagccttgttgctgttactactgctcagtccaccattgaggaacaggccaagacatttttggacaagtttaaccacgaagccgaagacctgttctatcaaagttcacttgcttcttggaattataacaccaatattactgaagagaatgtccaaaacatggtgagttctcatggctctattgggctatttgttgcctcttaaagattagattactgcccgtgaaactgaaaagggaaatcaaaaaaaatgctctgagttgtgagattggatgtttgcctccatatccctgattttggcgtcgacgataactaaacttaattgaccctggggttcattcagaaaattgttacaaaataattcactggtctcaatccagatgcttggaaacaagagaatattctttataaagttaccaccataattctcatcacagtcaggatttcaaagcatttcatggaaatgccataagttattgagttaataatttcctttagcctacaatatcaataggaatatctaaagattctctacaaatgatatattaactgaaaaatgcaactggaggcttagtgaaagaactagtaattaagctaatcagggcttgggtgaggctggacttgggaattcctttcctctttgtcacagacctccaagggactccaaaaggtcacagaatccaggcagagccctccaaaacaaaaacaaaaacaaacaacaaaaaaactatgattaaaccagttctgacaaatatcagaatgctctgggaaagccagatgctttaacaagtgcaaggatttaggaatttcttctctcacagatcccaaaacagtatccttaagtggtgatccttatgtcaagctgtgagaatatacctcaaaataatagaaggcatccaaacttgatcagtatagtgtcataaagctgctgatgtagaagtgtggagaggtccatcagatagagatatggaaaaaaaagcttggttaaaacaggtttccaaagttctctccctttctctgccattgtggtatgtgtggttgaccatttctcatcatgttttcttacaagactttcaggatcaagcaattcctcgccaaaaaacaaaatcaaggacacgttgtagctatttcttggtgagttaccagaagctgtgaagagagagcatgaggcccatacttttttttttttttgaagggtctcgctctgttgcccaggctggaatggagtggcatgatcatgattcattgcagcttgacctcctgggctcaaatgattctctcgcatcatttaacatttttttttgtagagatgagatctcactttgtctcccaggctggtcttgaactcctgggctcaagtgatcctcttgcctcaggctcccaaagtgctgggatgacaggcatgagccactgcacctagcctaccacacttactctttgcgtggtctctagagcttcttcagcccccctgctgccagggtccctagggtcttgggcatgccccatttctgaacctattcctgtctccagggatagagtcacagtgaactcagaccagggtaattctaaactgctattctgtatatacccaaggaaatttcatccctgtctctaatgagtctgtcccatgccccttaatacacatgaacacctacacacaggtacaagtcggggaagatcactgtgcagcacataaaataaaattcaggggcgagcactttttttcccatgagataaagttaggaaaaaataaaaatgttcatacagattaaaaattttaaatgttaaaatattaaacccccaatctaaaataaagaaaaaataatttcttaaatcctaagcttgagttatggaattcacattcttataatttacattacctattttgttaaatggaagacttttttttttaatatgcaagtacctgggcatatttagaaagtaaccaaggcagcttttgtctttgaacctgatgcggttttgacagctctatctggaaattgagccagcatatgggagccaagggtgaaagttttcctgggagatttgggctccaaggtactggatagatgctaaagattccattcagtttttcattttctgtcttcatggaactctcacacctacaggggaagaaatggtttagaggttactaaaagtatatagcctggtttaggtgatagtgagctatagaaaaagagaaaataagttgtcagtactttaaatctcaaacaacttcttacaaaaggattttaatagtttaaatacaattatcacaagttgggtaaagacaaccagacctatggcctgataaatcatgtatttaatttatgcatatagttagggccaaaaagagcagagtatctttttgaatgttttaaaatataatctaggattttatgcctacagtccttgaaattacaaaggatctctctcaagttgggaatatgctatccattgcagttacggatgggatttagagactgttttaatttcttgtgattatctcaaacaacgtgttccttactcccaaaactcccttgtatggctacagaaactgttgcagttttagttgcttctcccctgcctttgcccagaggtagtgtggaatgccaccaattttatctgtaatggttcctttcaagcctggggctatgtaagctgctatatatgcttcattccattcatcattcagtctaaatatagctatcaaccactaaattatctgataggtatgcatgaactgactgattctttgcataactactaagctatctgttttatttaggcttgggagccctagggtaaggatacggaataaattgtaaatgatattaaaagaatagcttggcaaggaataatacctcctcaggggccaaaatgcaaggcaacacaaagtgatttttttggtgatataattataacacatgtagtatggtagtgataactaaaataaaattggatagcctcaaaattcctacatgtagtatgataaacaatgttctaagactgaggcctttgggaggtagtgagttatttcctatagatagagagtaaaattattcctataatttggtatgaaagttttaccagaaattttgcttcacagtaaatcatctggattgggtgaagccaggtagggattgagaagtgagtcaggtgggttgcatagatgagtttgtgctgcagaccaaactcctcttcccatagctcacttcttcccactcctgcatgttagagagaagactagaatgtgtcaaatacaacctataggataatagttcagagcattagcttcggaagtaaacactaggttgatgccctggttcttctaatttctagctgtgtgacctagggaaagttacctgctgtgtgcctcatttcccttatttgtaaagttgagacaataacaactccctcacaaggttgttgtgaagattaatgaaaaagcagtttctgagaggttgtgagtgttcagtaaagaaagaaattgctttcctttttttttttttttgagacagattctcattctgtcattcaggctggagtgcagtggcgcgatcatggctcactgcgcctcgacttccccaagctcaggcaatcttcccacctcagcctcctgagtagctgggactacaggtgcgagccaccacacctggctagtttttgtattatttgcagagatggggtctcgccttgttgcccaggcttgtctcaaacttctgggctcaagcgatcttcccacctcaacctcccaaagtgctgggattacaggcatgaaccactacacacagcctattttccattttcactgccattattttctctgtctcttgtgtggctcattactgccacttctgtgcaccatttccatcatgtcacatgcgtagtcaaaaccctcagtgactgcctaattctcagagggtaaaagtgaaattccttggcctggaaagtgcaatcatgcctgaagttctacctgtgggagctgtactagttatctattgtgtaaaaagttaccacaagcttagcagattaaaacaacacatatttaatatcccacagtatctgtgggctaggagtccaggcacagttcagctggatcttctgcttctaagtctcatatggttgcattcaaggtgctggctagagctatggtctcttctgaggcttgactggggatggatatacttccaagccaatgcaattactcagaggccgtctttaattatttgccatgtggccttttccatagggtagctcacaacgtggcatcttgctgcaacaagtcagcaaaggagagagtcttctatcaagattaaagttaaaatcttatgtaacgtaaacatgacatcctgtcacctgtgttgtatgctattggttgaaagtaaatcacgggtctgctcatacttatggggaggggatcacacaagggtgtgaccaccaaaggcagagatcatgggagcattcttagagtctgtctgccacagggccaggatcaaatgctggtggctgattgccagctccactgtttatttctctcagccgctttctgtatgtacaaaaaaggagagagagtgattttctacctcatagtgttgttaagaaaatcaaatgaggtaaattattttgcaaagtattctctactcatctgactcctacctactttcaggtctgtcttaagttgcaacttctgcatgagggcttggccaagtatggtcacctgcatgaatttcctctttcttgtaacagaaactgcttggcccactcataatttatacattattatatactcttatgtttcattaatgaaacatttataatgcatatcttatctgtctgataaaatggaaagcaatctaaggacaatgacttttcccctatacataacaaaatataaaataaatcataacataataaataataaataaataataaaataactaacatttgttgaatgattaggatgtgttaggcactcttgtaagcatttcatttttttgttacttcttcaaatccttacaacagtccttggaggtaagtcctattattactttcattttgcagataagacaactgtggctcagagaggctaaggtacttgagtccaggagcagaattcttactttcctcaaacgttatctcttttgtgtggtctagggcagtacaatataccaagtagttgctcattttggtctttggtaaatggaaatgaatttgtaagttgagaaaagactacactacagtagtttggagaagtccactttccaactgatgttcaagattcaggagggggggaaatgtgtcctggcatcatcacttgcttgatgatttcttttgctggtagaaaatcaccatggctaccaatggccattttctgggaagccttttttgggaatcctaacttaagatcaaaatccctgggctaagtcacggcagctaggaggttactgtgaaccggttctcatttcttatggtttccattgttattagggggctgtattctcattaagtccactctgagctctactggattctctttttagagcactcgtctttgaattcttggccaactcatctatgtcacagcactaaggctataaattccagggccccacaatgtggattatctgtgagaattcatttccagttctggggacaagacccttgggaaaaccaggcaataggccagagagagacagagagagagagagtggtcaaaagtggcctggtcactcttaacctaaaccttgaccttcagcggagtagaggactaaatcactcaggaaatattaaatgtttcaccagatattatttggactatatcttctgttctatctcttcaagcaatgccattccaacttcaattttcttttctgtcatttcagaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacgggtacgtttgtgaacattttagcattgatcccaagagttgaaatatttgggaaatattaaacattaatatcacccacagcagctgcctttatttgaaaaaatagtaatgctgatgttagaaaaatctctctctatgcctaggtaaggtgtccatataatttattattcaaactggaatctttgagagtgaaagacactggtaattattatattcaaacacagagttaaactgagattgtccctggcactgtggtgtgtatggacaccccctagtgttaggtcaccttcctgaaggatcaggcagcctgaagaatacagttagtttatgtcagtggttctcaactgggggtgatttagccctccggggatatttgacaatgtctggagacgtttttggttgccacaaatggggatactgttgacatctagtgggtagggatgctgcagaacatttcacaatgcatgggatggcccccaacaacaaagaatgatcttgtccacaatgtcaacagcaaacttttttttctgtaaaggaacagatgacagtaaatattttaggttttgtgtatttaggtattgtctgtgttgcaactagtcaactgtgctgttgtatcaggaaatcagctgtagctaatatataaatgaacaagtatggctgtgttcaaataaaactttacaaaaacaggcagcagacaggatttggtctgcagaacacagtttgcagacctctgatatagcctattgaaatatttttaacctaaaatatttagctatttaaaaagaaagtcttctttctcagttgcttgtcctctggaactttgtatgttctaatctgtgaatataaaaataaaaatgcatcaaaaaggcaaagataaaaccatgtgaaatgtatctttctacaagttaacatttatttttctgcattctttgggtccaattatccaactaattatcaatgcactttactttatccattctatgtttctccattttttcccccaacaaggacactataagaaaccttgcccattcgcatatagacaatctggtctagcctaggtggaacctagaaattctattttttaaaaaatccccagataattctaatgtgtagccaggactgaaaaccactgaaatgcatgtaagagcttaaagtgtttgggaagaggagggactcaaggttgcttagatttgatggagaggaagccgtggagattgaagaagagctggagagagaccatgaggggttttgtgccatggtaaggaatttaggtttgttcctttggactatgggatcgtgtggaagttttaaacataggatctatttcatgttttaggatgactctcaaggcagaaccaaagggaaataggagaaggagaaacagatcctttaatccagtgcttcttaaatcaatctgagggagagaaccatttccttccttccttccttcctttcttctcttcttccttccctccttctctcctccctcccgtctttcctttctccctcctttattcctttatttccaatacattgcagatcaatacttgcttaagttcattgattacatgcttagatgtcatggaaatatcaaattgttctccaaagtttataaacttttattctcttaattcctgtacttatcacagaatggaaacaaactgtcacttgcactggtccacggattacacctggagtagcactgcttcactatatcaccttcactttatctgcagggtaaagtttaaaaactttggtatggcatagacggtccctcgtgatctggcccctgcttgtctctccattctcatgtctttcatttcccctaatgaacaccattgctccagtttcattcaacatctcactgtcccctgagaatgccttgtcttttccaacctccttccatcttttagactcttccctctacctcccttctctacatgaccagtgcctacttcttctccaagacacagctcttatcttcccttctctttggacccattcttgaatctcctggttattagttattaatttctccctgctcctatactaccgcatcactttttggtgacagttatagcacttgtcattctgttatgtattaatagttattgatttcatggctgcctcactgtcctatgactttatggtctttgacttgttcacttcagaattgtcagagtgtaggacatattccaggttcaataaatgctgaatgaaacaaaaaatgcacctctctgtgtatgtgtggataagaaaacttctttgggttttggtagatctggttatttcaattactccatatatatttattatgtgcttatagtactttgtgctagtcgacagtggggaaacttaactgggctattccttctccacatacagaaagatacatggcaaagaagtaaattgctgtattttgagtaatcaaaaaatcatgtggtcaaaaggatatctttatattagcattctcttcagcaaaatttccattgttaacattgtttattatctttaatttgcagttgaacacaattctaaatacaatgagcaccatctacagtactggaaaagtttgtaacccagataatccacaagaatgcttattacttgaaccaggtaggctactaatttttagtagtgattatgaaatttacttttctctcagattttaaaaatgattgcagatatgtgtgtttcaacacatagatgctcttttaacttttagtaaaactttcttgtaaattgtatcacatttacttaatttgatcaagtcagtaagtaagtcatttcagtggtttattttctctgcagacatattttggaactataagggcaaggacatgaatgaatcatgagattttatttaaccactttatgacctgatgtctgccaaccttacttttaattctttaaagttcatgtagttctttacaaataaaatttttacaaaggcatcttttcttgtctttgtgtgctttgggataacaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcgtaagtttgctgatctgtagaggtcctgaagctctctgtgtggccacggggctatttcttctgcattaccaagcctgccctttgatatcagggatacccctcaattaacaatcatgtcctcaacaattcagattacctgttttcaaaagaaattactggggaaaaagaaaaggtgcactggcattctacaattcaagaagctcaagaatgagactcaactacaaggcctaaagtaaattatccaggaattatgaattataagtaatctaggaagatctcaggcccctaaaagttcccgagggagtctggggattaagggggggaatcccatttctcaggatgaaagacagagtcactgccttttgtctaaagcccccacagttcactttcaaaggtcatttgtgtgtctaccttagggtgataccccagaaagaaaaaatatcagattagtttaggctggaaatgtattattttctgcagttgtaattatatctcagattatgtggtcataaaaaatcttaacataggaaggaatttccatatctcttaacttattctcttccctgaatagtgagctgaagtcatggaatcatgattcacattttgctttgatgatttactgcccactgaggttgagtaaggaagaatttttttcccgcatttgttgttttaacatttctttcactgccacttaaattcagtattcttaagccttaggcaagctggagacaatagcttatcaccatttttaaaacaatatcctgcatgtcctttcatccaaatatttatttctctcctcatcttcctgatcattaattgccaatgtgaatgtaaaaataaaaattattaaattatgtttacaaactttattagctaatatcctaacacaagcttgtgttttctgatggtgagatactaaaggaagcacatagaaacatgctattgatacatataatttgcaaacaaacacaggttcactgtggcaaagtctaagatgagttgggtctgatctaaaaataaaaggccatgaacaaaagaagaatttaaaaacttagagatccagtcacatattttaaagctaaagatacaaactgatgtatcaagagagtacacacccagggttaagagactaggaatgtgcttctttcgcttaccagctgtgtgatcttggacaagtgatttaaccactctaagcacaggttgttggtgggaatataaactggtgaagctgttatggaaaaatagtatgaggattcctaaataaattaaaaatagaattaccgtatgatccagcaatccctcttctgggtatatacctaaattagatgaaattaccccttcataaagatatctacactgccatgttcactgcagcattattcgcaatagccaagatagggaaacaacctaagtgtttgtcaagggatgaatgaataaagaaaccgtggtaggtatatggaatgggatattattcagccttaaaaaagaggagatcctgtcatttgcctcaacatggatagacctggaggacattatgctaagtgaaataatccagacataaaaagaaaaatatggcatcatctcactcaaaagtggattctacaaaaaaatcaaatatacagagatagagtaatagtgattaccaggggtgaggtggagcggggtgggatatggggagaagtgggttaaaggatacaaagtagcagatatgcaggatgaacaagtctaaagatctaatgtacaacataaggactgtagttagtaataatgttttgtattcaggatttttgcaaaatgagtagattttagctgctcttccatgggggggataatgcttaactatgtgagatgatggatatgttaatttgttttactatagtaaccattttattatataagtatctcataacatcatgttgtacaccttaaatatacacaataaaatttatttaaaaataaacataggctgttactctgcacagtgggagttataataatgacaatcttgtatggttcttgtgcttattaaataaaataatacatttaaactcctggcacagagcaagtgcttaaacagagattacttaatgttattattcatttgcatccctttgagttgatgtttatcacaataaaaatgtgataagcctttaaatttcttttaacctcccagcaaggctaatctgtgtattttgtgctttctctctttctttcccttatgttcttccctttctctttaaaaaaaaaaacaaacagattatgaggactatggggattattggagaggagactatgaagtaaatggggtagatggctatgactacagccgcggccagttgattgaagatgtggaacatacctttgaagaggtaagcaaggaactgtacacacaaacagttgtggggcagtgtatgggaaagagggacagtgactaaaggcattctggctcattcttaacacattggtcagccattcccaggcctcataaagttatggaatattagagtttgaaagattttagagaataataggacagcccctacagatgatcagaattgcgctgctttcctccttctttcagtcactgaccattaattaccaatagaaagactataagaatgaaatattataagaacaaatggtcctttagattataagtaggaatgatcttcttgagctacctggggctggcactgtcttcctgtgttagcagagctatggactttgctttcttcctcaggacaattgatactattactttgagtaagctagtcttcatagggaagagagagttttgtgaacagaaatgtcccatggatgtttttccttcattttggccccatgcctcccctttacgtgaaccttcaaacttctgatgtgagttaccttgtcacagccatagtttggcagagctatcaaaaatggaagtaagacaaacaactccccaaaaaggaaatgggaagaagacttgaataagtacatcatcaaagaaattacacaaatggcaaataggcaaataaaaagttattagggaaatgcaaattaaagctatgatgaaaatccactccacacccaccaggatagctatgattaaaaagatggacaataccaagtattggggagaatgtggagacactggagccctcttacagtgcaggaagaaatgtaaaatggggcagtcactttggaaaacagtttggcgatttcttaaaaagttaaacacacacttaccatgactcttaggtttctatatgaaataaatgaaaacatgtgtccttatccatagaaagacatacacaaatgttcatagcagcaatattcataatagccccaaactggaaaacccagctgtccttcaactagtgaatgaataaacagactctgatatactcaaagtagccggagataggagtggaactgactacaagaaaccttttgtggttatagaaatattaaaaaactagatagtggtgatggtttcccggtagttttaatttattgaaactcatggaactacacacttgaaaatgggtggcttttatagtatgtaaattatacctcaatgtaaaaacaaaggaagtaggtaacaatcttaaaaatgaatatatattttttgcatctgcttttattttgggacctgtgttctcccaagtatgtttattggtttatgcagtggagttatcatcaagttctaggaagaaggaatacttttaaattttccagaaatttgagagaagaagaattataatttggggaaattctacttgtttaaagaatctcctatttctagtgttgtggaatggaaattagaattggttactctttgtcttaaactttcttcctgggcttttcagattaaaccattatatgaacatcttcatgcctatgtgagggcaaagttgatgaatgcctatccttcctatatcagtccaattggatgcctccctgctcatttgcttggtaagaagccccatgaattcttcgtgtacttggctctttgttatttctaacaataaaattgcctttttgagtgagccttaacataaaacttttgaaaaattctgtaagtggttagtaacattgtagaaatcgcattttgaaagcattttcttcaaccaaaagtctattttaaacaattctctattgtattactttttttgtacataaagagttattattttggctgcaattcaaatgataaaggggttatttaagataactagagaaagaattacaggctcaaatgagagtggaaatggggaaaataatttttagtattcaagttgattggctaagctcttaaggtggaatttgagattttagtatcatattgtcagagcttggttggctgggggtggtcaggggtaacagaaggttatagcttgagcgaaagcttcctaagttcattctagacttcaccaattactcttgctgaaaagtgcttggcttggaatcgtataagtgaagaatgaatgaaagtgaccatcttcccctctcccaccgcagcagctgaggccaaataaaatataaatatttgaaaatttcatgccatgcatatatagcatttctactgtagtcaaactaagttgctggaagctaatctcctccacagccatacctcgtgtctgtcagcaggagagaatgcggagaaaggacctgtggaggcttttaactctaaagtaatatggaaatccttcctacaatgtcaatggcaagcagtatttagtaattcagtttctgcttaaacctgtctggtgtgatgggataatttcttcattccaatatagactaatttactgctacatggaaccaatgtaatacaattctaccttatgtggaacttaagcaacacccctctgatgccttccattcattagccctatttccatgctatgaggttacccagaatgtcaattcctattctacaagatgaccttccagatattcataaagaaccatcatataagatatagcatagtacctggtgcaatacattttagtgactattattgttattgtcatgtttaccctattttcttttgtcaagttaaatatcttatacattatcaaccattccttaaaacctcacttttctataattgttctcctttcaatgagctgtactttgtcaatgtccctcctcaattatcatgcttagagatgaacagttactccagatgaggtttgaccaacataaagaaagacatggtaccatgactacctttgttctagacactatacttctattgatttagctcaagttcacatttatgctttagaagccaggtcatactgtttgttggctcatttttggtcttttacatgtggactgtgctttagcctgatgtcacatatatgtttttatgatggataggtttttctttaaaatttaagcataggacttcacatgtatcactactaaattttatgttttaaaatataagtttagggagtacttttatttttttcagagtctcgctctgtcgcctaggctggagtgcagtggctgatctcagctcaccgcaacctctgcctcctgggttcaagcgattctcctgcttcagcctcctgagtagctgggattacaggctcaagccgccatgcccagctaatttttgtatttttagtagagacagggcttcaccatgttggccaggctggtctcgaactcctgatctcaagtgatctgcctgcttcagctctgcaaagtgctgggattacaggagtactcttcatgaagtttgttgccccatttttttggcaccaggattctggtcctgctcttggattgactttttctagatacatcttcttttttatggtccctggccttggaatacaacttaacatttgtttgagatgtttacaaagtcaccaagttaagtacacgaatataggaataaaagtggaatttagttaccagttagtagaagttcctgaatgatgatcttatatgatctcttttgaagccaacactaggaattactaacagcttaatttgtaatattttgtaccatggatggtagattatagaaatctctttctaatttacagtgtcaatgatggtccatatatttaataaattatgtctacatttctgctgtgttgtcatatactaacagattcttttttaaatataggtgatatgtggggtagattttggacaaatctgtactctttgacagttccctttggacagaaaccaaacatagatgttactgatgcaatggtggaccaggtaggaaaaagagcccttaaaaactaaatctaaattctcaacttcatttatttttatgtcatccttctatttttatttttatgtgaaaccatgtaaataaaaaatttaagctaattgaaatttatttcttatgtagctatagtttcatggaaaagcagtttacaatggcctcagataacttgaattgcaggtgtagtattgatctcattgaaaggtatttgaaatggagattacctgagttttccattaagcggtaagatgtagaattgtcaactggttggtatcactcattccaaagtttgtcattgctaagtaaccagccagtaatctctatggatagtaaaaacaagaatattatgaggggatttttgcatatttaaaaaattattagtgttaaaaatattacaagaatctttaaaacaattttaagtaacatctttattactactttggtttaattatttaaatgtgctaggttcctcaaaacacaagtaagtacatagcccatggacccaataaagcaactacacaatcgagactacaaagcaaccagctaacaacagcatgacaggaacgcttcacctaacagacagagagtggcaaattggagagaaaaacaaaacctaaccttctactgtcttcgagattcatctcacgtgtaatgacacccatgggctcaaagtaaagtgatgcagaaagatctatcatgcaaatagaaaacaaaaaagagcagggggctgctattcttgtatcagataaaacagactttaaaccaacaacagtaaaaaaggacaaagagggtcattacataatgataaagggttcaatttaacaagaagacttaattatcctaaatatatatgcacccaacattggagtatccagattcataaacatagcacttttagacctaggaaaagacttagacatccagaaaataatagtgggggacttcatcaccccactgacagtgttatacagatcattgaggcagaaaactaacaaagaaattctggaaattctggacttaaacttgacacttgaccaattgaacctaagagacatctatagaatattccacccaacagctatggaatatacattcttctcatctgtatacgaaacatactctaagactggcaacatgctcagtcataaagcaagtcttaataaattcaaaaaattaaaattatgccaaacatattctcagaccacagtagaataaaaatagaaatcaataccaggaggaactctcaaaaccatgaaataacctggaaactgaacaacttattgctgaatgacttttgagtaaacaatgaaattaaggcagatatcaaaaaattctttgaaacaaatgaaaacagagacacagcataccaacaacatatctgggatgtggcaaaagcaatgttaagaggaatgtttatagtgctaaatgcctacatcaagaacctagaaagatatcaaattaataatctaacaccacacttaaaggaactagaaaaacaagaacaaactagttccaaagctagaaaaaaagagataactaaaatcagggcagaattaatgaaattgagacctaaaaaatcatacaaaggatcaacaaaatgaaaagtcagttttttaaaaggataaacaagatggaaagacctctagctagattaacagagaaagagagaagatccaaataagcacaatcagaaatgacaaaggtgacattgcaactgatcccacagaagtacaaaccttcctattgagtactatgttcatatctggttgataggaccaatagaaacccaacgtcagtatcacacaatatacccttgtaacaaacctgcacatgtactccctgaatttaaaattaaaattaaaattaaagttaaaaaattatcaggccgggcacggtggctcacagctgtaatcccagcactttgggaggccgaggtgggtggatcacgaggtcaggagatcgagaccagcctggccaacatggtgaaaccccgtctctactaaaaatacaaaaaaaaaaaaaaaattagctgggcatagtggtgtgcgcctgtagtcccagctatttaggaggctgaggcaggagaatcacttgaactcgggaggcagaggttgcagtgagccgagatcatgccactgcactccagcctgggtgacaaagtgagactctgtctcaaaaaaaaaaattctctgtgttcccttctgttgatgaaatggtattatagttataattatgtatatttgttcattcaactaaaattctacgtttctgagtagaatatctaatcatatttctatttgccttataaaatttattcagataattgcaccataatttactatttgtgcattgctgaacctttgcttgttaatagttttacatctgtaagaagcaatgcaatgaacatcttcacatacataacatactcctgggaaaagtccactgcagaataaaaagtgaaactgcaagggcctctaaactcttgtagctgttgtagctccactttgaagttgagcagcagtatagggccatcttgatgtagatttttagtctttctaaagggtttttacacccgtctcttctattgtcatctctgactcaggataggtattgtgacagctccatgctataggtgaggaaactgaggctcaggtaaatgatttgcccaaagtcacaaatcacacagcctcattctttctgttttttttgtaccaattaaccatcctcacctgcccctgccaccttcccactactctttccagcctctggtaaccatccttccactctgtatgtccatgagttcaactcttttgatttttagatgccataaataagtgagaacatgtaatgtttgtctttctgtgcctggcttatttcacttaatataatgaccttcagttccatctacgttgttgcaaatggcaggatttcgtttttttatggctgaatagtattccaatttgtatgtgtaccacattttctttatccattcatctactgatggacacttaggttgtttccaaatcttggttattgtgaatagtgctgcaacaaacatgggagtgcagatatctcttcaaaatactgatttcctttcttttgggtatatacccagcagtggaattgttggatcatatggtagctctatttttacttttttgaggaacctccaaactgttttccatagtggttgtactaacttacattccttccaacagtgtaggagggttcccttttctccacaccctcatcagcatttgttattgcctgtcttttggatataaaccaaaaaatttttcataaatataatttgggtggctgcacattattctatttttcaaaggtatcattatttatttaaccattctgatggtcgtttcaactttttctctattaaaaataattctcaatgtaattgttgaatctaatttctgataattttttaggttagttccttagagacagcattactgagacaaagagaagaatttcaaatgctatccgtgtgtgttatcaaattaccttttggaaaggttatgccaatgtatatatccaacagaaataatgctgctattttaaaaatgacattagcacttttttccattttaaaattttattgtaaattgacatttattattgtatatatttatggggtacaaggtgatttcatgatttatgaatacaatgaaaaataattaaataaagctaattaacatatcaattacttcacatactttttttgcagtgagaacatttgaaatttactctcttagcaatttcaaaatgtacaatgttctattattaactatatttactatgctgtgcagtagatctcaaaaaaataaaaccaacttattcatcctgtttgagactttgtacgctttgaccatcatctccccgttgcccccaccccatagcctctgtaaccaccattctactctctgcttctaccagttccattgttttagattccacacataagtgagaacatgtggtatttgtttttcggtgcctggcttatttaatttaagaatgctgttgttttgatgctggaattaatactgttctcttttcccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggtagtggggctgatacttacacaactgatactaaatgggagacaacagaggcaggctgaagtttaacttgaattctttctctgcttttctgagcacagaaaagataagttaattctccttgaactaaggctgggagtccaggagagcacatttgggttttcccaggaaaagaggattgccagcaggctctaacttgaacctgtggacaatctgctttcttggaaatatcatgcatccattggcgttgtatggtaatggggatggttgcacccatatcaggaaagatttggtgctaagatttttggaagatggacatatgaatcacacattgatcttcccctccatgtttcactcttgtgaatgagacagaataggtgaataaatacgcttggatcgaagggagaaagtggtacatgtcgtaagagagacacagggcatgctctatggagaactggaagaaaactgaccacatttgcaataggagataggatcagaccgtgctttacaagtgggatttgaattaggtttggaaagacaagaaggattcagatacacagagtcgggaggaggacccaagctgtgagaacagcaggatcaaatacagagaggcaggacctgacctgcatactgaagtcggcaagttaggctagaatgagaaataagtgaaggagagtttgtgtaatgtggcagaatgagcacaggcttcagaatcctaggtgtgtcacttaatgactatgcaaccttggacaaggtatttaactttctttggtttcagtttccttattttataaagtagaatagtaattcccaggttgcaggcttgtgagagccttaggttggattccctagcttgaaaaggagatcgttttacaagtgcttcattgaggagagctctgaggcagaggggaatgagggaagcaggctgggacaaaggagggaggtaagtaagaatgtgatctttactggaaacttggcctcagccttatcccatgaagctctctggagcataacttgagccacagactaggccacacctcaagacaagggggctagccttttatatcaggcagtctttggctgtggactgccccagagtatgggggagcataacccccatttagccaaaggcagtgaagggagcagctgtaggcccttatcagctgtgactcaggcagctaggggatggtggataaagtcttggcaggcccgcaccagcattgactagggtagggattggatctacagtccttttctgtaaagggccagatagtaaatagtttaagctttgcagatctctgttacaactactcagctctgcccttgtgacatgaaaacagccatagacaatacacaaaccaatggatgtggctgtgtgccaataaaaatttatttataaaaacaatggctggccagcaggccatagtttactgatgccagtactagagtgaggcttaaaaatgagaaagtataagtaagttttcaagcacagaatcttacatacagtaggcagtaaatatatagtagttatttttataattatttcaaaagagaatagagagaaaccaaattagaaaagtaggttggggttgtgaaaggaaaatagatttactgagcatgagatgaatacataactgaatatttccttaccttccccctttctcttgcaatgtgtgaattaccatgccctcccactatcctctccagctcacttttctcctataaatgttgaagccctcaaaattatttttggagaaaggcacaaaccacagactgtttctataaattctgtgttcttttcttctgggcatgttcttaaccttggcaaaataaacttgtgaattgattgagacctatctcaaatagtttttggtttacagggtcatattggtgtataaaaggtggctgagtttattcttgagcaatgaggaggcctcgcaggatattggagggggtggtgacatgatccaagtggtatgaaatacatttaggaatctactgatgtatgacccctacacccatctgaatgaaaatgctcttgctaagttaaaaaggaccaccatctcattgccaaatgcagttcttatcccactgggctatagcagcatttgacaagatttacaagttgtctttcaaatgctttttttttttaaccgacttccacattgtcattcttttccagatttccttgacctctctgattgtactttctccgtctcccctgaataatcttcctctccccactccttaggtacttggaccttctaaaggtttatctttggtcctcttttcttgcattctatacctgttccatggcccatcatatctatttcccaacttacatgctaatgattcccaagtttcaatttgcagccccttctactatttctctatcctggcaataccaaacagcgtgcagtgtcctgaatgccacatttccacccccctgcccctaagcctttctcattgagcactagaacgtcaacttccatgtcatctaccgggcaaactcctgcacattgattaaggtccaaattgctcattgtatcctcagtcatgcccttcttggtgcttctagggaactgtgcctacattcactcatttttacgctcaaggctgtgtgtactgatcacttatagagtacttgcctcagagttcttgtagcggagaactctgcttatctgtcttcctgttagtctatgagcaagagaacaggattcagctgtgcccattgctttatattgagcaactagtattgtctctaggacataagaggtgggtactcaagattcactggtgaataaatcttggaaagatgattcatgctaccaaaaccccatacaactccactgtaatggttaaatgaaagtcagtgagattcattttcatcttgtccattttcatgcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatggtatggacattttctcatggcttgttttggaggttctttaatctgatatgggaaaaggtatttattatcttatgtaggaaatatttactttttgccacataaggtatgaggtatttatgggtaactggaaattactactggtgaaaaaatttaagtatatatcagaacatgtcaacttttggttttgtattattgaaaagggaataagagaggtaactgaatgaatggacttgggatcctgtgaaatatctaacagaagtttttttcttaaagatgatgaaacagtctcacagaactgaaatggcttgtccaattagtgtcagcattctaactagacccctctggttctaggtaaaaacttcccaaaactttagagttggaatatattctaaaaaccattttatagaggagtaaactgaagacaagagagggaaagtagcccgaagtcaaaacgcaatgtagtggcggaaccagaacaagagccctaatctaaagtcttttccataatattatgcaaagcaaagtgtagctttaaattttggaattcttaaagaacaggtgtttaatatcaagattagaagggattgaaacattttgttcatcctgctgaagcgtgaatttcccctgtaacatccccttagatctgtgctatccttcaacatttctgcctgaggcagcccatttccttttagatgccttggacttgatggaacatgacaaatgttcaggagatccaatttcttcagtgaggatgccatggcttttgattcttcctcataaatggaggacaaatagagcctaataccttcttctcttctgtatattctctcccctatttttagttatttgtgacataaagatcagctcctctgtctggagcatttggtcgatgaggtcaaggtcatgagctccatccttgagtagacaattcactttgccttgatctctggttccagatacttggtgatcccacaagggcctgggagtgagccagtgtaaagcctatcatcatgtttgaaatagttactgtaagcacagtcctagtggattacagaccagtagcagcatcctcccatgaagacagtaacaaacaaaggtagttaacagtaaccatacctgcttcctaattttaaaatatcattaattcaagtcaatgaacagatattttattatttttaaatattccctgacagcaggtaaaaatggttgatgactatgtttcttaagatgatattagatcagtgatgaaataatctgtacaacaaaccccatgacacgagtttacctatataataaacctggacacatacccctaaacctaaaataaaagctttttttgtttttttttataaaaaagatagtattagacatggttaaaccaccggtttggaaaactcctgctttttttcttgactctgcccctgatttgtgaataatgttaaataaattcctttattccttccattcctccctccctctttctctttctttcttcttccaaacatatttatcgagcacatactgtgtgcaagacatttttctagacactggagacacagtgatgtgaaaagtagataacatcccctgcttacatttcaatgcatttccttcatacactagagatgaactccactgcctctttatgtcactaataaaggataaaatgaaataacatgatttgttaaagcattttaaattcttggtaaggaagatatccttacatttaaagtacattgtacttttcaccatccagtggatagtgccctccaggagggctcttttgtttcatttcccctccctggtttacaggcttttcactgaatctgtttagtatttgtttatttaacaaagactcacagaaggcatcacgtagcagacatgggactaagttatacagtcttaaatgagacattaacagtcactgacttcatggagctttccagaataaacagtgtgactgggaaagtggatgaaatagctctaatgaactctgataagaggtatttaagggaggaaactgaaactaatgaagaaaatgaggattagaagtaactttgggttttatttacctgttctgttccagggcttcaacctactccaaatcccttagctgagaaaataaatatatacatatcattatgaatactgtcaatttttctcatgtatccttatagtaagtacatcttgaggaataatattttgaggttaggaaaattctctttaacacaaaacatccactgtcatcttcatcgtaatatttatccttttctattttactttcagaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagtaagtcaatgaatatgcaatcagtaaaatgttttctaatgagtttgctgtgtatttaaaagcatttgactggcgtgtacacacacacacagaggcacacattaactaactactttttgtatttttagtttgttatattttatattttagtctccatttggataccatctcctcagataaaatttccttgggtcaggtgacactacttcatactctatattcttaagtattggcttactcattaaactatgatattatgttgaaagtgatcaatttattcattttacatattcttttatccagccttttaaacaaaacggtgaccccacattttaggcaaagcactggggatccaatgttaataatttcaggtatggtcctcaaccccaaaaaacgtttagtttatggcaggatgtggaaaattaaaccactgcaatttgtagtgttgtgatgggagggtacagggtccgttagcgtccaggggagatccaaagcccaactgggggtgtcagatgaggagaggcatttaagccctgagaaaggtgtgggtgtagaggggaacacagagaattccaagctagagagaacctgacacatttgggcaacagtgaccatttccatggttgcagcactacgttacttattgctgtgtcacaagtgcctcacactgtgcctgtcacatgcaagtatgaaataaataggtttttggaggaaggaattataagagaaaaagaaaaagaaggaaaaaaagaaaggagagagggagggagagaatggaaacaagaaagaaatctatcttctttttaccatcatagaccctggtcaattatttctttctttatttttcttatattttctttttgaagtgaagaactcatgtgtaacaagcagttccacatgcacaggtttaaaacagtacacttcttctgtcaagattctctctgttaacataaatcgtacttgggaggaaagacgcagcacctataagaactgttgccagtgtttaaatattactgaagttatgtctacaatgatcctgcaattttaaaggaaaaatgtattccatttaatcaaagccaaatacatgattttgctacctttttttctggttggattaatcttttgacccctcttagcttattttaaaggcacatggaatcagatccttagagtgattcattaatgcatcaattgttcctttgttcagtaaatataaattaattctttctgtagtgtcaggcaccatgtttgacactggaattagagagaagaccaagacacagccccaccctctaggagctcaagtctctagggcaggaagttactatagttgaataactacaaaacggggagataaatggcaaaggaggcacaactagagaaggctaaaggagattgaggggttaattctgccagaaggtggatgggatcagggccaggcttcacagaggagggaacacttgagctaggtaggcgtggagaataagtaagatttcaattttattaaattctctctgccaggagtacatgaagtgtggtgcctgagaggaattattagggctttcactgtacatttttggccatgcaatgcactttatgaatgttattaaattagtcttttgaagacatagctattttccaagggtatattctggctgaattaattaaaactaagagataactacataatgagctttactgtctgattttttaaaaatttttatttattttttatttttgtaggtgatttttttttacattgtttctatttcacccaacaaccatttctaatgtaaataccattgattggctggacacggtggcacctgtaattccagcattttgggaggccgaggtgggcagatcacttgaggccaggagttcgagaccagcctggccaacatggtgaaaccctgtctctattaaaaatacacaaattagttagatgtggtggtgcacacctataaaccaagctacttgggaggctgagatgggagaatcgcttgaacctgggaggcggaggttgcagtgagccaagatcgtgccactgcactacagcctgggtgacggagtgagaccctgtctcaaaaaaaaaaaaaaaaaatttgattgatgaaactgcactagttatgcccacctgcttgtgatgcttgtgatgggtgatccacagctaatgtattgttttcttttctccccaaaggcgagagatagttggggtggtggaacctgtgccccatgatgaaacatactgtgaccccgcatctctgttccatgtttctaatgattactcattcattcggtaaattacagttttcttgtttctgtttaacttctagggtttgtggacagctgtgctaataacgacaaaaagtagggataaatgaagtgataattataatttttctatttccacccataactaccaagctcaatacagatgctccttgctttacaatggggttacatcccaataaactcatcataagttgaaaataccttaagaatgcgctgaatacacctaacctatggtacaccaaagcatagcctagtctaccctaaacatgctcagaacactcacattagcctactgttgggcaaaatcatctaacacaaaccttattttataataaagtgttcaatatctcatgtaattatttaatactgtgctgaaagtgaaaaactttgggtacttaccattaacatatacagctgaaagcgctattgtaaagtcgaaaaatcaaaagtccaaccgttgtaagttgggaactgtctgtatagatgatcaatttcaattcaatttaaactaacacaatttattgggttaaattaaacttgtgtaagatcttgtccccgtcatcagcaatcaatagtatcattgggagaaataaaaagtagttttgtcttcttgttactggcagtttattgtacattgtgatagagaatttttaacttgaagtccaaaaacatatgttcttcacctactaaccccagtccttgaatttgctggagctcagtttatttgtaaaatgaaaataatattatcgacctgttataaggatggatttgcatcatttatgtgaaagggctattaatctgtaaaacacaaaacaaatgttagctaacatttaacagagtaatattgccatgtttatcaacagatacataaaacacaaataggttatgatgagtgtagagttcatgtctggagaaagagatttcagagatttgatcagtatctctttggttacttgggctccagatttaaatatatgccctaatctataactctaagacagtaagtaaacacgggactgcttttttgttgctttgtctcctgtgcagatattacacaaggaccctttaccaattccagtttcaagaagcactttgtcaagcagctaaacatgaaggccctctgcacaaatgtgacatctcaaactctacagaagctggacagaaactgttgtaagaaatacctcaaaatgttgaacctctcctagtattcagtattactcatttccatgcctaggtttgtatttgatttctttgttctaaaaagaaaattttatggcctcaaaatgtcctcatttacaaaccaaacatttaatttgtggtcagacaggaacctagaccatacaacaattgggtgggccacctcttttctccctatcataactacagccctctcttcctggtaattggaaggaaagagcggtttagggtggaatatatctgttaatatgcattcttttcttatctgccagaagcaaatttagccaagtcaaagagaagaaaccatagatcatagatgtaaatatatgtacatctggaacccctcaaaaggccctgaaccccctttttttgtgtagcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatgtgagtacacccagttgacaagtttcacacccattccttgtctacttcgtctagtctcctttggccccagtctcatgaggacttgtgacacagctgagaatgtttctttctctatagtacctgcttctttctctgctatggtgacagctgccattttatgggagaaaaatcacaactgttgagcacctatgtatgagacatagtgaaagcaactgtacagacaaacacacacagatgcatgcacatgtgtgcatacacatacaagagtgtgtactctttcaagtctgttttattagtctcatctttaaaatacatattataactgtctccaaccaactgtactcgaatcctgctttccaaagacaaatactttttttaaaacaattttaatgctattatctcttctttctggcagtcactttcatatttctaaataattttcttatattgctattttgttattgctcagttttaaaccttcgttttggtctactcttccacttccatttcttgctcttccaataaggccttataatgatttttagtaaaacagttatcaatgtttacatcactatgcctgaagagccaagtaatgtattattttgtgtcctttcttatgtggcttgttgtttgtctcaaagtttctacttgcctgaatttttcggcttacctaatttgctatctacgttgtatttgtttttctcccaaatatttcaacatatctattcttttcagtctcattttttttccctagagacttccctcttggagtcttatattctcctttcccaacatttgttgttctcctgggttgaatccactgcttcctagattccatgtcttctttcttggattacctcttgtttttctggagcatagcctcccacagactgtccaagcaggggcacaaggggtaggaggatcaactgttctccaaagtggttttaaccacaccctgacttccattcctcttcttttcagcccagtattattttcccaccctcacctgtgcttgaattccaaacctcttttggattttttggctctttgtgggtctgtgcctcctctgtgtacaaattaggtcatggcttcctgtccctgagaaagcagttacccctgtctcatcatttctcgttttccaaaagcctgttgatacctttaaaagatccctttactatcactttagtgggatctttggaggaaaaggaaataagcacatgtgggcaatctgttgtctgtaaccagcagtctgctatttctctttaatcagatgcagaccaaagcatcaaagtgaggataagcctaaaatcagctcttggagataaagcagtgagtattctggacagtgaattgattatttttgagtgcacagtcttcacttaaataggacacaataaacccatttcagtgtgaactgaaagaagagaggccatgtggttcttttcaaaactataagttccagattctggcctttgcctttgggtttttaaagtaacctattccttattggataagctattcctacttcttattgccttgaaggtgttatgaacttcaatgagaaaatatctgtatcaatccttaaatatcagataccaatcctttcaattgactctcgtcaccattacttttattctagaaagagaatatggttatattttgaaaggtagtttgtttttagaagcggtacagttaggtttttttagtttgttttgttttagtcaatcacctgtatcctggcttcactcagagtgtggcccatggtccagcagcatttgaatcacctggaagcttgttaaaaatgcgtaatttcaagcctagccctggaccacttaatcagaatctgcattttagcaagcctccaggtgatttgtacgtacaatcaagactgggaagctctaatttagaacactgcttctcaaacttggctgcacagtggaatcacatgggaagtttaaaaaatattaatgcctaggttatacacctccagagattctgatttaattgttctggggtatggagtcaagagttgggcatcaagagtgtttaaagctccccaggtgattctagtcacagcaaagtttgagaaccacttatttagggaaatgcattttgggggtaacaaaatattttacaggaaattatgttatttacaccttaatgtgaattatttacttagctattgctctgtcgttaacacctgtctcagatgatccagtctgaggaagtggtagctttgattgggaaagaggagaggaaagagtagtctcgggtagatggggctcaaaagcctgagagtgggtatgacaccctgaaggctttgcagggttagaagtcaatctgggactattggataaaatggtccaagatgctacagcaaattgttgtctgcattggatagatccaaagattgtgtttctacccccttacctttcctgaaaagttaaacattgtccaagattcaataacacttttagtataattttcatagttaagacatcaaaacataaataactcaggagtaaaaagatgaacattcaagaaagaaaaaatgaaaatggaagtgcatttctttccaatcattaagagttcaagttcctatttcataataatcattgggtgattgtgggactatttctctgtcatatttttattagctcttccttggcttaatacattttaattagtctcggcagatcaggatattttcaattatcttttttcttgacatttatactcatcctctttccatgcagtgagggttggtaaatagtgttcagggagtttgattctgtaatgttctgaggccaattcactgagcaacaatctgttttgatgaaacgagatcatatatttagtgcatctacacaacagggtcagaatatctgctcagttcaaactatttcatggtgaattccaatggatctatggcattattttgagaactcagcctttaaaatcttttatattttatatttatagagatagtagccagactgttattagtaacaatattaataatattattactattaaaggaactcctctaaacctcggtgaaaccaagctgaagtgaatgtaacaattcttttgtgaaatattataattttttaaagggaaaggagaaacatacaactagtgccattcattgattcctactgccagctactggaggaaagagtgaatgctttcttttaagtctttaaaagctatgcaaacataagctatttgtttagcactggaaaacaagaacaaacagattagcttgctgtttacaaagtgttatttttcatttgaacgtcaagtttttcttttacacttatagataagtacatttctgagaacgtaccataaaaatagtaaaaactgattttgtccacaaggtacagacattttgctacaaaacccacaacttttgtttgactcccatcggtaaacctacactcaaggtgttaatttcagtttacatcaaactaaagtaaaaggacccgtgaacccaatggttcctaaagcatggttgcctgaccagcagcattaccaccacctgggagtttgttagagatgtaaattattgggtttcatctcgggcttcctgaatcagacgctctaggggtggacccagcactcagtgttgtaactaacccttcaggtgatacggatgcacacttacattgaaggtcactgacttaatgaatagcaagaatctctggaatattaagaataattcttgaggagtatcagattataaatgtgtcttacgagtccctctgagcagtgtctttcttctgaatttgcagtatgaatggaacgacaatgaaatgtacctgttccgatcatctgttgcatatgctatgaggcagtactttttaaaagtaaaaaatcagatgattctttttgggtgagttgatttgctgggttctcaaattactccaactaggatttctcttactcttgagtctgaggagatactctgcctaaacttttcttcaagtgaaattgataaaaatctcctagtaacttatacttctttggtattgtggaatttgggtgactccatagagggctgatcgtgccttatttgactggtaaattgcaagagtattgttcttactaaacagctgtcactctcttctcagaatgctctcatacaccctcagcactgaaagaaaaaaaaggcagaacttcctgccttcattgacctatattctagtatgtatgttgtgtatgtgtctatgtgtgtaatatgtaagtgaaatatagagttacaaggtactaagtgctaggagaaaaatataaaagggtctggggagggctggtgtgtgtgtgtgtgtgttgggcagggggtaggtcaattttaaccaaaatggtcagaaaagacgtcactaatacttgaacaagtatatgaagcaggagagagrgagmgagagagcgagagagcgagagagcgagagcgagagccagcaatgcagacaatggggaaagtgtctcagctgggggaaacagcaggtgcagaccctgaggaaggaatatcctcagcctgttaaaggaacagcaaggaggccagtgtggctggagcagattagaagacaagtggagacatggccaggacgatgttagggcatagaggaaggagggagagacaaaaggtcatgtgggccttgaaggccattctaaggactttggcttttattctgagttagatgagtagtcactcaagggctatgagccacatgatctaccttattttgtgctaggatcgcttgacagctgtgagagcacagtcctgtcacatcatatggtagaccccactcattaccttcatccatgggtgcaacacgacgttttctgctgggtaggaccaggttgtgggtgtaaatccgtgtgtggtactcagccatttctgcccaccacattgtttgccacaatcctgggccaccgattattccttgttgattctcctgctgttttcagtttggaaattttctataattgaaaaagatgtgggttttatataaaggcagaacaaatagtgccaaaacttgatatcacaatttttatgtagttgatttcatgtgctttgggcagcattaatgttttttttgacaattacatcctctcattgtttgccctcccccatgtctctctatcctgtgattctctctctaatctccttttcaccagcaggggaagtcttcattctcttgattgtgtcctctgtgccacaagtgaagatgtttgttttgtttctctacagggaggaggatgtgcgagtggctaatttgaaaccaagaatctcctttaatttctttgtcactgcacctaaaaatgtgtctgatatcattcctagaactgaagttgaaaaggccatcaggtgacattttactttcatctaagggtgagggggttaccaaatacaacaacaataaccagtattttgtgtgttcttcctgtgtgccaagcattattctgagtcatttacatgtgttacctcatgctacaaactccacgtaattccagaaggatcccacagcaggggaataatcttgatttgagttccgccagatgtaggctcttacaaaagggaagaaaaactatagattacatgttatcaagcaagttaccactatgggcaactagagctcagtcctgctgggaaatgtggagagactgtgtagaatgtgcacctgaattgtcccatcagagggctgaggaagctggggtatttatttaccacctcccatcagttattggctgagaactgcttccaggtggcattaattccccagcatttcagccctgcaggcagggcagatcttgtggtctgagaatgctctctggtaaaggggagcaggtgctggttgaagaaagtctggctagaactcatggaaaatagtaagtgcagaagggacgtggacaggtattgccatcatttgctctcaggactttgatgtaggattcatttaactgcccaaatcctgaaacaaggtaggcaagtacaggattccttacatattgcttggcctctatgctaggtgagggacttgcaatgaatgtttagaagaaacagtttgtccaatttgcctcaattgtttcccacaacagacagcaggagcgaagaatggattgaccttgcagatggtttcctgtcattataaaaaggcttaatatacctactgcttctgggcagctcactaggtggccctctcgagctgcgtcagagtcctgtttaacttcggaagcagatctaagacctgctgggcagggtgtgcctagaaaatctggcttggttggagtgtatgacccaactgtgcccagagactcactcacttcctccagaagacttgtgtctgtcttgggaaaaacttctggctgagatctttatgaaaaagctcagccttcctggatactcagctgcaagctacaggggacctcagactctctgtttccaggatgactcttttcagatcttttctggctgaggttgccaggggttacctgactaattctgcgttgatctctctctctctctctggtggactctgacctcctgacctgggctatacccaagggtaggaagatgagatgccagacactttggtctttgatccagaatgttctccatatcattgtttttttttttctttaaacaatctttatccttttgcctccataaaagtaagccatttcccatcccagtgctgaagaaactggcaaaggtccctttcttatgtgcctccccagtgctacctccaaatgccaataccttttatttggaaaatattactatagagacttggtcataggacctgattcatttgtataatagaagaggtgtatccaattatccgtttccattatttgatataaagtcattgtagatgtactctgacagaagggaaattcttgccaaatatgataactttgcccttaaacacagcagtcacaaatgaataaatgccaaccatttatacatttccacacttacaactcaattttccaatggagctgttgatgaacctaatctcgagataacttcgtataatgtatgctatacgaagttatgctaggtaactataacggtcctaaggtagcgagctagcctaggttgcaaggcatgaaagatgcataattgtcaaagacttatatctttaattagacctatttactttgctcttatgattttaggtgtatattccttttttttttttttgaaacagagtcttgctctgtcacctaggctagagggcagtggcacaatctcggctcactgcaacctctgcctcccgggttcaagcgattctcctgcctcagcctcccaagtagctgggactacagacatgcgccaccatgcctggctaggtgtatattcttatgataagctctattatatcctttcaggaacaatgatataaacagtaaataactacacacaattttattttgtctaagtgtcccctttgctgttttttgcttttgcaaataggatgtcccggagccgtatcaatgatgctttccgtctgaatgacaacagcctagagtttctggggatacagccaacacttggacctcctaaccagccccctgtttccatatggctgattatttttggtgttgtgatggcactggtagtggttggcatcatcatcctgattgtcactgggatcaaaggtcgaaagaagtgagtatcctttcctagatttatctgtctttttagaatgcatgaattggaaattttgcaacaaataacactgtatccacacaggataattacttacttggacatttatctccctaaatattctgatatatcccatagaatcttgtcagatgctagcagtaccttaccttactagcaagctcacatattatagatgtctagcctgaggtttattgggatgctgtagtcaagtgcatcctatccacagtggcatagccagactgcaaactcagtaaggcattgttctcactcatgactcttgcttcatccacagagactgacatggagcacaaagtgtaatgtcatctaagaatgaggtgcttcaaactactactctttagttactttcattcctccttgggaaactcaaatttatcatttgagttcatgggcaggcaccctttaatttgttctagagctgagttgattgagggctttaatcaccagggactgacttctagctcctgtcttgagaaagtattaactcttgttggtctgttcaaataacttagtattttgtatcactctgacaacattatctgcatgttttcagttgtttaaacatactctcagttgaatctttacacaaattagtttattttatttaattttctttactatagagatctctcgattctaccaaataacttcatttgaaaaagcaaggctgtggagatgggtaaaatcacctgccccacaagcatgaggacctgactttgaatccccagagcccatatataaagcagatacaggaggcaagcatctataatcccagcactcctccagggagatggaaggcagaggcaggaaagtccccagaagtctgtaggccactcagcctggtgtacacagcagaggcaacagaccccatctcaatcacaatgggtgttgagaatcaacactggatattgctgtctgatccccacgtgtgcaatgtagcatacacacttacacacatgcacatgtgtacgcatacacagatgtgcacaaagcactttctaggtagtgcagtcaacatggttacaatgttcaaaaatacaagggggtcatagagttccgttaccattctttgtttctgagtttccttttaaccaaagttatcagtttttagtaaaatgaactatgcactacatattcttatttaccaactagtatgttttagaggtatttccagatttttcaaaaaattcccaagcaatatattttagagattctttcagattggctatgtaaagcacttccatttgcctggtaatgtagttgcccactttgtagcaaatgggtactcccatggttaccaaatgtttgtgtctatttaaaaaatacaacaatttcaaataggcaatttcctgtgcatgctaaagtaggtgcaagatatgtcccagcaggtaaaaggctaggttaaaatcatcatgaattctaattttaattaatacttctgaactgttctcataggggcagaattaatgtacaaacagcaccagcttatgaaagctctccttttctccaactcagcatccttagcacagggtgccatcagactttttgacagttatcctttgggtcagtgtaagtaagtgtctttctaccatcactttaataaggagactttgttttatttgacaggaaaaatgaaacaaaaagagaagagaacccttatgactcgatggacattggaaaaggagaaagcaatgcaggattccaaaacagtgatgatgctcagacttccttttagcaaagcacttgtcatcttcctgtatgtaaatgctaacttcatagtacacaaaatatgagagtatacacatgtcattagctatcaaaactatgatctgttcagtaaacgttgtccaaagagcatcagacttgagtggacatcttcactgacattgctttcagtatttatttctgcctaaggatttgacatctcttctgtttattaatagagatgtttatcttagcataaaagagggaaatgtgcctttggcctcacagtctatccagggtgatatggttgggtaactggagttagaagatgagatgatgtctcttgggggcaagtgttggcttcggtgtggcatctgggctgtgaactggtgggactgttgaggttgagaatggtgctcgctggtcacttgaatccaagtgtgacgtcatgctctgtggcttctgccttcacacttctcacttcaagtactgtaggaatttgttcacagtaatacttgaaatggactgtcccttctttggaggtgcagttcaacggagaaagaagccagacatcaggtagagaccatgaccttttctcttccaaacttgatcaacatctctctaacaagacacagctagcacaggaaactccacgaacccagagcatgcctgtcagaaactacttccattattctcccattgtggagtaagggaaaattccagatgaatgctcgatctgtgagatgggtgcccagtctctgaaattgtttgtattttctcacagggtctgagcaatggtgaacacaaagccgacctcaataaatacttattagatttagacactcctct

SEQ ID NO: 23 - DNA, 199 bp, synthetic construct
  misc_feature 1..199: note = Human //XhoI/ (loxP)/ICEUI//NheI// Human
  source 1..199: mol_type = other DNA
catttatacatttccacacttacaactcaattttccaatggagctgttgatgaacctaatctcgaataacttcgtataatgtatgctatacgaagttatgctaggtaactataacggtcctaaggtagcgagctagcctaggttgcaaggcatgaaagatgcataattgtcaaagacttatatctttaattagacctat

SEQ ID NO: 24 - protein, 805 aa, synthetic construct
  REGION 1..805: note = 24. Humanized human/mouse ACE2 protein
  SIGNAL 1..17: note = Signal sequence of chimeric human/mouse ACE2 protein
  REGION 1..19: note = MISC_FEATURE - Mouse portion of chimeric human/mouse ACE2 protein
  REGION 18..740: note = MISC_FEATURE - Extracellular Domain of chimeric human/mouse ACE2 protein
  REGION 20..740: note = MISC_FEATURE - Human portion of chimeric human/mouse ACE2 protein
  REGION 741..805: note = MISC_FEATURE - Mouse portion of chimeric human/mouse ACE2 protein
  REGION 741..761: note = MISC_FEATURE - Transmembrane domain of chimeric human/mouse ACE2 protein
  REGION 762..805: note = MISC_FEATURE - Cytoplasmic Domain of chimeric human/mouse ACE2 protein
MSSSSWLLLSLVAVTTAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIIFGVVMALVVVGIIILIVTGIKGRKKKNETKREENPYDSMDIGKGESNAGFQNSDDAQTSF

SEQ ID NO: 25 - DNA, 2418 bp, synthetic construct
  misc_feature 1..2418: note = Humanized human/mouse ACE2 CDS
  sig_peptide 1..51
  misc_feature 1..57: note = Mouse portion of CDS encoding chimeric human/mouse ACE2 protein
  misc_feature 52..2220: note = Extracellular domain encoding portion of CDS encoding chimeric human/mouse ACE2 protein
  misc_feature 58..2220: note = Human portion of CDS encoding chimeric human/mouse ACE2 protein
  misc_feature 2221..2418: note = Mouse portion of CDS encoding chimeric human/mouse ACE2 protein
  misc_feature 2221..2283: note = Transmembrane domain encoding portion of CDS encoding chimeric human/mouse ACE2 protein
  misc_feature 2284..2418: note = Cytoplasmic domain encoding portion of CDS encoding chimeric human/mouse ACE2 protein
  source 1..2418: mol_type = other DNA
atgtccagctcctcctggctccttctcagccttgttgctgttactactgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattatttttggtgttgtgatggcactggtagtggttggcatcatcatcctgattgtcactgggatcaaaggtcgaaagaagaaaaatgaaacaaaaagagaagagaacccttatgactcgatggacattggaaaaggagaaagcaatgcaggattccaaaacagtgatgatgctcagacttccttttag

SEQ ID NO: 26 - protein, 17 aa, synthetic construct
  REGION 1..17: note = Signal sequence of chimeric human/mouse ACE2 protein
MSSSSWLLLSLVAVTTA

SEQ ID NO: 27 - protein, 723 aa, synthetic construct
  REGION 1..723: note = Extracellular domain of chimeric human/mouse ACE2 protein
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 28 - protein, 21 aa, synthetic construct
  REGION 1..21: note = ransmembrane domain of chimeric human/mouse ACE2 protein
IWLIIFGVVMALVVVGIIILI

SEQ ID NO: 29 - protein, 44 aa, synthetic construct
  REGION 1..44: note = Cytoplasmic domain of chimeric human/mouse ACE2 protein
VTGIKGRKKKNETKREENPYDSMDIGKGESNAGFQNSDDAQTSF

SEQ ID NO: 30 - DNA, 21 bp, synthetic construct
  misc_feature 1..21: note = 90034mTM Fwd
  source 1..21: mol_type = other DNA
ggcaacctttcctgctaagaa

SEQ ID NO: 31 - DNA, 24 bp, synthetic construct
  misc_feature 1..24: note = 90034mTM Probe (BHQ)
  source 1..24: mol_type = other DNA
cggagccaatgaagggttccatga

SEQ ID NO: 32 - DNA, 23 bp, synthetic construct
  misc_feature 1..23: note = 90034mTM Rev
  source 1..23: mol_type = other DNA
gctgcagaaagtgacatgatttc

SEQ ID NO: 33 - DNA, 23 bp, synthetic construct
  misc_feature 1..23: note = 90034mretU Fwd
  source 1..23: mol_type = other DNA
ccctcctcctccagtgtatcttt

SEQ ID NO: 34 - DNA, 20 bp, synthetic construct
  misc_feature 1..20: note = 90034mretU Probe (MGB)
  source 1..20: mol_type = other DNA
cagcttttaaggaacatatt

SEQ ID NO: 35 - DNA, 24 bp, synthetic construct
  misc_feature 1..24: note = 90034mretU Rev
  source 1..24: mol_type = other DNA
gctaacattttatggccaaatcaa

SEQ ID NO: 36 - DNA, 20 bp, synthetic construct
  misc_feature 1..20: note = 90034mretU2 Fwd Primer
  source 1..20: mol_type = other DNA
tcgcgagaagcaaggagatc

SEQ ID NO: 37 - DNA, 26 bp, synthetic construct
  misc_feature 1..26: note = 90034mretU2 Probe (BHQ)
  source 1..26: mol_type = other DNA
tctgtgcttggaaaagcatcattgcc

SEQ ID NO: 38 - DNA, 23 bp, synthetic construct
  misc_feature 1..23: note = 90034mretU2 Rev
  source 1..23: mol_type = other DNA
ggagagaagagctggtgtagatg

SEQ ID NO: 39 - DNA, 22 bp, synthetic construct
  misc_feature 1..22: note = 90034mretD Fwd
  source 1..22: mol_type = other DNA
cgttgtccaaagagcatcagac

SEQ ID NO: 40 - DNA, 27 bp, synthetic construct
  misc_feature 1..27: note = 90034mretD Probe ((BHQ)
  source 1..27: mol_type = other DNA
tggacatcttcactgacattgctttca

SEQ ID NO: 41 - DNA, 20 bp, synthetic construct
  misc_feature 1..20: note = 90034mretD Rev
  source 1..20: mol_type = other DNA
gaggccaaaggcacatttcc

SEQ ID NO: 42 - DNA, 24 bp, synthetic construct
  misc_feature 1..24: note = 90034mretD2
  source 1..24: mol_type = other DNA
tctggctataaggaagcaaggatg

SEQ ID NO: 43 - DNA, 28 bp, synthetic construct
  misc_feature 1..28: note = 90034mretD2 Probe (BHQ)
  source 1..28: mol_type = other DNA
agagcaactggagttcagagatccaagg

SEQ ID NO: 44 - DNA, 23 bp, synthetic construct
  misc_feature 1..23: note = 90034mretD2 Rev
  source 1..23: mol_type = other DNA
ccactgcctatggaacaattaca

SEQ ID NO: 45 - DNA, 22 bp, synthetic construct
  misc_feature 1..22: note = 90034mretD3 Fwd
  source 1..22: mol_type = other DNA
gggatttgccacttgatccttt

SEQ ID NO: 46 - DNA, 25 bp, synthetic construct
  misc_feature 1..25: note = 90034mretD3 Probe (BHQ)
  source 1..25: mol_type = other DNA
cccttgagtagagaagaccctggcg

SEQ ID NO: 47 - DNA, 21 bp, synthetic construct
  misc_feature 1..21: note = 90034mretD3 Rev
  source 1..21: mol_type = other DNA
cagccaccaggacatctacac

SEQ ID NO: 48 - DNA, 24 bp, synthetic construct
  misc_feature 1..24: note = 90034mretD4 Fwd
  source 1..24: mol_type = other DNA
actgacatgagagtgctgaaggtt

SEQ ID NO: 49 - DNA, 17 bp, synthetic construct
  misc_feature 1..17: note = 90034mretD4 Probe (MGB)
  source 1..17: mol_type = other DNA
atgtggaggaagaagat

SEQ ID NO: 50 - DNA, 26 bp, synthetic construct
  misc_feature 1..26: note = 90034mretD4 Rev
  source 1..26: mol_type = other DNA
acaaaagatgctcaggtagaaatgaa

SEQ ID NO: 51 - DNA, 23 bp, synthetic construct
  misc_feature 1..23: note = mGU
  source 1..23: mol_type = other DNA
ggatgggatcttggcgcacgggg

SEQ ID NO: 52 - DNA, 23 bp, synthetic construct
  misc_feature 1..23: note = mGU2
  source 1..23: mol_type = other DNA
tcttggcattttcctcggtgagg

SEQ ID NO: 53 - DNA, 23 bp, synthetic construct
  misc_feature 1..23: note = mGD
  source 1..23: mol_type = other DNA
aagtctccttattaaagtgatgg

SEQ ID NO: 54 - DNA, 23 bp, synthetic construct
  misc_feature 1..23: note = mGD2
  source 1..23: mol_type = other DNA
agagaacccttatgactcgatgg

SEQ ID NO: 55 - DNA, 396 bp, synthetic construct
  misc_feature 1..396: note = 90034 Border A
  source 1..396: mol_type = other DNA
atctgtcctctccaggattaacttcatattggtccagcagcttgtttactgttctcttctgtttcttcttctgctttttttttcttctcttctcagtgcccaacccaagttcaaaggctgatgagagagaaaaactcatgaagagattttactctagggaaagttgctcagtggatgggatcttggaaaaggagaaagcaatgcaggattccaaaacagtgatgatgctcagacttccttttagcaaagcacttgttatcttcctgtatgtaaatgctaacttcatagtacacaaaatatgagagtatacacatgtcattagctatcaaaactatgatctgttcagtaaacgttgtccaaagagcatcagacttgagtggacatcttcactgacat

SEQ ID NO: 56 - DNA, 24 bp, synthetic construct
  misc_feature 1..24: note = hEx3-4 Fwd
  source 1..24: mol_type = other DNA
aacccagataatccacaagaatgc

SEQ ID NO: 57 - DNA, 21 bp, synthetic construct
  misc_feature 1..21: note = hEx3-4 Rev
  source 1..21: mol_type = other DNA
gcccagagcctctcattgtag

SEQ ID NO: 58 - DNA, 29 bp, synthetic construct
  misc_feature 1..29: note = hEx3-4 Probe
  source 1..29: mol_type = other DNA
ttgaaccaggtttgaatgaaataatggca

SEQ ID NO: 59 - DNA, 21 bp, synthetic construct
  misc_feature 1..21: note = hEx16-17 Fwd
  source 1..21: mol_type = other DNA
ttgaaaaggccatcaggatgt

SEQ ID NO: 60 - DNA, 22 bp, synthetic construct
  misc_feature 1..22: note = hEx16-17 Rev
  source 1..22: mol_type = other DNA
gtcattcagacggaaagcatca

SEQ ID NO: 61 - DNA, 14 bp, synthetic construct
  misc_feature 1..14: note = hEx16-17 Probe
  source 1..14: mol_type = other DNA
ccggagccgtatca

SEQ ID NO: 62 - DNA, 23 bp, synthetic construct
  misc_feature 1..23: note = mEx11-12 Fwd
  source 1..23: mol_type = other DNA
gagcctctgcctcatgatgaaac

SEQ ID NO: 63 - DNA, 22 bp, synthetic construct
  misc_feature 1..22: note = mEx11-12 Rev
  source 1..22: mol_type = other DNA
tgcttgacaaagagcttcttga

SEQ ID NO: 64 - DNA, 23 bp, synthetic construct
  misc_feature 1..23: note = mEx11-12 Probe
  source 1..23: mol_type = other DNA
tgtgaccctgcatctctgttcca

SEQ ID NO: 65 - DNA, 23 bp, synthetic construct
  misc_feature 1..23: note = mEx17-18 Fwd
  source 1..23: mol_type = other DNA
ggttggcatcatcatcctgattg

SEQ ID NO: 66 - DNA, 24 bp, synthetic construct
  misc_feature 1..24: note = mEx17-18 Rev
  source 1..24: mol_type = other DNA
tcgagtcataagggttctcttctc

SEQ ID NO: 67 - DNA, 27 bp, synthetic construct
  misc_feature 1..27: note = mEx17-18 Probe
  source 1..27: mol_type = other DNA
tcactgggatcaaaggtcgaaagaaga

SEQ ID NO: 68 - DNA, 3175 bp, Mus musculus
  source 1..3175: mol_type = other DNA
gcctttcctggccgttccctccttctggccgaggtgcctgcgtttaggggtgtcaccctggctcccgggacgccgcctccggagatttaagcgagaactggagtaggtcgtgtacttggagcggacgaggaagccaagagctcggacagaggcggagaggggcgggaagcgcaacaggtcacctggaggaagccccatactgacctcctcatgctgctgacacaggcaggatggcattgaactcagggtcacctccaggaatcggaccttgctatgagaaccacgggtatcagtctgagcacatctgtcctccgagaccaccagtggctcccaatggctacaacttgtatccagcccagtactacccatctccagtgcctcagtatgctccgaggattacaacgcaagcctcaacatctgtcatccacacacatcccaagtcctcaggagcactgtgcacctcaaagtctaagaaatcgctgtgtttagccctcgccctgggcactgtcctcacgggagctgctgtggctgctgtcttgctttggaggttctgggacagcaactgttctacgtctgagatggagtgtgggtcttcaggcacatgcatcagctcttctctctggtgtgacggggtagcacattgtcccaacggagaagatgagaaccgttgtgttcgtctctacggacaaagcttcatcctccaggtttactcatctcagaggaaagcctggtatcccgtgtgccaggatgattggagtgagagctacgggagagcagcatgtaaagacatgggatacaagaacaatttttattctagccaagggataccagaccagagcggggcaacgagctttatgaagctgaatgtgagctcaggcaacgttgacctctataaaaaactctaccacagtgactcatgttcatcccgcatggtggtttctttgcgctgtatagaatgcggggttcgctcagtgaaacgccagagcaggattgtgggtggattgaatgcctcaccaggagactggccctggcaggtcagcctgcacgtccaaggcgtccacgtctgcggaggctccatcatcacccccgagtggattgtgacggccgcccactgtgtggaagaacccctcagcagcccgaggtactggacggcatttgcgggaattctgagacagtctctcatgttctatggaagtagacaccaggtagaaaaagtaatttcccatccaaattacgactctaagaccaagaataacgacattgctctcatgaagctgcagacacctttggcttttaatgatctagtgaagccagtgtgtctgccgaacccaggcatgatgctagacctagaccaggaatgctggatttcggggtggggggccacctatgagaaagggaagacctcggacgtgttgaatgctgccatggtacccttgatcgagccctccaaatgtaatagtaaatacatatacaacaacctaatcacaccagccatgatctgtgccggcttcctccaggggtctgtcgactcttgccagggagacagtggagggccgctggttactttgaagaatgggatctggtggctgattggggacacgagctggggctcgggctgtgccaaggcactcagacctggagtatacgggaacgtgacggtatttacagattggatctaccagcaaatgagggcgaacagctaatccacgtggctttgtcccagacttcctttgtcttcaacaaccttctgcaagaaaaccaagggcctgaattttaacttcctgtgcacaatgtaccttttgagatgattcgaagggcctttcacttttattaaacagtgacttgtttgactgtgctccctggtcctgtgagggcttcagtgccccacccctgggccacttctgcagctcccaccagaatggatgaccagattctgttgggtttgggcacatagggccaaaggcagaggagggtggcactctcatgttggaacttcttttgggctcatgctcaggccttttttggatcactaaggactatgacctctgagtaacctgatgacctgagaaagagtaaggaggccaggcagggccttgggcccaggaacaggtaccttgagagtgagagctacccattgcctgtggcctaaatctgctgtgcaggttgggctggtcatactgtcatgatttcattaacagcctgggtgaacatggctgggagtaaagggcttgctctcctgcatgttgacatgacggccctttccaagggtgatggaggctttcccaagctaagggcctaggcagatctctcagagcaagaagctaatgccggcatgtcccttgggtgagctctacatggtgttattcagtctggttcttggctccccactactgtttctctcagcctctcagagcctgaaacttacctcttagctttggctacaggcatggcctagtacctgatggagcctgtatagctcagctaatcaaatggaggctcaggtccatcagaatcagggacttgtgatttcagtcaccttgcttctgggttgtgtttcttctcttactacctcactgcacctggacactagagtggatgaatgtctggagttcacctgcatttggactgtgtgattgtgcctcagacactagacctcttccagatggttaggttgttctgtagactggcaatgagattagaagttcctagcttcagataaagatgaaagagaggagatcattgtcttctgtcttcttctggccctgggtttataccaggaaagccatgccagaattaccaaatatgaagtatgaatgtcttacccacggtgaggctctgcctccttctctctgcctggttcttcagaaggcagtgaatgggtcataactgggactccatctttgctggggaaagtctcccacctagggaatggttaccactccatgtaaagaaaactccctcatgcgtcctctgggaccttcttagatgctgtaaggtacctacatacagactaaatgtgcaagcaccttgaagtgtgagaacctgtcccctccttagctctccttgtctttgctgttggttggttatttcctgctttgtgtctgttctgagctgtgagattccactgtgaaatatatgaataaagtatataattcttttaaaaaaaaaaaaaaaa

SEQ ID NO: 69 - protein, 490 aa, Mus musculus
MALNSGSPPGIGPCYENHGYQSEHICPPRPPVAPNGYNLYPAQYYPSPVPQYAPRITTQASTSVIHTHPKSSGALCTSKSKKSLCLALALGTVLTGAAVAAVLLWRFWDSNCSTSEMECGSSGTCISSSLWCDGVAHCPNGEDENRCVRLYGQSFILQVYSSQRKAWYPVCQDDWSESYGRAACKDMGYKNNFYSSQGIPDQSGATSFMKLNVSSGNVDLYKKLYHSDSCSSRMVVSLRCIECGVRSVKRQSRIVGGLNASPGDWPWQVSLHVQGVHVCGGSIITPEWIVTAAHCVEEPLSSPRYWTAFAGILRQSLMFYGSRHQVEKVISHPNYDSKTKNNDIALMKLQTPLAFNDLVKPVCLPNPGMMLDLDQECWISGWGATYEKGKTSDVLNAAMVPLIEPSKCNSKYIYNNLITPAMICAGFLQGSVDSCQGDSGGPLVTLKNGIWWLIGDTSWGSGCAKALRPGVYGNVTVFTDWIYQQMRANS

SEQ ID NO: 70 - DNA, 3212 bp, Homo sapiens
  source 1..3212: mol_type = other DNA
gagtaggcgcgagctaagcaggaggcggaggcggaggcggagggcgaggggcggggagcgccgcctggagcgcggcaggtcatattgaacattccagatacctatcattactcgatgctgttgataacagcaagatggctttgaactcagggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcaggtgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcatgcaatcccatttgcaggatctgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcaatttttttttcaaagtaaaaaaaaaa

SEQ ID NO: 71 - protein, 492 aa, Homo sapiens
MALNSGSPPAIGPYYENHGYQPENPYPAQPTVVPTVYEVHPAQYYPSPVPQYAPRVLTQASNPVVCTQPKSPSGTVCTSKTKKALCITLTLGTFLVGAALAAGLLWKFMGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG

SEQ ID NO: 72 - DNA, 27947 bp, synthetic construct
  misc_feature 1..27947: note = Recombinant polynucleotide
  source 1..27947: mol_type = other DNA
gcagagtctaagaaatcgctgtgtttagccctcgccctgggcactgtcctcacgggagctgctgtggctgctgtcttgctttggaagttcagtaagtgcagggagcctcgatcccaccatgtgctcctgcagtccccagtgctctgagccagaccctgctctctgggctattgagacctctggaggccctccgtgaggttcctctcttacataacgaggctgtctctcttcccttctcttgtttagctatgagattgacacatcatggggaaagcatttagaatgtacccagtgctttggggtgcttggtgccacccagcactgtgagcacaggttcttctaccttggggccacacccagttacctgtatctcactgcacagcagtggctgttggggaccaggcccacccctccatgtcccacctcctgcaactgcagcctgagccttcccatcagcctggggtggtgcagacccatgtgccattgtggatccttcaagttacctgtgtggcagagaggacgtgtgagtgccgtccaaacccaaacactgagagggtccttcccattgcccccacggaagtaaggtgccccagtgctaattccacttatacttgctggtggcaaggacacttctcctccttattaaagtgggggattggctgggtgaggtggctcacgcctgttatcccagcactttaagaggccaaggcaggtggaccacctgaggtcaggagtttgagaccacaagcctggccaacatgttgaaactccatctctactaaaaatacaaaaattagtcaggcgtggtggcgtgcacctgtaatcccagctacttaggaggctggggcaggaggatcacttgaacccaggagttggaggttgcagtgagccaagattgtgcccctgcactccagcctgggtgacagaatgagacttcatctcaaaaacaaaacaaaacaaaacacagtggggccaggagttggaggctgcagcgagctacagtaatgccacggtgttcctcactccatgaggctcattgcgtttctcagcctgaagggcacctctcttctgttttctctgcaagtgggcagcaagtgctccaactctgggatagagtgcgactcctcaggtacctgcatcaacccctctaactggtgtgatggcgtgtcacactgccccggcggggaggacgagaatcggtgtggtgagtcagccttgaccttgggaagggactcctctgctcaccttggagacagcagccgggtccaggggcctttgggtgactgggcctggcgtgcgtccagtacgctgacacatgatgtcattgaatccctgctccaggctgagccctggggctcagagaggttgtgtttccggcccaacctcacccagcaggtgggagatgacagggccaccgaggactgtgtcattggaaccacacgtgctctgaactgccacaggaagtcagttaagatgagcaaactgtttataaagttggagatgcaggctaggaacggtggctcatgcctgtaatcccagcactttgggaggccgaggcagatggatcacctgaggtcaggagtttgagaccagcctgaccaatatggtgaaaccttatctccactaaaaatacaaaaattagccaagcgcggtggcgggtgcctgtaattccagctattcaggaggctgaggcaggagaatcacttgaacctgggaggcggaggttgcagtgagctgagatcacgccactgcattccagcctgggagacagagctggctcaaaaaataaattaattaattaaaaacaaaattggagatgcactatgttattttcaaaacaagctgcctttaaagatctatctgttgtcacagggtgggctcatctgtttcattttattttctgtggtttatctatttattcattttaatgaactaggaagcattgctcctatttatggcataccacatgatgtttggatacgtgtatgcctgtggcatggctaagtcaagctagaacatgggccttacctcatatacgtgtcttattaagaacacataaaacctactcttgtagtgattttcaaatatgcaacatatagtttattaactgcagtcactatgatgtacaatagattgctcgaacttattcctcctgtctaactaagattttgtgacctctgaccaacatctccccagtgttgtcaccccccgcccccagcctctgatagctgcctttctactctctgcttctgtgagtttgatgtttatacattccacatgtaagtggcctcatgcagtgtttctgtctctgtgtctggcttgttcacttagcgtaatgtcctccagcttcatctatgttgttggaaatgacaggatttccttctttcttgtggctgaatagtattgccttgtgcatatacaccacattttctttatcccttcattcactgatggactcttaggttgatgtcatgtcttggctgttgtgaaaaatgccgcagtgagcgtgggcgtgcaggtccctcttcaacacacggatttcctttcctttggatataaacccagcagtgagattgctggatcacatggcagttctgtttctcaccttttgaggaaactccatactgttttccataatggctgtagcaacttccactcccacccccacggtgcaaagtctccatttctcttctacaacctcaccaactcctgttattttccatctttctgatagtagccatttgaagaggtatgagatgatacctcattgtggttttcatttgcatttttatttgtatttttcatgaatttttgagggtgatttcaagggtagttagtgactcgaacagggaaacgatcctgagtatgagggttgtgctaatcatccccctcctgccagctgcgtacggaatggggctctgcagatggcagggagctggctcgtttctctttaagagctgccttttacttttcttcctcttcctttaaaacttatttcctggccggacgcagtggctcatgcctgtaatcccagcactttgggaggccgaggtgggcggatcacgaggtcaggaattccagaccagcctggccaacatggtgaaaccccgtctctactaaaaatacaaaaattagccagacgtggtggtgcgggcctatagtcccagctactcgggaggctgaggcaggagaatcacttgaacctgggaggagggggttgcagtgagccgagattgcgccactgcactccagcctgggcgacagagccagactccatctcaaaaaacaaaaaaaagttatttcccaagcacagccatgtattccaggcttgtggatcagcgttggtggtggtgtgtgctctcatatcttagttccagctaagcacactctgacatgtttacactagaaccatttgttttttctagaaatagaaatttcagaattgtagagtcagaggacttaccagaaatctcttaggtagttctcctcccctccctcaagtgcagtcctaacctcctggagttttctgtagaaaccacaagcctcagagctggccgagaattctagccaaagatttttccatgccaaagtaatcccccctctcctaagggccatccttggtggggactggtttcctgttaagccctcgctgtcagtcctggctgtggaatttcctggtgaggagcactggcccgtggagctcggccctcgtgccggccttgagcaggcccaagtgttccgtgttcttgatacctttcctccagcacagtcttgcttcccagaaaaaggtttgcacttgaaaatgatgcatttgctgattaaacatagttcttttgctttatttggtttctaaaataaagtgggagtttttgagattgagtaacgtgaggttaagatagcacgtggaatggctttttcttttctttctattttttttttttttttcctggagacagggtttcactctgttgcccaggctggagtgcagaggcatgaccatggctcactgcaacttcgatgtcctggggttaagcgatcccccagcctcagccccccaagtggctgggactacaggtgctcgccaccacacctggctaatttttgtattttttgtagaaaatgggtttcatcaatgttgtccagactggtctcgaactcctgacctcaagcaattctcctgcctcagcctcccagactgctgggattacaggcgtgaactaccacgcctggcctggaatggcttttgatgttctcctatgtgcacatgtgggtgaataaacaccaacaaagtccttatgttacctgaagagttgctctcttcttaatatttaagtcgtatttatttaaatactttaatagttgtacactattaaagtattattaggtcaaaatcaaggaagtacaaaagggtatgctgtgaaaaatctcttcttccttgctctgcttacttacctaccccgcatccccccatacaccccagacacacacacacacacacacacacacacacacacacgcatcactcccatacatgcccacctgtttaccagccaatcacatttcttggggcaactcatctgagttgcttctctttccagagagtttttgcataaagaagcacaggtatttctgcgttaccatgaccctatttcccagtggttcctagccagttgactctcctgcactggataccatcctggacagcattccttagggaaatgagccccctgttttttcccaccatggcacagttggtcctttgcatggacgcaccattattgcccctgtctcttcttggtggaccttaaggttttctccatccttttgctgtaacacacactgctccaagtgtgtgagcatatcagtaggaaacgcttccaggagtagaactgctaggtcagagggcgtgtggatctgtaacctgacagacctagaccggcttcagtttggttttatccagtttccatattgattattcatataaaaggaaacagacaaacataacgctgtgcatgtattctctcttagaccagaacaggcatagggtgcacttttaatttgtccatttcgtagagtagaaattgtttttgctgaaatgaacaccttaggatgctgaagaatatgacccgtcccatggaaaacattcaaaaatgtgtgtagcgctttcttcccaagggtgtgtgtgcgcatattttaacactaattcactttctacttccgttgctatcctttctgtgagtctttctcagaatctcagaaaagaaactaaattgttcactctagttatcaatgctgtactctatacctggaatttgctaaaagggcagattttaagtattctcaccacagaaaagagaaaagaaaatggtaattatgtgacgtggtggacatgttaactagctttattatggtgagcatttcacagcggatatccagtcatcacgctgtacacattaaacatgtacaattgggtttttttgagacaaggtctccttctgtcacccagtctggagtgcagtggctcagtcatggctcattgcagcctcgacctcctgggctcaatccatccttccccctcagcctcctgaaaagctggggccacaggcatgtaccatcatgccaggctaatgcatatatatttatattttttggtggagatggggttggtctcgaactctgggctcaagtgatcctcccgccttgcccttccaaagtgctgagattacaggcatgaaccacagcaccaggcctacatgtaaaatttttatttgtcaactatactttgacaaagctgagaaaaaaaatcctaatatttaaaaaaaaaaaaaaaaggactagcttgagaccttttccagctctctggcttatcagctgccgtctcttccgggtgcagatagctggaagggaaagaaaatccctaaaattacccacaagccaagaatgaagtgtctccctttgagccacagtggcagttttgtttttaatcatagaagtgtattttgagccgggtgtgctggctcacgcctgtaatccccgcactttgggaggccgaggtggggggcggagggggtggggatcgcctgaggtcaggagttcgagaccagcctgaccaacatggagaaaccccgtctctactaaaaatacaaaattagccggcgtggtggtgcatgcctgtaatcccagctactcatgaggctgagtcaggagaatctcttgaacccaggaggtggaggttgcggtgagctgagatcatgccattgcactccagcctgggaacaagaaaaaaaaagaagaagaagaagaagtgtattcatttcagttacttttaaaaaagtgaacagactttatattttagagcggttttaggtttacagaaaatgaaacagacagggcagcgagctccttgtactcctccccagcacacagttgccctgttatgaacatcccacatcagtgctgtgcgttcattaacaccgatgaacctgatgcatacattatgatgaactgaagtcctggacttcaccctttctcttgtacagttctgtgggatttgacaaatgcataatgctgtacagccacaatgatagtatcgtccagagtagttctcctgccttaaaacctcttttgctgcacctgtttctctctccccactcaccccagctatctgatcttcttagtgcctccgaagttttggtcttttcaggatgttgtagcgttggaatcatggagtatgtagccttcaccacatacaccttccttcactttgttggcttcctttacttagtaatatgcattcaagtttcctccatgccttttcatggcttgatagctcatttctttttagcaccaaataatattccgttgtccagatgtagcacaatgtttatccattcatgtaacctgtgaccgactcacagataggatgtggaatcactcaccacagaggcattagacaataatcagacccaagtcatttcatgggggaacaagcccacaggtaccagactgtccagtgagtcagggccactcgtaggaagtaagaagagaggctagagcatagccaggtcctcactttatactttaagcccatgtgtatttctcccaaaccacacagcattgtttccatgctttcagctttgcatgaataacgtgatacttgaacgcatcatttatcacttgctctctttcccacagcgctgttttcaagcttcttcctgttcatgatgctctgcttaacccttaagctgcatgggattctgttctgtgaatacgcccaccccatgtattatcctgcccagcaaaaagtccccaaaactctggatggtggttacctctagggagggagagaagagattgggaatagggagcgacttcaacggtgtttgtaatgttttgtttctttaaataaaagagctgagatcatttcagcagaatgttgatttagagtctcctggacaatttgttgctcaaagtgctctcttaaagagcactttaaaaaaaaaaaccttttatcttattatttatttatttatttattgagacggagttttgctctgtcacccaggctggagtggagtggtgtgatctcagctcactgcaacctttacctcctgggttcaagcaattcccctgcctcagcctcccaagtaggtgggattacagatgcgtgccaccacacttggctaatttttgcattttagtagagatcggtttctccatgttggccaggctgatctcaaacgcctgacctcaggtgatctgcccgccttggcctcccaaagtgctggtattacaggcgtgagctaccatgcctggcttatcttatatatttttaaaaacagcttattgagatctaatttatgtaccataaaattcaagtatataattcagtgcttttatatataaaacatatatatgaaatagcttattgagatataattttttatataaaacagcttattgatatgtaatgtatgtaccataaaatttaaatatataattcactggcttttatatattcacgaatatgtgcaactatcaccacagtcaattttagcatattttcatcagctcataaagaaaccccaagcccttgaactatcaccccatatccctcctcccagcccgtccctcctactcataagcaaccactaatctacttagtgtctatagatttcctactctaggcattccatgtgagcgggatcatgcaatacgtgggctcacacaatataagtggcattccatgtgagtcggctcatgcagtatgtccggctcctttcactgagcataaggtcttcagcactcatccaggttgcagcctgtgtctgaatttcattccctcttctggctgaatcgtattccattgtgtatcttggacatatcctattctgctcacccagccgttggtgggcgtttggagtgttttcgcctttcagctgttttaagagggttgcagtgaacatttgtacaagttttggacccaatgcctgttttcaattctcttgtgtagagagcactttttagcagaaaaagaatagatttgtggcctccctttgtgtgcggtcagtgccttgagaagagtgaactgtgctgccacctccggagccgtggagagcgcggggcttgggtagcagctaggacgatacaagttgggacaaggccaggtgcaatggctcacgcctgtaattccaacactttgggagaccgaggcagggggatcacctgaggtcaggagttcaagaccagcctggccaacatggtgaaaccccatctctaataaaacagaaaaattaactggacggggtggtggacgcctgtaatcccagctactcgggaggctgaggcaggagaatcacttgaacctgggaggcggaggctgcagtgagtggagatcagaccactgcacttcagcctaggtgacagagcgagactccgtctcaaaaaaaagaaaaaaaaagaaagaaactcatggataatcctccctctcgtgcagttcgcctctacggaccaaacttcatccttcaggtgtactcatctcagaggaagtcctggcaccctgtgtgccaagacgactggaacgagaactacgggcgggcggcctgcagggacatgggctataagtgagtatggggcagcacccgccgagtgacagtaacagacagcagaaacacgagaagaccctctctctgcctccctgtgaaagcaccggcacatgagtgctggggacaattgtcaccttccaaaagctgagccctataaccagcaggtggaatttgtcctgctagggctgtgcccagcacacagaccttggctcactgccaccttgccctgcctcctccttggcctctatagactcctggttgctcgggagtgcccagtgctgtggtcatctggtcagaggggtaggctgagggcgttaggtgcctctttttccaaggtgcctctcagccagggtccattcacctccctgggtagaggttggaccagaacagctggcgaggagggttgggctggggagagcagcagagacaaatcctgtgccagtttcacttcattcgggagccatggaagccttttgagctggggagagaatcaatcaatcagactgatacttaaaaaatgtcattcctgctcgtagctctgagggaaggtgggaaggcttaacagggtgtgtgtcgcctgacagtgattcctaacgggggtggggcggtggttaccatttaccagcactgcctggggagatgcggcagccctcaggcatcgggggagagggtggtaggatgctactgccactttgttttccatgggagggtccccaggtgatttctatgcaactttagggtattcaatatgccagttttcagaatgaattaccactcggtgagaaagttggcatcttagctagtcactgtgacatccctaaacagcaggggtgaattacacagcaaagcccccccatcacagtccaggaacctggtggaattgataactggggccatgttaacatctgtaccttttattagattaaatgtgtgtatgattatacaatcctatgtccttctcatagtttcttgatcctaacctggataagaaacacgaccaatgaaggaattttgtctgacactttagggttattgaatcgaaaaatcgttacaatattctagcacttggttagaacgtgtgattttttttcctaaatgctaaggtttttccctcttattctgaatgtcgtatgagcggtattatgacatagtataggatttgtgtttgcttatgccttaaccattatcacaaataaggttttcttttttaggaataatttttactctagccaaggaatagtggatgacagcggatccaccagctttatgaaactgaacacaagtgccggcaatgtcgatatctataaaaaactgtaccacaggtatgcagcaatttcttcttgaaaaattttggaatgaaatcaactaggagacaccatggggaatcgttgtcctgagtctgatttctctgagctgcaatactcggtctggatgggttttgcattgggaggagattagagtctgaccaggcctggttactctaagcagcccttggtttattcataggaagtggctgaggtttctctgctatttcattttcagcctctaccgtctgcccttgttggtagcggctcacacttgcaacatcgacattcaactctatttagttttctttcctcttcagacatttagaggtgtacctattttgtcagggcgtggttctaggaatccaagataatgtctcagtgtcccagccagggtgaccggctcattccagtttgccagggacttcactggcttgagcaagggaagtcctgctccattccaggcagctgggctggctggtcccgttagccccaaccccgggacagcagtgccagagggtgctctgtgagggatgggcagcattctggcggcctgggaatgagttgtggtgtttccagggggtagaagtgggtacaagccacaggtcacatgatgagtggctgacctggctgggagggcagaagaggggatggacttaggctcttccttttgctttgcacatatttaggatgtttgcagacttgctatgattgttgctgttatgtgttttctgatgtgaaagatacacagtgtcctttgcccatgagctctccttgcctcccaggtccccagggcttatgcctggtgtctaggcatcacctccctgcctgccaggtgccaggtgctgcatttcgggggaggatgaactaatcaccccgcgccacctttcctctgagtgggagcctggggcaggtttgcattcctggaggccgctggtggaggggtctgggggcctgacttccactgcagcctgctgtcctggggaatgtggcagggcaagcccagtggggagggctgtgcacggccaggtgcacccatcaaaacagcagggctgcggtttgtccctgtggagaagctaaacacagctgcctgggcactttgtaaatgctgagtggttctttgtctttctgggttacacacggaatcagggagccaagtccagccgggcagggacggggggaggggaggaggtgctgccgtcccttggcaagagccttgggaactcacaaggaggctggagggcttggaagaaagaagagaaggccattgtctggtaggctctattctatctcggtggtggtggtggggggaggcgcacttcttttcctctttctgtgcagcagttgccctttgatgcctgagttcttggcttgttttctgtcgggcttctgtgaataaccacatgtgccctggcgctgtgaccacacagggctatccctaccgaccttaggattcttaggaaatgtcttctcttaaaggggacatgtcttcacttggccgtgtcagtgccccagagccagagtccacctggaatgcacctgtagtcactgagaacccggggggtgtgccttagtaagaaggtgtcaggaaggacctattattgtagggcctgggctcctgcaaggtggtttgggggtggttggaggaagcagagatttgctctggattggatgctgtcaggaagcaggggtaattctgtgaggctgctttattattttttttctaggaggaggttggaatgaggctaggctaaagctgtgattggtaaagaaacgtccgtcgctcaagttagccaggacaggaggagacatcagatcgtgattttgtggttgtgagcacaaggttcctgttctgtctgttcagacatcatttcggaggaggctccttgtgtcttgccccatctcaggcatggaggggcctagtccgatattgacgctcagtgaaataattcaggttccgcagagcacacggcccagctatcagggcgggccagctctgcatgccaggggccgcgtcttcccttctcagcatagcctgggaaattcactgcaggacaaaatgcatcagttacttcctcttcatccataacctgggatgtttgactcccaaatgagtaactcttacgtttcttctaatcctagggaaactattggttatattgctttcaacactacaaatttaaagcagttataggagcccagaggtttccaaatggcttccttaaaaattagaagatgattttaaattccaagaggaaaaacaaaactagcattattgtatacttaccctcacaaccgtcctaggagctggtacaattttaagagaggttaagtaacttgcccaaggtcacactgtggggatgtgagccgcgtaccttggctcagtgtctggtctttgccactgtccctatatggatttacttaccttattggagttgtaactagcagacccttctatgtctcagaagacaggagagggaacatcggaagaaatgactgatttctaagcatgtgagaggcaggtgactccgcactatcgtgaccagaatttcccctgttctttttgcagtgatgcctgttcttcaaaagcagtggtttctttacgctgtataggtaagttcatctggagtcccccttttgatacttctaactaggaaaagctctctactttcagaacagtactccctgtgtctctgggggcgtgggagggaagaaggtggggtcacgggttggaatgtgcccagcggcgtctcgctctttccaaggagctcctggtttagatttccatggcctgtagacaccttcagccttgggtccaagggacaccccctgagatcaggcacgctcaagaagctgacaaagccctacactttatgccacccatgagctggaggcccggcaggtctctttctccagaaagcaaaggggggtggcgttagtgagccctggcagccacctaacgtggacttggagcatctgcggggctgtggtccagcaccaccgtgtggccaccaggtgctcatcagccagtgggacccgggaggagggacaagaccagagaacaacagtgctcttgcctcttctctcctgaattttggacggtggcttagacttgggtgtccccatctctgtgtttagagtgcttacagtttccaaactgtttgcaaatgtggaagccaccgtccctctcctctgggatggcccagtgctgtcgtggggccgtggtcctgagctcagcttttcatttgaagaggtggaaggagctgacaccgtcccatcccggcagggctggctcaggtcttctttaggtcctgagtgggggtccagcacagccccaagggtgcgtggcacccgccctgccctctgcccatgcactcatctcctggtggagaagacactcacacacaggaagcagggaaggcagcagacctcactcacccctcaccccctcactcaccccctactcaccccctcaacctctcattcaccacccaccccctcgccccctcactcaccccctcactccctcaaccctcactcacctcctcactccctcaaccctcactcacctcctcacctcctcactctccccctcatccctccctcaccccaccccgtcacctcctcactcacctcctcaccccctcactcacccttcaccccctcactcaccacctcacctcctcactcaccccctactcaacccctcattcacccctcaccccctcactcacccctgcaccccctcactcaccccttcatccactcacccacctgctcacctcctcactcaacccctcaccccctcactaatccctcactccctcaccccctcacgccctcactcacaccttcacctcctcactcaccccctcaccccctcaaccccttacttaccccctcactcatcccttcacccctcactcaccccctctctcacccattcaccccctcactcatgccttcaccccctcactcacctcctcactcacaccttcacccctcagtcaccccctcactcaccccttcaccccctcaatcatgccttcactccctcactcaccccttcaccctctgaattactccctcatcccctcactcaccccctcactcaccccttcaccccctcacccaccacctcacccacccctcacccaccccctcacctccttacccctcacccccctcactcacccctcaccccctcactcaccacctcacccacccctcacccaccccctcactcactccctcatcccctcactcaccccctcaccccctcactcaccccctcacccacccctcacccaccccctcaccccctcactcaccccttcaccccctcactcaccccctcactcaccccttcaccccctcactcaccacctcacccacccctcacccaccccctcactcactccctcaccccctcactcaccccctcaccccctcactcaccccctcatctcctcactcaccccctcacctcctcactcacccgctcacctcctcactcaccccctcgccccctcactcacccctcaccccctcaccccctcactcacccctcaccccctcgccccctcactcaccccctcgccccctcactcacccctcaccccctcaccccctcactcatcccctcacctcctcactcaccccctcacctcctcactcaccccctcacctcctcactcaccccctcacctcctcacccaccccctcactcactccctcaccccctcaccccctcactcaccccctcacctcctcactcaccccctcacctcctcacccaccccctcactcactccctcaccccctcaccccctcactcaccccctcacctcctcactcaccccctcacctcctcactcaccccctcacctcctcactcatgccctcaccccctcactcaccctttcacctccttgctcatcccctcacttaccccctcacttcgtcaatcacccccccacctcgtcaatcaccccctcaccttttcactcaccccctcactcacccccttacttcctcacttacctcctcaccccccactcaccccctcaccccccactcaccccctcaccccacactcaccccctcaccccccactcaccccctcacccctctcacctcctcactcaccccctcacctcctcacttatcccctcaccccctcaattaccccctcaccccctcaattactccctcatcctttcaattacccactcaccccctcacctcctcactcctcactcactccctcactcaccccttcaccttctcactcacctcctcgtctcctcaccccctcactcacttccagccctgcccctcccatcttccttttctttgtgtgagaatctggggtccctgagtggtgtcagtccctccaagactcaaggagtccccagggccttgttatccagaacacccccacctgggtcccgggagaccccatgggatcacaggagtgttcagggaagtggtgcttcctgggtctgggtgggctggaggggcatcctcccttccccaagaggagacccccaggagccccctaagtccatccccagcagtggtgcccctgccctgtccttgcagcctgggagacccttgggaggggcgggcgctgggtggctgggcggcttctgctggtctcaccccactggcctcctgtttgtcatcctcagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaagtatgccaggggcggcgcgggccgggtgggggctcagggctggcctacagccaccctgtgaccttgagcaggtctcaacccttgcagccccggcatccttgtgtttaaatggggagagtattgcacctgcttcctagggctgtgagacatcaagtgcgctcatgccaggcagtgcatggctgtatgcactgagtgtcccctgcacgcagggcacagggtgcaggtggaacattctccacgatgtcgccgtgaccagcgttccttccagccactgtcctctgagctctgtcctgcccttgagcaaagcccctgccccctgaggtatcctgtctccgggacgctagtcccaggagagggcacactcagacaggcttcaggctgccctgctggaaggtccctggggttaagcgttcttggccacagcattgctcatgcagagggttaggtaggggtgaggctagccgtgacagtattagcatttatggacgctaccaccccctccccttttccttaaacacatagtgcttttggtcacatgctgctttggaggaggcctcacttggcggatgtatttttctgccttagagagaggctgaactgggtttgactgttggcccagccctctcttgctgcgtgcccttagacgattcactcaacgtctctgatccatggcatgtacaactataagatgggcatgcccttctcctctcgggctgttatgaaggtcaaggaagcaagggctgttacccaagggtgctcccttctctccccctcttcacacccccaggtgctctgggccctctaggaactgggtttctctcaagggctgttacccaagggtgctcccttctctccccctcttcacaccactgggtgctctgggcccactaggagctgggattctcttaagagggaaactcttggataaaggaaatggtttgattgatatcggacaagtctgttcattagtatccatttattaagcacctaccatgtgccaggaaatgctttggcgtacaaaggaaaataagggccagtcctgctagaaatggccttgaaaccccagggagggatgtcggcccattgtgggtgctgcagattccttgaaggtgatgcaagagccagaaagaaggatgatgtggggggctgaggcagggagtcggggttgggggagtgtgggggagaaggggagaccgagcacctcttccactatctccctgtgtggtttttggtgaaccatcctgcctctgggtgtcttgcctccagcttctgacgttggaagttcatccactgagagctctgtgtttatggctctgagatactgagtccttcttctctcccagacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacggtacgtgtggctcaggcttggcaagcaggttggcagaatcttaaagagatgttgattggaaatgacacttgtgctatgccaaatggaagggaggcatttgcgttgagcgagggtagcgtgcagcgggtggccaatgggagaggctcacagaggctaagagcacctgccgcattttgggggaggcagcagccaccacatctgttctgtactgtactgagtggtggtgattcaagccaggcatggaaaaggctagaacagggctttcccactgcagcacccttgacatctgggtggttctctgttgtagggctctcttgtgccttgtaggatgtttaacagcgtccccagcctctacccactggaggccagtagctaccaagctgtgacaaccagtgttgcctgctgacattgccaaacatccgctttgaggcaaagtcacttccagttgagaactactggcctaaaatgtgtaaagatccttgatttttaaagatacattctaaaaccaagttgcttaattcaggacaaacatgctttctcttagcctcttattcggtcccactctggtccatccaagggtctggaatgttctagccccatgtggatacagaagaagcaaaacctcagccctccctacagcatgtctgtattcacattgggaaatggttcacatatagaagagcgaatgcctgagcaatggcgtggtgcctctggggcgaaagctgactccattgactccatcggctttttggctgttgcctcctgtgtgtctttcccgtcttgatcacctggagatatgtaattttggaagcagagctagcaaataattcctcttataagcagagctagcaaataattctacttataagtagcataacgtcttgcctgccagaaggagaggtctggcagggggagaaagtgagaatgtgggacttgttgggatgcagggtcctctgggcagggtggccagggtgccaggcccagcagcctgcatgtgggaaggccaggtggagacataggtgatacccgcctggctcactgtgttttctcttcttgaaacagacctagtgaaaccagtgtgtctgcccaacccaggcatgatgctgcagccagaacagctctgctggatttccgggtggggggccaccgaggagaaaggtgaggctgctcctgggcacacaggactgcagggcccacagatggagcattgggttcggaagtgggaggtccaggttttaatcccagttctactactcaatgactggatgactttggttgattcccccagtccttgtgcctcagtttctccatctgctaagtgggagaaatcctgcccagcctacctaatacactgtgttcttatcgtgatcacacagagcagcatgtggaatggcttttgaagtatctgggccatacgagtttagaggtgcaggatctcctgtgttgcactcattgtgagtttagagctgccctggagatcccaccaaggcctgcgtggctgagtgacagggggcttggtgaggacgggcatcctggacccatggtggccacatctaagcctgtcctctgccctgataaccacagagagaggctctctccacccacttcctttgcaatctgcatttctctctgacagtctttcaaatgaagggagcctggctgcttcatttttatggagggttggaagtgcttagtggcaggcacaaaggttcattttacatattgtttatatccttctcaaaagcgtctaggccatacagacaacaaatcctttcaaacaaggggaaaagtacaaaggttgggtgatttctggggagcgtcagggaaggtagtggggggcatcctggctcctcatcagcagaaacttactacagtagagccacaggctgggcaaaagacctcatggaatccaagatgaagggaatatcgacaaatatttgtgcgcacctgcacctagtacaggctgggtgctactcaggtgctgggaatgcagaagtgaacagagtaagacaaatgtctctgctgtcaggagctttacctctcttctggatgtcggtggtggggacggggcaggtgtggtcagacagatgggagacaaacaactgagcgaggtacttccaaacatctgagggtggggatcacaaggtcccggctattttgaaggggtggtcaggaaaggcttctcggaagaggtggcatttgagctgagactcaaatggcaaaaatgtgtacacatcaaaaaggctagtgcatgtatcttcaggtgtggtcaaggggccaaggaggtgggctggggccagattgcataggtccttgtggattatggtgaagacaccagcttctcatctgcttgaggtggggagatcgtgagccggggagtgccatgatctggcagctgcgtggggagtggggatgaatggatggagacgaggatgatggtgacaagtccattgctgtggttccttgagacaggaagccagctcatagcagagtgcgggcgtggatgtgaagagatgagggtacactagggctagagccaccagacttactgatgggttgcatgtctgtgggagagagagtgagaagtcagggacgatggctttccactctgtggctgaagccccagggtggcgggtggtgccatttttcaagccaggaaatattggttggtgagaatttggggtgggagaaggtgtgacggagggttctggttttgcacactaagcccacggtgcccagaagatgcccgaggggaggcagcaaagcgagagtgggaaatgcagaggtggcaagtgcaggccgtgtcttgagaagctctaatgtgcaggggagccgagaagcaggcggcctagggagggtcacgtgtgctccagaagagtgtgtgcatgccagaggggaaacaggcgcctgtgtgtcctgggtggggttcagtgaggagtgggaaattggttcagcagaaccaagccgttgggtgaataagagggggattccatggcactgatagagccctatagtttcagagctgggaatttctttccctgaagctgaactccagagctgcattcagcacaggcaccgccagttgtaaggagaatccaggtttcccaggagaggggttggtgctgggatgagctgaccggggcagggctggaaaatagggctgtgaccatctgtgtagtgcgtgtggaggtctcagggagggaagtgtgctctccctgcgagagctgcaggcaacactgggagctcaacaagtctccctgtccttagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccaggtaattcaacatttttattctacctttggtccttaccagatcctactgaaccccccatgagagagagggcattcttggggtcagcagagcctcctcagtgacacggagccagctcggggcagtcatgggaagtgacggccacaaacagtgcgaacgcttctggtggcagaaggaagtacagtcaacaaatcacacacaccctctgaaaaaccggtatttggtaaaagtgccagtggaacagaaacaagtatttagactattttaaattatgaacggcaatttatttagtaacttttagcttgaacagattaaaattcaggatgggggctatctctttgggggttacatctctgttaccatcaccccttgatggtggagattcgaagcccacacagtcactcgtaactcacactgcgacccccgccccccaactcctctaggcctggtcagtggtgtgcggcagattgtgacttgattttctgctctctgtaccttgctgtgtcccacagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggtaactatcctgtcctccttctgactgtgttctccgattcctcgagccaaagccagacatctgttaggcgtggttctgctgctggaagctgactggtgaccactggtcagcatgaagcaaactctgcttcctccagccacagccccatccccccagtgtccacccattgcccattgcctctcactggcttcacttgcatatttcccctggtgtttggatgaaaagcgctggggctcagcttgtgtgaaattccttggtgctctgccaaccacacttcgttctggctcagctgactcagctgttccacccaggccacctcacatcaaactttttttttttttttttgagatggagtctcactgtgtcgcccaggctggagtgcagtggcacaatctcgactcactgcaacctttgcctcctgggttcaagtgattctcctgcctcagcctcccaagtagctgggactacaggcatgcgccaccacgcccagctactttttgtatttttagtagagatggggtttctccatgttggccaggctggtctcgaagccctgacctcaggtgattcacccacctcagcctcccacagtgctgggattacaagtgtgaaccacggtgcccggcctcacatgaaacttttgatttatagagagcagagggaagagccggctgtgcccatccttttctggggccatcgagtggctcctgggcagcccccaaggttaggaagggcaggagcagccagggttctctgatgccccagactcaagcacgagggaaggtctcaggggttccatgtgagcctcatggatgtctctgcttagcagagccctggctttgggcattgtccagatagggggtgagaaccagatcttctcatctccaggacctcagacgtatagttttctcagatttctgtgctttctggggctgggctactagtggaagaaagcagtctattctgtcttctcccaaatctcccagatgcccagtctgttgaaggaggagcagaaccagggggcctttcccgctgaggcccgacctgtgtctccttcaaatgacacgcgggactcagggccttcccatgaccatggggcccagggggcgtcacctggcccagggcccagtgctagaaacagatgaccccaggaggaggaggcagggcaggagggaagctggcagggctgggatggtcagccaggctgaggggcggactcgcaccaggatggagctaggaaatgatccaggtgtgtttggcggctgcaggtgggtccgcatggctgtgcagggagggaagggctgcgtggcaggagagcagccgggggaggcccagactctgctgaagagatgcctgttgtgccggcctccacatccgctgcccgctccttccggagctcctgccccgccatgctcagcctgactctgaccaacacgttggagagaagaatgatccctttgtgctattaagcttgcttatttggtttctaagtgcttcatgcgaacctagaggaaaaaattattttccacctttgtttgtcttaagaaaataacacacttttttttttcctatttgaacaggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcatgcaatcccatttgcaggatctgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcaatttttttttcaaagtaactactgcatctttgaagttctgcctggtgagtaggaccagcctccatttccttataagggggtgatgttgaggctgctggtcagaggaccaaaggtgaggcaaggccagacttggtgctcctgtggttctcgagataacttcgtataatgtatgctatacgaagttatatgcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagataacttcgtataatgtatgctatacgaagttatgctagtaactataacggtcctaaggtagcgagctagctccacgtggctttgtcccagacttcctttgtcttcaacaaccttctgcaagaaaaccaagggcctgaattttaacttcctg

SEQ ID NO: 73 - DNA, 25333 bp, synthetic construct
  misc_feature 1..25333: note = Recombinant polynucleotide
  source 1..25333: mol_type = other DNA
gcagagtctaagaaatcgctgtgtttagccctcgccctgggcactgtcctcacgggagctgctgtggctgctgtcttgctttggaagttcagtaagtgcagggagcctcgatcccaccatgtgctcctgcagtccccagtgctctgagccagaccctgctctctgggctattgagacctctggaggccctccgtgaggttcctctcttacataacgaggctgtctctcttcccttctcttgtttagctatgagattgacacatcatggggaaagcatttagaatgtacccagtgctttggggtgcttggtgccacccagcactgtgagcacaggttcttctaccttggggccacacccagttacctgtatctcactgcacagcagtggctgttggggaccaggcccacccctccatgtcccacctcctgcaactgcagcctgagccttcccatcagcctggggtggtgcagacccatgtgccattgtggatccttcaagttacctgtgtggcagagaggacgtgtgagtgccgtccaaacccaaacactgagagggtccttcccattgcccccacggaagtaaggtgccccagtgctaattccacttatacttgctggtggcaaggacacttctcctccttattaaagtgggggattggctgggtgaggtggctcacgcctgttatcccagcactttaagaggccaaggcaggtggaccacctgaggtcaggagtttgagaccacaagcctggccaacatgttgaaactccatctctactaaaaatacaaaaattagtcaggcgtggtggcgtgcacctgtaatcccagctacttaggaggctggggcaggaggatcacttgaacccaggagttggaggttgcagtgagccaagattgtgcccctgcactccagcctgggtgacagaatgagacttcatctcaaaaacaaaacaaaacaaaacacagtggggccaggagttggaggctgcagcgagctacagtaatgccacggtgttcctcactccatgaggctcattgcgtttctcagcctgaagggcacctctcttctgttttctctgcaagtgggcagcaagtgctccaactctgggatagagtgcgactcctcaggtacctgcatcaacccctctaactggtgtgatggcgtgtcacactgccccggcggggaggacgagaatcggtgtggtgagtcagccttgaccttgggaagggactcctctgctcaccttggagacagcagccgggtccaggggcctttgggtgactgggcctggcgtgcgtccagtacgctgacacatgatgtcattgaatccctgctccaggctgagccctggggctcagagaggttgtgtttccggcccaacctcacccagcaggtgggagatgacagggccaccgaggactgtgtcattggaaccacacgtgctctgaactgccacaggaagtcagttaagatgagcaaactgtttataaagttggagatgcaggctaggaacggtggctcatgcctgtaatcccagcactttgggaggccgaggcagatggatcacctgaggtcaggagtttgagaccagcctgaccaatatggtgaaaccttatctccactaaaaatacaaaaattagccaagcgcggtggcgggtgcctgtaattccagctattcaggaggctgaggcaggagaatcacttgaacctgggaggcggaggttgcagtgagctgagatcacgccactgcattccagcctgggagacagagctggctcaaaaaataaattaattaattaaaaacaaaattggagatgcactatgttattttcaaaacaagctgcctttaaagatctatctgttgtcacagggtgggctcatctgtttcattttattttctgtggtttatctatttattcattttaatgaactaggaagcattgctcctatttatggcataccacatgatgtttggatacgtgtatgcctgtggcatggctaagtcaagctagaacatgggccttacctcatatacgtgtcttattaagaacacataaaacctactcttgtagtgattttcaaatatgcaacatatagtttattaactgcagtcactatgatgtacaatagattgctcgaacttattcctcctgtctaactaagattttgtgacctctgaccaacatctccccagtgttgtcaccccccgcccccagcctctgatagctgcctttctactctctgcttctgtgagtttgatgtttatacattccacatgtaagtggcctcatgcagtgtttctgtctctgtgtctggcttgttcacttagcgtaatgtcctccagcttcatctatgttgttggaaatgacaggatttccttctttcttgtggctgaatagtattgccttgtgcatatacaccacattttctttatcccttcattcactgatggactcttaggttgatgtcatgtcttggctgttgtgaaaaatgccgcagtgagcgtgggcgtgcaggtccctcttcaacacacggatttcctttcctttggatataaacccagcagtgagattgctggatcacatggcagttctgtttctcaccttttgaggaaactccatactgttttccataatggctgtagcaacttccactcccacccccacggtgcaaagtctccatttctcttctacaacctcaccaactcctgttattttccatctttctgatagtagccatttgaagaggtatgagatgatacctcattgtggttttcatttgcatttttatttgtatttttcatgaatttttgagggtgatttcaagggtagttagtgactcgaacagggaaacgatcctgagtatgagggttgtgctaatcatccccctcctgccagctgcgtacggaatggggctctgcagatggcagggagctggctcgtttctctttaagagctgccttttacttttcttcctcttcctttaaaacttatttcctggccggacgcagtggctcatgcctgtaatcccagcactttgggaggccgaggtgggcggatcacgaggtcaggaattccagaccagcctggccaacatggtgaaaccccgtctctactaaaaatacaaaaattagccagacgtggtggtgcgggcctatagtcccagctactcgggaggctgaggcaggagaatcacttgaacctgggaggagggggttgcagtgagccgagattgcgccactgcactccagcctgggcgacagagccagactccatctcaaaaaacaaaaaaaagttatttcccaagcacagccatgtattccaggcttgtggatcagcgttggtggtggtgtgtgctctcatatcttagttccagctaagcacactctgacatgtttacactagaaccatttgttttttctagaaatagaaatttcagaattgtagagtcagaggacttaccagaaatctcttaggtagttctcctcccctccctcaagtgcagtcctaacctcctggagttttctgtagaaaccacaagcctcagagctggccgagaattctagccaaagatttttccatgccaaagtaatcccccctctcctaagggccatccttggtggggactggtttcctgttaagccctcgctgtcagtcctggctgtggaatttcctggtgaggagcactggcccgtggagctcggccctcgtgccggccttgagcaggcccaagtgttccgtgttcttgatacctttcctccagcacagtcttgcttcccagaaaaaggtttgcacttgaaaatgatgcatttgctgattaaacatagttcttttgctttatttggtttctaaaataaagtgggagtttttgagattgagtaacgtgaggttaagatagcacgtggaatggctttttcttttctttctattttttttttttttttcctggagacagggtttcactctgttgcccaggctggagtgcagaggcatgaccatggctcactgcaacttcgatgtcctggggttaagcgatcccccagcctcagccccccaagtggctgggactacaggtgctcgccaccacacctggctaatttttgtattttttgtagaaaatgggtttcatcaatgttgtccagactggtctcgaactcctgacctcaagcaattctcctgcctcagcctcccagactgctgggattacaggcgtgaactaccacgcctggcctggaatggcttttgatgttctcctatgtgcacatgtgggtgaataaacaccaacaaagtccttatgttacctgaagagttgctctcttcttaatatttaagtcgtatttatttaaatactttaatagttgtacactattaaagtattattaggtcaaaatcaaggaagtacaaaagggtatgctgtgaaaaatctcttcttccttgctctgcttacttacctaccccgcatccccccatacaccccagacacacacacacacacacacacacacacacacacacgcatcactcccatacatgcccacctgtttaccagccaatcacatttcttggggcaactcatctgagttgcttctctttccagagagtttttgcataaagaagcacaggtatttctgcgttaccatgaccctatttcccagtggttcctagccagttgactctcctgcactggataccatcctggacagcattccttagggaaatgagccccctgttttttcccaccatggcacagttggtcctttgcatggacgcaccattattgcccctgtctcttcttggtggaccttaaggttttctccatccttttgctgtaacacacactgctccaagtgtgtgagcatatcagtaggaaacgcttccaggagtagaactgctaggtcagagggcgtgtggatctgtaacctgacagacctagaccggcttcagtttggttttatccagtttccatattgattattcatataaaaggaaacagacaaacataacgctgtgcatgtattctctcttagaccagaacaggcatagggtgcacttttaatttgtccatttcgtagagtagaaattgtttttgctgaaatgaacaccttaggatgctgaagaatatgacccgtcccatggaaaacattcaaaaatgtgtgtagcgctttcttcccaagggtgtgtgtgcgcatattttaacactaattcactttctacttccgttgctatcctttctgtgagtctttctcagaatctcagaaaagaaactaaattgttcactctagttatcaatgctgtactctatacctggaatttgctaaaagggcagattttaagtattctcaccacagaaaagagaaaagaaaatggtaattatgtgacgtggtggacatgttaactagctttattatggtgagcatttcacagcggatatccagtcatcacgctgtacacattaaacatgtacaattgggtttttttgagacaaggtctccttctgtcacccagtctggagtgcagtggctcagtcatggctcattgcagcctcgacctcctgggctcaatccatccttccccctcagcctcctgaaaagctggggccacaggcatgtaccatcatgccaggctaatgcatatatatttatattttttggtggagatggggttggtctcgaactctgggctcaagtgatcctcccgccttgcccttccaaagtgctgagattacaggcatgaaccacagcaccaggcctacatgtaaaatttttatttgtcaactatactttgacaaagctgagaaaaaaaatcctaatatttaaaaaaaaaaaaaaaaggactagcttgagaccttttccagctctctggcttatcagctgccgtctcttccgggtgcagatagctggaagggaaagaaaatccctaaaattacccacaagccaagaatgaagtgtctccctttgagccacagtggcagttttgtttttaatcatagaagtgtattttgagccgggtgtgctggctcacgcctgtaatccccgcactttgggaggccgaggtggggggcggagggggtggggatcgcctgaggtcaggagttcgagaccagcctgaccaacatggagaaaccccgtctctactaaaaatacaaaattagccggcgtggtggtgcatgcctgtaatcccagctactcatgaggctgagtcaggagaatctcttgaacccaggaggtggaggttgcggtgagctgagatcatgccattgcactccagcctgggaacaagaaaaaaaaagaagaagaagaagaagtgtattcatttcagttacttttaaaaaagtgaacagactttatattttagagcggttttaggtttacagaaaatgaaacagacagggcagcgagctccttgtactcctccccagcacacagttgccctgttatgaacatcccacatcagtgctgtgcgttcattaacaccgatgaacctgatgcatacattatgatgaactgaagtcctggacttcaccctttctcttgtacagttctgtgggatttgacaaatgcataatgctgtacagccacaatgatagtatcgtccagagtagttctcctgccttaaaacctcttttgctgcacctgtttctctctccccactcaccccagctatctgatcttcttagtgcctccgaagttttggtcttttcaggatgttgtagcgttggaatcatggagtatgtagccttcaccacatacaccttccttcactttgttggcttcctttacttagtaatatgcattcaagtttcctccatgccttttcatggcttgatagctcatttctttttagcaccaaataatattccgttgtccagatgtagcacaatgtttatccattcatgtaacctgtgaccgactcacagataggatgtggaatcactcaccacagaggcattagacaataatcagacccaagtcatttcatgggggaacaagcccacaggtaccagactgtccagtgagtcagggccactcgtaggaagtaagaagagaggctagagcatagccaggtcctcactttatactttaagcccatgtgtatttctcccaaaccacacagcattgtttccatgctttcagctttgcatgaataacgtgatacttgaacgcatcatttatcacttgctctctttcccacagcgctgttttcaagcttcttcctgttcatgatgctctgcttaacccttaagctgcatgggattctgttctgtgaatacgcccaccccatgtattatcctgcccagcaaaaagtccccaaaactctggatggtggttacctctagggagggagagaagagattgggaatagggagcgacttcaacggtgtttgtaatgttttgtttctttaaataaaagagctgagatcatttcagcagaatgttgatttagagtctcctggacaatttgttgctcaaagtgctctcttaaagagcactttaaaaaaaaaaaccttttatcttattatttatttatttatttattgagacggagttttgctctgtcacccaggctggagtggagtggtgtgatctcagctcactgcaacctttacctcctgggttcaagcaattcccctgcctcagcctcccaagtaggtgggattacagatgcgtgccaccacacttggctaatttttgcattttagtagagatcggtttctccatgttggccaggctgatctcaaacgcctgacctcaggtgatctgcccgccttggcctcccaaagtgctggtattacaggcgtgagctaccatgcctggcttatcttatatatttttaaaaacagcttattgagatctaatttatgtaccataaaattcaagtatataattcagtgcttttatatataaaacatatatatgaaatagcttattgagatataattttttatataaaacagcttattgatatgtaatgtatgtaccataaaatttaaatatataattcactggcttttatatattcacgaatatgtgcaactatcaccacagtcaattttagcatattttcatcagctcataaagaaaccccaagcccttgaactatcaccccatatccctcctcccagcccgtccctcctactcataagcaaccactaatctacttagtgtctatagatttcctactctaggcattccatgtgagcgggatcatgcaatacgtgggctcacacaatataagtggcattccatgtgagtcggctcatgcagtatgtccggctcctttcactgagcataaggtcttcagcactcatccaggttgcagcctgtgtctgaatttcattccctcttctggctgaatcgtattccattgtgtatcttggacatatcctattctgctcacccagccgttggtgggcgtttggagtgttttcgcctttcagctgttttaagagggttgcagtgaacatttgtacaagttttggacccaatgcctgttttcaattctcttgtgtagagagcactttttagcagaaaaagaatagatttgtggcctccctttgtgtgcggtcagtgccttgagaagagtgaactgtgctgccacctccggagccgtggagagcgcggggcttgggtagcagctaggacgatacaagttgggacaaggccaggtgcaatggctcacgcctgtaattccaacactttgggagaccgaggcagggggatcacctgaggtcaggagttcaagaccagcctggccaacatggtgaaaccccatctctaataaaacagaaaaattaactggacggggtggtggacgcctgtaatcccagctactcgggaggctgaggcaggagaatcacttgaacctgggaggcggaggctgcagtgagtggagatcagaccactgcacttcagcctaggtgacagagcgagactccgtctcaaaaaaaagaaaaaaaaagaaagaaactcatggataatcctccctctcgtgcagttcgcctctacggaccaaacttcatccttcaggtgtactcatctcagaggaagtcctggcaccctgtgtgccaagacgactggaacgagaactacgggcgggcggcctgcagggacatgggctataagtgagtatggggcagcacccgccgagtgacagtaacagacagcagaaacacgagaagaccctctctctgcctccctgtgaaagcaccggcacatgagtgctggggacaattgtcaccttccaaaagctgagccctataaccagcaggtggaatttgtcctgctagggctgtgcccagcacacagaccttggctcactgccaccttgccctgcctcctccttggcctctatagactcctggttgctcgggagtgcccagtgctgtggtcatctggtcagaggggtaggctgagggcgttaggtgcctctttttccaaggtgcctctcagccagggtccattcacctccctgggtagaggttggaccagaacagctggcgaggagggttgggctggggagagcagcagagacaaatcctgtgccagtttcacttcattcgggagccatggaagccttttgagctggggagagaatcaatcaatcagactgatacttaaaaaatgtcattcctgctcgtagctctgagggaaggtgggaaggcttaacagggtgtgtgtcgcctgacagtgattcctaacgggggtggggcggtggttaccatttaccagcactgcctggggagatgcggcagccctcaggcatcgggggagagggtggtaggatgctactgccactttgttttccatgggagggtccccaggtgatttctatgcaactttagggtattcaatatgccagttttcagaatgaattaccactcggtgagaaagttggcatcttagctagtcactgtgacatccctaaacagcaggggtgaattacacagcaaagcccccccatcacagtccaggaacctggtggaattgataactggggccatgttaacatctgtaccttttattagattaaatgtgtgtatgattatacaatcctatgtccttctcatagtttcttgatcctaacctggataagaaacacgaccaatgaaggaattttgtctgacactttagggttattgaatcgaaaaatcgttacaatattctagcacttggttagaacgtgtgattttttttcctaaatgctaaggtttttccctcttattctgaatgtcgtatgagcggtattatgacatagtataggatttgtgtttgcttatgccttaaccattatcacaaataaggttttcttttttaggaataatttttactctagccaaggaatagtggatgacagcggatccaccagctttatgaaactgaacacaagtgccggcaatgtcgatatctataaaaaactgtaccacaggtatgcagcaatttcttcttgaaaaattttggaatgaaatcaactaggagacaccatggggaatcgttgtcctgagtctgatttctctgagctgcaatactcggtctggatgggttttgcattgggaggagattagagtctgaccaggcctggttactctaagcagcccttggtttattcataggaagtggctgaggtttctctgctatttcattttcagcctctaccgtctgcccttgttggtagcggctcacacttgcaacatcgacattcaactctatttagttttctttcctcttcagacatttagaggtgtacctattttgtcagggcgtggttctaggaatccaagataatgtctcagtgtcccagccagggtgaccggctcattccagtttgccagggacttcactggcttgagcaagggaagtcctgctccattccaggcagctgggctggctggtcccgttagccccaaccccgggacagcagtgccagagggtgctctgtgagggatgggcagcattctggcggcctgggaatgagttgtggtgtttccagggggtagaagtgggtacaagccacaggtcacatgatgagtggctgacctggctgggagggcagaagaggggatggacttaggctcttccttttgctttgcacatatttaggatgtttgcagacttgctatgattgttgctgttatgtgttttctgatgtgaaagatacacagtgtcctttgcccatgagctctccttgcctcccaggtccccagggcttatgcctggtgtctaggcatcacctccctgcctgccaggtgccaggtgctgcatttcgggggaggatgaactaatcaccccgcgccacctttcctctgagtgggagcctggggcaggtttgcattcctggaggccgctggtggaggggtctgggggcctgacttccactgcagcctgctgtcctggggaatgtggcagggcaagcccagtggggagggctgtgcacggccaggtgcacccatcaaaacagcagggctgcggtttgtccctgtggagaagctaaacacagctgcctgggcactttgtaaatgctgagtggttctttgtctttctgggttacacacggaatcagggagccaagtccagccgggcagggacggggggaggggaggaggtgctgccgtcccttggcaagagccttgggaactcacaaggaggctggagggcttggaagaaagaagagaaggccattgtctggtaggctctattctatctcggtggtggtggtggggggaggcgcacttcttttcctctttctgtgcagcagttgccctttgatgcctgagttcttggcttgttttctgtcgggcttctgtgaataaccacatgtgccctggcgctgtgaccacacagggctatccctaccgaccttaggattcttaggaaatgtcttctcttaaaggggacatgtcttcacttggccgtgtcagtgccccagagccagagtccacctggaatgcacctgtagtcactgagaacccggggggtgtgccttagtaagaaggtgtcaggaaggacctattattgtagggcctgggctcctgcaaggtggtttgggggtggttggaggaagcagagatttgctctggattggatgctgtcaggaagcaggggtaattctgtgaggctgctttattattttttttctaggaggaggttggaatgaggctaggctaaagctgtgattggtaaagaaacgtccgtcgctcaagttagccaggacaggaggagacatcagatcgtgattttgtggttgtgagcacaaggttcctgttctgtctgttcagacatcatttcggaggaggctccttgtgtcttgccccatctcaggcatggaggggcctagtccgatattgacgctcagtgaaataattcaggttccgcagagcacacggcccagctatcagggcgggccagctctgcatgccaggggccgcgtcttcccttctcagcatagcctgggaaattcactgcaggacaaaatgcatcagttacttcctcttcatccataacctgggatgtttgactcccaaatgagtaactcttacgtttcttctaatcctagggaaactattggttatattgctttcaacactacaaatttaaagcagttataggagcccagaggtttccaaatggcttccttaaaaattagaagatgattttaaattccaagaggaaaaacaaaactagcattattgtatacttaccctcacaaccgtcctaggagctggtacaattttaagagaggttaagtaacttgcccaaggtcacactgtggggatgtgagccgcgtaccttggctcagtgtctggtctttgccactgtccctatatggatttacttaccttattggagttgtaactagcagacccttctatgtctcagaagacaggagagggaacatcggaagaaatgactgatttctaagcatgtgagaggcaggtgactccgcactatcgtgaccagaatttcccctgttctttttgcagtgatgcctgttcttcaaaagcagtggtttctttacgctgtataggtaagttcatctggagtcccccttttgatacttctaactaggaaaagctctctactttcagaacagtactccctgtgtctctgggggcgtgggagggaagaaggtggggtcacgggttggaatgtgcccagcggcgtctcgctctttccaaggagctcctggtttagatttccatggcctgtagacaccttcagccttgggtccaagggacaccccctgagatcaggcacgctcaagaagctgacaaagccctacactttatgccacccatgagctggaggcccggcaggtctctttctccagaaagcaaaggggggtggcgttagtgagccctggcagccacctaacgtggacttggagcatctgcggggctgtggtccagcaccaccgtgtggccaccaggtgctcatcagccagtgggacccgggaggagggacaagaccagagaacaacagtgctcttgcctcttctctcctgaattttggacggtggcttagacttgggtgtccccatctctgtgtttagagtgcttacagtttccaaactgtttgcaaatgtggaagccaccgtccctctcctctgggatggcccagtgctgtcgtggggccgtggtcctgagctcagcttttcatttgaagaggtggaaggagctgacaccgtcccatcccggcagggctggctcaggtcttctttaggtcctgagtgggggtccagcacagccccaagggtgcgtggcacccgccctgccctctgcccatgcactcatctcctggtggagaagacactcacacacaggaagcagggaaggcagcagacctcactcacccctcaccccctcactcaccccctactcaccccctcaacctctcattcaccacccaccccctcgccccctcactcaccccctcactccctcaaccctcactcacctcctcactccctcaaccctcactcacctcctcacctcctcactctccccctcatccctccctcaccccaccccgtcacctcctcactcacctcctcaccccctcactcacccttcaccccctcactcaccacctcacctcctcactcaccccctactcaacccctcattcacccctcaccccctcactcacccctgcaccccctcactcaccccttcatccactcacccacctgctcacctcctcactcaacccctcaccccctcactaatccctcactccctcaccccctcacgccctcactcacaccttcacctcctcactcaccccctcaccccctcaaccccttacttaccccctcactcatcccttcacccctcactcaccccctctctcacccattcaccccctcactcatgccttcaccccctcactcacctcctcactcacaccttcacccctcagtcaccccctcactcaccccttcaccccctcaatcatgccttcactccctcactcaccccttcaccctctgaattactccctcatcccctcactcaccccctcactcaccccttcaccccctcacccaccacctcacccacccctcacccaccccctcacctccttacccctcacccccctcactcacccctcaccccctcactcaccacctcacccacccctcacccaccccctcactcactccctcatcccctcactcaccccctcaccccctcactcaccccctcacccacccctcacccaccccctcaccccctcactcaccccttcaccccctcactcaccccctcactcaccccttcaccccctcactcaccacctcacccacccctcacccaccccctcactcactccctcaccccctcactcaccccctcaccccctcactcaccccctcatctcctcactcaccccctcacctcctcactcacccgctcacctcctcactcaccccctcgccccctcactcacccctcaccccctcaccccctcactcacccctcaccccctcgccccctcactcaccccctcgccccctcactcacccctcaccccctcaccccctcactcatcccctcacctcctcactcaccccctcacctcctcactcaccccctcacctcctcactcaccccctcacctcctcacccaccccctcactcactccctcaccccctcaccccctcactcaccccctcacctcctcactcaccccctcacctcctcacccaccccctcactcactccctcaccccctcaccccctcactcaccccctcacctcctcactcaccccctcacctcctcactcaccccctcacctcctcactcatgccctcaccccctcactcaccctttcacctccttgctcatcccctcacttaccccctcacttcgtcaatcacccccccacctcgtcaatcaccccctcaccttttcactcaccccctcactcacccccttacttcctcacttacctcctcaccccccactcaccccctcaccccccactcaccccctcaccccacactcaccccctcaccccccactcaccccctcacccctctcacctcctcactcaccccctcacctcctcacttatcccctcaccccctcaattaccccctcaccccctcaattactccctcatcctttcaattacccactcaccccctcacctcctcactcctcactcactccctcactcaccccttcaccttctcactcacctcctcgtctcctcaccccctcactcacttccagccctgcccctcccatcttccttttctttgtgtgagaatctggggtccctgagtggtgtcagtccctccaagactcaaggagtccccagggccttgttatccagaacacccccacctgggtcccgggagaccccatgggatcacaggagtgttcagggaagtggtgcttcctgggtctgggtgggctggaggggcatcctcccttccccaagaggagacccccaggagccccctaagtccatccccagcagtggtgcccctgccctgtccttgcagcctgggagacccttgggaggggcgggcgctgggtggctgggcggcttctgctggtctcaccccactggcctcctgtttgtcatcctcagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaagtatgccaggggcggcgcgggccgggtgggggctcagggctggcctacagccaccctgtgaccttgagcaggtctcaacccttgcagccccggcatccttgtgtttaaatggggagagtattgcacctgcttcctagggctgtgagacatcaagtgcgctcatgccaggcagtgcatggctgtatgcactgagtgtcccctgcacgcagggcacagggtgcaggtggaacattctccacgatgtcgccgtgaccagcgttccttccagccactgtcctctgagctctgtcctgcccttgagcaaagcccctgccccctgaggtatcctgtctccgggacgctagtcccaggagagggcacactcagacaggcttcaggctgccctgctggaaggtccctggggttaagcgttcttggccacagcattgctcatgcagagggttaggtaggggtgaggctagccgtgacagtattagcatttatggacgctaccaccccctccccttttccttaaacacatagtgcttttggtcacatgctgctttggaggaggcctcacttggcggatgtatttttctgccttagagagaggctgaactgggtttgactgttggcccagccctctcttgctgcgtgcccttagacgattcactcaacgtctctgatccatggcatgtacaactataagatgggcatgcccttctcctctcgggctgttatgaaggtcaaggaagcaagggctgttacccaagggtgctcccttctctccccctcttcacacccccaggtgctctgggccctctaggaactgggtttctctcaagggctgttacccaagggtgctcccttctctccccctcttcacaccactgggtgctctgggcccactaggagctgggattctcttaagagggaaactcttggataaaggaaatggtttgattgatatcggacaagtctgttcattagtatccatttattaagcacctaccatgtgccaggaaatgctttggcgtacaaaggaaaataagggccagtcctgctagaaatggccttgaaaccccagggagggatgtcggcccattgtgggtgctgcagattccttgaaggtgatgcaagagccagaaagaaggatgatgtggggggctgaggcagggagtcggggttgggggagtgtgggggagaaggggagaccgagcacctcttccactatctccctgtgtggtttttggtgaaccatcctgcctctgggtgtcttgcctccagcttctgacgttggaagttcatccactgagagctctgtgtttatggctctgagatactgagtccttcttctctcccagacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacggtacgtgtggctcaggcttggcaagcaggttggcagaatcttaaagagatgttgattggaaatgacacttgtgctatgccaaatggaagggaggcatttgcgttgagcgagggtagcgtgcagcgggtggccaatgggagaggctcacagaggctaagagcacctgccgcattttgggggaggcagcagccaccacatctgttctgtactgtactgagtggtggtgattcaagccaggcatggaaaaggctagaacagggctttcccactgcagcacccttgacatctgggtggttctctgttgtagggctctcttgtgccttgtaggatgtttaacagcgtccccagcctctacccactggaggccagtagctaccaagctgtgacaaccagtgttgcctgctgacattgccaaacatccgctttgaggcaaagtcacttccagttgagaactactggcctaaaatgtgtaaagatccttgatttttaaagatacattctaaaaccaagttgcttaattcaggacaaacatgctttctcttagcctcttattcggtcccactctggtccatccaagggtctggaatgttctagccccatgtggatacagaagaagcaaaacctcagccctccctacagcatgtctgtattcacattgggaaatggttcacatatagaagagcgaatgcctgagcaatggcgtggtgcctctggggcgaaagctgactccattgactccatcggctttttggctgttgcctcctgtgtgtctttcccgtcttgatcacctggagatatgtaattttggaagcagagctagcaaataattcctcttataagcagagctagcaaataattctacttataagtagcataacgtcttgcctgccagaaggagaggtctggcagggggagaaagtgagaatgtgggacttgttgggatgcagggtcctctgggcagggtggccagggtgccaggcccagcagcctgcatgtgggaaggccaggtggagacataggtgatacccgcctggctcactgtgttttctcttcttgaaacagacctagtgaaaccagtgtgtctgcccaacccaggcatgatgctgcagccagaacagctctgctggatttccgggtggggggccaccgaggagaaaggtgaggctgctcctgggcacacaggactgcagggcccacagatggagcattgggttcggaagtgggaggtccaggttttaatcccagttctactactcaatgactggatgactttggttgattcccccagtccttgtgcctcagtttctccatctgctaagtgggagaaatcctgcccagcctacctaatacactgtgttcttatcgtgatcacacagagcagcatgtggaatggcttttgaagtatctgggccatacgagtttagaggtgcaggatctcctgtgttgcactcattgtgagtttagagctgccctggagatcccaccaaggcctgcgtggctgagtgacagggggcttggtgaggacgggcatcctggacccatggtggccacatctaagcctgtcctctgccctgataaccacagagagaggctctctccacccacttcctttgcaatctgcatttctctctgacagtctttcaaatgaagggagcctggctgcttcatttttatggagggttggaagtgcttagtggcaggcacaaaggttcattttacatattgtttatatccttctcaaaagcgtctaggccatacagacaacaaatcctttcaaacaaggggaaaagtacaaaggttgggtgatttctggggagcgtcagggaaggtagtggggggcatcctggctcctcatcagcagaaacttactacagtagagccacaggctgggcaaaagacctcatggaatccaagatgaagggaatatcgacaaatatttgtgcgcacctgcacctagtacaggctgggtgctactcaggtgctgggaatgcagaagtgaacagagtaagacaaatgtctctgctgtcaggagctttacctctcttctggatgtcggtggtggggacggggcaggtgtggtcagacagatgggagacaaacaactgagcgaggtacttccaaacatctgagggtggggatcacaaggtcccggctattttgaaggggtggtcaggaaaggcttctcggaagaggtggcatttgagctgagactcaaatggcaaaaatgtgtacacatcaaaaaggctagtgcatgtatcttcaggtgtggtcaaggggccaaggaggtgggctggggccagattgcataggtccttgtggattatggtgaagacaccagcttctcatctgcttgaggtggggagatcgtgagccggggagtgccatgatctggcagctgcgtggggagtggggatgaatggatggagacgaggatgatggtgacaagtccattgctgtggttccttgagacaggaagccagctcatagcagagtgcgggcgtggatgtgaagagatgagggtacactagggctagagccaccagacttactgatgggttgcatgtctgtgggagagagagtgagaagtcagggacgatggctttccactctgtggctgaagccccagggtggcgggtggtgccatttttcaagccaggaaatattggttggtgagaatttggggtgggagaaggtgtgacggagggttctggttttgcacactaagcccacggtgcccagaagatgcccgaggggaggcagcaaagcgagagtgggaaatgcagaggtggcaagtgcaggccgtgtcttgagaagctctaatgtgcaggggagccgagaagcaggcggcctagggagggtcacgtgtgctccagaagagtgtgtgcatgccagaggggaaacaggcgcctgtgtgtcctgggtggggttcagtgaggagtgggaaattggttcagcagaaccaagccgttgggtgaataagagggggattccatggcactgatagagccctatagtttcagagctgggaatttctttccctgaagctgaactccagagctgcattcagcacaggcaccgccagttgtaaggagaatccaggtttcccaggagaggggttggtgctgggatgagctgaccggggcagggctggaaaatagggctgtgaccatctgtgtagtgcgtgtggaggtctcagggagggaagtgtgctctccctgcgagagctgcaggcaacactgggagctcaacaagtctccctgtccttagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccaggtaattcaacatttttattctacctttggtccttaccagatcctactgaaccccccatgagagagagggcattcttggggtcagcagagcctcctcagtgacacggagccagctcggggcagtcatgggaagtgacggccacaaacagtgcgaacgcttctggtggcagaaggaagtacagtcaacaaatcacacacaccctctgaaaaaccggtatttggtaaaagtgccagtggaacagaaacaagtatttagactattttaaattatgaacggcaatttatttagtaacttttagcttgaacagattaaaattcaggatgggggctatctctttgggggttacatctctgttaccatcaccccttgatggtggagattcgaagcccacacagtcactcgtaactcacactgcgacccccgccccccaactcctctaggcctggtcagtggtgtgcggcagattgtgacttgattttctgctctctgtaccttgctgtgtcccacagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggtaactatcctgtcctccttctgactgtgttctccgattcctcgagccaaagccagacatctgttaggcgtggttctgctgctggaagctgactggtgaccactggtcagcatgaagcaaactctgcttcctccagccacagccccatccccccagtgtccacccattgcccattgcctctcactggcttcacttgcatatttcccctggtgtttggatgaaaagcgctggggctcagcttgtgtgaaattccttggtgctctgccaaccacacttcgttctggctcagctgactcagctgttccacccaggccacctcacatcaaactttttttttttttttttgagatggagtctcactgtgtcgcccaggctggagtgcagtggcacaatctcgactcactgcaacctttgcctcctgggttcaagtgattctcctgcctcagcctcccaagtagctgggactacaggcatgcgccaccacgcccagctactttttgtatttttagtagagatggggtttctccatgttggccaggctggtctcgaagccctgacctcaggtgattcacccacctcagcctcccacagtgctgggattacaagtgtgaaccacggtgcccggcctcacatgaaacttttgatttatagagagcagagggaagagccggctgtgcccatccttttctggggccatcgagtggctcctgggcagcccccaaggttaggaagggcaggagcagccagggttctctgatgccccagactcaagcacgagggaaggtctcaggggttccatgtgagcctcatggatgtctctgcttagcagagccctggctttgggcattgtccagatagggggtgagaaccagatcttctcatctccaggacctcagacgtatagttttctcagatttctgtgctttctggggctgggctactagtggaagaaagcagtctattctgtcttctcccaaatctcccagatgcccagtctgttgaaggaggagcagaaccagggggcctttcccgctgaggcccgacctgtgtctccttcaaatgacacgcgggactcagggccttcccatgaccatggggcccagggggcgtcacctggcccagggcccagtgctagaaacagatgaccccaggaggaggaggcagggcaggagggaagctggcagggctgggatggtcagccaggctgaggggcggactcgcaccaggatggagctaggaaatgatccaggtgtgtttggcggctgcaggtgggtccgcatggctgtgcagggagggaagggctgcgtggcaggagagcagccgggggaggcccagactctgctgaagagatgcctgttgtgccggcctccacatccgctgcccgctccttccggagctcctgccccgccatgctcagcctgactctgaccaacacgttggagagaagaatgatccctttgtgctattaagcttgcttatttggtttctaagtgcttcatgcgaacctagaggaaaaaattattttccacctttgtttgtcttaagaaaataacacacttttttttttcctatttgaacaggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcatgcaatcccatttgcaggatctgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcaatttttttttcaaagtaactactgcatctttgaagttctgcctggtgagtaggaccagcctccatttccttataagggggtgatgttgaggctgctggtcagaggaccaaaggtgaggcaaggccagacttggtgctcctgtggttctcgagataacttcgtataatgtatgctatacgaagttatgctagtaactataacggtcctaaggtagcgagctagctccacgtggctttgtcccagacttcctttgtcttcaacaaccttctgcaagaaaaccaagggcctgaattttaacttcctg

SEQ ID NO: 74 - protein, 491 aa, synthetic construct
  REGION 1..491: note = Recombinant protein
MALNSGSPPGIGPCYENHGYQSEHICPPRPPVAPNGYNLYPAQYYPSPVPQYAPRITTQASTSVIHTHPKSSGALCTSKSKKSLCLALALGTVLTGAAVAAVLLWKFMGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG

SEQ ID NO: 75 - DNA, 2267 bp, Mus musculus
  source 1..2267: mol_type = other DNA
ccggttgtgttataggacttgaccagccccaatagtcctcaagtcactcctagatacagtggcaggtggtagctggcttgcggaaggaagaggaagaagagaatgtgggccatcaaggagcaaggccagccttgcacttgggccccctctgctcagtgctgaccagggctttctgagccgcttcctaatgaggctcatttgaagacccccccccacccccctcctgctgtcttgggtggcagagctagctccaggctgtaagaaaattaggaggattaccaaagcagtatggagtcagacagtggccaacccctcaacaaccgtgatattgttccctttcgcaaaccccgaaggccccaggagaccttcaaaaaggtggggatccccatcattgcagtgctgctgagcctgatagccctcgtgattgtggcccttctcatcaaggtgattctggataaatactacttcatctgcggcagtcccctgaccttcattcagaggggccagttgtgtgacggccaccttgactgcgcctcaggggaggatgaggaacactgtgtcaaggacttccctgaaaagcccggagtggcagtccggctctccaaggacagatccaccctgcaggtgctggatgcagccacagggacctgggcctcagtctgtttcgacaacttcacagaagcactggccaagacagcctgcagacagatgggctatgacagccagcccgctttcagagcagtggagatccgtccagatcagaacctccctgttgctcaagtcacaggaaacagccaggaacttcaggtgcagaatggaagcagatcctgcctctcaggctccctggtttccttgcgctgccttgactgtggaaagagcctgaagactcctcgtgtggtgggtggggtggaggcccctgtggattcttggccgtggcaggtcagcatccagtacaacaagcagcatgtctgtggtgggagcatcctggatccccactggatcctcacagcagcccactgcttcaggaagtatcttgatgtgtcaagctggaaggtcagggcaggctcaaacatactgggtaactctccatccttgcctgtggccaagatcttcatcgctgaacccaatcctctgtaccccaaagagaaggacattgcccttgttaagctgcagatgccactcacattctcaggctcagtcaggcccatctgcctgcccttctctgatgaggtgcttgtcccagccacaccagtctgggtcattggatggggctttacagaagaaaacggaggaaagatgtctgacatgctactgcaggcatcagtccaggtcattgacagcacacggtgcaatgcagaggatgcctacgaaggggaagtgaccgctgagatgctgtgtgcaggtaccccacagggtggcaaggacacctgccagggtgacagtggtgggcctttgatgtaccattctgacaagtggcaggtagtaggcatcgtgagctggggccatggatgcggcggcccaagtactcctggagtgtataccaaggtcactgcctatctcaactggatctacaatgttcggaagtctgagatgtaacgctgccgtcccccacatccagaagctgcttcccttcagacctacctacggcatgacccctcaaagtcagatatgggacaagagcctccttgaacaaactctggtatccctgcagcaagcaaggatacattgcagaggtgcccggagtggagtcagatgggctagctcagccacccctgcatctcccaaaccctgggagacatgtggcccatgggagtaaatccaggacattgactcaactctcagaagtgttattcagtcaaggaggctctcccttccactgaaggaaggaaagtcagctctctcctgaaaggccagatcactggctgagtagatgagacaagggtatgaaaggcctttgccatcttctttgcccagtcctgaaagcactgacgtaagagaccagtcagttctaatgtaaggtgtatattttagtgtcagggtattgcaattgtcacctctgtggtcaatatcattaaacaggtatgagaattcgctggcatagacttcctggtctgcttaataagaatccaactaaggatgtcacatgacagtttcccagaaaatgtgaacaagtgtccatctgacacacggcaccaatgacaaaccaaagaagttattctgcctgagtctcagttgctgaactaataaattagctgcggtttcttgca

SEQ ID NO: 76 - protein, 435 aa, Mus musculus
MESDSGQPLNNRDIVPFRKPRRPQETFKKVGIPIIAVLLSLIALVIVALLIKVILDKYYFICGSPLTFIQRGQLCDGHLDCASGEDEEHCVKDFPEKPGVAVRLSKDRSTLQVLDAATGTWASVCFDNFTEALAKTACRQMGYDSQPAFRAVEIRPDQNLPVAQVTGNSQELQVQNGSRSCLSGSLVSLRCLDCGKSLKTPRVVGGVEAPVDSWPWQVSIQYNKQHVCGGSILDPHWILTAAHCFRKYLDVSSWKVRAGSNILGNSPSLPVAKIFIAEPNPLYPKEKDIALVKLQMPLTFSGSVRPICLPFSDEVLVPATPVWVIGWGFTEENGGKMSDMLLQASVQVIDSTRCNAEDAYEGEVTAEMLCAGTPQGGKDTCQGDSGGPLMYHSDKWQVVGIVSWGHGCGGPSTPGVYTKVTAYLNWIYNVRKSEM

SEQ ID NO: 77 - DNA, 3543 bp, Homo sapiens
  source 1..3543: mol_type = other DNA
atcattccagtttggcaacttcacttgtagggctgttttaatcaagctgcccaaagtcccccaatcactcctggaatacacagagagaggcagcagcttgctcagcggacaaggatgctgggcgtgagggaccaaggcctgccctgcactcgggcctcctccagccagtgctgaccagggacttctgacctgctggccagccaggacctgtgtggggaggccctcctgctgccttggggtgacaatctcagctccaggctacagggagaccgggaggatcacagagccagcatggatcctgacagtgatcaacctctgaacagcctcgatgtcaaacccctgcgcaaaccccgtatccccatggagaccttcagaaaggtggggatccccatcatcatagcactactgagcctggcgagtatcatcattgtggttgtcctcatcaaggtgattctggataaatactacttcctctgcgggcagcctctccacttcatcccgaggaagcagctgtgtgacggagagctggactgtcccttgggggaggacgaggagcactgtgtcaagagcttccccgaagggcctgcagtggcagtccgcctctccaaggaccgatccacactgcaggtgctggactcggccacagggaactggttctctgcctgtttcgacaacttcacagaagctctcgctgagacagcctgtaggcagatgggctacagcagcaaacccactttcagagctgtggagattggcccagaccaggatctggatgttgttgaaatcacagaaaacagccaggagcttcgcatgcggaactcaagtgggccctgtctctcaggctccctggtctccctgcactgtcttgcctgtgggaagagcctgaagaccccccgtgtggtgggtggggaggaggcctctgtggattcttggccttggcaggtcagcatccagtacgacaaacagcacgtctgtggagggagcatcctggacccccactgggtcctcacggcagcccactgcttcaggaaacataccgatgtgttcaactggaaggtgcgggcaggctcagacaaactgggcagcttcccatccctggctgtggccaagatcatcatcattgaattcaaccccatgtaccccaaagacaatgacatcgccctcatgaagctgcagttcccactcactttctcaggcacagtcaggcccatctgtctgcccttctttgatgaggagctcactccagccaccccactctggatcattggatggggctttacgaagcagaatggagggaagatgtctgacatactgctgcaggcgtcagtccaggtcattgacagcacacggtgcaatgcagacgatgcgtaccagggggaagtcaccgagaagatgatgtgtgcaggcatcccggaagggggtgtggacacctgccagggtgacagtggtgggcccctgatgtaccaatctgaccagtggcatgtggtgggcatcgttagttggggctatggctgcgggggcccgagcaccccaggagtatacaccaaggtctcagcctatctcaactggatctacaatgtctggaaggctgagctgtaatgctgctgcccctttgcagtgctgggagccgcttccttcctgccctgcccacctggggatcccccaaagtcagacacagagcaagagtccccttgggtacacccctctgcccacagcctcagcatttcttggagcagcaaagggcctcaattcctataagagaccctcgcagcccagaggcgcccagaggaagtcagcagccctagctcggccacacttggtgctcccagcatcccagggagagacacagcccactgaacaaggtctcaggggtattgctaagccaagaaggaactttcccacactactgaatggaagcaggctgtcttgtaaaagcccagatcactgtgggctggagaggagaaggaaagggtctgcgccagccctgtccgtcttcacccatccccaagcctactagagcaagaaaccagttgtaatataaaatgcactgccctactgttggtatgactaccgttacctactgttgtcattgttattacagctatggccactattattaaagagctgtgtaacatctctggcataggctagctggaatgcttgataagaactgagctgggatgattgaactttcattctttggcttggggagaaaagaagtcctggggaagcaattgagtctcaaagtagaggcaggggaaaaaagagttagggagaccagatctgctgagtggcagcaagagtgagctgcagattacagaaaccagggtgagcaagtttgagtcccacacagggccttctccctttgcctctttccctccctccctgcctgtgataatcagccaggagccagggataacctatgacttgggaaagagatgagttaggcagtcaagggtgacattcaatcagggatccacaagtggctggaaagaaatgctggtcctgtgtcctaactttttccgcctggagagccctcagtgtggcttcttacatttaaaaaacaaaaaggatcagctgccaggtgtgaggcagtccccaagctgagttgtgaggatgtaagcatgaataagtccctgcactcaaaatggtcaaagaattaaaccccatggacttttttggcatctgtatgaaagcttgggttttctgaggactgtcttgctatagttaagtcagatcctagatgaaatatacttgttcatactgtactaggttcttaggaaacaacagaattcctcaaatgccaaaaacaaagaaaatagaaacccagaaaacaaaacaaaataaaacaaaaccatcagaactgtgagtggaaactaaggtgatgatctgggagcaatacactaaaatcttgggtcgagacctatatgaaggctggcagtggagctaaacctggacacactgaagacaagggagctgaaccagggctcctacatgaagcagggataactgatggcagtaaatgtggtctcaaattgcagatggtctggaggaaaatttcccaaatttagagcctcaggattcccaaagatcctccaaatatgagctcacaatcaaagatcagagacgttgaaaaataaaaaacaccttaagtgggcagcataaaaaacagctaatttagaaccccaaaggcttcagatgtcagaatattagagacttatgataataagcaatatttgcagagtatttgtatgtgccagacactattgtaagtgcttcatcatgtactgattcatttaatactcacagaaatctgtgagatgggtattattcttatcctcactctatggattaaaaaaactaaggcacaaagtggttaagctccttgcctgagattatagactgtaagttgaacgtgagcacttggaatacagagttcatgctgtaaactaccacactatagggcctccaatatgataatttataaaatatttgaataaaaaatgaatactagttccacattttaaaaaaaaaaaaaaaaaa

SEQ ID NO: 78 - protein, 437 aa, Homo sapiens
MLQDPDSDQPLNSLDVKPLRKPRIPMETFRKVGIPIIIALLSLASIIIVVVLIKVILDKYYFLCGQPLHFIPRKQLCDGELDCPLGEDEEHCVKSFPEGPAVAVRLSKDRSTLQVLDSATGNWFSACFDNFTEALAETACRQMGYSSKPTFRAVEIGPDQDLDVVEITENSQELRMRNSSGPCLSGSLVSLHCLACGKSLKTPRVVGGEEASVDSWPWQVSIQYDKQHVCGGSILDPHWVLTAAHCFRKHTDVFNWKVRAGSDKLGSFPSLAVAKIIIIEFNPMYPKDNDIALMKLQFPLTFSGTVRPICLPFFDEELTPATPLWIIGWGFTKQNGGKMSDILLQASVQVIDSTRCNADDAYQGEVTEKMMCAGIPEGGVDTCQGDSGGPLMYQSDQWHVVGIVSWGYGCGGPSTPGVYTKVSAYLNWIYNVWKAEL

SEQ ID NO: 79 - DNA, 20078 bp, synthetic construct
  misc_feature 1..20078: note = Recombinant polynucleotide
  source 1..20078: mol_type = other DNA
ccacccgcacacactacagtcgagataacttcgtataatgtatgctatacgaagttatatgcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagcccgggcgccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccatgggtacccccaagaagaagaggaaggtgcgtaccgatttaaattccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagtgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggtaaatataaaatttttaagtgtataatgtgttaaactactgattctaattgtttgtgtattttaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattgatctagataagtaatgatcataatcagccatatcacatctgtagaggttttacttgctttaaaaaacctcccacacctccccctgaacctgaaacataaaatgaatgcaattgttgttgttaaacctgccctagttgcggccaattccagctgagcgtgcctccgcaccattaccagttggtctggtgtcaaaaataataataaccgggcaggggggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggaataacttcgtataatgtatgctatacgaagttatgctagtaactataacggtcctaaggtagcgagctagccaagtctgtgtgctaccaagtagcaaaactgagcctggaactcacacatgcgtgtctgagagcccagcactatcgccaggaaaacccagcgtctccctgctcaagcctgaccctcagccctctctgcctctccctgcacttgccttccagtcaaggtgattctggataaatactacttcctctgcgggcagcctctccacttcatcccgaggaagcagctgtgtgacggagagctggactgtcccttgggggaggacgaggagcactgtgtcaagagcttccccgaagggcctgcagtggcaggtgagtgcagggtctgaggcacaagagaagtgggcccagcaggaggtctgctcaggcccccacggcccactgcatagtatctgccccctacttgtcacttttcatccttgttgtataaggttctttgtttgtttgtttgttgttgttttgaggcagagtgctctgtggcccaagatggagtgcagtgtcttggtctcggctcactgcaacctctgcctcccagtttcaagtgattcttctgcctcagcctcatgagtagctgggattacaggtgccagccaccacgcctggctaatttttatatttttagtagagacggggttttgccacattggtcaggctgatcttgaactcctgacctcaggtgatctgcccgcctcagcctcccaaagtgctgggattacaggcgtgagccaccgtgcccagctgtgtaagtttcttgagagcaggaccctgtcttgtctacctttaaatcctagtacttaacacacagcaaacagtaactatttgatgaccaaatgtgagccagaaaggacaggaaattgtaactgaggctgccccatgcgtgctgcgcctggtggatttcaggcagagggctagactgggtgaccttggggcattcctcctttctatgaaatttgttatttcaaggagactagaaaagagacttctcagccacttcgccagctattggtccttctattcattagtgtttgctgagacatgctatgtgacaggactgagccaggtcctttcaatggataggagatgttttgagcataaaatccacgttctctcttgggctgggctcttctaccttcttccccctggtgcttgggctctgaagaaaaaaagataggtaggagatgagtgatggggcttctgagggcagggctgagtgactttctgtgtatttgctctttctttatcagaagtcaaatgcccacaggcacctgtcatcctactgccagtaggacttctcactcaaccttcccctctgaccttacttggagaaggacttaggtccctctctcagacatttccccaggctgggcaagttgtgtggaccatggatgggtatgtggtccatacaatttaaacaagctgtatatggtcgctgggtagagtgaccacataattgatcatcaaaactgatacctgtaagagcaaaagggggcactattaaccattgggtcagggcaacaggtcaaaatggagacctaccctgggacttctggtcacactagctactgtcaaaatggggcccaaatagacaaagccaaatggaagaaattcccttgacattgaaagtgttggggctctgtggcacccccagttctaggttgggggagcttgggctggtctcatgatgagttctgagggggatgggccagttgggccccccgttccatctaactcaggttcctttcctcccagtccgcctctccaaggaccgatccacactgcaggtgctggactcggccacagggaactggttctctgcctgtttcgacaacttcacagaagctctcgctgagacagcctgtaggcagatgggctacagcaggtaaccaacctgggcctctctcctttttccctccttcctccttcctcctcttcctcctttccttcctcccttcttctctctttcctaaaaattacgggcattggagccaggcagaatggcttttgaatcccagcatttcacttataagcaacatgaagttaaatttcctaagcctcaggttcctcaggagttaattgggggaactaatgccaacctcataggatagttttgcaatgccagtgagagaatgtgtgctgccctccaacacacacacacacacttctagcgtctatgcagtcctctcctttcctttactcctcaaccttcactcctttgtgctggctttgcaagaaactgttcctgcccagtaatacaaaagctaagttaacttattcaaagtttcgttagttaagatttagcttaagtgagcctagtttcagtggggccccatcttcagcaatcccagctctctctgcaaatttcaaaagcagttccaaatctggagtggatgaaaaggtgtaagatgatagtaagagtaatttgcattctatatatttatattcacttgattttggcagaaaaccaaaaagatagttattatatcttatatatagatatatattatatctatttcataaataggctcaaacaaagtaagtaacttgctagggtactagctgggaggtagagggctagaatttgagcccaagacccctaattcttgcgcattaggagttcccacattgtttctgtttctagactgagtaattctttattctcatgtaggacatcatctctaagggaaggggctaatgagatggttgatcactcagagagtttagctggagaggatggaaaagaacccatacattcagttgcagattgagatagcctatctctggcaggcctcagatttcttcaggattctaacagactggacccagagactaggccaaacaaacaaacaaacaaaaactctactaggcagacatcaccaaccaatcacagaactctctcccatggatccctaatacagcctcaaagtccttttcagtaaatgctccaggcagccattacaaatcaatcagaattatttgcctttctcttctctgctcaacgggcttctgctgctctctactttccatagggggcaacttccattaccctctagaaagcacaccccaccaccttcatttcaaggagagtgaggaactcatgcccagcacctgctattctcccctcttcctgcagccacggagcccagcctcgctgcagccagccctgcctccccactgtagtccagtcaactgctgcatcagccgttcctggcacagcaggctgagccttgattatgaaacctgggtgtctccaggggttcttaagatgataggctcctggaatttctgtccttttggagctcagtaaggcaccaaaccacctgagtcttgtgcttcacaaaatcaaagttcatcagaatcattcattgggatggaattggtgaacagaagttaactttcctgggaatgtccatttccaccatattccgtccttctaggtctcagacttctctactttctttcctctctctagatcggaggcccttcttgtcctagaaccataggcatttcaagatgtgggagaccctagggatcatctagtccacgcatcttttttttttttttttgacagagtctcactctgtcacccaggctggagtgcaatggcaccatctctgcttactgcaacctccacctcccaggttcaagtgattctttcgcctcagcctcccaagtagctgggattacaggcacgcaccatcatgcccagctaatttttatatttttgtagagaccgagtttcaccatgttggccaggctggtcttgaactcctgacctcaggtgatccacccacctcggcctcccaaagtgctgggattacaggcgtgagccactgcacccagccccgtgcatctttttatagagggggaaactgaggcttggagagacccagaaaaagaatatgacctgcccaaggccacacatcaaactagtgccagagccagggacagaacctagatcatgaggactcttaaaatgcactctagtcctcccaggtctgagacttgggtccttccaggaagtgccagcattcctgcctgagaatgtgccaatccaccagtattgccaatgactcagccctccatggagagcttctactaacattactagcatagttagggatggaaggaaaagatttagaagaggcagattcagtaaaggaacaatcagagagatggaattaatcaaggaaggcttcctggaggaggaaaaacttcaacccaaggtttgaaagtagcaagcatggattagcagggagaaagagggagagtggtccagttgagagaaacgtttgtctggattcatatgaagacagatctagtcctgttctattaaatatctctaagggggccaaaaacatacccccgctatcaaagtcagaccagatgctttgtttggagaacgaaatatccacattccaactccctcccaggtgagaagggagctaacctgagcccctatgcctctttgtttccctgctgtgaaccagaagacattgctgggatatttgaaatagggacagagctgggaatatggaaaggagacccctaacatttctccagggctctgggttctggatttggattccccacccaagaaagcaagttacatcagcaatgcactgagggttgagtcctgggatgccaagggtcggttctttattgtatagcaaagcaggccccatcttcactgactaagaccatctccactccctggccactccccaccaagcattctctgccactctttctcctgaaagtgggggccaactctaccatcttgttctaaccccctgccccagctcacaactctctctccctcttgatgtgagcagcaaacccactttcagagctgtggagattggcccagaccaggatctggatgttgttgaaatcacagaaaacagccaggagcttcgcatgcggaactcaagtgggtaagtgaggggacaccttctggcctacagaaggcccccacatggacgctgctcttcaggttgcaaccagctcacctggaaccccaagcagccaggggaatgtaagcagacatcaggaagaactcctagccagatggatcattcaatgccaagagctatagactcacattttggagaggttttctgtgttgacttgtttttaatacaatggacagctggacaaagtgtgttgtcctactcagagccagagggatggataatgtgacctttccatcaatctggatagtaaatagtttttgctactgctgtaggttttctaataaattgcccaataggcaagattccaaagtcactttgtccttccctaccacttacccagccagagctccccaccttcttgatgctccagggaagaggctccatggcccttgtgggtggcctgttcctgagcctcgccaccctgtgttagagcagagcatccagatgaaatctgtcacactgtggcaaagtggctcagagaggaggctggcttcctagcattcagggacgttgctgagggccgcttattcaccgaaaataaatcttgaaaaggacagggctggtagcagaatgatcctttacctaaaattctatcaaaatcccattcttccatttggaaagcccacagtgtcacagactctgttccgggctctgtcctcttccctcttgggtcccaggagcccaggctgggctttgaagcaggcagggcccagcacacagtaggtactcagcagtgggggtgttgaatccaatcaaacggaagtgtcaatgcaggaaatgcaatggatgtcaatgcagtctccaaatgttccccactgtgcagcttccacattcccgaggtattgggaggggacttgaattaacagcttcgggaggcctgagtccctgcctcccagctgaggaagaagcttaaatcacagggcgctgtgtctgtcttccaggccctgtctctcaggctccctggtctccctgcactgtcttggtgagtacccccaatctctgagggtttggggcctgggccagcaatgagcagggaggaagaccttcatcttcactcctaaatttctgggactccaagtttcattctgccttggtctacagcccttgggcttgtcggtcaatgccccctcgagttgttggtggccttgggcaggtcacattctttttctgggtctttccaagccccagtttcccccttctaccatctgtgcatggctccatgacctaagtggagacctgggagagagtgttaggaagaccgaaaagggcaggacggggcctccactgcctcccatccctggtccgggcccacatagccttctttgtcacaatcagctcaggtatccaagatcagattacccacattcattatttgagcaactattcattgaacagttagaatatgtctcactctgtcagttgctggctagaagtagaaagtaccagatgagtgaaataattggccactatccttggtagctgatgactaagtaagagagagatgcaagacaacatgtggaaaatgccaaactgagtagcagtcacagttgacatgctgcagagagagctggccgggggtcagaagacctgggcaccagtcctgttcatttccagtgtggcctcgagtcattcacctgacctccctgaagttcattttcccaagaagttgtttagtccaactgcccatcaaggatctttagggacccttctagctctaacagaggagatcagaaaagaaaacaagcaatgtggctcagctcatcctacaagcttcatagagaactgagactggcctggaagcatagccagaaattagaacgcctaagggaagaaggtcacaacgctgcctctgcaatttaggagtgtatatgctttcctgcaggatgttgagagtttcattcattatcgtatgccccctaccccggccccacaatacctagtgcgtgggatctgacacgtggtggctggtcaatgaatgaatgaatgaatggtcacaccatctgaggttctgcactgagtagccctgaaggcttgaagcagcataagtgacaggtcctcccttgaggggcctctgttttaccaataagccaagacctaagctcaacaacactgaaagggtggccaatacccaggacagcctgtgggaattccagagaaagggagattcccagggactgggggcccaggctaaacactgaaaaatgcatctgtaggctcaaggaggaaaagcccatgtctgtctgtcttgcccaccactctctcccagcacccagcactgccccaggacagagagcacttgacacaagttggttagattaatgaatgatttagagttcagtggtccccaacctttttggcacaagagactggttgcatggaagacaatttttccgcaaaccaagagggggatagagagcattagattctctcttttttttttttttgagaccaagtctggctcttgtcactcagcctggagtaaagtgttgcgatctcggctcactgcaacctccgcctcctggattcaagcgattctcctgcctcagccccctaaatagctgggattacaggcacccgtcaccagcccagctgggactataggcatgtgccaccatgcccggctaatttttgtatttttagtagagacggcgtttcaccatgttggccaggctagtctcgaactcctgacctcaggtgatctgcccgcctgagcctcccaaagtgctgggattacaggcatgagctgcctcacccagcctaaagtctcataaggaacgtacagcatagatccctcacatgtgcagttcacaataaggttgtgctcctacaagaatctaacgccacctctgatctgacaggaggtgaagctcaggtggtcatgctcgcttgtccctgccactcacttcctaatgtacagccaggttcctaacaggccacgaaccagtgggaagggcatctttttggatcaaaaacagaattactttttagagaactacaagcagatcaatttggctagacagagactttatatgaaacagcaggaggctgctaggaggagtggaaactctactttgccctcaagggagatcccgaagggctttgcaggagcgggcaaggtggcatgaagaaagcagtgtttgaaatcaggtggtatttgaaaagcccagcccttccccttagaatggcccttctaccatctgtgcatggctccacaaccgtggtggtggctgccagaagaattggaaaggcagagcatgggtggagaggggggacctgagggctttacaggagttccgggggtggtgagggtgtgaaagccaggtcagtcagtaggaagacaggatgtcagattgagagactcccctggccggggaaacagacttggagaagggggagttttggatgagacagtccacttccgagtcacaaaatagcttgtgggtgtctgtttactgttactcagtgggagtggctggggacacgccacctgggcagggctttcgtaattctgcatcacttgtgaaggtcacagattcccagcacaacggacacacccatgttcatagtctgaactcctaaacacatcttaaaccaaaataaaaaaaaaagaaagaaagaaagaaaaaggagagggaggtttgaggaaagcctatggtctgggacactcaatacctcccatgaatatctcatattgggctggtcctctctccactctggccccagccataagggccctgcttagagcagattttgggtgctgagtggaggcagcctcatccccaacagcctgacttcctgcctcctccctgcctctgcctgtgtccagcctgtgggaagagcctgaagaccccccgtgtggtgggtgtggaggaggcctctgtggattcttggccttggcaggtcagcatccagtacgacaaacagcacgtctgtggagggagcatcctggacccccactgggtcctcacggcagcccactgcttcaggtaagaccccagctgtaaggaggtctctggggaccaaggccagtcagggaccagagagcttggggtcctgtctcctggcaccgtccttctcttcactctcccactagagacgttttccaggttgtggtggccccaatgagacaatggccatgatgccctttgttaggcttttgggtgtctgagcagagggtgctggtcaccaagcatggcctcttcctggtgggacaccagcagatacccagagtcctcaccccacccccatatcgttcaagctacaaaagctcttcccacctgcctcaacttccaagaactcactctctttttgcttgtttccaggaagttgttccagggtctagagtcatagccacgtcctcattatgtctggaaactttaaaaaaattaaagagcataggttcctttcagtccacagagaagcctggccttacctcagggaagggctactcccagacccccttcactttttttttttttttttttttttttttttttgagacagagtcttgctctgttgcttaggctggagcgcagcagcatgatcttggctcactgcaacctccgcctcctgagttcaagcaattctcctgcctcagcttcccaagtagctgggactataggcatgggccaccatgcccggctaatttttgtatttttggtagagacagggtttcaccatgttggccaggctgatctctaactcctgacctcaagtgatctgcccacctcagcctcccaaactgctgggattacaggcatgagccagggcatccggcttttatttattcattcattcaatatctaatgagcacctaccaggtaccaaacaccagatgatgcgcccaagttcattagaccccaccgctgtcttcaaggcactcatgatctaggccagcgttttttaaccactttttttttttttttttttgagattctggtgagagctataaattctttcctggaaaaacatctctgcacactaagctgtgcctggcattgggaaaaagaaagcacgtaatgtaactgacagcatgagtaacacagtgagaaaggttggaggagagagcgccaggacctcagaactcaggcattagaggagccccttccccagccctccttgaggtttcgttgggcaggtttcactgaggaaaaagggtcaaatccctttttcgaatttgacttcttgtaagtgccagaagactgccccttctccaccatccctgcctcaccatcatctttcctcccaaggcagtgacatccagcaccccgatccctagggccctggggacccagcctttggcaaagtctcctcaggcttggatcaggcctgaacccagctgtctctacccccaggaaacataccgatgtgttcaactggaaggtgcgggcaggctcagacaaactgggcagcttcccatccctggctgtggccaagatcatcatcattgaattcaaccccatgtaccccaaagacaatgacatcgccctcatgaagctgcagttcccactcactttctcaggtgagaagcagggcccaaggccactcaagcctcttacatcagttttcacgcccactctgctattagctcactgaccgcccttggcacataatgtctcctctcaagtcctcagcttgcccatttgtctctaatacgtcagcctaacatcactgatgccatgaggcctcctcaagctgtcagctaacacctccactccattccctgccagagattcttccaaggcctgtcttccctatgtggagcccctcgagtgagaactggagtttcatccaatcttggagttttaggagaccttttaaaaagattatcgagctaattccccaccactgaccaacacgcaagagcctgctcagtatccctgccaaggagtcattgtgcccctgtttgctctcctccaggggcagggaacccattacctgtgaggcagcccacagagtctttgaacagctctgttggatgccttgtgcttatactgaaatgtatttagatcaggattcccaactgtggggtccacaagacactggccccttggagaagagaggattccattgtcaaataagtttggggaacattttcatactacagctcccttcttggaacacattagtttattaaaggtaggagaagtttttaaaataatctgttttattgcgtttaacctacattttttaaatttatttgaccacagaatccttttttcatgctacttctattagcatcccatagaacaagtgttctagagaccctggtgtgacccctttcagagagcttaactgccaggctctcctgagccctggtgtgtgtttcaagatttgtgcctgggaattgttttaatcaggtatggcaaggtgacagatacagacacagctatctttgaaagaagagtttattatttataattcctgagagaaagggacataccccaccccccaacacagggacacccggggaagcagctgggtccaccaggaggcaggagtgaggggaaggcatggcccagagccacctgtggcttccatgggcaggtctggccaaggtagggtaggcaagattgagcatgctcaggattggatagtgtggacaattctctaggctatagatgtcagcctctggttgtctagtatctgtccctggggtgatttagggcagggaaaatattggcttggtgtctgagagtcagataaaggaagtggttggggatatgggctttgggttggctggtttgcctattaaaggcgtgcccaaagccaagttgtttactatctgcaggaattagctaacccagtctctcccagaccagcaagatccccataatcataaagcatcataatttacagaaaattaacacttatgatgaataaaagatctccttcttcctctgtgctcctggcaggcacagtcaggcccatctgtctgcccttctttgatgaggagctcactccagccaccccactctggatcattggatggggctttacgaagcagaatggaggtaagtcctgggtgcaggaccacagggcaggagatgcccttgtatgagggagcagcttccagaagtaatgggaaggaggaccacccttcagagaaacccatcctggaggaccaagcaccaaggcgccaggcagaaagcaaagtggtttggcaatccagggctgggggatagaaggcaaggatgggaatgtgagtgtttttaccctcccagggaagatgtctgacatactgctgcaggcgtcagtccaggtcattgacagcacacggtgcaatgcagacgatgcgtaccagggggaagtcaccgagaagatgatgtgtgcaggcatcccggaagggggtgtggacacctgccaggtggggcctccaagaatcatggggagttctaagaatagggtttaggtcctagagagatgagaaaacccagaggctgcatgccctacaggaagccttgcatatcatgggcactcaatgtgtgatgatgggaggaagagagggagggaaggaaaggatagtcagataaaagtgtaccaatagatgagtgggtggatggatggatgcagacaagcagagagatttcaaatgtctctttcacattcgaagatgatgttactggcctggcatggtggctcacgcttgtaatcccagcactttgggaggctgaggcgggcaggtgatttgaggtcaggaattcaagaccagcctggccaacatggtgaaatcccatctctactaaaaagaatacaaaaattagctgggcgtggtggcacgtgcctgtaatcccagctacttgggaggctgaggcaggagaattgcttgaacccaggaggcagaggttgcagtaagctgagattgcgccactgcactccagcctgggtgacccagcaagactccatctgaaaacaacaacaacaacaaagatgacattactcatccaccccacccacccttctcactagctacagaatgattagccccttgaggtcaggaatcccaggtctattttctctgtgactctccccaagctgctgaactacactaggaaagaattaccgcctgcagaatgctggaagcacatctgtgtgtgccctcaccccggcctcattggccatcaggactgcttagcaatccctgtagaccttcttcctcccccatacttccagaggatcttctgaactattttctttttttattttttcttttatgttttttaacagagacagggtcactatgttgcccagtctggtctcaaactcctgggttcaagggattctcccacctcagctttccaaaatgctgggattacaggcatgagccatcgtgcttggcctgaaccattttcattaaaacccctaccctactctcacctccatttccagtcattaaattccttcatttaagaggcatctcttagtcatcgcatgtgtgccatgaacatggtagtctttggagacccctcagggagctcacagtggttgggggaaaggggggcattaaacagacatttaagctatagttttgggttcagagggaggaagccccaggggctaaaacagctgataaggactcccagataagtgcacttttcactatctggcattttcttgttttgttatttgcttgttcactgtctctcaccccatttgatcctaagctttctgagggcagggatctttgttttttttcatcagttggatcccaattgcttagaacactacctggcacaaaataggcactctataagtgattacacaaattttggaacgactaggttaaacaatgataaccaggcttttttttttttttttgagactgagtctcactctgttgcccaggctagagtgaagtggtttgatctcggctcactgcagcctccgcctctgggttcgaatgattctccacctcagcctcctgagtagctgggattacaggtgcctgccactatgcccagctaatttttgtatttgtagtagagacgggtttcaccatgttggccaggctggtcttgaactcctgacctcaagtgattcacccgcctcagcctcccaaggtgctgggattacaggtgtgagccaccgctcctggccaacaaccaggcttttttaagacatcactcagagcctttaatttgctaatgtgagttgtgaatctctgagagaaggctaacggcatgcttgcaacttacttgtccacagacaagcctttctgccccagaagagaagaccattctagggtgctaatgagcaaagagggtgagggtggaatatcggagagcagcagggagtgcaggggaacagataggccagttcagggagcagagaaggagaagcccccccacctcacctgccctccccagcagtctctgttctggtctctcacagggtgacagtggtgggcccctgatgtaccaatctgaccagtggcatgtggtgggcatcgttagttggggctatggctgcgggggcccgagcaccccaggagtatacaccaaggtctcagcctatctcaactggatctacaatgtctggaaggtaaggtacctttgccctacccactgtgccttccctccagtcctctacctggggggtgccaatccatcctcaggtttgatttaaatggttctgacaactctttacatcccaaataactttccctccaagcaagggacagcctgagattgcactattaaggctgaaattccttaggtcagagatttctgataaatgcaaataccttagggaatagaacacaccaagcctttctttctcttttctgacagaatgagactatcagatcctttctagagagaagattctgataaggaagagagtggaaaggctcatgagacctcctggccctctgcagggtagggagagaagcaaagtgtttcagaaaaggaagactcacgttacacatgtcaccactttgtccagtttcagataatctgactttctcttcatcggtctctcttattctaggctgagctgtaacgctgccgtcccccacatccagaagctgcttcccttcagacctacctacggcatgacccctcaaagtcagatatgggacaagagcctccttgaacaaactc

SEQ ID NO: 80 - DNA, 15159 bp, synthetic construct
  misc_feature 1..15159: note = Recombinant polynucleotide
  source 1..15159: mol_type = other DNA
ccacccgcacacactacagtcgagataacttcgtataatgtatgctatacgaagttatgctagtaactataacggtcctaaggtagcgagctagccaagtctgtgtgctaccaagtagcaaaactgagcctggaactcacacatgcgtgtctgagagcccagcactatcgccaggaaaacccagcgtctccctgctcaagcctgaccctcagccctctctgcctctccctgcacttgccttccagtcaaggtgattctggataaatactacttcctctgcgggcagcctctccacttcatcccgaggaagcagctgtgtgacggagagctggactgtcccttgggggaggacgaggagcactgtgtcaagagcttccccgaagggcctgcagtggcaggtgagtgcagggtctgaggcacaagagaagtgggcccagcaggaggtctgctcaggcccccacggcccactgcatagtatctgccccctacttgtcacttttcatccttgttgtataaggttctttgtttgtttgtttgttgttgttttgaggcagagtgctctgtggcccaagatggagtgcagtgtcttggtctcggctcactgcaacctctgcctcccagtttcaagtgattcttctgcctcagcctcatgagtagctgggattacaggtgccagccaccacgcctggctaatttttatatttttagtagagacggggttttgccacattggtcaggctgatcttgaactcctgacctcaggtgatctgcccgcctcagcctcccaaagtgctgggattacaggcgtgagccaccgtgcccagctgtgtaagtttcttgagagcaggaccctgtcttgtctacctttaaatcctagtacttaacacacagcaaacagtaactatttgatgaccaaatgtgagccagaaaggacaggaaattgtaactgaggctgccccatgcgtgctgcgcctggtggatttcaggcagagggctagactgggtgaccttggggcattcctcctttctatgaaatttgttatttcaaggagactagaaaagagacttctcagccacttcgccagctattggtccttctattcattagtgtttgctgagacatgctatgtgacaggactgagccaggtcctttcaatggataggagatgttttgagcataaaatccacgttctctcttgggctgggctcttctaccttcttccccctggtgcttgggctctgaagaaaaaaagataggtaggagatgagtgatggggcttctgagggcagggctgagtgactttctgtgtatttgctctttctttatcagaagtcaaatgcccacaggcacctgtcatcctactgccagtaggacttctcactcaaccttcccctctgaccttacttggagaaggacttaggtccctctctcagacatttccccaggctgggcaagttgtgtggaccatggatgggtatgtggtccatacaatttaaacaagctgtatatggtcgctgggtagagtgaccacataattgatcatcaaaactgatacctgtaagagcaaaagggggcactattaaccattgggtcagggcaacaggtcaaaatggagacctaccctgggacttctggtcacactagctactgtcaaaatggggcccaaatagacaaagccaaatggaagaaattcccttgacattgaaagtgttggggctctgtggcacccccagttctaggttgggggagcttgggctggtctcatgatgagttctgagggggatgggccagttgggccccccgttccatctaactcaggttcctttcctcccagtccgcctctccaaggaccgatccacactgcaggtgctggactcggccacagggaactggttctctgcctgtttcgacaacttcacagaagctctcgctgagacagcctgtaggcagatgggctacagcaggtaaccaacctgggcctctctcctttttccctccttcctccttcctcctcttcctcctttccttcctcccttcttctctctttcctaaaaattacgggcattggagccaggcagaatggcttttgaatcccagcatttcacttataagcaacatgaagttaaatttcctaagcctcaggttcctcaggagttaattgggggaactaatgccaacctcataggatagttttgcaatgccagtgagagaatgtgtgctgccctccaacacacacacacacacttctagcgtctatgcagtcctctcctttcctttactcctcaaccttcactcctttgtgctggctttgcaagaaactgttcctgcccagtaatacaaaagctaagttaacttattcaaagtttcgttagttaagatttagcttaagtgagcctagtttcagtggggccccatcttcagcaatcccagctctctctgcaaatttcaaaagcagttccaaatctggagtggatgaaaaggtgtaagatgatagtaagagtaatttgcattctatatatttatattcacttgattttggcagaaaaccaaaaagatagttattatatcttatatatagatatatattatatctatttcataaataggctcaaacaaagtaagtaacttgctagggtactagctgggaggtagagggctagaatttgagcccaagacccctaattcttgcgcattaggagttcccacattgtttctgtttctagactgagtaattctttattctcatgtaggacatcatctctaagggaaggggctaatgagatggttgatcactcagagagtttagctggagaggatggaaaagaacccatacattcagttgcagattgagatagcctatctctggcaggcctcagatttcttcaggattctaacagactggacccagagactaggccaaacaaacaaacaaacaaaaactctactaggcagacatcaccaaccaatcacagaactctctcccatggatccctaatacagcctcaaagtccttttcagtaaatgctccaggcagccattacaaatcaatcagaattatttgcctttctcttctctgctcaacgggcttctgctgctctctactttccatagggggcaacttccattaccctctagaaagcacaccccaccaccttcatttcaaggagagtgaggaactcatgcccagcacctgctattctcccctcttcctgcagccacggagcccagcctcgctgcagccagccctgcctccccactgtagtccagtcaactgctgcatcagccgttcctggcacagcaggctgagccttgattatgaaacctgggtgtctccaggggttcttaagatgataggctcctggaatttctgtccttttggagctcagtaaggcaccaaaccacctgagtcttgtgcttcacaaaatcaaagttcatcagaatcattcattgggatggaattggtgaacagaagttaactttcctgggaatgtccatttccaccatattccgtccttctaggtctcagacttctctactttctttcctctctctagatcggaggcccttcttgtcctagaaccataggcatttcaagatgtgggagaccctagggatcatctagtccacgcatcttttttttttttttttgacagagtctcactctgtcacccaggctggagtgcaatggcaccatctctgcttactgcaacctccacctcccaggttcaagtgattctttcgcctcagcctcccaagtagctgggattacaggcacgcaccatcatgcccagctaatttttatatttttgtagagaccgagtttcaccatgttggccaggctggtcttgaactcctgacctcaggtgatccacccacctcggcctcccaaagtgctgggattacaggcgtgagccactgcacccagccccgtgcatctttttatagagggggaaactgaggcttggagagacccagaaaaagaatatgacctgcccaaggccacacatcaaactagtgccagagccagggacagaacctagatcatgaggactcttaaaatgcactctagtcctcccaggtctgagacttgggtccttccaggaagtgccagcattcctgcctgagaatgtgccaatccaccagtattgccaatgactcagccctccatggagagcttctactaacattactagcatagttagggatggaaggaaaagatttagaagaggcagattcagtaaaggaacaatcagagagatggaattaatcaaggaaggcttcctggaggaggaaaaacttcaacccaaggtttgaaagtagcaagcatggattagcagggagaaagagggagagtggtccagttgagagaaacgtttgtctggattcatatgaagacagatctagtcctgttctattaaatatctctaagggggccaaaaacatacccccgctatcaaagtcagaccagatgctttgtttggagaacgaaatatccacattccaactccctcccaggtgagaagggagctaacctgagcccctatgcctctttgtttccctgctgtgaaccagaagacattgctgggatatttgaaatagggacagagctgggaatatggaaaggagacccctaacatttctccagggctctgggttctggatttggattccccacccaagaaagcaagttacatcagcaatgcactgagggttgagtcctgggatgccaagggtcggttctttattgtatagcaaagcaggccccatcttcactgactaagaccatctccactccctggccactccccaccaagcattctctgccactctttctcctgaaagtgggggccaactctaccatcttgttctaaccccctgccccagctcacaactctctctccctcttgatgtgagcagcaaacccactttcagagctgtggagattggcccagaccaggatctggatgttgttgaaatcacagaaaacagccaggagcttcgcatgcggaactcaagtgggtaagtgaggggacaccttctggcctacagaaggcccccacatggacgctgctcttcaggttgcaaccagctcacctggaaccccaagcagccaggggaatgtaagcagacatcaggaagaactcctagccagatggatcattcaatgccaagagctatagactcacattttggagaggttttctgtgttgacttgtttttaatacaatggacagctggacaaagtgtgttgtcctactcagagccagagggatggataatgtgacctttccatcaatctggatagtaaatagtttttgctactgctgtaggttttctaataaattgcccaataggcaagattccaaagtcactttgtccttccctaccacttacccagccagagctccccaccttcttgatgctccagggaagaggctccatggcccttgtgggtggcctgttcctgagcctcgccaccctgtgttagagcagagcatccagatgaaatctgtcacactgtggcaaagtggctcagagaggaggctggcttcctagcattcagggacgttgctgagggccgcttattcaccgaaaataaatcttgaaaaggacagggctggtagcagaatgatcctttacctaaaattctatcaaaatcccattcttccatttggaaagcccacagtgtcacagactctgttccgggctctgtcctcttccctcttgggtcccaggagcccaggctgggctttgaagcaggcagggcccagcacacagtaggtactcagcagtgggggtgttgaatccaatcaaacggaagtgtcaatgcaggaaatgcaatggatgtcaatgcagtctccaaatgttccccactgtgcagcttccacattcccgaggtattgggaggggacttgaattaacagcttcgggaggcctgagtccctgcctcccagctgaggaagaagcttaaatcacagggcgctgtgtctgtcttccaggccctgtctctcaggctccctggtctccctgcactgtcttggtgagtacccccaatctctgagggtttggggcctgggccagcaatgagcagggaggaagaccttcatcttcactcctaaatttctgggactccaagtttcattctgccttggtctacagcccttgggcttgtcggtcaatgccccctcgagttgttggtggccttgggcaggtcacattctttttctgggtctttccaagccccagtttcccccttctaccatctgtgcatggctccatgacctaagtggagacctgggagagagtgttaggaagaccgaaaagggcaggacggggcctccactgcctcccatccctggtccgggcccacatagccttctttgtcacaatcagctcaggtatccaagatcagattacccacattcattatttgagcaactattcattgaacagttagaatatgtctcactctgtcagttgctggctagaagtagaaagtaccagatgagtgaaataattggccactatccttggtagctgatgactaagtaagagagagatgcaagacaacatgtggaaaatgccaaactgagtagcagtcacagttgacatgctgcagagagagctggccgggggtcagaagacctgggcaccagtcctgttcatttccagtgtggcctcgagtcattcacctgacctccctgaagttcattttcccaagaagttgtttagtccaactgcccatcaaggatctttagggacccttctagctctaacagaggagatcagaaaagaaaacaagcaatgtggctcagctcatcctacaagcttcatagagaactgagactggcctggaagcatagccagaaattagaacgcctaagggaagaaggtcacaacgctgcctctgcaatttaggagtgtatatgctttcctgcaggatgttgagagtttcattcattatcgtatgccccctaccccggccccacaatacctagtgcgtgggatctgacacgtggtggctggtcaatgaatgaatgaatgaatggtcacaccatctgaggttctgcactgagtagccctgaaggcttgaagcagcataagtgacaggtcctcccttgaggggcctctgttttaccaataagccaagacctaagctcaacaacactgaaagggtggccaatacccaggacagcctgtgggaattccagagaaagggagattcccagggactgggggcccaggctaaacactgaaaaatgcatctgtaggctcaaggaggaaaagcccatgtctgtctgtcttgcccaccactctctcccagcacccagcactgccccaggacagagagcacttgacacaagttggttagattaatgaatgatttagagttcagtggtccccaacctttttggcacaagagactggttgcatggaagacaatttttccgcaaaccaagagggggatagagagcattagattctctcttttttttttttttgagaccaagtctggctcttgtcactcagcctggagtaaagtgttgcgatctcggctcactgcaacctccgcctcctggattcaagcgattctcctgcctcagccccctaaatagctgggattacaggcacccgtcaccagcccagctgggactataggcatgtgccaccatgcccggctaatttttgtatttttagtagagacggcgtttcaccatgttggccaggctagtctcgaactcctgacctcaggtgatctgcccgcctgagcctcccaaagtgctgggattacaggcatgagctgcctcacccagcctaaagtctcataaggaacgtacagcatagatccctcacatgtgcagttcacaataaggttgtgctcctacaagaatctaacgccacctctgatctgacaggaggtgaagctcaggtggtcatgctcgcttgtccctgccactcacttcctaatgtacagccaggttcctaacaggccacgaaccagtgggaagggcatctttttggatcaaaaacagaattactttttagagaactacaagcagatcaatttggctagacagagactttatatgaaacagcaggaggctgctaggaggagtggaaactctactttgccctcaagggagatcccgaagggctttgcaggagcgggcaaggtggcatgaagaaagcagtgtttgaaatcaggtggtatttgaaaagcccagcccttccccttagaatggcccttctaccatctgtgcatggctccacaaccgtggtggtggctgccagaagaattggaaaggcagagcatgggtggagaggggggacctgagggctttacaggagttccgggggtggtgagggtgtgaaagccaggtcagtcagtaggaagacaggatgtcagattgagagactcccctggccggggaaacagacttggagaagggggagttttggatgagacagtccacttccgagtcacaaaatagcttgtgggtgtctgtttactgttactcagtgggagtggctggggacacgccacctgggcagggctttcgtaattctgcatcacttgtgaaggtcacagattcccagcacaacggacacacccatgttcatagtctgaactcctaaacacatcttaaaccaaaataaaaaaaaaagaaagaaagaaagaaaaaggagagggaggtttgaggaaagcctatggtctgggacactcaatacctcccatgaatatctcatattgggctggtcctctctccactctggccccagccataagggccctgcttagagcagattttgggtgctgagtggaggcagcctcatccccaacagcctgacttcctgcctcctccctgcctctgcctgtgtccagcctgtgggaagagcctgaagaccccccgtgtggtgggtgtggaggaggcctctgtggattcttggccttggcaggtcagcatccagtacgacaaacagcacgtctgtggagggagcatcctggacccccactgggtcctcacggcagcccactgcttcaggtaagaccccagctgtaaggaggtctctggggaccaaggccagtcagggaccagagagcttggggtcctgtctcctggcaccgtccttctcttcactctcccactagagacgttttccaggttgtggtggccccaatgagacaatggccatgatgccctttgttaggcttttgggtgtctgagcagagggtgctggtcaccaagcatggcctcttcctggtgggacaccagcagatacccagagtcctcaccccacccccatatcgttcaagctacaaaagctcttcccacctgcctcaacttccaagaactcactctctttttgcttgtttccaggaagttgttccagggtctagagtcatagccacgtcctcattatgtctggaaactttaaaaaaattaaagagcataggttcctttcagtccacagagaagcctggccttacctcagggaagggctactcccagacccccttcactttttttttttttttttttttttttttttttgagacagagtcttgctctgttgcttaggctggagcgcagcagcatgatcttggctcactgcaacctccgcctcctgagttcaagcaattctcctgcctcagcttcccaagtagctgggactataggcatgggccaccatgcccggctaatttttgtatttttggtagagacagggtttcaccatgttggccaggctgatctctaactcctgacctcaagtgatctgcccacctcagcctcccaaactgctgggattacaggcatgagccagggcatccggcttttatttattcattcattcaatatctaatgagcacctaccaggtaccaaacaccagatgatgcgcccaagttcattagaccccaccgctgtcttcaaggcactcatgatctaggccagcgttttttaaccactttttttttttttttttttgagattctggtgagagctataaattctttcctggaaaaacatctctgcacactaagctgtgcctggcattgggaaaaagaaagcacgtaatgtaactgacagcatgagtaacacagtgagaaaggttggaggagagagcgccaggacctcagaactcaggcattagaggagccccttccccagccctccttgaggtttcgttgggcaggtttcactgaggaaaaagggtcaaatccctttttcgaatttgacttcttgtaagtgccagaagactgccccttctccaccatccctgcctcaccatcatctttcctcccaaggcagtgacatccagcaccccgatccctagggccctggggacccagcctttggcaaagtctcctcaggcttggatcaggcctgaacccagctgtctctacccccaggaaacataccgatgtgttcaactggaaggtgcgggcaggctcagacaaactgggcagcttcccatccctggctgtggccaagatcatcatcattgaattcaaccccatgtaccccaaagacaatgacatcgccctcatgaagctgcagttcccactcactttctcaggtgagaagcagggcccaaggccactcaagcctcttacatcagttttcacgcccactctgctattagctcactgaccgcccttggcacataatgtctcctctcaagtcctcagcttgcccatttgtctctaatacgtcagcctaacatcactgatgccatgaggcctcctcaagctgtcagctaacacctccactccattccctgccagagattcttccaaggcctgtcttccctatgtggagcccctcgagtgagaactggagtttcatccaatcttggagttttaggagaccttttaaaaagattatcgagctaattccccaccactgaccaacacgcaagagcctgctcagtatccctgccaaggagtcattgtgcccctgtttgctctcctccaggggcagggaacccattacctgtgaggcagcccacagagtctttgaacagctctgttggatgccttgtgcttatactgaaatgtatttagatcaggattcccaactgtggggtccacaagacactggccccttggagaagagaggattccattgtcaaataagtttggggaacattttcatactacagctcccttcttggaacacattagtttattaaaggtaggagaagtttttaaaataatctgttttattgcgtttaacctacattttttaaatttatttgaccacagaatccttttttcatgctacttctattagcatcccatagaacaagtgttctagagaccctggtgtgacccctttcagagagcttaactgccaggctctcctgagccctggtgtgtgtttcaagatttgtgcctgggaattgttttaatcaggtatggcaaggtgacagatacagacacagctatctttgaaagaagagtttattatttataattcctgagagaaagggacataccccaccccccaacacagggacacccggggaagcagctgggtccaccaggaggcaggagtgaggggaaggcatggcccagagccacctgtggcttccatgggcaggtctggccaaggtagggtaggcaagattgagcatgctcaggattggatagtgtggacaattctctaggctatagatgtcagcctctggttgtctagtatctgtccctggggtgatttagggcagggaaaatattggcttggtgtctgagagtcagataaaggaagtggttggggatatgggctttgggttggctggtttgcctattaaaggcgtgcccaaagccaagttgtttactatctgcaggaattagctaacccagtctctcccagaccagcaagatccccataatcataaagcatcataatttacagaaaattaacacttatgatgaataaaagatctccttcttcctctgtgctcctggcaggcacagtcaggcccatctgtctgcccttctttgatgaggagctcactccagccaccccactctggatcattggatggggctttacgaagcagaatggaggtaagtcctgggtgcaggaccacagggcaggagatgcccttgtatgagggagcagcttccagaagtaatgggaaggaggaccacccttcagagaaacccatcctggaggaccaagcaccaaggcgccaggcagaaagcaaagtggtttggcaatccagggctgggggatagaaggcaaggatgggaatgtgagtgtttttaccctcccagggaagatgtctgacatactgctgcaggcgtcagtccaggtcattgacagcacacggtgcaatgcagacgatgcgtaccagggggaagtcaccgagaagatgatgtgtgcaggcatcccggaagggggtgtggacacctgccaggtggggcctccaagaatcatggggagttctaagaatagggtttaggtcctagagagatgagaaaacccagaggctgcatgccctacaggaagccttgcatatcatgggcactcaatgtgtgatgatgggaggaagagagggagggaaggaaaggatagtcagataaaagtgtaccaatagatgagtgggtggatggatggatgcagacaagcagagagatttcaaatgtctctttcacattcgaagatgatgttactggcctggcatggtggctcacgcttgtaatcccagcactttgggaggctgaggcgggcaggtgatttgaggtcaggaattcaagaccagcctggccaacatggtgaaatcccatctctactaaaaagaatacaaaaattagctgggcgtggtggcacgtgcctgtaatcccagctacttgggaggctgaggcaggagaattgcttgaacccaggaggcagaggttgcagtaagctgagattgcgccactgcactccagcctgggtgacccagcaagactccatctgaaaacaacaacaacaacaaagatgacattactcatccaccccacccacccttctcactagctacagaatgattagccccttgaggtcaggaatcccaggtctattttctctgtgactctccccaagctgctgaactacactaggaaagaattaccgcctgcagaatgctggaagcacatctgtgtgtgccctcaccccggcctcattggccatcaggactgcttagcaatccctgtagaccttcttcctcccccatacttccagaggatcttctgaactattttctttttttattttttcttttatgttttttaacagagacagggtcactatgttgcccagtctggtctcaaactcctgggttcaagggattctcccacctcagctttccaaaatgctgggattacaggcatgagccatcgtgcttggcctgaaccattttcattaaaacccctaccctactctcacctccatttccagtcattaaattccttcatttaagaggcatctcttagtcatcgcatgtgtgccatgaacatggtagtctttggagacccctcagggagctcacagtggttgggggaaaggggggcattaaacagacatttaagctatagttttgggttcagagggaggaagccccaggggctaaaacagctgataaggactcccagataagtgcacttttcactatctggcattttcttgttttgttatttgcttgttcactgtctctcaccccatttgatcctaagctttctgagggcagggatctttgttttttttcatcagttggatcccaattgcttagaacactacctggcacaaaataggcactctataagtgattacacaaattttggaacgactaggttaaacaatgataaccaggcttttttttttttttttgagactgagtctcactctgttgcccaggctagagtgaagtggtttgatctcggctcactgcagcctccgcctctgggttcgaatgattctccacctcagcctcctgagtagctgggattacaggtgcctgccactatgcccagctaatttttgtatttgtagtagagacgggtttcaccatgttggccaggctggtcttgaactcctgacctcaagtgattcacccgcctcagcctcccaaggtgctgggattacaggtgtgagccaccgctcctggccaacaaccaggcttttttaagacatcactcagagcctttaatttgctaatgtgagttgtgaatctctgagagaaggctaacggcatgcttgcaacttacttgtccacagacaagcctttctgccccagaagagaagaccattctagggtgctaatgagcaaagagggtgagggtggaatatcggagagcagcagggagtgcaggggaacagataggccagttcagggagcagagaaggagaagcccccccacctcacctgccctccccagcagtctctgttctggtctctcacagggtgacagtggtgggcccctgatgtaccaatctgaccagtggcatgtggtgggcatcgttagttggggctatggctgcgggggcccgagcaccccaggagtatacaccaaggtctcagcctatctcaactggatctacaatgtctggaaggtaaggtacctttgccctacccactgtgccttccctccagtcctctacctggggggtgccaatccatcctcaggtttgatttaaatggttctgacaactctttacatcccaaataactttccctccaagcaagggacagcctgagattgcactattaaggctgaaattccttaggtcagagatttctgataaatgcaaataccttagggaatagaacacaccaagcctttctttctcttttctgacagaatgagactatcagatcctttctagagagaagattctgataaggaagagagtggaaaggctcatgagacctcctggccctctgcagggtagggagagaagcaaagtgtttcagaaaaggaagactcacgttacacatgtcaccactttgtccagtttcagataatctgactttctcttcatcggtctctcttattctaggctgagctgtaacgctgccgtcccccacatccagaagctgcttcccttcagacctacctacggcatgacccctcaaagtcagatatgggacaagagcctccttgaacaaactc

SEQ ID NO: 81 - protein, 435 aa, synthetic construct
  REGION 1..435: note = Recombinant protein
MESDSGQPLNNRDIVPFRKPRRPQETFKKVGIPIIAVLLSLIALVIVALLIKVILDKYYFLCGQPLHFIPRKQLCDGELDCPLGEDEEHCVKSFPEGPAVAVRLSKDRSTLQVLDSATGNWFSACFDNFTEALAETACRQMGYSSKPTFRAVEIGPDQDLDVVEITENSQELRMRNSSGPCLSGSLVSLHCLACGKSLKTPRVVGVEEASVDSWPWQVSIQYDKQHVCGGSILDPHWVLTAAHCFRKHTDVFNWKVRAGSDKLGSFPSLAVAKIIIIEFNPMYPKDNDIALMKLQFPLTFSGTVRPICLPFFDEELTPATPLWIIGWGFTKQNGGKMSDILLQASVQVIDSTRCNADDAYQGEVTEKMMCAGIPEGGVDTCQGDSGGPLMYQSDQWHVVGIVSWGYGCGGPSTPGVYTKVSAYLNWIYNVWKAEL

SEQ ID NO: 82 - DNA, 2046 bp, Mus musculus
  source 1..2046: mol_type = other DNA
cagaaacaaggacctcttcattattcaagagtaaaatgtataggccaagaccaatgctatcaccgtcaagattcttcactccctttgcagtagctttcgttgtcataataacggtagggctcctggccatgatggcaggtctacttattcactttttagcttttgacaagaaagcttacttttatcatagcagctttcaaatcctaaacgttgaatacactgaggctttaaactcaccagctacacacgaatacagaaccttgagtgaaagaattgaggctatgattactgatgaatttcgaggatcaagtctaaaaagtgagtttatcaggacacatgttgtcaaactaagaaaagaagggactggtgtggttgcggatgttgtcatgaaatttcgatctagtaaacgtaacaacagaaaggtaatgaaaaccagaattcaatctgtgctacgaagactcagcagctctggaaacttggaaatagccccttcgaatgagataacatcactcactgaccaggatacagaaaatgttttgactcaagaatgtggagcacgtccagaccttataacactgtcagaagagagaatcattggaggcatgcaagctgagcccggtgactggccctggcaagtcagtctacagctcaataatgtccaccactgtggaggtgccctgatcagtaacatgtgggtcctgacagcagctcattgcttcaaaagctatcctaatcctcaatattggacagccacctttggggtttctacaatgagccctaggctgagagtgagagtaagggctattttagcccacgacgggtacagctccgtaactcgtgacaatgacatcgcagttgtacaacttgacagatctgtcgccttttccagaaatatccatagggtatgtctcccagcagcaacccaaaatatcatccctggttctgtcgcatatgttacaggatggggatctctcacatatggaggcaacgcagtcacaaatctacggcaaggagaggtcagaataataagttcagaggaatgcaatacgccagctggttacagtggaagtgtcttgccaggaatgctgtgtgctggaatgcgttcaggggccgtggatgcatgccagggtgattcaggtggcccgctagtacaagaagactcaaggcggctttggtttgttgtgggcattgtgagctggggatatcagtgtggcctcccaaataagccaggcgtgtatactcgagtgacagcctaccgcaactggatcagacagcagacgggaatctagtgcaaccgaggaaaaaacgtgccatgaggtctctgtatccaagtgtgactgactcggatgccatggcttcacatttcaactgcaaaggagactggaaatgccccttctgaacgtcccattacataaatatggtttaactgtttagtatttctttgtcggtacagatttttactttcttgaggaaaaaaaaaacatgaacatggctaagtaagaattatgttaggctagtaacaggaagacatttattacatgggtggtcaggtgtagtagtgagaagtcaggtaagttaagtcaataatttacagaaaataatgtcaggtagtcctaacgttaaatatgtgaggccacagaacaaatagtgttagaactgaagccatcccaagtatttaacatttgttttcaagtgaaactaagaaacagacttacatatagttttaatggtgaattttcattttaaatattttatctacatagaaaagacatatctccttcatgaagaagctgaggtgatgaatcaacacagcctcttcagctatgtttgcaaccacaagatttgtgggaaagaaatccctactaccaacttcctactgttggcattattttttagagtaacacgacgcacaatagcaaaatttaagtaacaaattaaaagttaatgatgaagaagaagtaaagagtttgtttgcaaagacaaaaattaaacagattaatatcaataaatctggagacagaagggtctcagattcatattctctct

SEQ ID NO: 83 - protein, 417 aa, Mus musculus
MYRPRPMLSPSRFFTPFAVAFVVIITVGLLAMMAGLLIHFLAFDKKAYFYHSSFQILNVEYTEALNSPATHEYRTLSERIEAMITDEFRGSSLKSEFIRTHVVKLRKEGTGVVADVVMKFRSSKRNNRKVMKTRIQSVLRRLSSSGNLEIAPSNEITSLTDQDTENVLTQECGARPDLITLSEERIIGGMQAEPGDWPWQVSLQLNNVHHCGGALISNMWVLTAAHCFKSYPNPQYWTATFGVSTMSPRLRVRVRAILAHDGYSSVTRDNDIAVVQLDRSVAFSRNIHRVCLPAATQNIIPGSVAYVTGWGSLTYGGNAVTNLRQGEVRIISSEECNTPAGYSGSVLPGMLCAGMRSGAVDACQGDSGGPLVQEDSRRLWFVVGIVSWGYQCGLPNKPGVYTRVTAYRNWIRQQTGI

SEQ ID NO: 84 - DNA, 2800 bp, Homo sapiens
  source 1..2800: mol_type = other DNA
atttgagtgggaatctcaaagcagttgagtaggcagaaaaaagaacctcttcattaaggattaaaatgtataggccagcacgtgtaacttcgacttcaagatttctgaatccatatgtagtatgtttcattgtcgtcgcaggggtagtgatcctggcagtcaccatagctctacttgtttactttttagcttttgatcaaaaatcttacttttataggagcagttttcaactcctaaatgttgaatataatagtcagttaaattcaccagctacacaggaatacaggactttgagtggaagaattgaatctctgattactaaaacattcaaagaatcaaatttaagaaatcagttcatcagagctcatgttgccaaactgaggcaagatggtagtggtgtgagagcggatgttgtcatgaaatttcaattcactagaaataacaatggagcatcaatgaaaagcagaattgagtctgttttacgacaaatgctgaataactctggaaacctggaaataaacccttcaactgagataacatcacttactgaccaggctgcagcaaattggcttattaatgaatgtggggccggtccagacctaataacattgtctgagcagagaatccttggaggcactgaggctgaggagggaagctggccgtggcaagtcagtctgcggctcaataatgcccaccactgtggaggcagcctgatcaataacatgtggatcctgacagcagctcactgcttcagaagcaactctaatcctcgtgactggattgccacgtctggtatttccacaacatttcctaaactaagaatgagagtaagaaatattttaattcataacaattataaatctgcaactcatgaaaatgacattgcacttgtgagacttgagaacagtgtcacctttaccaaagatatccatagtgtgtgtctcccagctgctacccagaatattccacctggctctactgcttatgtaacaggatggggcgctcaagaatatgctggccacacagttccagagctaaggcaaggacaggtcagaataataagtaatgatgtatgtaatgcaccacatagttataatggagccatcttgtctggaatgctgtgtgctggagtacctcaaggtggagtggacgcatgtcagggtgactctggtggcccactagtacaagaagactcacggcggctttggtttattgtggggatagtaagctggggagatcagtgtggcctgccggataagccaggagtgtatactcgagtgacagcctaccttgactggattaggcaacaaactgggatctagtgcaacaagtgcatccctgttgcaaagtctgtatgcaggtgtgcctgtcttaaattccaaagctttacatttcaactgaaaaagaaactagaaatgtcctaatttaacatcttgttacataaatatggtttaacaaacactgtttaacctttctttattattaaaggttttctattttctccagagaactatatgaatgttgcatagtactgtggctgtgtaacagaagaaacacactaaactaattacaaagttaacaatttcattacagttgtgctaaatgcccgtagtgagaagaacaggaaccttgagcatgtatagtagaggaacctgcacaggtctgatgggtcagaggggtcttctctgggtttcactgaggatgagaagtaagcaaactgtggaaacatgcaaaggaaaaagtgatagaataatattcaagacaaaaagaacagtatgaggcaagagaaataatatgtatttaaaatttttggttactcaatatcttatacttagtatgagtcctaaaattaaaaatgtgaaactgttgtactatacgtataacctaaccttaattattctgtaagaacatgcttccataggaaatagtggataattttcagctatttaaggcaaaagctaaaatagttcactcctcaactgagacccaaagaattatagatatttttcatgatgacccatgaaaaatatcactcatctacataaaggagagactatatctattttatagagaagctaagaaatatacctacacaaacttgtcaggtgctttacaactacatagtactttttaacaacaaaataataattttaagaatgaaaaatttaatcatcgggaagaacgtcccactacagacttcctatcactggcagttatatttttgagcgtaaaagggtcgtcaaacgctaaatctaagtaacgaattgaaagtttaaagagggggaagagttggtttgcaaaggaaaagtttaaatagcttaatatcaatagaatgatcctgaagacagaaaaaactttgtcactcttcctctctcattttctttctctctctctccccttctcatacacatgcctcccccaccaaagaatataatgtaaattaaatccactaaaatgtaatggcatgaaaatctctgtagtctgaatcactaatattcctgagtttttatgagctcctagtacagctaaagtttgcctatgcatgatcatctatgcgtcagagcttcctccttctacaagctaactccctgcatctgggcatcaggactgctccatacatttgctgaaaacttcttgtatttcctgatgtaaaattgtgcaaacacctacaataaagccatctacttttagggaaagggagttgaaaatgcaaccaactcttggcgaactgtacaaacaaatctttgctatactttatttcaaataaattctttttaaaataaaaaaaaaaaaaaaaaaa

SEQ ID NO: 85 - protein, 418 aa, Homo sapiens
MYRPARVTSTSRFLNPYVVCFIVVAGVVILAVTIALLVYFLAFDQKSYFYRSSFQLLNVEYNSQLNSPATQEYRTLSGRIESLITKTFKESNLRNQFIRAHVAKLRQDGSGVRADVVMKFQFTRNNNGASMKSRIESVLRQMLNNSGNLEINPSTEITSLTDQAAANWLINECGAGPDLITLSEQRILGGTEAEEGSWPWQVSLRLNNAHHCGGSLINNMWILTAAHCFRSNSNPRDWIATSGISTTFPKLRMRVRNILIHNNYKSATHENDIALVRLENSVTFTKDIHSVCLPAATQNIPPGSTAYVTGWGAQEYAGHTVPELRQGQVRIISNDVCNAPHSYNGAILSGMLCAGVPQGGVDACQGDSGGPLVQEDSRRLWFIVGIVSWGDQCGLPDKPGVYTRVTAYLDWIRQQTGI

SEQ ID NO: 86 - DNA, 38992 bp, synthetic construct
  misc_feature 1..38992: note = Recombinant polynucleotide
  source 1..38992: mol_type = other DNA
gagggagggtggtgctttgctaatggtgaattactaactcctcaataaagaatattatttgaaataatttttgaaatttcataattactttgggttctttcttaatgataaataaataatagtatattacaaacatacattaatatttcctgaatgaatacaccacaaatctcccttaaaatatagcaagaataaaaattatactatttctgacaatttttaatttctcaaataataataccactctgatttttaaacatctacaccactctggctttgccaatctttttaaaaattgaaaagataataattttatcataattacactgaagcatagaactttttctttcaaggaaagcaaatttttgaaattctataatataacctcccataatcctgaataaattaaaggttcaacaacttagtaaagtaagactgaccttcccttttatttctttttcagatcaaaaatcttacttttataggagcagttttcaactcctaaatgttgaatataatagtcagttaaattcaccagctacacaggaatacaggactttgagtggaagaattgaatctctggtaagttaatatttgtctttgctctttattccattataaaatgaatatgataataaacctaatgttttgtaatatattttcagttgctaagtgctctacatattttccttccttgaatggtgaaacatgtgtttctctctgcttttatccagttagtttactcatatactggttcttattcacatctttgtcatgagtaaaaagtgttagaaaggccacgagtaaatatgcattttatttgtttatgaattcaaatactaaaagttttttatttgtttaattaagcattgacattgtctttttaaattcttttcattttaccttcttccctcttccttatccaactaaagacgcaaagcaggaggtgttaaaaaacaggtttaccatatcagcagtaacatagtttggacaacattacactttggttcaatgatagacatagaagtttgaacagaaatatgcaaagcaagtttgagctctaacttgaagagagcctctgggtgcctgccaggaaacctcacgagtggacccttaacattcatgtgtcaccacaaactaggggctgccctttagttttgaccagtctcagtgtcactcacttacccttaccttttcaaaaaaaagtcctaagaatataaagtaattcaatggttctacaattttagcatgtaactgagtcacctggcagggttgctttggtgagctcaagataaaattttatcagcatttctacattttctggaatattccttaatccaggcttttaatcccttggtgcttttctgaaccactgcaatgagcttctaactgttctcactgtgtgcaggctcttttccttctaatctaatttacacacttctgaacacaaatctctcacagcctgtttccttcatgttacctccagctcaagactttttgcctacaaaataaaattcaaacttgttagctaagcaccttctcatgtctatgctttggctcatatttcagccatcgtgtgccccacttattcttatagccaacctgaaaagccatcttttataagaaactacctctgctctccatgattggatataattaatcctccttccacatcacctcgccacaaaattgtatctgtgttgatctcatgccacatacctgtatgtattttatattataaatatttgcagacttgtttaatttgccatgttagactaagttccatgaagacagctccatatccattccatttttatatatccacaacatttggtcgggttgatgcttaataaatgtttattgaaggaacaggagtctcccacttctgacataatgaacttatttcccccagtgttaaccctacatctggttcctgtccaagagtctcttcccaaatcattctgattcaactgttcattctgatctcattaaacatttaaatgatatatctaacttcgcttgctttattctatgctcatcctgcagtctcctcataacttggtttcaatgatgcttgcttctagagaaaaaaatgtattaaataagcttatgattcagtcctccagctgtgatggttctcactgaacattagctcagtggttttcgaagtatggtctctagcataacctagaaacttgttagaaatgcaaattcttgggctcaccaagacatactaaatcaaaaattctgacattggggcctagaaatctgtgttttaacaagcctgccagtgcagcctggtcccttttcttctcggagccccactcaaagctttcagtgctcatctcccaccaatgacagggtcctctatggaaaccggcaggacggtttccaactctaactacgttttagagtttgcttcctagggctatccaggcaccaagtatcacaggttagtttcccagggaagcagactctgagacttgcatgcagggagtgtctctggggtgctctcaaccaacaccttcaggaagagaaggaagcagcattgggcagaggcatagtcaaactacagtgctgttggcacagaagactgaagggagtcagagccagggggtagaggtgggcccttagcatccatccttcaccattaggtgtgagttgccccacctccttgatggtgtaacctcagtcccaaggtgggtgggagtgcagcagagcagcccctacaagggccaaaccagagatacaccaggcgccagaagtgctgccagggaatagagaggaaaggatgggcttaaggtaggatccacagaacttggcaatggattagaagacaggatgagaagtgacaggttaacactaacacagaaatgtctaacttcggtagataatggtgccattggctagaagaggaaaccgaaatgaaagcaggttgttcagggagacaaaagttcactgtggacatctcagcagagtgattcagtggggaaaggaatggatgcccagaccacctcagaggaagatctaagctggagccagcaataaagatacaagatgaacaatccctaacgaactgctcctcagccatgctccccagacacgctgcttcagatttatagtccgggtgaggctaggaggtgcgcctccctcagtggaggacagcaaagcaccagtggctccagggagttaaaatcttttgataatttttgttctagcatctgtctgcagagctgtctctcagccattgcctgcctttacacaggagtgcagtccgaaattgggagatgagtgaaatttattatgcctagagatctggatccccagttgtttgggagtatattttctgaaccacttgttggtttaagtaatgcagatttattgatgccacttctcttgaatctgtgactctggacccaccatctaagtgaatgtgcagagggaacggaatggctgcaatagatctccattaaaaccagtgcatcctcccagacacatacagtagtagggaggtgagtcaatgtcaggacagcaccagctcccgcttcggtacatttccaaagttctcagtctgtgtacaaaggtttgctctggggcagcagaaatagccctgggcaggtagtcaaaggcctggtttgatttcctccacttccaggcaagtcactcgaaggctcacaggctttttcctcacctgccacatgggtccagtgagatctactgagctgtaaataatgaaatgagtgtgtgtgcagtcatctataagttgtaaagtactagaaaatggtgaaactttgggatttgggctatttaaggctgaatgctaaaaatgtcaggcattgtggagaaaggaatttaaatataagattgattgactgggatttaaagacaaatgaaggcacacacgcaagtgcacacccacactgacactgcacagctcccgttggaggcatatcctgaccatgcagacctggggctctgcctgtccaagtgcactcctttactacataaaccctccttctcttttggggctgtcaccccaccagagctggcaccgagcccttgctgctgcgcttccctggggtgtcagcttttgacagggtgtttcctccctctgcaggagccttaacatcccttggacttccttccccccacccacccccagcagttttatctcttcctaactcgggaccctttttttcccacacaaagtttattgtcagttgctggtttcatctgtttgagcggctgcaacaaaataccatagactgggtggcatatgcacgacaaaaatttatttctcacaggagaagtcaaagattaatgcaccagcagatctggtgtctgaggggccaccttctggtttgtagatgatgctttctagttaaaacacctatttaacacactattaaacactaagtgtgttaaatagtgcagttgatgtatttgtcatgtcacctttatcatacactaaatccttctttgtctttttttctgtactctaatctctttctgtaagtaatctttgcttgcagcagtaggatatttagagtactgtggcttgacaatatatttagtatttcaagatttccatgaaattcttctgatgtatgagttccctagttaatcttacatatgtatccctttgtaaaaacactttgaacatttaaaatgatacatgaatagtactctaatacaatgccataaaaattataaatcatttgtatagactggtaagtaaagattgtgagattaagaaacgcatcaaaggccattgagctggaaagtggtataatgagaattcaaaccagggtctcttgactcaaaatctaaggatcataccatttctcatgataatatgagtattattgttatctctatcccatagacaaagtgttaacactgaatgagcagtgaaatagtctcagaattttttattttatttagcaattcacttgtcatttctggtcctcagtttattcacgagtaaaataaaatagttggactagataatttctatagtacattcttacacaaaaaatctatgattttgttatttttaatgtgatatactcatggcactcattcacctcattttcccagcctgcctcactggtcattacttctctgtgttctttacaggctccccctcctctacactgccattaaatattgaaacacctcaaagctttacttatgtccacctctcctctgacactatcattctgtctagatgatcccatacatacatgcccattacttcaacctgtatttatacgccaatgattcactatatttccagcctagacattcttttgtactctagttaccagcttgatatccttacatggctgtttcaaaacaactcaaatatattatctctcaaaatcaaactcatgatgtccccacaccatcctagctttccaccaacaatacctatccctattaatagcaataccatttattcagttatccaaatcaaaaacctagaattcatccttaaaattctactatcattccaaatatcctatccatcagcagccactgtattcttaatcccctgtatttccttcaaatccattcacctctctccatatccattgctgcatgactatccaagccatcgcctctaccctagggtaccaaaatagcaacaaacctaatctgttcatttgcattattttttctccaaaactgattatctatatgtagcaagacagattgttctcaaattgcaaatcccactatattatcctcttgcttcaaacacttccatggtttcccattgtttatgataaaaccaaatgcttcaagttcgaagaccggcatgattgggaatttcctgtcaccctagcctacttgctctccatggtacagttgcactggctttctttcattccttaagtacaacctgtttcctcccacctcaggactgtgcatgtgccattcattctgctgaggagcctttttccttccacttcaatcagctaagtctgattcttcctgacaatctcagctcaataagcatttcctctaagaaatgtctctaatatcattaattggctcaggtccctctactgtattgctgcacttttcacagttataattttacttaattatgaatgattatttgattaggtctatttccatccattagacataagcttcatgatggccagattactgttttctatccatcgttgtattccaatacctgacagaaggagggcgggaggtggtggcacacaagagatgctcaaaaacaattgttgaataagtaaatgaatgaggccatttagaaataacgaaagtacctgtttacaaagtacatgtatcaaaactatgaatgcattctacttacatggttttctccaaataaaacaaaagacttcaatcaggattaatacctgggataaactgagtcattaaatctctcctttgccatcaggagtgacattgaaacaaatgtctgcaaacaacaaatacttttttcccaaaatatattgaatggcatttccataaacaaactagaacatgggaggagaaagaaagcaatattaatttaaaattaatcttatcacataacttataccatcagggatttcgggtaaaattcctttcaggcacatccatttaacaagaattgattgttactgaaagcctagaagagaatttggcacatacttggtgttcaaatatttgttgactgagtgaataaatgatgcaagtgtctaagaaacacaaaataaggacatgattacagtcacggtggagttcacagtcatctccaaaatgaggatatgcatcccagggaggaccaacaattcattggagtgctgaaataaaatactcaaaggtcattttacatgtattttttctctaaattacttttcttaagacacagaaaacaaaaaaagaaacttagctttgttactttctaacaaatagttaaatcattaaacaggattgacactagcatccttgtttggtcttatgccttaggggaacatgaaatgtgtgaagacattctgagatctgagggaagggtagacagtaatacagtgggactgaccaggcttcagcacacctttacctcctctcagcagatttcagtgatgagcagtttacaactagattgaaagattatattatctagttctaaaagaaaactaagcctcccaaaagcaacaagggaactgagaggaatcctgcaaaacaaaaacaaattttaaaacttgcactttgtaataaccctaatatgtaatcacagtaatgaacagtaagataatgacagaactgacatatttccttatctattaaagccatattaacaggtaaagcaatgccagtcagtggtacacttcttagaagatatttaatacatactagacacatacacacacacaacattttccttcaaggtgtatgtatcagaaaatcactttttaaggccggatgcagtggctcaggcctgtaatcccagcactttgggaggccgacgtgggcggatcatctgaggtcaggagttcaagaccagcctgcccaacatggcgaaaccccatctctacaaaaatacaaaaattagccagggatgatggtggatgcttgtagtcccagctactcaagaggcagaggcaggagaatcacttgaacctgggaggcagaggttgcagtgagccaagatcacccattgcactccagcctgggcaacagagtgagactctgtctcaaaaaaaaaaaaatcactttttagataaaattcatgctatagagagaagactatgaaaatatgtttagcaatgtgtccatcattaggtgattgagtttccttttgttttgttttactgaaaatcatataaagtatgttatctgtaaaagttctctgacatgcacacataaaaatttgggagaaaagattaactataatgtttaatagattttgtacacatttctttaaaaatatataaaacacaacacctttcaattggtttgcaagaataaccaattgacatcatggaaaatggaaattcacttgctgaattttaacaaaaatttgcatgatgagtgagactgacaacttagtgtcatgatttaatgaattatgccaatggtaaacttcatgcacatggggccaggtaattatgtggaaactttttcaatgcttaaagccaagtattgaaattaaacttagaatcagacctttgaaccattttatgacaatgttcaaaaattataaattctatccacttatattataatattaaaaatatcattacaaaaaaaacctgtgtttattttataactcagcctttttaatttctaatttcataaatatattataatggatattgttagtaatgtagtattattacatgtatataatttataagtaaatatacatgttttggctactcatgcataaaatgtttcacccataggagcacataatcagaaatgtctggagaccattatagtaatagatagatcatattgccacatattttatctcctccttgacaactgagctttccagatcttctggtgaaacgaaagagaaagttgtaacagaagagtgattaaaatgacaaaagcattacttctattacttctattctaataatatgagcaaagctataactatcaagtaataatgcactaaagaaggtgattaatctgatatattcacaggcaactaataagacctttctattgcagccatgaaaaatatgtgacaattatagatatcctgtgtgcagtgtttcaacctttatgtgacctgttctactaacagatttagtgatgttcactttgttagaattttcttacacatgccataacttgcttcagtcttttgattatgaatattatggatattaaggattctagactattctagatttaaaaaataatattgtcacctcaatcagaagggaaatattaaatagttctcattttttcaatgtttactcagtttttgtccaatgtaatgaaagtgtcagcagtacaggttacaaaataaaatgtgtattaaagtaaactcatttgaacaggttaataattgtagagggagggaaaaggctaaaagattgaatgtaaaacttatgaaaagtagatacatcgtctctatgatttgcagtagtcaactgcatacagatgaatcattttaatacacgttaactactttccttttacagatggagaaactgagaggaagaaagtttatatggttcattaaactttgtgatgcaagctaaactaacctgtctctgtattttccatctactgcccttatcactatctcattagaatactcttcaagcatctccttactgattttcttaccaagcatttgttaagttctaatgagagttggtagtaacattttcacccactctgtgaaatatgaaatcttattcataggcctcttcttttattcttgtatttgcatatcaaccaattaatcaacttgctttctttatgttgcttattatcttagtccttactaaattgcctcttaatgttgtccacataacagaaatgttaaggtggatacttaacattttagtccagtctagccggtgccagtgcaatgccaaatcatgaattaaaatataattacaagaaccacttatcaaattttaacaattccttcagctttgtgacagttttttctacttcgattaaagtcaagtaaaattaaagttaaatatttttattaaaatatctcctttaacattccatattaataaacatattaaagctcatgcttctaagtagattactagaagttactttatcgaattacagcaatggttaattctagatcatagaatttagaatgactttttgccttcttcttttttttccttttttttaaacagagtcttgctctgttgtccaggctggagtgtactggcgcgatcttgactcactgcgacctctgccctgcaggttcaagtgattctcctgccccagcctcttaagtagttgggattacaggtgcctgccaccacacctggctaattttttttttgtatttttaggagagacagggtttcaccatgttggccagactggtctcgaactcctgacctcaagtgatccacttgcctcagcctcccaaagtgctgggattacaggtgtgagccactgtgcctggcctgactttttgctttcttcttaatacttactagtatttcttgaatttttaaaaaagaaacataaagtactttgataaaaccaacagtctcattgttcttaaaattgttcaaaggttctctggaaaaaaaaaagaaaattatcatttggttaagaatcatgttggtctgacatcaatcatcctataggagtgaatattgaaaaagtaagatatattgtggtataatcgagattgcataaattttaccatttttgagaagaatctgctccaaatcctggcttaatgtaatatccagcatgctacttaattttcttgtcttcaccttttcatatccacatccacctaggtgccacctcacagtataagccagcataatccattcttctcaatgaaaccacaatacatctgaccctgcatctcaggagaactgtatcagccacagcacttccagttgactatgaatctgaatgttatgcctcaggagaaacatccttgctgggactgagtagtgattcaaggagatagttatgattcagtcaagaaattaataattagtgttatttttattattgagacagagtctcgttctgtagcccaggctggagtacagtggcatgatctcggctcactgcaacctctacctccccggttcaagtgattctcctgcctcagcctcccaaataactgggacagcaggcacttgccaccacgcctagctaattttttgtatttttagtagagacggagtttcaccgtgttagccaggatggtctcgatctcctgacctcaaggtccacctgcctcagcctcccaaagtgctgggattacaggcgtgagccactgcgcccggccataaattattaactgagccaggcacagtggtacacacttatagtcccagatactcaggagactgaggttggagtatccttttttatgttattttatttttaattattatgggtacataataggtgtacatacccatggagtacaagtcatgttctgatacagacacataatgtttaataatcacatcagggtaattgggatatccatcacctcaagcatttatctttctttgtgttaggaacattccacctccactcttggaataggcaccctgttgtgctattaaatacgaggtcttattcatttcatctaactatatttttctacccattaaccatcacctcttttcccctcttccccactacctttcctgtgaggctgcaggattcttaagcacaacagttagaggccagcctggacaacatagtgagactcaatttctaaaaaataaaaaagaaattaccaactaatgctaaaaaaatagtctctgatgcttaggtatgaattagaaatgaccaaaaaaaaaaaaaaaaaaaagactgccctttgcttccttctccccttctcttcaagttttccattgctactcattttagtctggtttaatcaggtttcatccattaaaagcaattgttgggatcacacattttgagttgtgtcagtggacttccctcatgctggcatgattcctgccccaagcccttagtaaaagccaccaagccatataacataatctctcattgagtaaaacatctgatgtgtttagaatgacttctagcaaaaaaccagcctgtccagcatcatctctgtataacagataaaggaataggtactgcatcaaaaggttatagaacctgcccaaatcaatcccatgtgttttgcaatggaattaggttgaactaaagtgaaaattcagttttctactcctcattaacatgtctcatgttgcaaggttgagaggaaggagaagaagaactgtatttacagagagattccccctctctttctttctacagattactaaaacattcaaagaatcaaatttaagaaatcagttcatcagagctcatgttgccaaactgaggtgagtggaactgtagaaaaaatatttaagtatagatacaatgtggcatacttgactttttgtcacagaatgaatagtaaatgacatgttcagataagttgttgtaatattatgaaaatagtattttagtcagcttaaaaaccaatgccaaaaaagccaaacatatgatctatttagctactaatgtaaataaccatattatatctattcttattgggaagaggaagaaggggtggagagagagttggggtgaaggtacagtaacaaggccatcctattgtaaaactccagtggatatcattcacagtgcagcctatgtaaacagtccctcctggagttgtacaatgctgtggtttgggtgtatccatccaagatcaagacactatgaccaacatcaaaagtggctttttggttttatctgcctgatgtgctataataaaagggtattatggccaaatccaaggcatgtctatcatgaattaataataggaggagtagcagcatgcatgctagttatttgccattcctgccttagttaaatatgatgtgataaaaccagcctttccaactgaaatagtcacctttactgactctcccgcaaatgtctcaaatgaccacattgctctagtctttaaataatatgcaatagttctttggtagaagaggaattatactaattctttctcaaatactagcatcacaagaaaattaattcttgttctctggagagtcacctagtaagtatctggagcacagatgtctggtcaggtaagttttgatgaggagttaaagggataagaagagtccatgagaagggtattttccaaaacacctttcggtcaattcagtgcacattcacttagtactttcttgtcagtatctgtatcagccactaatgttcaaaagtgagtaagccctgaaaacctgtaggactacatgagccttctgccttttctctccttttgttcacttcccacttatcactcaatcctctgcaacctggcttcaataccaccataaaatatcaactgctcttgccgattcaacaatgacatccagataacaaaatccaaagaaaccacatcagtcctattcttggacctttcaacagtatttggtcctgttggcctgtcactccttgaaataggactatcccttggtttgcatggccttgtataccctgattttccccttacctccctagctattccttcttagtttcctttactaggtcttacttctttgtatattccttaaatgttgctgaacatcaggctgtgctctaggcctctcatcttctcaggtcacactctctcctttccttggccttcactgccacccatatgctgagtgctctcaaagttgtatctctaggccagtcctcttttgcctccaaacatgaatatatgcagccatctacttggtaccatcacatggataattctcatgatctcttccagtatgactgcttctttatttttttctgggctcttttttagcattgctttacatggaactttatcatgtctctcaacctctattttatcttttatctatgtatgtagagtctgtgtaatttcttcatctcttttagataactaatatctcttcagctttgacttgtattctgtgtaacccatttattgcgttttcaatttcaatgagtatgttttcctatctgcaagttctatttgtttcttttgagaatcttcctggtcttttaaacacatttcttattttaatttttgggggtacctagtagttgtatgtatttttggagtacatgagatgttttgatacaagcaaacaatgcataataatcacattgtgtaaaatggggtatccatcccctcaagcatttatcctttgtgttacaaacaatccaattatattcttttagttatttttaaatgtacaattaaattattattgaccatagtgactctgttgtgctatcagatactaggtgatcttttaaaaataatgttttctacttaatctcatttttatgattccctcttttacgtcatttgtcatttcaaatacagtcacttgtctgttgattctattatgtgaagtttttgaggataatctttttgttactttgattccaccttggtatggtttggctgtgcccccactaaaatctcatcttgaactctggttcccataatacccacatgttgtgggagggaccttgtgggaggtgattagattatagggacgtttcccccctttgctctgttctttttcctgccaccatgtaagaaagatgtgtttgcttccccttctgccatgattgtaaatttcctgaggcctccgcagccatgcaggacctcttttctttgtaaattacccagtctccggcggttctttatagctccgtgagaaaaaactaatacacacctcatgatgtattgtttaccactgaaattgtatgcttaaatttaatctcacttgggaccctgtacaacctagacttaacatatctacctccagagcagttacatctgtcagacattctagaggaatcagcagcacatggactttgttgttgttaatttgttgtcgggggaggggggagggatagcattaggagatacacctaatgctaaatgacgagttaatgggtgcagcacaccaacatggcacatgtatacatatgtaacaaacctgcacgttgtgcacatataccctaaaacttaaagtataataataataaaattaaaaaaaaaaaggttctgggagtattcaggtagtattaatgaagattcagacatcgtgcagccaggcccatgcttatgaattttcaggtgatacttctttttcttttttcttaatttaaagctggatctcggaaacagataaatttatttttttatgacatgacgagcatttttttcattctagttcatgctgttattgggtgtttagttctttgagactcctggcctttttctaaaacctcaagttcaacttcctattttgcactggcccaaggtcccatctccagtctctatgtaaatgctaaacataagcctgtggaatattctagtctcaccacatactattcacattcttctttgtttttggtcttccaggattttccttacttttctatgaacccagtcttgcatttgaaatggaatttattatatattatctatcctttctatttgttttatgcagaaagtgttttctaaaattatttaggcttccatattgctagacatggaagttgtaattatttgttcagtgcctgtttctacatctaaactgcaagacccatatggcaactgtgaatcttagtcccagctaatttctgaagcttagaatagtgcctagcacaagaagttgtttatctaacatttttaaaaataaatattaaattcatatctggaatgaatattaagttagagctggtcattgaggtgagaggaggaagccaagagagaatatgagagcctcaaagccaaatatctttaatgtactttttcagaaaagaagacagccaatgtcaggtggaggaactggtttatgaggtaactttcctggaagaaaatagaaattactgaggttttagataatccaaatatttaatcaagtcaccaaggtttattgtggggaatctttattattaattaaaatgagtgatgaaatcttaatatacgacaaaagttaaaatttgcttttgcaggcagatgaatggtctaggtatcaaaaaattaagttgagtctctaactcacacaaatttacaaccctatcactttatgaatttgtttaggagattatttttaataacactggtgaagtctaagaatagctaaaatttatagtacacttattgtgtgctattgactcttctttgaagttttgcatatagtgattcatctaatcttcataacccattttacatgtgaagaaacttagatatagaaagattaagaaacttacataacttatccaaagttacacagtaaaactctggcattataacttcaaaatcagctatcctacagtgagtacagtgttctgtgcattgaaatcaaataagtgagatagcatcgtgatatagtattacgtatgcaaacactgttacagagatctgtctaaagttaaattccacaaatgaattctttaaaagggtttaatcaagaagaatatataaacaggatggtgaaaaattgtcatattatttgttttttaaaatatctttatgatttacaggcaagatggtagtggtgtgagagcggatgttgtcatgaaatttcaattcactagaaataacaatggagcatcaatgaaaagcagaattgagtctgttttacgacaaatgctgaataactctggaaacctggaaataaacccttcaactgagataacatgtaagtataatttttcataaacaattttatttcaatatatccctcaagtttaccaattcaaattcatattttaattgagaggctgacttttctttctttgaaactaaactgtgaaaacaatccattaaaaagctaaatataccatatagctccctaacgtaaatcattctaagacttaaagaatcatttggcatttatatagtaaattttatttgctaaaaattctcattaattatccctgcaacattccttatgagtgatgttactgtcagatgtcattagtggataggccataggaggggtacatagatgctcaaggtcagagaactatttaattaatgatccacctcagaggcttcttcatttttctttgtaacatttatcacaattgaaattacaaagttatctgtgtaaattttgtattgtttggcttcatcctacactgtaatcatcctaaaagaaagaaccagtcaaccttcttcatcctactaccctcctaccacccagtctccatcatataacacatattcaataaataattcttgcatgactgaaagaaaagaaataatatatgcatagaatttaaggacattcctccaagttggttacattctgctagtttaataagccattatttcttctcgatgagctcaagattaaaaggattttgatgattcccatactagactggtaggtaccagttacagatgtactaactgttaaatattgaaatgctttcctatttgttggtaaacaattactgcatcaggcccacaaagttgtcttccgagatgtttcaaatccactgcccctgctgctaaagagttatgcttagcaaagcaaagcactctaagacactgctccaactccatggcctgattgcatcttttatgactggccaatgctcacgcactgcagtttgttaggtagttgaatattacctctgcttccacacattaaggaatgctcccgaacgcacttcccaagtgtttatttatttatcattatactagacaatatggtgatacgatggtcacagaatagcggtttccacctccagagcccataatctagttgaagggaaagatattccaacacaagagtgttgacaatcaagatagaatatgatcaagggcccagtgtgaggcccaggcaatgatcactgcaggaatctggggaagaaagagaccagcgtgcttgggatatctagcaaaagtttcatgaaggagaatggactttgactttgaaatatgggtaggatttacatattttgagatgagaaaaagaaagttcccagagaaggaaagcatgaaaaggcaaacagtctgtactgaacgcgatgctttgacagaataatgaagaaagggacctgctggaatgattgatcagtgttcatcattcacaccatcatcatcaaaacacttatttaatgagaacttactgttttttaggcatggctttaatgccctatatgaatttttttcttgattaatccttacaacaaacatatcccatagatagttttattgtcccccttagaaaagataaattgcctaggctgacacagtcagtatatgaggcagtcaggattcaaactaagtctgtttgttcaaaaaattaagaatggccagctttttaaaattttctgtctccagaagtatgatttggctccactgaagtttgcaaaacaaatgtgatacccaaaccttgtgaaacttttagtgggaaataactttgcataagtcggtttgagagagcgtggaaacctgtcttgaaaagttttaatttaacttgcaggaaataaaaatgatgggtttctcaattaaaaatttcaatcaaggaaggatatgagctaacataacatttttttaaaaagatcagtctggtaaggtagaggtgcataaactgaaaaggagcaaaagtggtggaattcagttagaaaattattgtaactgtactgatgtcaaatgatgaaaccatgaactaaagtagtaccaaaaggagtgaggaggatggaataattcaaaagatagaggacagatgtgcagaacctggagattataagatgtgaaaggaggagtttgagaaaatttcagattttggaagtggtgtcattttactaaaaggatataataagtagcaaattttggataaagttgggtcccactgagtttgagatggctgttggacatgcagagaaaactgtcttgtatgctgttcttaaattgaaatagacagacctttaccctctgatactgacatattttcctttccaggctcaccctccatttccctaaacacaacacatgcactagctctccttactttattgctccacaaacatcttacacctccaagcatttgtgcccactgtaccttctatctggaatctcttttgtcctcttgtgtgcctgaaaaattcctttcagatcttcaaaatacagtgcagatgctatttcttctagctcaaatattatctcctccatataatttaattactctcttttttcttttctctactttgcacttacatttatttgaatgattgcttgattaatttctacctgtaaattatgtgagggcaggtcctctatattttgctcgcagttaaatctgcagcacttattatagagtggtatcattagagtaatatacatatatttgaggacatgataaattaacttcccctatagtatttatcacattgcatctcaatgacttgcttatgtttctgttttcccatataaattgagtaacttgaaaaaagagatatctattaagtatttaatgagaaattaaagtacaaactttagtatgcataacaacaaattgggaaaaggttgtaaacaaagagatttgtagggcccatgagttagagatcgtttcagcaggtctgaaaggaagcctaggaatctgcattttagaggaccacctcccaaccccaacaagtaattctgcttcttgttgtctgggtactgtactttaagaaattatggtgaaatgatatcagcctttattgtatttatcttattctcattttttaatactagcacttactgaccaggctgcagcaaattggcttattaatggtaagttttaatattattttgtaactgtaatttgccaaatcataaagagtaaaagtgcaagtcttttgtgtacttttggccaaggcagtatctatcaagttgatgtctttgttcttagttcgctcaggtggtgttgaaacaagacagtgctgatcccaagtgtcccatggagtggactttaggtttcccctttccttttagaaaaaggaagaagttgtagtggaggactacccactctgcactcaaaattgccctcatgaaaatttctttggcagctttgagaaccttttactgccctggttctaaggtggcatttctgtagacttacaaattatgtttgatgacaccgtttatgtagcttctcctaaccaccagagtagcttgctttgttgtgaattcaggttaatcacaaagtataataaaaaagaattgtcagaagtcttcccagctttgggtctataacctgaaggaaaagtcactactcttcaacatcatcctatgtactctcaggctaggatagcagaaatgcaatccctagaaaacagcaacttacttctctgaccaaaaaaatgcagttaaaaattagttcaatgtacctggtagctggcctatcttaggtacttcagtgattttacaaagtgatggtagtcctatgggtgtttttcagcttcactacgtatttaattcatgcttattgttaatgaaactgtgataagcaatttactagggtatttgtttgggagatgccacaaaggaacacatgtatctcttaatggaagcctggtcctcctttatccaggaaatttgctaggaaaaaaaagcctttaggtggttgtgctattaaaccagggcactacttaaaagccagcccagcaatagttgtgtgatttaccattaatttcttagtaatagaccacacaaaagaagaaaattatgggaatgcgagttgagaggaattgggtgatcagcctaccccagcccgtttcagctctggccagtagactattcacgagctctttgaaaacatttaaataaaccttatttagatactagaaaccctctgtcaccctcaagaatattctgtggtatagcgactcctttatgagggcatgtttggtaatacagcatcagtcttggaggtggactggattctacaaggtgaactgcagtcactaaggagtcttttggatgagaccagttttcctccaacttcaatgtgtgcatgaacctcacatcaaaatgtagctttagatttgtcccatgatgtggttccaagaatcagcacttctaataagtttccaggggatgcccatgctgcaggcccacaaaccacactgagcatagcaagactattgagaaaaaggaaatttcccaggagtctgtggcctgagctggcacatccaataatgacctatcttaacctcaactcatgaggaattccagggaactctgaagctgctcaaaatttgaagcctatatgccaactaaattcagaaatgttctccaaaatgctatctataagcaacagtagtcacaaatgcattgtagaaatatatcgatcatgctttttggaaaatccagcatgtcctgaggaagaatgtataagacataaaagtcataaattatggaaagactcttcagcttcttccaaatgtaaaggaatcatgatcttcccagcacattaatgccctttctcattagaatgtggggccggtccagacctaataacattgtctgagcagagaatccttggaggcactgaggctgaggagggaagctggccgtggcaagtcagtctgcggctcaataatgcccaccactgtggaggcagcctgatcaataacatgtggatcctgacagcagctcactgcttcagaaggtgaggccaccactacctacccatctgggaacaattagaatagacaggtcatgaagactgcaccctctaccctaggattgaattgagccagaaataattcaatgcaaaaaaatcagtaagaattttcttcctattcatgaaaggaaaaggatttttcccctttagcatgctaatttagtgctatttctctgtttcaggtaataatatattagcacagtaaagaacaaagatttatatgtcagaatgttttttaaatcctagctataaaagcttaagaaatttactaaatctccataagctttattttttttccaaattaagggacaacactgttatctgtgacttagtgttactggtagcattgagtacactaatgtaaacatacgttaaatgttagcgaaacgaattgctgtggaagatttgcacattatatcatgggagctgatggctaacctagagactgccccatgccattaatttattcattcataaagattattgagtatctagtatgagcacagtgttatatattgtagaagctactagtataaacaaagtattgcctctgccttcaaagagcttacactcgaatgttggaatcagaatgcacaaaaataatgatcaattacaatgagtagcataaataaaattaatgtaggcaacttacaagaattcttaattgaggtgactaaactattgccaacactagggtgatatgctaccagtggcgagtaggttgcataaacttaccttattggtaaaaagaaaagttcacattgctcataaaagaaggattttagatttcagcataactaaaatctgtttcaaacctgccttgttactggggcatcgcagaccacaacagttgttgggaacttaactcaaaaagttcacccagaaaaataatggagatttgaactcgtgtgcccctgaccatatcaattttcttctcagactcttactctaaactggacctccttatcacacacacaaagccttccataggcagatcaatccagtcttatttctcaaagcatgtaccttgagcttcagataaacagcattgttctcttcccctggactcttcctacatttccctacctatgagtatctgatcaatctgcttatccttgaaatgttaatatatttaccacatctctatttgaattttatgaaatttttgataatttctaagtagttttttcagatttataggcactacttcatggtacagtgactgttacaaacgtatttgttaaatttagaaggaataaagatttaaaagactagggtagttactgaactaaagttttaggaaatcccaaattatttcaaatttttcttatggtaattttatgacttaatatttttatatgcagtgaacaaatttgaaactttaaaagatactcccagaattatcagttttctgatgtagattggcaaatttattactatatcccaaataacccaagagacaaaattcacaaaaacatttcaattttcattgccacttgaaaggccaaaaagcagaaatggcacgcattgatttcaatcgtactcttgagtgtgggaaccaggaattaaaatacctggacttatcaggcacttagcataaccaagaacggaatagaaacctccctggattctaagccctattcagtcccaatcaccaaaaaccaagtaaacgatatcactataatgaaagccacagttataaatatcgacaacgattaccaaaggaatccatggaactttgaattttgccaccccacatccttctattcattaccatgattgatccactaaagctaacagactctgtgaaccttgtattggacccctccctaaagacctgattgtcactgagaaccatcagtgaggatttgtttggggcatgaccagccttacatcaaagtacatagaagtgatgaggtcttatcaaagaggattattgaattatcacctcttctatgtagctttccctgatactctctttcctctccattgagttccacagaaatttttttatctgcctttaacagttgtcctcatgatttgtgatatttgacttacctcttgtcagtttccttcactagtgtagagttcctcaaagaaagagaccataattacttatatttttattcctggagactcatactattccttatacaaagtagacacttaacaatggcttgttgaactataattaatgaaaataatagctaccttcatgaaagttcactttgtgccaaacactatagttgacataatacatttgtctcattaatacttaacaattgtgtgagaaggtatcaccaatcacattttatatgtaaataaaccccagagctattaattaacttgtcataaataacacttttcatatgtggcatagccaagatttaaatataaatgttactggttccaaaatgatgctctaattcacttgctggaaagaaggaaaggaagaaaataaacgagtggaaggaagagagggagggaagagagaaaaggaaggaaagaaaaaagagtctcttcagaaccttcactgtaaagactccgagcaaaagaagttgaatataaaaacaacataggtttgtttgttttctaatattttttcttcaaaatttttaactcaggttcactcttacacaaactactgtgtcttataaaagtatttccggtcatagaatttttattttctgtattaactccactatctaatctccataaaactcctaaattggtattatcggtaacattttgtttttactcaacccttaggaacaatgttaagttaatcagccctccacatcacagatccttattttcatcagtctgtacaaggcatttctctcattttaattttttttcctcctgtcatccctggatttcactttcactgccctccttccacccatatgcctcatactaatatattcgaaatatacatgtcttaaaggtacatgcacgcacctacaaaacctatagtgtttttttgtatgtatatgtctttaatttaaataagtagcattgtgtaaaagtctaatattgtttcttactgttttcactcaattcttggaattttcatctgatgcactgctgcatagcaccccatggtatgcagccaccatatttccttcatccaattaggttgcatgacctaccttcccattgccacaaagagtacacacaaaatatttgtacttatctttctgtaaaccttcaggaatttcagaagcacacatgcaggctgctaaatataccagaatactttccagccacttaaatctttaccagtattgcaaaagaggccccatttccctccacatcaacatttagtattattcttttgtttaagttttatcaatcttttaaatgtacacaagatgctcatttttataattttaatttctcagattactagtttgagtatcttttcatatatctaagagctgttttgatctcccctaccatgaactgccactaatattctttgcctattttacaatggtttttctgcttatttattactggtttacagacttttaaaatatattctacaaaaattttagacattaaacattaccaatattttcccatggttcctcatccatctggtaaacttgtctatggtatatctaattttgatttaatagaattcattctatttttaccttttagtttgtgtttttgttgtttagccaaaaagtccccattcctaggtcataaaggtaatgtcctttttttttttttaacgctactgttctctctctgtctccccctatgtatataggtgcacatatacttgtacacacatacatatacctatatatgaggggagttcgataagtttatggaaaataaaattaaaagataaaataaaaaattataaactttatttctcaacataagctccttcaagttcaagacacttttgtaagcaataataccagccatatcgtccatccctaaagaactgagggtcctgagaatttaactatgtcaatgcagtcttttttacattacttttttacagtacttattgatgaaaaatgggtgccttttaaagattgttttaagattagggaacaaaaataagtcagaggaagtcaaatcaggactgaaaggtggatgcctagtgatttattgctgaaactttcataaaactaaccttatttgatgagaggaatgagcatgagcatggttgtgatggagaagaactctggtggagctttcctggacactttttctactaaagctttggctaactttcttactctcataagaagaagatgttatttttcactgaccctttagaaggtcaacaagcaaaatgccttcagcatcccaaatgtctgttgtcatgacttttgttcttgactagtctggttttgctttgactggaccacttctacctctttatagccattgctttgatggtgctttgtcttcaagattgtattagtaaagccatatttcatcttctgttacaattcttcaaagaaatacttcagaatcttgatctgacatgtttaaaatttctattggaagctctgaccttgggtgcagctgatctgggcgaaacagttttggcatccatcaagtagaaagtttgctcaactttagtttttcagtcagaattgtataagctgaaccagttgagatgtctatggtgttgtctattgtttctcacagttaattgttggtcctctttgagacatgaacaagatgaaatttttcctagcaaactgatgtggatgatctgttgctgcgggcttcaccctcaacaacatctctttctttcttgaaacaaattatccattagtaaactgatgattgggggagatgctgtccccataaactttttgtaaggcataaataatttcaccattcttccagtttcaccataaatttgacgtttttttgcttcaattttagcagcattcatgttgctttgataagagctcttttcaaattcatgtcttattcctcttagtgcctcaaactagatcttgttcagtatgacaagttagtatgagtttatctgcatgcaaaaatctttgaaatccatgcatagtttgtttataatatacattttcaatgaacttttgaagaccccatacatacatatgtatatatatgcacacacacacacacacacacaccaaaatcttcaaccattatcagacttagtgcagaaaaattattcatccattaacaagataagaatgccccttatcatcactactatttaaatggagctcctggctaaaggaaaagacagggattgaaaaaaattagttaaatctaaaatgtttattatttcaggtttcttagttgcttaaatgggaagggaggtatggacaaaagagaaatcaaagatatttgtgttatgctacttatcattaaagtatcagaataacttcattggaatagaaaaacaccaagatcaccccacgatatgttttctaaaatcttctccatttctttagacaagtgaccatgtattcggccagtgaagaattaaactcacttgccagcttataatgcaggaaaatatagcaaagagatgtggatccaatagtttctagatagtggtacaggatggctaagatgaatttatatatctgaaatgttcacaaattccctactcatatagcatgttttcataatgttttagcaactctaatcctcgtgactggattgccacgtctggtatttccacaacatttcctaaactaagaatgagagtaagaaatattttaattcataacaattataaatctgcaactcatgaaaatgacattgcacttgtgagacttgagaacagtgtcacctttaccaaagatatccatagtgtgtgtctcccagctgctacccagaatattccacctggctctactgcttatgtaacaggatggggcgctcaagaatatgctggtaagtgtctcggaaaaaaaaattaacaatagaaatgtcttatatttgctattaggtaattttttaaattaggaaacatctggaataggtgtttctattcttctacagacagaaccattctatattctgctcagcccaagctctggctacccctgagtctccttagcaaagcaaagcaatgctccagaaactatgggaattctcaaatatagtaataggaaaatgtaaaagaaagttatgaagacacgagttctttaataatccagagattctataagattcaaatagcttccctataaacaataaaaaagattttgtttgtttgtttgtttgcttgttttttagagacaaagactttctcagactggagtgcagtggtgcaatcatggcttactgcagcctcaaactctggtcttaagaaatcctcttgcttcagcctcccaagtagctagaattataaataagtgtgtaccaccatacccagctttttttttttttttctacagacaggttcttgctctgttgcccaggctggtctggaattcctgccctcaagccatcctcctgccttgttggcctcccaaagcaatgggaggatttagattagacattgtatgagggcttaataatccttaaggtattaactgccctttaaagtattctgggatatggcaaaaactcgatgtgtatataaacattggtcatatttgtttattgaatgaataaaatggaaactaaaatgaggacaatgcacaagagctactagaaccagtaagagtatcagcgaaggagtggaagggtagcattgacaatttccctgggcttttacccatgttgtagattgtctctccaaggaataatacaaagccttaatagtcctagaacacattctattgtgttcttatggcccaaagtaaattggtgtagtagataacatttgcaccagtcatgaaaaactattggtgtcattctgagagtacatcaatataaaatagactagttctttagccttgaaactagactggtttctcttttgctgctaggttaaaggttattcaatatgtaatcttccaatccaaaatctgtcagtggataatttaaaagcttttagtcaattttaagatatttgttttcttaaaattttaaggggcactgtgtcacaaagctaaagaaaaaaaagaaaaaaaaactgatctgtgaaaggggttatcctcatctacttggggaattttggctgcgaagaaactccaaagtaaatctttagaagccttcattgttaaatatgaaataatgtttggagtacatttatttcttctcaaatttattatagggtcaataatgtacacatcttgaagtccatttttttcctgcttttataacaaacaggccacacagttccagagctaaggcaaggacaggtcagaataataagtaatgatgtatgtaatgcaccacatagttataatggagccatcttgtctggaatgctgtgtgctggagtacctcaaggtggagtggacgcatgtcaggtaagctcaagacaatctcatccatgtcatcatccaagaagtgtataagcacttcctagtatgtgataatgtgatagacataagtgtaacagttacaatacacagccctgttcctctaaaatttataatctagattttagaaataaatttttttatgaatgaagtttatctatcatgaaagcattaactctgagaggccaaattacagagtagttaaccatccaaagctcaagaatcagaaagacctcgatttgaattccttaacctctattaccaagtctctaactaaaagctggggataatcataatagcacctaactttttgggtactaagaaaagttaaatgaagactaaatatatcaggcacatggtaaacaacaaagaaatctcatctatttcactattattaatgtagaccatggtcactcgtgttaataactttaacctcaaccttttaactgctgtgaaggattaaataaaaaattaatcactatattataaaaattaattgatatataataaatgaattttaagagatacgtaataattcatggactccttgaagatagaaaatttatacaaaatcctagtaatttgagtcacaaaagctcctacaataatgaaacagtatgaatgaaaaagaaaagaaataactattatatttggatctagcccataatttttaaccaaatgcacaaaaacaaacaacaaatatgaaattctcactgtaaagtgattaaaatcaaatttgaattctaaaattttaaattaaattatctaaacataattgatgcagttatatgttttaataggttttgttcacatatctgaaatccaactccacacagtagcaggaacagctggtgtcagaaattaaatattcttttagtctggagttttaaaaaatcaatctgtttacttgagtaatttgttgctgttttcatgggtgaattgtatacagaaggataagaattattcttcgcatcaaaaggtcactgactttcatatttagtgctcatggtctttaaaaagtggataaaaagtagttctcacatttcatggaaagcccccaatccatgagcacatttcccaaaatgaaacatttttatcaactgcaagttgtgtgtaggtggagatttgtttttcaattgtcaagatactgttaattacccagtcctttatctccttttggtggagatgtctctgtgctaggaaacccttcttgctctccttcctgtttctcttttactactggccctgaaacaacaaattctcaagtttcatgacagctttccaaagaatccatcaatcaaataagcaacacaactcgacactgacaattccagacctactaagagcattaattaagacttaaaaataaacatgagttttaaaagggtgttattcattattttcccatttataacgtcccttaccttctgtccttcagtgcatacaaattattatcttccttgaagcccagttcaagccgtacctcaccatgataccttccatgtatattccactctaggcctcactgatttttaactgaaatactataatgcatagttcacacttaaaaaaaaaaaaaaaacacagcactttacataagagcttacaggatcctatttgttttatccattcttttgttcatttttacaatcattaattcaaaggaattatattaattactttctatgcacccgacgttgtgttaacacaacaatactatccctgcattcagcaagtctatggtctacaagagaggacacaaattcaaatgtctgtagtcaagcagtgaagctggctagatatggaaaaattacaagtccctcttgctttaacatttgcttgcccacatttggtcagacatcatgcaaaataatttctcactatagaaaaaaaaacactacaaaaacaataatataaagaactgagaactggttaactgaagcatgcatatgtcatctaaaagaagcaggtgacgaccagcttcatgaagtacttgccatgcatattggcacttcacacactgacccttctccccacctagaccagtaattaaacaggtatggatgagctagctactaagagcagccaactgaatagctgactaacttagaagcacacttggtaataatagctgacttttattagtactgactatactatatgctaagctgtactcaaagtgctttgagttttaaactgatacaaacattatatgaggaaacagaggtacagagagctattcaccagcttaccaaaggtcacatagctggtaagtggaggacttaaacccagactatctagtttcagaacccacagacttaatccatcgtgcagaacataagacatactccatctgtctccccaactaggttattatgtgcacaaatatttattggttggttggttcattattatgactgggtggtaagtatgtcattaggagtgttttgcttatgactatataaatttcttcaccaaaagaagactttctgatgatatactatgcatcagacaccacgcagggtgctaaggttaggaagataagtgagacttctagaaactcattcattcaacaaatatctcctaagggctagaagcttaggtttcagcagtgaacagaataggtatgttctctttcgtgttggaccttatagtatatctgggaaaacagacattgaataaatatcacaaatgcaagtgagtgtttcagagacatgcagctgctacatcaaaacaaaacagaacaaaacaaacaaacaaaaactgaccagtgggattaagtgtaaataggcacacaaatgcacaaatatgcttttataaaatagtgaagcagtgacagagacacacacaagatataaagacacaatgaagaacaattgagcccaaagctggaaagggtgagagtgtgaaggaaaaaggttgatcagagaagttttcccgaaggagagaaagcctggatgattaggaggcaaccactcggtgactgagggaaatctgaaaaatgtatttgtcatcttctcagacttgctgaaggaatgacttgggtactttgaggatttcagtaatttttccatgacttggtataatatttcaaaaggaaataggctgactttatttgtataatgaatgtgactccttcctcgactgccatagaaataaactccttaatattttgggtttgtctttgcacttaagtaatcagtcattctgtttttttacagggtgactctggtggcccactagtacaagaagactcacggcggctttggtttattgtggggatagtaagctggggagatcagtgtggcctgccggataagccaggagtgtatactcgagtgacagcctaccttgactggattaggcaacaaactgggatctagtgcaacaagtgcatccctgttgcaaagtctgtatgcaggtgtgcctgtcttaaattccaaagctttacatttcaactgaaaaagaaactagaaatgtcctaatttaacatcttgttacataaatatggtttaacaaacactgtttaacctttctttattattaaaggttttctattttctccagagaactatatgaatgttgcatagtactgtggctgtgtaacagaagaaacacactaaactaattacaaagttaacaatttcattacagttgtgctaaatgcccgtagtgagaagaacaggaaccttgagcatgtatagtagaggaacctgcacaggtctgatgggtcagaggggtcttctctgggtttcactgaggatgagaagtaagcaaactgtggaaacatgcaaaggaaaaagtgatagaataatattcaagacaaaaagaacagtatgaggcaagagaaataatatgtatttaaaatttttggttactcaatatcttatacttagtatgagtcctaaaattaaaaatgtgaaactgttgtactatacgtataacctaaccttaattattctgtaagaacatgcttccataggaaatagtggataattttcagctatttaaggcaaaagctaaaatagttcactcctcaactgagacccaaagaattatagatatttttcatgatgacccatgaaaaatatcactcatctacataaaggagagactatatctattttatagagaagctaagaaatatacctacacaaacttgtcaggtgctttacaactacatagtactttttaacaacaaaataataattttaagaatgaaaaatttaatcatcgggaagaacgtcccactacagacttcctatcactggcagttatatttttgagcgtaaaagggtcgtcaaacgctaaatctaagtaacgaattgaaagtttaaagagggggaagagttggtttgcaaaggaaaagtttaaatagcttaatatcaatagaatgatcctgaagacagaaaaaactttgtcactcttcctctctcattttctttctctctctctccccttctcatacacatgcctcccccaccaaagaatataatgtaaattaaatccactaaaatgtaatggcatgaaaatctctgtagtctgaatcactaatattcctgagtttttatgagctcctagtacagctaaagtttgcctatgcatgatcatctatgcgtcagagcttcctccttctacaagctaactccctgcatctgggcatcaggactgctccatacatttgctgaaaacttcttgtatttcctgatgtaaaattgtgcaaacacctacaataaagccatctacttttagggaaagggagttgaaaatgcaaccaactcttggcgaactgtacaaacaaatctttgctatactttatttcaaataaattctttttaaaataatttccctgcctaattatttatggaagttatgacttttgaaggacaattcaaaaccatttatttaattggttctgcaatgaaagaactgccccatatactctactaaaggcttggcactttctgctgccttttaatccagcgctataattgaggcaagcgtccagcttgacacctcgagataacttcgtataatgtatgctatacgaagttatatgcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagcccgggcgccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccatgggtacccccaagaagaagaggaaggtgcgtaccgatttaaattccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagtgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggtaaatataaaatttttaagtgtataatgtgttaaactactgattctaattgtttgtgtattttaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattgatctagataagtaatgatcataatcagccatatcacatctgtagaggttttacttgctttaaaaaacctcccacacctccccctgaacctgaaacataaaatgaatgcaattgttgttgttaaacctgccctagttgcggccaattccagctgagcgtgcctccgcaccattaccagttggtctggtgtcaaaaataataataaccgggcaggggggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggaataacttcgtataatgtatgctatacgaagttatgctagtaactataacggtcctaaggtagcgagctagctgcaaccgaggaaaaaacgtgccatgaggtctctgtatccaagtgtgact

SEQ ID NO: 87 - DNA, 34073 bp, synthetic construct
  misc_feature 1..34073: note = Recombinant polynucleotide
  source 1..34073: mol_type = other DNA
gagggagggtggtgctttgctaatggtgaattactaactcctcaataaagaatattatttgaaataatttttgaaatttcataattactttgggttctttcttaatgataaataaataatagtatattacaaacatacattaatatttcctgaatgaatacaccacaaatctcccttaaaatatagcaagaataaaaattatactatttctgacaatttttaatttctcaaataataataccactctgatttttaaacatctacaccactctggctttgccaatctttttaaaaattgaaaagataataattttatcataattacactgaagcatagaactttttctttcaaggaaagcaaatttttgaaattctataatataacctcccataatcctgaataaattaaaggttcaacaacttagtaaagtaagactgaccttcccttttatttctttttcagatcaaaaatcttacttttataggagcagttttcaactcctaaatgttgaatataatagtcagttaaattcaccagctacacaggaatacaggactttgagtggaagaattgaatctctggtaagttaatatttgtctttgctctttattccattataaaatgaatatgataataaacctaatgttttgtaatatattttcagttgctaagtgctctacatattttccttccttgaatggtgaaacatgtgtttctctctgcttttatccagttagtttactcatatactggttcttattcacatctttgtcatgagtaaaaagtgttagaaaggccacgagtaaatatgcattttatttgtttatgaattcaaatactaaaagttttttatttgtttaattaagcattgacattgtctttttaaattcttttcattttaccttcttccctcttccttatccaactaaagacgcaaagcaggaggtgttaaaaaacaggtttaccatatcagcagtaacatagtttggacaacattacactttggttcaatgatagacatagaagtttgaacagaaatatgcaaagcaagtttgagctctaacttgaagagagcctctgggtgcctgccaggaaacctcacgagtggacccttaacattcatgtgtcaccacaaactaggggctgccctttagttttgaccagtctcagtgtcactcacttacccttaccttttcaaaaaaaagtcctaagaatataaagtaattcaatggttctacaattttagcatgtaactgagtcacctggcagggttgctttggtgagctcaagataaaattttatcagcatttctacattttctggaatattccttaatccaggcttttaatcccttggtgcttttctgaaccactgcaatgagcttctaactgttctcactgtgtgcaggctcttttccttctaatctaatttacacacttctgaacacaaatctctcacagcctgtttccttcatgttacctccagctcaagactttttgcctacaaaataaaattcaaacttgttagctaagcaccttctcatgtctatgctttggctcatatttcagccatcgtgtgccccacttattcttatagccaacctgaaaagccatcttttataagaaactacctctgctctccatgattggatataattaatcctccttccacatcacctcgccacaaaattgtatctgtgttgatctcatgccacatacctgtatgtattttatattataaatatttgcagacttgtttaatttgccatgttagactaagttccatgaagacagctccatatccattccatttttatatatccacaacatttggtcgggttgatgcttaataaatgtttattgaaggaacaggagtctcccacttctgacataatgaacttatttcccccagtgttaaccctacatctggttcctgtccaagagtctcttcccaaatcattctgattcaactgttcattctgatctcattaaacatttaaatgatatatctaacttcgcttgctttattctatgctcatcctgcagtctcctcataacttggtttcaatgatgcttgcttctagagaaaaaaatgtattaaataagcttatgattcagtcctccagctgtgatggttctcactgaacattagctcagtggttttcgaagtatggtctctagcataacctagaaacttgttagaaatgcaaattcttgggctcaccaagacatactaaatcaaaaattctgacattggggcctagaaatctgtgttttaacaagcctgccagtgcagcctggtcccttttcttctcggagccccactcaaagctttcagtgctcatctcccaccaatgacagggtcctctatggaaaccggcaggacggtttccaactctaactacgttttagagtttgcttcctagggctatccaggcaccaagtatcacaggttagtttcccagggaagcagactctgagacttgcatgcagggagtgtctctggggtgctctcaaccaacaccttcaggaagagaaggaagcagcattgggcagaggcatagtcaaactacagtgctgttggcacagaagactgaagggagtcagagccagggggtagaggtgggcccttagcatccatccttcaccattaggtgtgagttgccccacctccttgatggtgtaacctcagtcccaaggtgggtgggagtgcagcagagcagcccctacaagggccaaaccagagatacaccaggcgccagaagtgctgccagggaatagagaggaaaggatgggcttaaggtaggatccacagaacttggcaatggattagaagacaggatgagaagtgacaggttaacactaacacagaaatgtctaacttcggtagataatggtgccattggctagaagaggaaaccgaaatgaaagcaggttgttcagggagacaaaagttcactgtggacatctcagcagagtgattcagtggggaaaggaatggatgcccagaccacctcagaggaagatctaagctggagccagcaataaagatacaagatgaacaatccctaacgaactgctcctcagccatgctccccagacacgctgcttcagatttatagtccgggtgaggctaggaggtgcgcctccctcagtggaggacagcaaagcaccagtggctccagggagttaaaatcttttgataatttttgttctagcatctgtctgcagagctgtctctcagccattgcctgcctttacacaggagtgcagtccgaaattgggagatgagtgaaatttattatgcctagagatctggatccccagttgtttgggagtatattttctgaaccacttgttggtttaagtaatgcagatttattgatgccacttctcttgaatctgtgactctggacccaccatctaagtgaatgtgcagagggaacggaatggctgcaatagatctccattaaaaccagtgcatcctcccagacacatacagtagtagggaggtgagtcaatgtcaggacagcaccagctcccgcttcggtacatttccaaagttctcagtctgtgtacaaaggtttgctctggggcagcagaaatagccctgggcaggtagtcaaaggcctggtttgatttcctccacttccaggcaagtcactcgaaggctcacaggctttttcctcacctgccacatgggtccagtgagatctactgagctgtaaataatgaaatgagtgtgtgtgcagtcatctataagttgtaaagtactagaaaatggtgaaactttgggatttgggctatttaaggctgaatgctaaaaatgtcaggcattgtggagaaaggaatttaaatataagattgattgactgggatttaaagacaaatgaaggcacacacgcaagtgcacacccacactgacactgcacagctcccgttggaggcatatcctgaccatgcagacctggggctctgcctgtccaagtgcactcctttactacataaaccctccttctcttttggggctgtcaccccaccagagctggcaccgagcccttgctgctgcgcttccctggggtgtcagcttttgacagggtgtttcctccctctgcaggagccttaacatcccttggacttccttccccccacccacccccagcagttttatctcttcctaactcgggaccctttttttcccacacaaagtttattgtcagttgctggtttcatctgtttgagcggctgcaacaaaataccatagactgggtggcatatgcacgacaaaaatttatttctcacaggagaagtcaaagattaatgcaccagcagatctggtgtctgaggggccaccttctggtttgtagatgatgctttctagttaaaacacctatttaacacactattaaacactaagtgtgttaaatagtgcagttgatgtatttgtcatgtcacctttatcatacactaaatccttctttgtctttttttctgtactctaatctctttctgtaagtaatctttgcttgcagcagtaggatatttagagtactgtggcttgacaatatatttagtatttcaagatttccatgaaattcttctgatgtatgagttccctagttaatcttacatatgtatccctttgtaaaaacactttgaacatttaaaatgatacatgaatagtactctaatacaatgccataaaaattataaatcatttgtatagactggtaagtaaagattgtgagattaagaaacgcatcaaaggccattgagctggaaagtggtataatgagaattcaaaccagggtctcttgactcaaaatctaaggatcataccatttctcatgataatatgagtattattgttatctctatcccatagacaaagtgttaacactgaatgagcagtgaaatagtctcagaattttttattttatttagcaattcacttgtcatttctggtcctcagtttattcacgagtaaaataaaatagttggactagataatttctatagtacattcttacacaaaaaatctatgattttgttatttttaatgtgatatactcatggcactcattcacctcattttcccagcctgcctcactggtcattacttctctgtgttctttacaggctccccctcctctacactgccattaaatattgaaacacctcaaagctttacttatgtccacctctcctctgacactatcattctgtctagatgatcccatacatacatgcccattacttcaacctgtatttatacgccaatgattcactatatttccagcctagacattcttttgtactctagttaccagcttgatatccttacatggctgtttcaaaacaactcaaatatattatctctcaaaatcaaactcatgatgtccccacaccatcctagctttccaccaacaatacctatccctattaatagcaataccatttattcagttatccaaatcaaaaacctagaattcatccttaaaattctactatcattccaaatatcctatccatcagcagccactgtattcttaatcccctgtatttccttcaaatccattcacctctctccatatccattgctgcatgactatccaagccatcgcctctaccctagggtaccaaaatagcaacaaacctaatctgttcatttgcattattttttctccaaaactgattatctatatgtagcaagacagattgttctcaaattgcaaatcccactatattatcctcttgcttcaaacacttccatggtttcccattgtttatgataaaaccaaatgcttcaagttcgaagaccggcatgattgggaatttcctgtcaccctagcctacttgctctccatggtacagttgcactggctttctttcattccttaagtacaacctgtttcctcccacctcaggactgtgcatgtgccattcattctgctgaggagcctttttccttccacttcaatcagctaagtctgattcttcctgacaatctcagctcaataagcatttcctctaagaaatgtctctaatatcattaattggctcaggtccctctactgtattgctgcacttttcacagttataattttacttaattatgaatgattatttgattaggtctatttccatccattagacataagcttcatgatggccagattactgttttctatccatcgttgtattccaatacctgacagaaggagggcgggaggtggtggcacacaagagatgctcaaaaacaattgttgaataagtaaatgaatgaggccatttagaaataacgaaagtacctgtttacaaagtacatgtatcaaaactatgaatgcattctacttacatggttttctccaaataaaacaaaagacttcaatcaggattaatacctgggataaactgagtcattaaatctctcctttgccatcaggagtgacattgaaacaaatgtctgcaaacaacaaatacttttttcccaaaatatattgaatggcatttccataaacaaactagaacatgggaggagaaagaaagcaatattaatttaaaattaatcttatcacataacttataccatcagggatttcgggtaaaattcctttcaggcacatccatttaacaagaattgattgttactgaaagcctagaagagaatttggcacatacttggtgttcaaatatttgttgactgagtgaataaatgatgcaagtgtctaagaaacacaaaataaggacatgattacagtcacggtggagttcacagtcatctccaaaatgaggatatgcatcccagggaggaccaacaattcattggagtgctgaaataaaatactcaaaggtcattttacatgtattttttctctaaattacttttcttaagacacagaaaacaaaaaaagaaacttagctttgttactttctaacaaatagttaaatcattaaacaggattgacactagcatccttgtttggtcttatgccttaggggaacatgaaatgtgtgaagacattctgagatctgagggaagggtagacagtaatacagtgggactgaccaggcttcagcacacctttacctcctctcagcagatttcagtgatgagcagtttacaactagattgaaagattatattatctagttctaaaagaaaactaagcctcccaaaagcaacaagggaactgagaggaatcctgcaaaacaaaaacaaattttaaaacttgcactttgtaataaccctaatatgtaatcacagtaatgaacagtaagataatgacagaactgacatatttccttatctattaaagccatattaacaggtaaagcaatgccagtcagtggtacacttcttagaagatatttaatacatactagacacatacacacacacaacattttccttcaaggtgtatgtatcagaaaatcactttttaaggccggatgcagtggctcaggcctgtaatcccagcactttgggaggccgacgtgggcggatcatctgaggtcaggagttcaagaccagcctgcccaacatggcgaaaccccatctctacaaaaatacaaaaattagccagggatgatggtggatgcttgtagtcccagctactcaagaggcagaggcaggagaatcacttgaacctgggaggcagaggttgcagtgagccaagatcacccattgcactccagcctgggcaacagagtgagactctgtctcaaaaaaaaaaaaatcactttttagataaaattcatgctatagagagaagactatgaaaatatgtttagcaatgtgtccatcattaggtgattgagtttccttttgttttgttttactgaaaatcatataaagtatgttatctgtaaaagttctctgacatgcacacataaaaatttgggagaaaagattaactataatgtttaatagattttgtacacatttctttaaaaatatataaaacacaacacctttcaattggtttgcaagaataaccaattgacatcatggaaaatggaaattcacttgctgaattttaacaaaaatttgcatgatgagtgagactgacaacttagtgtcatgatttaatgaattatgccaatggtaaacttcatgcacatggggccaggtaattatgtggaaactttttcaatgcttaaagccaagtattgaaattaaacttagaatcagacctttgaaccattttatgacaatgttcaaaaattataaattctatccacttatattataatattaaaaatatcattacaaaaaaaacctgtgtttattttataactcagcctttttaatttctaatttcataaatatattataatggatattgttagtaatgtagtattattacatgtatataatttataagtaaatatacatgttttggctactcatgcataaaatgtttcacccataggagcacataatcagaaatgtctggagaccattatagtaatagatagatcatattgccacatattttatctcctccttgacaactgagctttccagatcttctggtgaaacgaaagagaaagttgtaacagaagagtgattaaaatgacaaaagcattacttctattacttctattctaataatatgagcaaagctataactatcaagtaataatgcactaaagaaggtgattaatctgatatattcacaggcaactaataagacctttctattgcagccatgaaaaatatgtgacaattatagatatcctgtgtgcagtgtttcaacctttatgtgacctgttctactaacagatttagtgatgttcactttgttagaattttcttacacatgccataacttgcttcagtcttttgattatgaatattatggatattaaggattctagactattctagatttaaaaaataatattgtcacctcaatcagaagggaaatattaaatagttctcattttttcaatgtttactcagtttttgtccaatgtaatgaaagtgtcagcagtacaggttacaaaataaaatgtgtattaaagtaaactcatttgaacaggttaataattgtagagggagggaaaaggctaaaagattgaatgtaaaacttatgaaaagtagatacatcgtctctatgatttgcagtagtcaactgcatacagatgaatcattttaatacacgttaactactttccttttacagatggagaaactgagaggaagaaagtttatatggttcattaaactttgtgatgcaagctaaactaacctgtctctgtattttccatctactgcccttatcactatctcattagaatactcttcaagcatctccttactgattttcttaccaagcatttgttaagttctaatgagagttggtagtaacattttcacccactctgtgaaatatgaaatcttattcataggcctcttcttttattcttgtatttgcatatcaaccaattaatcaacttgctttctttatgttgcttattatcttagtccttactaaattgcctcttaatgttgtccacataacagaaatgttaaggtggatacttaacattttagtccagtctagccggtgccagtgcaatgccaaatcatgaattaaaatataattacaagaaccacttatcaaattttaacaattccttcagctttgtgacagttttttctacttcgattaaagtcaagtaaaattaaagttaaatatttttattaaaatatctcctttaacattccatattaataaacatattaaagctcatgcttctaagtagattactagaagttactttatcgaattacagcaatggttaattctagatcatagaatttagaatgactttttgccttcttcttttttttccttttttttaaacagagtcttgctctgttgtccaggctggagtgtactggcgcgatcttgactcactgcgacctctgccctgcaggttcaagtgattctcctgccccagcctcttaagtagttgggattacaggtgcctgccaccacacctggctaattttttttttgtatttttaggagagacagggtttcaccatgttggccagactggtctcgaactcctgacctcaagtgatccacttgcctcagcctcccaaagtgctgggattacaggtgtgagccactgtgcctggcctgactttttgctttcttcttaatacttactagtatttcttgaatttttaaaaaagaaacataaagtactttgataaaaccaacagtctcattgttcttaaaattgttcaaaggttctctggaaaaaaaaaagaaaattatcatttggttaagaatcatgttggtctgacatcaatcatcctataggagtgaatattgaaaaagtaagatatattgtggtataatcgagattgcataaattttaccatttttgagaagaatctgctccaaatcctggcttaatgtaatatccagcatgctacttaattttcttgtcttcaccttttcatatccacatccacctaggtgccacctcacagtataagccagcataatccattcttctcaatgaaaccacaatacatctgaccctgcatctcaggagaactgtatcagccacagcacttccagttgactatgaatctgaatgttatgcctcaggagaaacatccttgctgggactgagtagtgattcaaggagatagttatgattcagtcaagaaattaataattagtgttatttttattattgagacagagtctcgttctgtagcccaggctggagtacagtggcatgatctcggctcactgcaacctctacctccccggttcaagtgattctcctgcctcagcctcccaaataactgggacagcaggcacttgccaccacgcctagctaattttttgtatttttagtagagacggagtttcaccgtgttagccaggatggtctcgatctcctgacctcaaggtccacctgcctcagcctcccaaagtgctgggattacaggcgtgagccactgcgcccggccataaattattaactgagccaggcacagtggtacacacttatagtcccagatactcaggagactgaggttggagtatccttttttatgttattttatttttaattattatgggtacataataggtgtacatacccatggagtacaagtcatgttctgatacagacacataatgtttaataatcacatcagggtaattgggatatccatcacctcaagcatttatctttctttgtgttaggaacattccacctccactcttggaataggcaccctgttgtgctattaaatacgaggtcttattcatttcatctaactatatttttctacccattaaccatcacctcttttcccctcttccccactacctttcctgtgaggctgcaggattcttaagcacaacagttagaggccagcctggacaacatagtgagactcaatttctaaaaaataaaaaagaaattaccaactaatgctaaaaaaatagtctctgatgcttaggtatgaattagaaatgaccaaaaaaaaaaaaaaaaaaaagactgccctttgcttccttctccccttctcttcaagttttccattgctactcattttagtctggtttaatcaggtttcatccattaaaagcaattgttgggatcacacattttgagttgtgtcagtggacttccctcatgctggcatgattcctgccccaagcccttagtaaaagccaccaagccatataacataatctctcattgagtaaaacatctgatgtgtttagaatgacttctagcaaaaaaccagcctgtccagcatcatctctgtataacagataaaggaataggtactgcatcaaaaggttatagaacctgcccaaatcaatcccatgtgttttgcaatggaattaggttgaactaaagtgaaaattcagttttctactcctcattaacatgtctcatgttgcaaggttgagaggaaggagaagaagaactgtatttacagagagattccccctctctttctttctacagattactaaaacattcaaagaatcaaatttaagaaatcagttcatcagagctcatgttgccaaactgaggtgagtggaactgtagaaaaaatatttaagtatagatacaatgtggcatacttgactttttgtcacagaatgaatagtaaatgacatgttcagataagttgttgtaatattatgaaaatagtattttagtcagcttaaaaaccaatgccaaaaaagccaaacatatgatctatttagctactaatgtaaataaccatattatatctattcttattgggaagaggaagaaggggtggagagagagttggggtgaaggtacagtaacaaggccatcctattgtaaaactccagtggatatcattcacagtgcagcctatgtaaacagtccctcctggagttgtacaatgctgtggtttgggtgtatccatccaagatcaagacactatgaccaacatcaaaagtggctttttggttttatctgcctgatgtgctataataaaagggtattatggccaaatccaaggcatgtctatcatgaattaataataggaggagtagcagcatgcatgctagttatttgccattcctgccttagttaaatatgatgtgataaaaccagcctttccaactgaaatagtcacctttactgactctcccgcaaatgtctcaaatgaccacattgctctagtctttaaataatatgcaatagttctttggtagaagaggaattatactaattctttctcaaatactagcatcacaagaaaattaattcttgttctctggagagtcacctagtaagtatctggagcacagatgtctggtcaggtaagttttgatgaggagttaaagggataagaagagtccatgagaagggtattttccaaaacacctttcggtcaattcagtgcacattcacttagtactttcttgtcagtatctgtatcagccactaatgttcaaaagtgagtaagccctgaaaacctgtaggactacatgagccttctgccttttctctccttttgttcacttcccacttatcactcaatcctctgcaacctggcttcaataccaccataaaatatcaactgctcttgccgattcaacaatgacatccagataacaaaatccaaagaaaccacatcagtcctattcttggacctttcaacagtatttggtcctgttggcctgtcactccttgaaataggactatcccttggtttgcatggccttgtataccctgattttccccttacctccctagctattccttcttagtttcctttactaggtcttacttctttgtatattccttaaatgttgctgaacatcaggctgtgctctaggcctctcatcttctcaggtcacactctctcctttccttggccttcactgccacccatatgctgagtgctctcaaagttgtatctctaggccagtcctcttttgcctccaaacatgaatatatgcagccatctacttggtaccatcacatggataattctcatgatctcttccagtatgactgcttctttatttttttctgggctcttttttagcattgctttacatggaactttatcatgtctctcaacctctattttatcttttatctatgtatgtagagtctgtgtaatttcttcatctcttttagataactaatatctcttcagctttgacttgtattctgtgtaacccatttattgcgttttcaatttcaatgagtatgttttcctatctgcaagttctatttgtttcttttgagaatcttcctggtcttttaaacacatttcttattttaatttttgggggtacctagtagttgtatgtatttttggagtacatgagatgttttgatacaagcaaacaatgcataataatcacattgtgtaaaatggggtatccatcccctcaagcatttatcctttgtgttacaaacaatccaattatattcttttagttatttttaaatgtacaattaaattattattgaccatagtgactctgttgtgctatcagatactaggtgatcttttaaaaataatgttttctacttaatctcatttttatgattccctcttttacgtcatttgtcatttcaaatacagtcacttgtctgttgattctattatgtgaagtttttgaggataatctttttgttactttgattccaccttggtatggtttggctgtgcccccactaaaatctcatcttgaactctggttcccataatacccacatgttgtgggagggaccttgtgggaggtgattagattatagggacgtttcccccctttgctctgttctttttcctgccaccatgtaagaaagatgtgtttgcttccccttctgccatgattgtaaatttcctgaggcctccgcagccatgcaggacctcttttctttgtaaattacccagtctccggcggttctttatagctccgtgagaaaaaactaatacacacctcatgatgtattgtttaccactgaaattgtatgcttaaatttaatctcacttgggaccctgtacaacctagacttaacatatctacctccagagcagttacatctgtcagacattctagaggaatcagcagcacatggactttgttgttgttaatttgttgtcgggggaggggggagggatagcattaggagatacacctaatgctaaatgacgagttaatgggtgcagcacaccaacatggcacatgtatacatatgtaacaaacctgcacgttgtgcacatataccctaaaacttaaagtataataataataaaattaaaaaaaaaaaggttctgggagtattcaggtagtattaatgaagattcagacatcgtgcagccaggcccatgcttatgaattttcaggtgatacttctttttcttttttcttaatttaaagctggatctcggaaacagataaatttatttttttatgacatgacgagcatttttttcattctagttcatgctgttattgggtgtttagttctttgagactcctggcctttttctaaaacctcaagttcaacttcctattttgcactggcccaaggtcccatctccagtctctatgtaaatgctaaacataagcctgtggaatattctagtctcaccacatactattcacattcttctttgtttttggtcttccaggattttccttacttttctatgaacccagtcttgcatttgaaatggaatttattatatattatctatcctttctatttgttttatgcagaaagtgttttctaaaattatttaggcttccatattgctagacatggaagttgtaattatttgttcagtgcctgtttctacatctaaactgcaagacccatatggcaactgtgaatcttagtcccagctaatttctgaagcttagaatagtgcctagcacaagaagttgtttatctaacatttttaaaaataaatattaaattcatatctggaatgaatattaagttagagctggtcattgaggtgagaggaggaagccaagagagaatatgagagcctcaaagccaaatatctttaatgtactttttcagaaaagaagacagccaatgtcaggtggaggaactggtttatgaggtaactttcctggaagaaaatagaaattactgaggttttagataatccaaatatttaatcaagtcaccaaggtttattgtggggaatctttattattaattaaaatgagtgatgaaatcttaatatacgacaaaagttaaaatttgcttttgcaggcagatgaatggtctaggtatcaaaaaattaagttgagtctctaactcacacaaatttacaaccctatcactttatgaatttgtttaggagattatttttaataacactggtgaagtctaagaatagctaaaatttatagtacacttattgtgtgctattgactcttctttgaagttttgcatatagtgattcatctaatcttcataacccattttacatgtgaagaaacttagatatagaaagattaagaaacttacataacttatccaaagttacacagtaaaactctggcattataacttcaaaatcagctatcctacagtgagtacagtgttctgtgcattgaaatcaaataagtgagatagcatcgtgatatagtattacgtatgcaaacactgttacagagatctgtctaaagttaaattccacaaatgaattctttaaaagggtttaatcaagaagaatatataaacaggatggtgaaaaattgtcatattatttgttttttaaaatatctttatgatttacaggcaagatggtagtggtgtgagagcggatgttgtcatgaaatttcaattcactagaaataacaatggagcatcaatgaaaagcagaattgagtctgttttacgacaaatgctgaataactctggaaacctggaaataaacccttcaactgagataacatgtaagtataatttttcataaacaattttatttcaatatatccctcaagtttaccaattcaaattcatattttaattgagaggctgacttttctttctttgaaactaaactgtgaaaacaatccattaaaaagctaaatataccatatagctccctaacgtaaatcattctaagacttaaagaatcatttggcatttatatagtaaattttatttgctaaaaattctcattaattatccctgcaacattccttatgagtgatgttactgtcagatgtcattagtggataggccataggaggggtacatagatgctcaaggtcagagaactatttaattaatgatccacctcagaggcttcttcatttttctttgtaacatttatcacaattgaaattacaaagttatctgtgtaaattttgtattgtttggcttcatcctacactgtaatcatcctaaaagaaagaaccagtcaaccttcttcatcctactaccctcctaccacccagtctccatcatataacacatattcaataaataattcttgcatgactgaaagaaaagaaataatatatgcatagaatttaaggacattcctccaagttggttacattctgctagtttaataagccattatttcttctcgatgagctcaagattaaaaggattttgatgattcccatactagactggtaggtaccagttacagatgtactaactgttaaatattgaaatgctttcctatttgttggtaaacaattactgcatcaggcccacaaagttgtcttccgagatgtttcaaatccactgcccctgctgctaaagagttatgcttagcaaagcaaagcactctaagacactgctccaactccatggcctgattgcatcttttatgactggccaatgctcacgcactgcagtttgttaggtagttgaatattacctctgcttccacacattaaggaatgctcccgaacgcacttcccaagtgtttatttatttatcattatactagacaatatggtgatacgatggtcacagaatagcggtttccacctccagagcccataatctagttgaagggaaagatattccaacacaagagtgttgacaatcaagatagaatatgatcaagggcccagtgtgaggcccaggcaatgatcactgcaggaatctggggaagaaagagaccagcgtgcttgggatatctagcaaaagtttcatgaaggagaatggactttgactttgaaatatgggtaggatttacatattttgagatgagaaaaagaaagttcccagagaaggaaagcatgaaaaggcaaacagtctgtactgaacgcgatgctttgacagaataatgaagaaagggacctgctggaatgattgatcagtgttcatcattcacaccatcatcatcaaaacacttatttaatgagaacttactgttttttaggcatggctttaatgccctatatgaatttttttcttgattaatccttacaacaaacatatcccatagatagttttattgtcccccttagaaaagataaattgcctaggctgacacagtcagtatatgaggcagtcaggattcaaactaagtctgtttgttcaaaaaattaagaatggccagctttttaaaattttctgtctccagaagtatgatttggctccactgaagtttgcaaaacaaatgtgatacccaaaccttgtgaaacttttagtgggaaataactttgcataagtcggtttgagagagcgtggaaacctgtcttgaaaagttttaatttaacttgcaggaaataaaaatgatgggtttctcaattaaaaatttcaatcaaggaaggatatgagctaacataacatttttttaaaaagatcagtctggtaaggtagaggtgcataaactgaaaaggagcaaaagtggtggaattcagttagaaaattattgtaactgtactgatgtcaaatgatgaaaccatgaactaaagtagtaccaaaaggagtgaggaggatggaataattcaaaagatagaggacagatgtgcagaacctggagattataagatgtgaaaggaggagtttgagaaaatttcagattttggaagtggtgtcattttactaaaaggatataataagtagcaaattttggataaagttgggtcccactgagtttgagatggctgttggacatgcagagaaaactgtcttgtatgctgttcttaaattgaaatagacagacctttaccctctgatactgacatattttcctttccaggctcaccctccatttccctaaacacaacacatgcactagctctccttactttattgctccacaaacatcttacacctccaagcatttgtgcccactgtaccttctatctggaatctcttttgtcctcttgtgtgcctgaaaaattcctttcagatcttcaaaatacagtgcagatgctatttcttctagctcaaatattatctcctccatataatttaattactctcttttttcttttctctactttgcacttacatttatttgaatgattgcttgattaatttctacctgtaaattatgtgagggcaggtcctctatattttgctcgcagttaaatctgcagcacttattatagagtggtatcattagagtaatatacatatatttgaggacatgataaattaacttcccctatagtatttatcacattgcatctcaatgacttgcttatgtttctgttttcccatataaattgagtaacttgaaaaaagagatatctattaagtatttaatgagaaattaaagtacaaactttagtatgcataacaacaaattgggaaaaggttgtaaacaaagagatttgtagggcccatgagttagagatcgtttcagcaggtctgaaaggaagcctaggaatctgcattttagaggaccacctcccaaccccaacaagtaattctgcttcttgttgtctgggtactgtactttaagaaattatggtgaaatgatatcagcctttattgtatttatcttattctcattttttaatactagcacttactgaccaggctgcagcaaattggcttattaatggtaagttttaatattattttgtaactgtaatttgccaaatcataaagagtaaaagtgcaagtcttttgtgtacttttggccaaggcagtatctatcaagttgatgtctttgttcttagttcgctcaggtggtgttgaaacaagacagtgctgatcccaagtgtcccatggagtggactttaggtttcccctttccttttagaaaaaggaagaagttgtagtggaggactacccactctgcactcaaaattgccctcatgaaaatttctttggcagctttgagaaccttttactgccctggttctaaggtggcatttctgtagacttacaaattatgtttgatgacaccgtttatgtagcttctcctaaccaccagagtagcttgctttgttgtgaattcaggttaatcacaaagtataataaaaaagaattgtcagaagtcttcccagctttgggtctataacctgaaggaaaagtcactactcttcaacatcatcctatgtactctcaggctaggatagcagaaatgcaatccctagaaaacagcaacttacttctctgaccaaaaaaatgcagttaaaaattagttcaatgtacctggtagctggcctatcttaggtacttcagtgattttacaaagtgatggtagtcctatgggtgtttttcagcttcactacgtatttaattcatgcttattgttaatgaaactgtgataagcaatttactagggtatttgtttgggagatgccacaaaggaacacatgtatctcttaatggaagcctggtcctcctttatccaggaaatttgctaggaaaaaaaagcctttaggtggttgtgctattaaaccagggcactacttaaaagccagcccagcaatagttgtgtgatttaccattaatttcttagtaatagaccacacaaaagaagaaaattatgggaatgcgagttgagaggaattgggtgatcagcctaccccagcccgtttcagctctggccagtagactattcacgagctctttgaaaacatttaaataaaccttatttagatactagaaaccctctgtcaccctcaagaatattctgtggtatagcgactcctttatgagggcatgtttggtaatacagcatcagtcttggaggtggactggattctacaaggtgaactgcagtcactaaggagtcttttggatgagaccagttttcctccaacttcaatgtgtgcatgaacctcacatcaaaatgtagctttagatttgtcccatgatgtggttccaagaatcagcacttctaataagtttccaggggatgcccatgctgcaggcccacaaaccacactgagcatagcaagactattgagaaaaaggaaatttcccaggagtctgtggcctgagctggcacatccaataatgacctatcttaacctcaactcatgaggaattccagggaactctgaagctgctcaaaatttgaagcctatatgccaactaaattcagaaatgttctccaaaatgctatctataagcaacagtagtcacaaatgcattgtagaaatatatcgatcatgctttttggaaaatccagcatgtcctgaggaagaatgtataagacataaaagtcataaattatggaaagactcttcagcttcttccaaatgtaaaggaatcatgatcttcccagcacattaatgccctttctcattagaatgtggggccggtccagacctaataacattgtctgagcagagaatccttggaggcactgaggctgaggagggaagctggccgtggcaagtcagtctgcggctcaataatgcccaccactgtggaggcagcctgatcaataacatgtggatcctgacagcagctcactgcttcagaaggtgaggccaccactacctacccatctgggaacaattagaatagacaggtcatgaagactgcaccctctaccctaggattgaattgagccagaaataattcaatgcaaaaaaatcagtaagaattttcttcctattcatgaaaggaaaaggatttttcccctttagcatgctaatttagtgctatttctctgtttcaggtaataatatattagcacagtaaagaacaaagatttatatgtcagaatgttttttaaatcctagctataaaagcttaagaaatttactaaatctccataagctttattttttttccaaattaagggacaacactgttatctgtgacttagtgttactggtagcattgagtacactaatgtaaacatacgttaaatgttagcgaaacgaattgctgtggaagatttgcacattatatcatgggagctgatggctaacctagagactgccccatgccattaatttattcattcataaagattattgagtatctagtatgagcacagtgttatatattgtagaagctactagtataaacaaagtattgcctctgccttcaaagagcttacactcgaatgttggaatcagaatgcacaaaaataatgatcaattacaatgagtagcataaataaaattaatgtaggcaacttacaagaattcttaattgaggtgactaaactattgccaacactagggtgatatgctaccagtggcgagtaggttgcataaacttaccttattggtaaaaagaaaagttcacattgctcataaaagaaggattttagatttcagcataactaaaatctgtttcaaacctgccttgttactggggcatcgcagaccacaacagttgttgggaacttaactcaaaaagttcacccagaaaaataatggagatttgaactcgtgtgcccctgaccatatcaattttcttctcagactcttactctaaactggacctccttatcacacacacaaagccttccataggcagatcaatccagtcttatttctcaaagcatgtaccttgagcttcagataaacagcattgttctcttcccctggactcttcctacatttccctacctatgagtatctgatcaatctgcttatccttgaaatgttaatatatttaccacatctctatttgaattttatgaaatttttgataatttctaagtagttttttcagatttataggcactacttcatggtacagtgactgttacaaacgtatttgttaaatttagaaggaataaagatttaaaagactagggtagttactgaactaaagttttaggaaatcccaaattatttcaaatttttcttatggtaattttatgacttaatatttttatatgcagtgaacaaatttgaaactttaaaagatactcccagaattatcagttttctgatgtagattggcaaatttattactatatcccaaataacccaagagacaaaattcacaaaaacatttcaattttcattgccacttgaaaggccaaaaagcagaaatggcacgcattgatttcaatcgtactcttgagtgtgggaaccaggaattaaaatacctggacttatcaggcacttagcataaccaagaacggaatagaaacctccctggattctaagccctattcagtcccaatcaccaaaaaccaagtaaacgatatcactataatgaaagccacagttataaatatcgacaacgattaccaaaggaatccatggaactttgaattttgccaccccacatccttctattcattaccatgattgatccactaaagctaacagactctgtgaaccttgtattggacccctccctaaagacctgattgtcactgagaaccatcagtgaggatttgtttggggcatgaccagccttacatcaaagtacatagaagtgatgaggtcttatcaaagaggattattgaattatcacctcttctatgtagctttccctgatactctctttcctctccattgagttccacagaaatttttttatctgcctttaacagttgtcctcatgatttgtgatatttgacttacctcttgtcagtttccttcactagtgtagagttcctcaaagaaagagaccataattacttatatttttattcctggagactcatactattccttatacaaagtagacacttaacaatggcttgttgaactataattaatgaaaataatagctaccttcatgaaagttcactttgtgccaaacactatagttgacataatacatttgtctcattaatacttaacaattgtgtgagaaggtatcaccaatcacattttatatgtaaataaaccccagagctattaattaacttgtcataaataacacttttcatatgtggcatagccaagatttaaatataaatgttactggttccaaaatgatgctctaattcacttgctggaaagaaggaaaggaagaaaataaacgagtggaaggaagagagggagggaagagagaaaaggaaggaaagaaaaaagagtctcttcagaaccttcactgtaaagactccgagcaaaagaagttgaatataaaaacaacataggtttgtttgttttctaatattttttcttcaaaatttttaactcaggttcactcttacacaaactactgtgtcttataaaagtatttccggtcatagaatttttattttctgtattaactccactatctaatctccataaaactcctaaattggtattatcggtaacattttgtttttactcaacccttaggaacaatgttaagttaatcagccctccacatcacagatccttattttcatcagtctgtacaaggcatttctctcattttaattttttttcctcctgtcatccctggatttcactttcactgccctccttccacccatatgcctcatactaatatattcgaaatatacatgtcttaaaggtacatgcacgcacctacaaaacctatagtgtttttttgtatgtatatgtctttaatttaaataagtagcattgtgtaaaagtctaatattgtttcttactgttttcactcaattcttggaattttcatctgatgcactgctgcatagcaccccatggtatgcagccaccatatttccttcatccaattaggttgcatgacctaccttcccattgccacaaagagtacacacaaaatatttgtacttatctttctgtaaaccttcaggaatttcagaagcacacatgcaggctgctaaatataccagaatactttccagccacttaaatctttaccagtattgcaaaagaggccccatttccctccacatcaacatttagtattattcttttgtttaagttttatcaatcttttaaatgtacacaagatgctcatttttataattttaatttctcagattactagtttgagtatcttttcatatatctaagagctgttttgatctcccctaccatgaactgccactaatattctttgcctattttacaatggtttttctgcttatttattactggtttacagacttttaaaatatattctacaaaaattttagacattaaacattaccaatattttcccatggttcctcatccatctggtaaacttgtctatggtatatctaattttgatttaatagaattcattctatttttaccttttagtttgtgtttttgttgtttagccaaaaagtccccattcctaggtcataaaggtaatgtcctttttttttttttaacgctactgttctctctctgtctccccctatgtatataggtgcacatatacttgtacacacatacatatacctatatatgaggggagttcgataagtttatggaaaataaaattaaaagataaaataaaaaattataaactttatttctcaacataagctccttcaagttcaagacacttttgtaagcaataataccagccatatcgtccatccctaaagaactgagggtcctgagaatttaactatgtcaatgcagtcttttttacattacttttttacagtacttattgatgaaaaatgggtgccttttaaagattgttttaagattagggaacaaaaataagtcagaggaagtcaaatcaggactgaaaggtggatgcctagtgatttattgctgaaactttcataaaactaaccttatttgatgagaggaatgagcatgagcatggttgtgatggagaagaactctggtggagctttcctggacactttttctactaaagctttggctaactttcttactctcataagaagaagatgttatttttcactgaccctttagaaggtcaacaagcaaaatgccttcagcatcccaaatgtctgttgtcatgacttttgttcttgactagtctggttttgctttgactggaccacttctacctctttatagccattgctttgatggtgctttgtcttcaagattgtattagtaaagccatatttcatcttctgttacaattcttcaaagaaatacttcagaatcttgatctgacatgtttaaaatttctattggaagctctgaccttgggtgcagctgatctgggcgaaacagttttggcatccatcaagtagaaagtttgctcaactttagtttttcagtcagaattgtataagctgaaccagttgagatgtctatggtgttgtctattgtttctcacagttaattgttggtcctctttgagacatgaacaagatgaaatttttcctagcaaactgatgtggatgatctgttgctgcgggcttcaccctcaacaacatctctttctttcttgaaacaaattatccattagtaaactgatgattgggggagatgctgtccccataaactttttgtaaggcataaataatttcaccattcttccagtttcaccataaatttgacgtttttttgcttcaattttagcagcattcatgttgctttgataagagctcttttcaaattcatgtcttattcctcttagtgcctcaaactagatcttgttcagtatgacaagttagtatgagtttatctgcatgcaaaaatctttgaaatccatgcatagtttgtttataatatacattttcaatgaacttttgaagaccccatacatacatatgtatatatatgcacacacacacacacacacacaccaaaatcttcaaccattatcagacttagtgcagaaaaattattcatccattaacaagataagaatgccccttatcatcactactatttaaatggagctcctggctaaaggaaaagacagggattgaaaaaaattagttaaatctaaaatgtttattatttcaggtttcttagttgcttaaatgggaagggaggtatggacaaaagagaaatcaaagatatttgtgttatgctacttatcattaaagtatcagaataacttcattggaatagaaaaacaccaagatcaccccacgatatgttttctaaaatcttctccatttctttagacaagtgaccatgtattcggccagtgaagaattaaactcacttgccagcttataatgcaggaaaatatagcaaagagatgtggatccaatagtttctagatagtggtacaggatggctaagatgaatttatatatctgaaatgttcacaaattccctactcatatagcatgttttcataatgttttagcaactctaatcctcgtgactggattgccacgtctggtatttccacaacatttcctaaactaagaatgagagtaagaaatattttaattcataacaattataaatctgcaactcatgaaaatgacattgcacttgtgagacttgagaacagtgtcacctttaccaaagatatccatagtgtgtgtctcccagctgctacccagaatattccacctggctctactgcttatgtaacaggatggggcgctcaagaatatgctggtaagtgtctcggaaaaaaaaattaacaatagaaatgtcttatatttgctattaggtaattttttaaattaggaaacatctggaataggtgtttctattcttctacagacagaaccattctatattctgctcagcccaagctctggctacccctgagtctccttagcaaagcaaagcaatgctccagaaactatgggaattctcaaatatagtaataggaaaatgtaaaagaaagttatgaagacacgagttctttaataatccagagattctataagattcaaatagcttccctataaacaataaaaaagattttgtttgtttgtttgtttgcttgttttttagagacaaagactttctcagactggagtgcagtggtgcaatcatggcttactgcagcctcaaactctggtcttaagaaatcctcttgcttcagcctcccaagtagctagaattataaataagtgtgtaccaccatacccagctttttttttttttttctacagacaggttcttgctctgttgcccaggctggtctggaattcctgccctcaagccatcctcctgccttgttggcctcccaaagcaatgggaggatttagattagacattgtatgagggcttaataatccttaaggtattaactgccctttaaagtattctgggatatggcaaaaactcgatgtgtatataaacattggtcatatttgtttattgaatgaataaaatggaaactaaaatgaggacaatgcacaagagctactagaaccagtaagagtatcagcgaaggagtggaagggtagcattgacaatttccctgggcttttacccatgttgtagattgtctctccaaggaataatacaaagccttaatagtcctagaacacattctattgtgttcttatggcccaaagtaaattggtgtagtagataacatttgcaccagtcatgaaaaactattggtgtcattctgagagtacatcaatataaaatagactagttctttagccttgaaactagactggtttctcttttgctgctaggttaaaggttattcaatatgtaatcttccaatccaaaatctgtcagtggataatttaaaagcttttagtcaattttaagatatttgttttcttaaaattttaaggggcactgtgtcacaaagctaaagaaaaaaaagaaaaaaaaactgatctgtgaaaggggttatcctcatctacttggggaattttggctgcgaagaaactccaaagtaaatctttagaagccttcattgttaaatatgaaataatgtttggagtacatttatttcttctcaaatttattatagggtcaataatgtacacatcttgaagtccatttttttcctgcttttataacaaacaggccacacagttccagagctaaggcaaggacaggtcagaataataagtaatgatgtatgtaatgcaccacatagttataatggagccatcttgtctggaatgctgtgtgctggagtacctcaaggtggagtggacgcatgtcaggtaagctcaagacaatctcatccatgtcatcatccaagaagtgtataagcacttcctagtatgtgataatgtgatagacataagtgtaacagttacaatacacagccctgttcctctaaaatttataatctagattttagaaataaatttttttatgaatgaagtttatctatcatgaaagcattaactctgagaggccaaattacagagtagttaaccatccaaagctcaagaatcagaaagacctcgatttgaattccttaacctctattaccaagtctctaactaaaagctggggataatcataatagcacctaactttttgggtactaagaaaagttaaatgaagactaaatatatcaggcacatggtaaacaacaaagaaatctcatctatttcactattattaatgtagaccatggtcactcgtgttaataactttaacctcaaccttttaactgctgtgaaggattaaataaaaaattaatcactatattataaaaattaattgatatataataaatgaattttaagagatacgtaataattcatggactccttgaagatagaaaatttatacaaaatcctagtaatttgagtcacaaaagctcctacaataatgaaacagtatgaatgaaaaagaaaagaaataactattatatttggatctagcccataatttttaaccaaatgcacaaaaacaaacaacaaatatgaaattctcactgtaaagtgattaaaatcaaatttgaattctaaaattttaaattaaattatctaaacataattgatgcagttatatgttttaataggttttgttcacatatctgaaatccaactccacacagtagcaggaacagctggtgtcagaaattaaatattcttttagtctggagttttaaaaaatcaatctgtttacttgagtaatttgttgctgttttcatgggtgaattgtatacagaaggataagaattattcttcgcatcaaaaggtcactgactttcatatttagtgctcatggtctttaaaaagtggataaaaagtagttctcacatttcatggaaagcccccaatccatgagcacatttcccaaaatgaaacatttttatcaactgcaagttgtgtgtaggtggagatttgtttttcaattgtcaagatactgttaattacccagtcctttatctccttttggtggagatgtctctgtgctaggaaacccttcttgctctccttcctgtttctcttttactactggccctgaaacaacaaattctcaagtttcatgacagctttccaaagaatccatcaatcaaataagcaacacaactcgacactgacaattccagacctactaagagcattaattaagacttaaaaataaacatgagttttaaaagggtgttattcattattttcccatttataacgtcccttaccttctgtccttcagtgcatacaaattattatcttccttgaagcccagttcaagccgtacctcaccatgataccttccatgtatattccactctaggcctcactgatttttaactgaaatactataatgcatagttcacacttaaaaaaaaaaaaaaaacacagcactttacataagagcttacaggatcctatttgttttatccattcttttgttcatttttacaatcattaattcaaaggaattatattaattactttctatgcacccgacgttgtgttaacacaacaatactatccctgcattcagcaagtctatggtctacaagagaggacacaaattcaaatgtctgtagtcaagcagtgaagctggctagatatggaaaaattacaagtccctcttgctttaacatttgcttgcccacatttggtcagacatcatgcaaaataatttctcactatagaaaaaaaaacactacaaaaacaataatataaagaactgagaactggttaactgaagcatgcatatgtcatctaaaagaagcaggtgacgaccagcttcatgaagtacttgccatgcatattggcacttcacacactgacccttctccccacctagaccagtaattaaacaggtatggatgagctagctactaagagcagccaactgaatagctgactaacttagaagcacacttggtaataatagctgacttttattagtactgactatactatatgctaagctgtactcaaagtgctttgagttttaaactgatacaaacattatatgaggaaacagaggtacagagagctattcaccagcttaccaaaggtcacatagctggtaagtggaggacttaaacccagactatctagtttcagaacccacagacttaatccatcgtgcagaacataagacatactccatctgtctccccaactaggttattatgtgcacaaatatttattggttggttggttcattattatgactgggtggtaagtatgtcattaggagtgttttgcttatgactatataaatttcttcaccaaaagaagactttctgatgatatactatgcatcagacaccacgcagggtgctaaggttaggaagataagtgagacttctagaaactcattcattcaacaaatatctcctaagggctagaagcttaggtttcagcagtgaacagaataggtatgttctctttcgtgttggaccttatagtatatctgggaaaacagacattgaataaatatcacaaatgcaagtgagtgtttcagagacatgcagctgctacatcaaaacaaaacagaacaaaacaaacaaacaaaaactgaccagtgggattaagtgtaaataggcacacaaatgcacaaatatgcttttataaaatagtgaagcagtgacagagacacacacaagatataaagacacaatgaagaacaattgagcccaaagctggaaagggtgagagtgtgaaggaaaaaggttgatcagagaagttttcccgaaggagagaaagcctggatgattaggaggcaaccactcggtgactgagggaaatctgaaaaatgtatttgtcatcttctcagacttgctgaaggaatgacttgggtactttgaggatttcagtaatttttccatgacttggtataatatttcaaaaggaaataggctgactttatttgtataatgaatgtgactccttcctcgactgccatagaaataaactccttaatattttgggtttgtctttgcacttaagtaatcagtcattctgtttttttacagggtgactctggtggcccactagtacaagaagactcacggcggctttggtttattgtggggatagtaagctggggagatcagtgtggcctgccggataagccaggagtgtatactcgagtgacagcctaccttgactggattaggcaacaaactgggatctagtgcaacaagtgcatccctgttgcaaagtctgtatgcaggtgtgcctgtcttaaattccaaagctttacatttcaactgaaaaagaaactagaaatgtcctaatttaacatcttgttacataaatatggtttaacaaacactgtttaacctttctttattattaaaggttttctattttctccagagaactatatgaatgttgcatagtactgtggctgtgtaacagaagaaacacactaaactaattacaaagttaacaatttcattacagttgtgctaaatgcccgtagtgagaagaacaggaaccttgagcatgtatagtagaggaacctgcacaggtctgatgggtcagaggggtcttctctgggtttcactgaggatgagaagtaagcaaactgtggaaacatgcaaaggaaaaagtgatagaataatattcaagacaaaaagaacagtatgaggcaagagaaataatatgtatttaaaatttttggttactcaatatcttatacttagtatgagtcctaaaattaaaaatgtgaaactgttgtactatacgtataacctaaccttaattattctgtaagaacatgcttccataggaaatagtggataattttcagctatttaaggcaaaagctaaaatagttcactcctcaactgagacccaaagaattatagatatttttcatgatgacccatgaaaaatatcactcatctacataaaggagagactatatctattttatagagaagctaagaaatatacctacacaaacttgtcaggtgctttacaactacatagtactttttaacaacaaaataataattttaagaatgaaaaatttaatcatcgggaagaacgtcccactacagacttcctatcactggcagttatatttttgagcgtaaaagggtcgtcaaacgctaaatctaagtaacgaattgaaagtttaaagagggggaagagttggtttgcaaaggaaaagtttaaatagcttaatatcaatagaatgatcctgaagacagaaaaaactttgtcactcttcctctctcattttctttctctctctctccccttctcatacacatgcctcccccaccaaagaatataatgtaaattaaatccactaaaatgtaatggcatgaaaatctctgtagtctgaatcactaatattcctgagtttttatgagctcctagtacagctaaagtttgcctatgcatgatcatctatgcgtcagagcttcctccttctacaagctaactccctgcatctgggcatcaggactgctccatacatttgctgaaaacttcttgtatttcctgatgtaaaattgtgcaaacacctacaataaagccatctacttttagggaaagggagttgaaaatgcaaccaactcttggcgaactgtacaaacaaatctttgctatactttatttcaaataaattctttttaaaataatttccctgcctaattatttatggaagttatgacttttgaaggacaattcaaaaccatttatttaattggttctgcaatgaaagaactgccccatatactctactaaaggcttggcactttctgctgccttttaatccagcgctataattgaggcaagcgtccagcttgacacctcgagataacttcgtataatgtatgctatacgaagttatgctagtaactataacggtcctaaggtagcgagctagctgcaaccgaggaaaaaacgtgccatgaggtctctgtatccaagtgtgact

SEQ ID NO: 88 - protein, 418 aa, synthetic construct
  REGION 1..418: note = Recombinant protein
MYRPRPMLSPSRFFTPFAVAFVVIITVGLLAMMAGLLIHFLAFDQKSYFYRSSFQLLNVEYNSQLNSPATQEYRTLSGRIESLITKTFKESNLRNQFIRAHVAKLRQDGSGVRADVVMKFQFTRNNNGASMKSRIESVLRQMLNNSGNLEINPSTEITSLTDQAAANWLINECGAGPDLITLSEQRILGGTEAEEGSWPWQVSLRLNNAHHCGGSLINNMWILTAAHCFRSNSNPRDWIATSGISTTFPKLRMRVRNILIHNNYKSATHENDIALVRLENSVTFTKDIHSVCLPAATQNIPPGSTAYVTGWGAQEYAGHTVPELRQGQVRIISNDVCNAPHSYNGAILSGMLCAGVPQGGVDACQGDSGGPLVQEDSRRLWFIVGIVSWGDQCGLPDKPGVYTRVTAYLDWIRQQTGI

SEQ ID NO: 89 - DNA, 257 bp, synthetic construct
  misc_feature 1..257: note = synthetic oligonucleotide
  source 1..257: mol_type = other DNA
agcacccctctcttccgcagagtctaagaaatcgctgtgtttagccctcgccctgggcactgtcctcacgggagctgctgtggctgctgtcttgctttggaagttcagtaagtgcagggagcctcgatcccaccatgtgctcctgcagtccccagtgctctgagccagaccctgctctctgggctattgagacctctggaggccctccgtgaggttcctctcttacataacgaggctgtctctcttcccttctcttg

SEQ ID NO: 90 - DNA, 190 bp, synthetic construct
  misc_feature 1..190: note = synthetic oligonucleotide
  source 1..190: mol_type = other DNA
ggtcagaggaccaaaggtgaggcaaggccagacttggtgctcctgtggttctcgagataacttcgtataatgtatgctatacgaagttatatgcatggcctccgcgccgggttttggcgcctcccgcgggcgcccccctcctcacggcgagcgctgccacgtcagacgaagggcgcagcgagcgtcctga

SEQ ID NO: 91 - DNA, 171 bp, synthetic construct
  misc_feature 1..171: note = synthetic oligonucleotide
  source 1..171: mol_type = other DNA
attgttttgccaagttctaattccatcagacctcgacctgcagcccctagataacttcgtataatgtatgctatacgaagttatgctagtaactataacggtcctaaggtagcgagctagctccacgtggctttgtcccagacttcctttgtcttcaacaaccttctgcaa

SEQ ID NO: 92 - DNA, 177 bp, synthetic construct
  misc_feature 1..177: note = synthetic oligonucleotide
  source 1..177: mol_type = other DNA
ggtcagaggaccaaaggtgaggcaaggccagacttggtgctcctgtggttctcgagataacttcgtataatgtatgctatacgaagttatgctagtaactataacggtcctaaggtagcgagctagctccacgtggctttgtcccagacttcctttgtcttcaacaaccttctgcaa

SEQ ID NO: 93 - DNA, 20 bp, synthetic construct
  misc_feature 1..20: note = synthetic oligonucleotide
  source 1..20: mol_type = other DNA
gccgtgactgtgaccttctc

SEQ ID NO: 94 - DNA, 22 bp, synthetic construct
  misc_feature 1..22: note = synthetic oligonucleotide
  source 1..22: mol_type = other DNA
tggaggagccacctgatgcctc

SEQ ID NO: 95 - DNA, 20 bp, synthetic construct
  misc_feature 1..20: note = synthetic oligonucleotide
  source 1..20: mol_type = other DNA
gccttgccctcaatggaaac

SEQ ID NO: 96 - DNA, 21 bp, synthetic construct
  misc_feature 1..21: note = synthetic oligonucleotide
  source 1..21: mol_type = other DNA
ggttgcacagcaaggaagaag

SEQ ID NO: 97 - DNA, 24 bp, synthetic construct
  misc_feature 1..24: note = synthetic oligonucleotide
  source 1..24: mol_type = other DNA
ccaggagttcctgtgagcctaccc

SEQ ID NO: 98 - DNA, 20 bp, synthetic construct
  misc_feature 1..20: note = synthetic oligonucleotide
  source 1..20: mol_type = other DNA
tggaatggaaggagctggag

SEQ ID NO: 99 - DNA, 19 bp, synthetic construct
  misc_feature 1..19: note = synthetic oligonucleotide
  source 1..19: mol_type = other DNA
gtcccacctcctgcaactg

SEQ ID NO: 100 - DNA, 22 bp, synthetic construct
  misc_feature 1..22: note = synthetic oligonucleotide
  source 1..22: mol_type = other DNA
tgagccttcccatcagcctggg

SEQ ID NO: 101 - DNA, 21 bp, synthetic construct
  misc_feature 1..21: note = synthetic oligonucleotide
  source 1..21: mol_type = other DNA
ccacaatggcacatgggtctg

SEQ ID NO: 102 - DNA, 18 bp, synthetic construct
  misc_feature 1..18: note = synthetic oligonucleotide
  source 1..18: mol_type = other DNA
ggtgcttgctccccaaga

SEQ ID NO: 103 - DNA, 20 bp, synthetic construct
  misc_feature 1..20: note = synthetic oligonucleotide
  source 1..20: mol_type = other DNA
cctaaaaggtgttgtaatgg

SEQ ID NO: 104 - DNA, 24 bp, synthetic construct
  misc_feature 1..24: note = synthetic oligonucleotide
  source 1..24: mol_type = other DNA
ggcaataaagaaggaagacgtttt

SEQ ID NO: 105 - DNA, 120 bp, synthetic construct
  misc_feature 1..120: note = synthetic oligonucleotide
  source 1..120: mol_type = other DNA
ccagtcagggacacacatgctcacacgcccgcccacccgcacacactacagtcgagataacttcgtataatgtatgctatacgaagttatatgcatggcctccgcgccgggttttggcgc

SEQ ID NO: 106 - DNA, 198 bp, synthetic construct
  misc_feature 1..198: note = synthetic oligonucleotide
  source 1..198: mol_type = other DNA
attctagttgtggtttgtccaaactcatcaatgtatcttatcatgtctggaataacttcgtataatgtatgctatacgaagttatgctagtaactataacggtcctaaggtagcgagctagccaagtctgtgtgctaccaagtagcaaaactgagcctggaactcacacatgcgtgtctgagagcccagcactatcgc

SEQ ID NO: 107 - DNA, 100 bp, synthetic construct
  misc_feature 1..100: note = synthetic oligonucleotide
  source 1..100: mol_type = other DNA
taatctgactttctcttcatcggtctctcttattctaggctgagctgtaacgctgccgtcccccacatccagaagctgcttcccttcagacctacctacg

SEQ ID NO: 108 - DNA, 177 bp, synthetic construct
  misc_feature 1..177: note = synthetic oligonucleotide
  source 1..177: mol_type = other DNA
ccagtcagggacacacatgctcacacgcccgcccacccgcacacactacagtcgagataacttcgtataatgtatgctatacgaagttatgctagtaactataacggtcctaaggtagcgagctagccaagtctgtgtgctaccaagtagcaaaactgagcctggaactcacacatg

SEQ ID NO: 109 - DNA, 19 bp, synthetic construct
  misc_feature 1..19: note = synthetic oligonucleotide
  source 1..19: mol_type = other DNA
gagcagggccatgacacat

SEQ ID NO: 110 - DNA, 24 bp, synthetic construct
  misc_feature 1..24: note = synthetic oligonucleotide
  source 1..24: mol_type = other DNA
accattagatcccagcactggaca

SEQ ID NO: 111 - DNA, 20 bp, synthetic construct
  misc_feature 1..20: note = synthetic oligonucleotide
  source 1..20: mol_type = other DNA
aaacccttcccgagagagaa

SEQ ID NO: 112 - DNA, 23 bp, synthetic construct
  misc_feature 1..23: note = synthetic oligonucleotide
  source 1..23: mol_type = other DNA
gaggaacactgtgtcaaggactt

SEQ ID NO: 113 - DNA, 22 bp, synthetic construct
  misc_feature 1..22: note = synthetic oligonucleotide
  source 1..22: mol_type = other DNA
cctgaaaagcccggagtggcag

SEQ ID NO: 114 - DNA, 19 bp, synthetic construct
  misc_feature 1..19: note = synthetic oligonucleotide
  source 1..19: mol_type = other DNA
gggcagagaccacatctga

SEQ ID NO: 115 - DNA, 22 bp, synthetic construct
  misc_feature 1..22: note = synthetic oligonucleotide
  source 1..22: mol_type = other DNA
ggaagccctctctcgatacttg

SEQ ID NO: 116 - DNA, 22 bp, synthetic construct
  misc_feature 1..22: note = synthetic oligonucleotide
  source 1..22: mol_type = other DNA
ttctaccctgagggcatgcagc

SEQ ID NO: 117 - DNA, 22 bp, synthetic construct
  misc_feature 1..22: note = synthetic oligonucleotide
  source 1..22: mol_type = other DNA
tgggatgtagaaggttgtcaga

SEQ ID NO: 118 - DNA, 22 bp, synthetic construct
  misc_feature 1..22: note = synthetic oligonucleotide
  source 1..22: mol_type = other DNA
ctgagcctggaactcacacatg

SEQ ID NO: 119 - DNA, 23 bp, synthetic construct
  misc_feature 1..23: note = synthetic oligonucleotide
  source 1..23: mol_type = other DNA
tctgagagcccagcactatcgcc

SEQ ID NO: 120 - DNA, 19 bp, synthetic construct
  misc_feature 1..19: note = synthetic oligonucleotide
  source 1..19: mol_type = other DNA
gctgagggtcaggcttgag

SEQ ID NO: 121 - DNA, 21 bp, synthetic construct
  misc_feature 1..21: note = synthetic oligonucleotide
  source 1..21: mol_type = other DNA
tctgcagggtagggagagaag

SEQ ID NO: 122 - DNA, 29 bp, synthetic construct
  misc_feature 1..29: note = synthetic oligonucleotide
  source 1..29: mol_type = other DNA
tgtttcagaaaaggaagactcacgttaca

SEQ ID NO: 123 - DNA, 24 bp, synthetic construct
  misc_feature 1..24: note = synthetic oligonucleotide
  source 1..24: mol_type = other DNA
gagaccgatgaagagaaagtcaga

SEQ ID NO: 124 - DNA, 100 bp, synthetic construct
  misc_feature 1..100: note = synthetic oligonucleotide
  source 1..100: mol_type = other DNA
gaccattttaaggttttgcttggttgttttggagggagggtggtgctttgctaatggtgaattactaactcctcaataaagaatattatttgaaataatt

SEQ ID NO: 125 - DNA, 190 bp, synthetic construct
  misc_feature 1..190: note = synthetic oligonucleotide
  source 1..190: mol_type = other DNA
gctgccttttaatccagcgctataattgaggcaagcgtccagcttgacacctcgagataacttcgtataatgtatgctatacgaagttatatgcatggcctccgcgccgggttttggcgcctcccgcgggcgcccccctcctcacggcgagcgctgccacgtcagacgaagggcgcagcgagcgtcctga

SEQ ID NO: 126 - DNA, 171 bp, synthetic construct
  misc_feature 1..171: note = synthetic oligonucleotide
  source 1..171: mol_type = other DNA
attgttttgccaagttctaattccatcagacctcgacctgcagcccctagataacttcgtataatgtatgctatacgaagttatgctagtaactataacggtcctaaggtagcgagctagctgcaaccgaggaaaaaacgtgccatgaggtctctgtatccaagtgtgact

SEQ ID NO: 127 - DNA, 177 bp, synthetic construct
  misc_feature 1..177: note = synthetic oligonucleotide
  source 1..177: mol_type = other DNA
ccagtcagggacacacatgctcacacgcccgcccacccgcacacactacactcgagataacttcgtataatgtatgctatacgaagttatgctagtaactataacggtcctaaggtagcgagctagctgcaaccgaggaaaaaacgtgccatgaggtctctgtatccaagtgtgact

SEQ ID NO: 128 - DNA, 21 bp, synthetic construct
  misc_feature 1..21: note = synthetic oligonucleotide
  source 1..21: mol_type = other DNA
tcctctccagacaagaaagct

SEQ ID NO: 129 - DNA, 30 bp, synthetic construct
  misc_feature 1..30: note = synthetic oligonucleotide
  source 1..30: mol_type = other DNA
tcatagcagctttcaaatcctaaacgttga

SEQ ID NO: 130 - DNA, 20 bp, synthetic construct
  misc_feature 1..20: note = synthetic oligonucleotide
  source 1..20: mol_type = other DNA
tcgtgtgtagctggtgagtt

SEQ ID NO: 131 - DNA, 22 bp, synthetic construct
  misc_feature 1..22: note = synthetic oligonucleotide
  source 1..22: mol_type = other DNA
catgcgatcacaggaggagatc

SEQ ID NO: 132 - DNA, 22 bp, synthetic construct
  misc_feature 1..22: note = synthetic oligonucleotide
  source 1..22: mol_type = other DNA
aattgggcccgaagccagatgc

SEQ ID NO: 133 - DNA, 20 bp, synthetic construct
  misc_feature 1..20: note = synthetic oligonucleotide
  source 1..20: mol_type = other DNA
cggaaggcttctgtgacttc

SEQ ID NO: 134 - DNA, 25 bp, synthetic construct
  misc_feature 1..25: note = synthetic oligonucleotide
  source 1..25: mol_type = other DNA
gtctcccacttctgacataatgaac

SEQ ID NO: 135 - DNA, 27 bp, synthetic construct
  misc_feature 1..27: note = synthetic oligonucleotide
  source 1..27: mol_type = other DNA
cccagtgttaaccctacatctggttcc

SEQ ID NO: 136 - DNA, 20 bp, synthetic construct
  misc_feature 1..20: note = synthetic oligonucleotide
  source 1..20: mol_type = other DNA
tgggaagagactcttggaca

SEQ ID NO: 137 - DNA, 25 bp, synthetic construct
  misc_feature 1..25: note = synthetic oligonucleotide
  source 1..25: mol_type = other DNA
atgagctcctagtacagctaaagtt

SEQ ID NO: 138 - DNA, 26 bp, synthetic construct
  misc_feature 1..26: note = synthetic oligonucleotide
  source 1..26: mol_type = other DNA
atgcatgatcatctatgcgtcagagc

SEQ ID NO: 139 - DNA, 21 bp, synthetic construct
  misc_feature 1..21: note = synthetic oligonucleotide
  source 1..21: mol_type = other DNA
tgcccagatgcagggagttag

(19) United States
(12) Patent Application Publication
(10) Pub. No.: US 2025/0049006 A1
(43) Pub. Date: Feb. 13, 2025

(54) NON-HUMAN ANIMALS COMPRISING HUMANIZED ACE2 AND TMPRSS LOCI

(71) Applicant: c/o Regeneron Pharmaceuticals, Inc., Tarrytown, NY (US)

(72) Inventors: Susannah Brydges, Scarsdale, NY (US); Christos Kyratsous, Irvington, NY (US); Alina Baum, Pleasantville, NY (US)

(21) Appl. No.: 18/721,743

(22) PCT Filed: Dec. 16, 2022
(86) PCT No.: PCT/US22/81833

§ 371 Date: Jun. 19, 2024

Related U.S. Application Data
(60) Provisional application No. 63/291,502, filed on Dec. 20, 2021.

(51) Int. Cl. A01K 67/0278; C12N 9/48; C12N 9/64

(52) U.S. Cl. CPC A01K 67/0278; C12N 9/485; C12N 9/6424; C12Y 304/17023; A01K 2217/052; A01K 2227/105; A01K 2267/0337

(57) ABSTRACT

Non-human animal cells and non-human animals comprising a humanized ACE2 locus and a humanized TMPRSS locus, and methods of using such non-human animal cells and non-human animals are provided. Non-human animal cells or non-human animals comprising a humanized ACE2 locus and a humanized TMPRSS locus express a human ACE2 protein or a chimeric ACE2 protein, fragments of which are from human ACE2; and a human TMPRSS or chimeric TMPRSS protein, fragments of which are from human TMPRSS. Methods are also provided for using such non-human animals comprising a humanized ACE2 locus and a humanized TMPRSS locus to assess in vivo ACE2 activity, e.g., coronavirus infection and/or the treatment or prevention thereof.

### CROSS-REFERENCE TO RELATED APPLICATIONS

This application claims benefit of priority to U.S. Provisional Application No. 63/291,502, filed Dec. 20, 2021, which is incorporated by reference in its entirety.

### FEDERALLY SPONSORED RESEARCH OR DEVELOPMENT

This invention was made with Government support under Agreement HHSO100201700020C, awarded by the U.S. Department of Health and Human Services. The Government has certain rights in the invention.

### REFERENCE TO A SEQUENCE LISTING

The Sequence Listing written in file “10833WO01_ST26.xml” is 459 kilobytes, was created on Dec. 15, 2022, and is hereby incorporated by reference.

### TECHNICAL FIELD

Described herein are (1) non-human animals, e.g., rodents (e.g., rats and mice), and tissues or cells derived therefrom, that comprise (a) a human or humanized Angiotensin-converting enzyme 2 (ACE2) gene, e.g., at an endogenous ACE2 locus and (b) a human or humanized transmembrane serine protease (TMPRSS) gene, e.g., at an endogenous TMPRSS locus, and that express therefrom a recombinant ACE2 protein and a recombinant ACE2 protein, respectively, (2) non-human animal embryonic stem cells and/or genomes comprising the human or humanized ACE2 gene, and the human or humanized TMPRSS gene, (3) methods of making and using the non-human animal embryonic stem cells and/or non-human animal genomes, including methods of making the non-human animals from the non-human animal embryonic stem cells, and (4) methods of making and using the non-human animals that comprise the human or humanized ACE2 gene, and the human or humanized TMPRSS gene. Such non-human animals express a recombinant ACE2 protein comprising at least an extracellular domain of a human ACE2 protein and the human or humanized TMPRSS gene, and thus, may be used to delineate the biological activity of ligand binding to human ACE2 protein, particularly viral ligands requiring a TMPRSS protein for viral infection. Such models may be useful, e.g., for understanding coronavirus infections, e.g., SARS-COV and/or SARS-COV-2 infection, and/or evaluating the efficacy of a vaccine or treatment protocol for same.

### BACKGROUND

Angiotensin-converting enzyme 2 (ACE2) is an enzyme found on the cell surface of cells in the lungs, arteries, heart, kidney, and intestines. A primary function of ACE2 is to cleave the carboxyl-terminal amino acid phenylalanine from angiotensin II and hydrolyze it into the vasodilator angiotensin. ACE2 also serves as the main entry point into cells for some coronaviruses (CoVs).

CoVs can cause diseases in animals. In humans, CoVs are responsible for many of the epidemics of recent years. In 2002, the Severe Acute Respiratory Syndrome Coronavirus (SARS-COV) was responsible for the SARS epidemic, which was contained in July 2003. Since 2004, there have not been any known cases of SARS reported. In 2012, the Middle East Respiratory Syndrome Coronavirus (MERS-COV) emerged as the second coronavirus resulting in a global public health crisis, although an outbreak with a 32.97% fatality rate did not occur until 2014. Cases of MERS continue to be reported, with a 34.4% case-fatality ratio. SARS-COV-2, identified in 2019, is the cause of the disease COVID-19 and infection with SARS-COV-2 reached pandemic levels within months of its first identification. The COVID-19 pandemic is currently ongoing.

Infection with SARS-COV-2 causes symptoms ranging from mild or non-existent to death resulting from severe damage to the lungs and possibly other organs. Due to variability in testing capabilities among different countries, reported fatality rates vary between 1 to 5 percent. If an infected patient is over 50 years old or if they suffer from an underlying condition such as asthma, diabetes, or heart disease, then their chances of surviving COVID-19 decreases. Since infected individuals who are asymptomatic may contribute to the spread of the virus, and since an effective vaccine or treatment protocol is not yet available, the only means by which to reduce transmission of SARS-COV-2 is requiring all individuals to practice social distancing. This had led to a demand for shortcuts in the regulatory approval process for a potential vaccine or treatment, with human trials of lead vaccine or treatment candidates set to proceed at an unprecedented fast pace.

Infection with SARS-COV and SARS-COV-2 is mediated through ACE2 and members of the transmembrane serine protease (TMPRSS) family. Accordingly, a non-human animal model for coronavirus infection, e.g., SARS-COV and/or SARS-COV-2 infection, may be beneficial for the testing of putative vaccines and/or treatments against current and future infection.

### SUMMARY

Disclosed herein are a non-human animal, non-human animal cell, or non-human animal genome comprising (I) a humanized ACE2 gene, e.g., at an endogenous non-human animal ACE2 locus and optionally under the control of an endogenous ACE2 promoter, that encodes a recombinant ACE2 protein and (II) a humanized TMPRSS gene, e.g., at an endogenous TMPRSS locus and optionally under the control of an endogenous TMPRSS promoter, that encodes a recombinant TMPRSS protein, optionally wherein the recombinant ACE2 and/or TMPRSS proteins are expressed by the non-human animal, non-human animal cell, or non-human animal genome.

In some embodiments, a recombinant ACE2 protein comprises in operable linkage: (i) an ACE2 signal sequence of a non-human animal ACE2 protein or an ACE2 signal sequence of a human ACE2 protein, (ii) an extracellular domain of a human ACE2 protein, (iii) a transmembrane domain of a non-human animal ACE2 protein or a transmembrane domain of a human ACE2 protein, and (iv) a cytoplasmic domain of a non-human animal ACE2 protein or a cytoplasmic domain of a human ACE2 protein. In some embodiments, a recombinant ACE2 protein comprises in operable linkage: (i) an ACE2 signal sequence of a non-human animal ACE2 protein, (ii) an extracellular domain of a human ACE2 protein, (iii) a transmembrane domain of a non-human animal ACE2 protein, and (iv) a cytoplasmic domain of a non-human animal ACE2 protein. In some embodiments a recombinant ACE2 protein comprises an amino acid sequence set forth as SEQ ID NO:24.

In some embodiments, a modified endogenous ACE2 locus of a non-human animal comprises a replacement of the nucleotide sequence encoding the extracellular domain of the endogenous ACE2 protein with a nucleotide sequence encoding the extracellular domain of a human ACE2 protein such that the nucleotide sequence encoding the extracellular domain of a human ACE2 protein is operably linked to an endogenous nucleotide sequence encoding (iii) the transmembrane domain of an endogenous non-human animal ACE2 protein, and (iv) the cytoplasmic domain of an endogenous non-human animal ACE2 protein. In some embodiments the modified endogenous ACE2 locus comprises a nucleotide sequence set forth as SEQ ID NO:5 or set forth as SEQ ID NO:22.

In some embodiments, the nucleotide sequence encoding the extracellular domain of a human ACE2 protein comprises part of the coding sequence of coding exon 1, all of the coding sequences of coding exon 2 to coding exon 16, inclusive, and part of the coding sequence of coding exon 17 of a human ACE2 gene.

In some embodiments, a humanized ACE2 gene as described herein comprises a nucleotide sequence encoding an extracellular domain substantially identical (e.g., at least 85%, 90%, 95%, 98%, 99%, or 100% identical in sequence) to the extracellular domain of a human ACE2 protein operably linked to a nucleotide sequence encoding a transmembrane domain substantially (e.g., at least 85%, 90%, 95%, 98%, 99%, or 100% identical in sequence) to the transmembrane domain of a non-human animal ACE2 protein, and a cytoplasmic domain substantially identical (e.g., at least 85%, 90%, 95%, 98%, 99%, or 100% identical in sequence) to the cytoplasmic domain of a non-human animal ACE2 protein. In some embodiments, an endogenous non-human animal ACE2 locus comprises the humanized ACE2 gene, e.g., the endogeneous non-human animal ACE2 locus comprises a replacement of the nucleotide sequence encoding the extracellular domain of the endogenous ACE2 protein with a nucleotide sequence encoding an extracellular domain substantially identical (e.g., at least 85%, 90%, 95%, 98%, 99%, or 100% identical in sequence) to the extracellular domain of a human ACE2 protein such that the nucleotide sequence encoding the extracellular domain substantially identical (e.g., at least 85%, 90%, 95%, 98%, 99%, or 100% identical in sequence) to a human ACE2 protein is operably linked to an endogenous nucleotide sequence encoding the transmembrane and cytoplasmic domains of an endogenous non-human animal ACE2 protein of an endogenous non-human animal ACE2 protein. In some embodiments, the humanized ACE2 gene is under the control of a non-human animal promoter, e.g., an endogenous non-human animal promoter, e.g., at an endogenous non-human animal ACE2 locus. In some embodiments, the nucleotide sequence encoding an extracellular domain substantially identical (e.g., at least 85%, 90%, 95%, 98%, 99%, or 100% identical in sequence) to the extracellular domain of a human ACE2 protein comprises part of the coding sequence of coding exon 1, all of the coding sequences of coding exon 2 to coding exon 16, inclusive, and part of the coding sequence of coding exon 17 of a human ACE2 gene, and degenerate variants thereof.

In some embodiments, a recombinant ACE2 protein is encoded by a humanized ACE2 gene and expressed by the non-human animal, non-human animal cell, or non-human animal genome. In some embodiments, a recombinant ACE2 protein comprises in operable linkage: (i) an ACE2 signal sequence of a non-human animal ACE2 protein or an ACE2 signal sequence of a human ACE2 protein, (ii) an extracellular domain substantially identical (e.g., at least 85%, 90%, 95%, 98%, 99%, or 100% identical in sequence) to the extracellular domain of a human ACE2 protein, (iii) a transmembrane domain of a non-human animal ACE2 protein or a transmembrane domain of a human ACE2 protein, and (iv) a cytoplasmic domain of a non-human animal ACE2 protein or a cytoplasmic domain of a human ACE2 protein. In some embodiments, a recombinant ACE2 protein comprises in operable linkage: (i) an ACE2 signal sequence of a non-human animal ACE2 protein, (ii) an extracellular domain substantially identical (e.g., at least 85%, 90%, 95%, 98%, 99%, or 100% identical in sequence) to an extracellular domain of a human ACE2 protein, (iii) a transmembrane domain of a non-human animal ACE2 protein, and (iv) a cytoplasmic domain of a non-human animal ACE2 protein. In some mature recombinant ACE2 protein embodiments, a recombinant ACE2 protein comprises only a mature sequence, e.g., (i) an extracellular domain substantially identical (e.g., at least 85%, 90%, 95%, 98%, 99%, or 100% identical in sequence) to the extracellular domain of a human ACE2 protein, (ii) a transmembrane domain of a non-human animal ACE2 protein or a transmembrane domain of a human ACE2 protein, and (iii) a cytoplasmic domain of a non-human animal ACE2 protein or a cytoplasmic domain of a human ACE2 protein. In some mature recombinant ACE2 protein embodiments, a recombinant ACE2 protein comprises (i) an extracellular domain substantially identical (e.g., at least 85%, 90%, 95%, 98%, 99%, or 100% identical in sequence) to an extracellular domain of a human ACE2 protein, (ii) a transmembrane domain of a non-human animal ACE2 protein, and (iiii) a cytoplasmic domain of a non-human animal ACE2 protein. In some embodiments a recombinant ACE2 protein comprises an amino acid sequence set forth as SEQ ID NO:24.

Also described herein are nucleotide molecules, e.g., targeting vectors, which may be useful in making non-human animals comprising a modified endogenous ACE2 locus that expresses a human or humanized ACE2 protein. In some embodiments, a targeting vector comprises an insert nucleotide that (a) comprises a nucleotide sequence that encodes at least an extracellular domain of a human ACE2 protein and (b) is flanked by 5′ and 3′ homology arms that undergo homologous recombination with an endogenous ACE2 locus of a non-human animal, wherein following the homologous recombination of the endogenous ACE2 locus with the 5′ and 3′ homology arms, the genetically modified endogenous ACE2 locus of the non-human animal encodes, under the control of an endogenous ACE2 promoter, a recombinant ACE2 protein that comprises in operable linkage: (i) an ACE2 signal sequence of a non-human animal ACE2 protein or an ACE2 signal sequence of a human ACE2 protein (ii) the extracellular domain of a human ACE2 protein, (iii) a transmembrane domain of a non-human animal ACE2 protein or a transmembrane domain of a human ACE2 protein, and (iv) a cytoplasmic domain of a non-human animal ACE2 protein or a cytoplasmic domain of a human ACE2 protein. In some embodiments, following the homologous recombination of the endogenous ACE2 locus with the 5′ and 3′ homology arms, the genetically modified endogenous ACE2 locus of the non-human animal encodes, under the control of an endogenous ACE2 promoter, a recombinant ACE2 protein that comprises in operable linkage: (i) an ACE2 signal sequence of an endogenous non-human animal ACE2 protein, (ii) an extracellular domain of a human ACE2 protein, (iii) a transmembrane domain of an endogenous non-human animal ACE2 protein, and (iv) a cytoplasmic domain of an endogenous non-human animal ACE2 protein. In some embodiments, following homologous recombination, the nucleotide sequence that encodes at least an extracellular domain of a human ACE2 protein replaces an orthologous sequence at the endogenous ACE2 locus. In some targeting vector embodiments, the nucleotide sequence that encodes at least an extracellular domain of a human ACE2 protein comprises part of the coding sequence of coding exon 1 and all of the coding sequences of coding exon 2 to coding exon 16, inclusive, and part of the coding sequence of exon 17 of a human ACE2 gene.

In some embodiments, a targeting vector or nucleic acid comprises a nucleotide sequence set forth as SEQ ID NO:5, SEQ ID NO:22, or SEQ ID NO:25.

In some embodiments, an insert nucleic acid further comprises a second nucleic acid sequence comprising a sequence encoding a selectable marker, preferably wherein the sequence encoding a selectable marker is operably linked to a promoter. In some embodiments, the insert nucleotide comprises site-specific recombination sites flanking the second nucleic acid sequence. In some embodiments, the second nucleic acid sequence further comprises a sequence encoding a site-specific recombinase, preferably wherein the sequence encoding the selectable marker is operably linked to a promoter. In some embodiments, a targeting vector comprises from 5′ to 3′ a nucleotide sequence comprising the nucleotide sequences set forth as SEQ ID NO:18, SEQ ID NO: 19, SEQ ID NO:20, and SEQ ID NO:21.

Also described herein are nucleic acids. A nucleic acid embodiment herein may comprises a sequence set forth as SEQ ID NO:5. In some embodiments, a nucleic acid comprises a sequence set forth as SEQ ID NO:22. In some embodiments, a nucleic acid comprises a sequence set forth as SEQ ID NO:25.

Also described herein are a non-human animal, non-human animal cell, or non-human animal genome comprising a genetically modified endogenous ACE2 locus. In some embodiments, a non-human animal, non-human animal cell, or non-human animal genome is modified at an endogenous ACE2 locus with a targeting vector as described herein or to comprise a nucleic acid described herein.

In some embodiments, a non-human animal cell as described herein comprises a modified ACE2 locus that expresses a human or humanized ACE2 protein, wherein the amino acid sequence of the extracellular domain of a human ACE2 protein is set forth in SEQ ID NO: 27. In some embodiments, a non-human animal, non-human animal cell, or non-human animal genome is heterozygous for the genetically modified endogenous ACE2 locus. In some embodiments, a non-human animal, non-human animal cell, or non-human animal genome is homozygous for the genetically modified endogenous ACE2 locus. In some embodiments, a non-human animal is a mammal, a non-human animal cell is a mammalian cell, or a non-human animal genome is a mammalian genome. In some embodiments, a non-human animal is a rodent, a non-human animal cell is a rodent cell, or a non-human animal genome is a rodent genome. In some embodiments, a non-human animal is a rat or mouse, tanon-human animal cell is a rat cell or a mouse cell, or a non-human animal genome is a rat genome or a mouse genome. In some embodiments, a non-human animal is a mouse, a non-human animal cell is a mouse cell, or a non-human animal genome is a mouse genome. In some animal, cell and/or genome embodiments, the animal, cell and/or genome comprises a sequence encoding a recombinant ACE2 protein and the animal, cell and/or genome expresses the recombinant ACE2 protein.

In some embodiments, a non-human animal comprising a genetically modified endogenous ACE2 locus as described herein expresses a recombinant ACE2 protein in an organ selected from the group consisting of colon, duodenum, kidney, heart, liver, lung, trachea, and any combination thereof. In some embodiments, the expression pattern of a recombinant ACE2 protein in a genetically modified non-human animal as described herein follows the expression pattern of a non-human animal ACE2 protein in a control non-human animal comprising a wildtype endogenous ACE2 locus.

In some embodiments, the recombinant ACE2 protein is expressed on epithelial cells. Accordingly, also described herein is a non-human animal cell expressing a recombinant ACE2 protein, optionally wherein the non-human animal cell (e.g., rat cell or mouse cell) is a somatic cell, optionally wherein the somatic cell is an epithelial cell. Non-limiting examples of epithetical cells that may express a recombinant ACE2 protein as described herein include respiratory and/or gastrointestinal epithelial cells, e.g., an alveolar cell of the lung, an esophagus upper and stratified epithelial cell, an absorptive enterocyte from the ileum or colon, etc. In some embodiments, a non-human animal cell as described herein expresses the recombinant ACE2 protein in the epithelium of small intestine villi, surface epithelium of the large intestine (colon), the epithelium of large to small bronchioles and bronchi of the lung, respiratory epithelium of the trachea, proximal tubular epithelium of the kidney, respiratory epithelium of th nasal cavity, and/or the stratum granulosum and/or stratum spinosum of oral mucosa/tongue in the oral cavity.

In some embodiments, a humanized TMPRSS gene comprises a nucleotide sequence encoding an extracellular domain substantially identical (e.g., at least 85%, 90%, 95%, 98%, 99%, or 100% identical in sequence) to the extracellular domain of a human TMPRSS protein operably linked to a non-human animal nucleotide sequence encoding a transmembrane domain substantially (e.g., at least 85%, 90%, 95%, 98%, 99%, or 100% identical in sequence) to the transmembrane domain of a non-human animal TMPRSS protein, and a cytoplasmic domain substantially identical (e.g., at least 85%, 90%, 95%, 98%, 99%, or 100% identical in sequence) to the cytoplasmic domain of a non-human animal TMPRSS protein, wherein the human TMPRSS and non-human animal TMPRSS proteins are orthologous. In some embodiments, a non-human animal, non-human animal cell, or non-human animal genome comprises am endogenous non-human animal TMPRSS locus modified to comprise the humanized TMPRSS gene, e.g., the endogeneous non-human animal TMPRSS locus comprises a replacement of the nucleotide sequence encoding the extracellular domain of the endogenous TMPRSS protein with a nucleotide sequence encoding an extracellular domain substantially identical (e.g., at least 85%, 90%, 95%, 98%, 99%, or 100% identical in sequence) to the extracellular domain of a human TMPRSS protein such that the nucleotide sequence encoding the extracellular domain is an extracellular domain substantially identical (e.g., at least 85%, 90%, 95%, 98%, 99%, or 100% identical in sequence) to the extracellular domain of a human TMPRSS protein, and is operably linked to an endogenous nucleotide sequence encoding the transmembrane and cytoplasmic domains of the endogenous non-human animal TMPRSS protein at the endogenous TMPRSS locus, wherein the human TMPRSS and non-human animal TMPRSS proteins are orthologous. In some embodiments, the humanized TMPRSS gene is under the control of a non-human animal promoter, e.g., an endogenous non-human animal promoter, e.g., at an endogenous non-human animal TMPRSS locus.

In some embodiments, a humanized TMPRSS gene comprises a humanized TMPRSS2 gene, which humanized TMPRSS2 gene comprises a nucleotide sequence of a human TMPRSS2 gene and a nucleotide sequence of the non-human animal TMPRSS2 gene. In some embodiments, a humanized TMPRSS2 gene comprises a nucleotide sequence of a human TMPRSS2 gene and a nucleotide sequence of the non-human animal TMPRSS2 gene, wherein the nucleotide sequence of the human TMPRSS2 gene encodes an extracellular domain substantially identical (e.g., at least 85%, 90%, 95%, 98%, 99% or 100% identical in sequence) with the extracellular domain of the human TMPRSS2 protein encoded by the human TMPRSS2 gene. In specific embodiments, the nucleotide sequence of a human TMPRSS2 gene is a contiguous genomic sequence of a human TMPRSS2 gene containing coding exon 4 through the stop codon in coding exon 13 of the human TMPRSS2 gene. In particular embodiments, the contiguous genomic sequence of a human TMPRSS2 gene further contains the 3′ UTR of the human TMPRSS2 gene. In some embodiments, the nucleotide sequence of the non-human animal TMPRSS2 gene included in a humanized TMPRSS2 gene encodes a cytoplasmic and transmembrane portion that is substantially identical (e.g., at least 85%, 90%, 95%, 98%, 99% or 100% identical) with the cytoplasmic and transmembrane portion of the non-human animal TMPRSS2 protein encoded by the non-human animal TMPRSS2 gene. In some embodiments, a humanized TMPRSS2 gene comprises coding exons 1-2 of the non-human animal TMPRSS2 gene, and coding exon 4 through coding exon 13 of a human TMPRSS2 gene, wherein the humanized TMPRSS2 gene encodes a humanized TMPRSS2 protein comprising an extracellular domain that is substantially identical with the extracellular domain of the human TMPRSS2 protein encoded by the human TMPRSS2 gene and a transmembrane and cytoplasmic portion that is substantially identical with the cytoplasmic and transmembrane portion of the non-human animal TMPRSS2 protein encoded by the non-human animal TMPRSS2 gene. In some embodiments, the humanized TMPRSS2 gene contains an exon 3 that in some embodiments is coding exon 3 of a human TMPRSS2 gene, and in other embodiments is coding exon 3 of an endogenous rodent TMPRSS2 gene. In some embodiments, the humanized TMPRSS2 gene comprises an exon 3 that includes a 5′ portion of coding exon 3 of the non-human animal TMPRSS2 gene and a 3′ portion of coding exon 3 of a human TMPRSS2 gene. In some embodiments, a non-human animal, non-human animal cell, or non-human animal genome comprises an endogenous non-human animal TMPRSS2 locus modified to comprise the humanized TMPRSS2 gene, e.g., the endogeneous non-human animal TMPRSS2 locus comprises a replacement of the nucleotide sequence encoding the extracellular domain of the endogenous TMPRSS2 protein with a nucleotide sequence encoding an extracellular domain substantially identical (e.g., at least 85%, 90%, 95%, 98%, 99%, or 100% identical in sequence) to the extracellular domain of a human TMPRSS2 protein such that the nucleotide sequence encoding the extracellular domain substantially identical (e.g., at least 85%, 90%, 95%, 98%, 99%, or 100% identical in sequence) to the extracellular domain of a human TMPRSS2 protein is operably linked to an endogenous nucleotide sequence encoding the transmembrane and cytoplasmic domains of the endogenous non-human animal TMPRSS2 protein at the endogenous TMPRSS2 locus. In some embodiments, the humanized TMPRSS2 gene is under the control of a non-human animal TMPRSS2 promoter, e.g., an endogenous non-human animal TMPRSS2 promoter, e.g., at an endogenous non-human animal TMPRSS2 locus.

In some embodiments, a humanized TMPRSS gene comprises a humanized TMPRSS4 gene comprising a nucleotide sequence of a human TMPRSS4 gene and a nucleotide sequence of the non-human animal TMPRSS4 gene. In some embodiments, a humanized TMPRSS4 gene comprises a nucleotide sequence of a human TMPRSS4 gene and a nucleotide sequence of a non-human animal TMPRSS4 gene, wherein the nucleotide sequence of a human TMPRSS4 gene encodes an extracellular domain substantially identical with the extracellular domain of the human TMPRSS4 protein encoded by the human TMPRSS4 gene. In specific embodiments, the nucleotide sequence of a human TMPRSS4 gene is a contiguous genomic sequence containing coding exon 4 through the stop codon in coding exon 13 of a human TMPRSS4 gene. In some embodiments, the nucleotide sequence of an on-human animal TMPRSS4 gene included in a humanized TMPRSS4 gene encodes a cytoplasmic and transmembrane portion that is substantially identical with the cytoplasmic and transmembrane portion of the rodent TMPRSS4 protein encoded by the non-human animal TMPRSS4 gene. In particular embodiments, a humanized TMPRSS4 gene comprises coding exon 1 through coding exon 3 of a non-human animal TMPRSS4 gene, and coding exon 4 through the stop codon in coding exon 13 of a human TMPRSS4 gene. In some embodiments, a non-human animal, non-human animal cell, or non-human animal genome comprises an endogenous non-human animal TMPRSS4 locus modified to comprise the humanized TMPRSS4 gene, e.g., the endogeneous non-human animal TMPRSS4 locus comprises a replacement of the nucleotide sequence encoding the extracellular domain of the endogenous TMPRSS4 protein with a nucleotide sequence encoding an extracellular domain substantially identical (e.g., at least 85%, 90%, 95%, 98%, 99%, or 100% identical in sequence) to the extracellular domain of a human TMPRSS4 protein such that the nucleotide sequence encoding the extracellular domain substantially identical (e.g., at least 85%, 90%, 95%, 98%, 99%, or 100% identical in sequence) to the extracellular domain of human TMPRSS4 protein is operably linked to an endogenous nucleotide sequence encoding the transmembrane and cytoplasmic domains of the endogenous non-human animal TMPRSS4 protein at the endogenous TMPRSS4 locus. In some embodiments, the humanized TMPRSS4 gene is under the control of a non-human animal TMPRSS4 promoter, e.g., an endogenous non-human animal TMPRSS4 promoter, e.g., at an endogenous non-human animal TMPRSS4 locus.

In some embodiments, a humanized TMPRSS gene comprises a humanized TMPRSS11D gene comprising a nucleotide sequence of a human TMPRSS11D gene and a nucleotide sequence of a non-human animal TMPRSS11D gene. In some embodiments, a non-human animal, non-human animal cell, or non-human animal genome comprises a humanized TMPRSS11D gene comprising a nucleotide sequence of an endogenous rodent TMPRSS11D gene and a nucleotide sequence of a human TMPRSS11D gene, wherein the nucleotide sequence of the human TMPRSS11D gene encodes an extracellular domain substantially identical with the extracellular domain of the human TMPRSS11D protein encoded by the human TMPRSS11D gene. In specific embodiments, the nucleotide sequence of a human TMPRSS11D gene is a contiguous genomic sequence containing coding exon 3 through the stop codon in coding exon 10 of a human TMPRSS11D gene. In particular embodiments, the contiguous genomic sequence of a human TMPRSS11D gene further contains the 3′ UTR of the human TMPRSS11D gene. In some embodiments, the nucleotide sequence of a non-human animal TMPRSS11D gene included in a humanized TMPRSS11D gene encodes a cytoplasmic and transmembrane portion that is substantially identical with the cytoplasmic and transmembrane portion of the non-human animal TMPRSS11D protein encoded by the non-human animal TMPRSS11D gene. In dome embodiments, a humanized TMPRSS11D gene contains coding exons 1-2 of an endogenous non-human animal TMPRSS11D gene, and coding exon 3 through coding exon 13 of a human TMPRSS11D gene. In some embodiments, a non-human animal, non-human animal cell, or non-human animal genome comprises an endogenous non-human animal TMPRSS11D locus modified to comprise the humanized TMPRSS11D gene, e.g., the endogeneous non-human animal TMPRSS11D locus comprises a replacement of the nucleotide sequence encoding the extracellular domain of the endogenous TMPRSS11D protein with a nucleotide sequence encoding an extracellular domain substantially identical (e.g., at least 85%, 90%, 95%, 98%, 99%, or 100% identical in sequence) to the extracellular domain of a human TMPRSS11D protein such that the nucleotide sequence encoding the extracellular domain substantially identical (e.g., at least 85%, 90%, 95%, 98%, 99%, or 100% identical in sequence) to the extracellular domain of human TMPRSS11D protein is operably linked to an endogenous nucleotide sequence encoding the transmembrane and cytoplasmic domains of the endogenous non-human animal TMPRSS11D protein at the endogenous TMPRSS11D locus. In some embodiments, the humanized TMPRSS11D gene is under the control of a non-human animal TMPRSS11D promoter, e.g., an endogenous non-human animal TMPRSS11D promoter, e.g., at an endogenous non-human animal TMPRSS11D locus.

In some embodiments, a recombinant TMPRSS protein described herein is encoded by a humanized TMPRSS gene as described herein. In some embodiments, a recombinant TMPRSS protein comprises in operable linkage: (i) a TMPRSS signal sequence of a non-human animal TMPRSS protein or a TMPRSS signal sequence of a human TMPRSS protein, (ii) an extracellular domain substantially identical (e.g., at least 85%, 90%, 95%, 98%, 99%, or 100% identical in sequence) to the extracellular domain of a human TMPRSS protein, (iii) a transmembrane domain of a non-human animal TMPRSS protein or a transmembrane domain of a human TMPRSS protein, and (iv) a cytoplasmic domain of a non-human animal TMPRSS protein or a cytoplasmic domain of a human TMPRSS protein, wherein the non-human TMPRSS protein and human TMPRSS protein are orthologous. In some embodiments, a recombinant TMPRSS protein comprises in operable linkage: (i) a TMPRSS signal sequence of a non-human animal TMPRSS protein, (ii) an extracellular domain substantially identical (e.g., at least 85%, 90%, 95%, 98%, 99%, or 100% identical in sequence) to an extracellular domain of a human TMPRSS protein, (iii) a transmembrane domain of a non-human animal TMPRSS protein, and (iv) a cytoplasmic domain of a non-human animal TMPRSS protein, wherein the non-human TMPRSS protein and human TMPRSS protein are orthologous.

In some embodiments, the orthologous non-human animal and human TMPRSS proteins are a non-human animal TMPRSS2 protein and a human TMPRSS2 protein, respectively. In some embodiments, a humanized TMPRSS2 gene encodes a recombinant TMPRSS2 protein comprising an extracellular domain substantially identical (e.g., at least 85%, 90%, 95%, 98%, 99% or 100% identical in sequence) with the extracellular domain of the human TMPRSS2 protein encoded by the human TMPRSS2 gene used in humanization. The recombinant TMPRSS2 protein comprises, in some embodiments, an amino acid sequence at least 85% identical (e.g., at least 90%, 95%, 98%, 99% or 100% identical) with the amino acid sequence as set forth in SEQ ID NO:71. In some embodiments, a recombinant TMPRSS2 protein comprises an extracellular domain substantially identical (e.g., at least 85%, 90%, 95%, 98%, 99% or 100% identical) with the amino acid sequence composed of residues W106 to G492 or the C-terminal 387 amino acids of a human TMPRSS2 protein as set forth in, e.g., SEQ ID NO:71. In some embodiments, the humanized TMPRSS2 gene encodes a humanized TMPRSS2 protein that further contains a cytoplasmic and transmembrane portion that is substantially identical (e.g., at least 85%, 90%, 95%, 98%, 99% or 100% identical) with the cytoplasmic and transmembrane portion of the non-human animal TMPRSS2 protein encoded by the non-human animal TMPRSS2 gene being humanized. An exemplary endogenous non-human animal TMPRSS2 protein is set forth in SEQ ID NO:69.

In some embodiments, the orthologous non-human animal and human TMPRSS proteins are a non-human animal TMPRSS4 protein and a human TMPRSS4 protein, respectively. In some embodiments, a humanized TMPRSS4 gene encodes a recombinant TMPRSS4 protein comprising an extracellular domain substantially identical (e.g., at least 85%, 90%, 95%, 98%, 99% or 100% identical in sequence) with the extracellular domain of the human TMPRSS4 protein encoded by the human TMPRSS4 gene used in humanization. The recombinant TMPRSS4 protein comprises, in some embodiments, an amino acid sequence at least 85% identical (e.g., at least 90%, 95%, 98%, 99% or 100% identical) with the amino acid sequence as set forth in SEQ ID NO:78. In some embodiments, a recombinant TMPRSS4 protein contains an extracellular domain substantially identical (e.g., at least 85%, 90%, 95%, 98%, 99% or 100% identical) with the amino acid sequence composed of residues K54 to L437 or the C-terminal 384 amino acids of a human TMPRSS4 protein as set forth in, e.g., SEQ ID NO:78. In some embodiments, the humanized TMPRSS4 gene encodes a recombinant TMPRSS4 protein that further comprises a cytoplasmic and transmembrane portion that is substantially identical (e.g., at least 85%, 90%, 95%, 98%, 99% or 100% identical) with the cytoplasmic and transmembrane portion of the non-human animal TMPRSS4 protein encoded by the non-human animal TMPRSS4 gene being humanized. An exemplary non-human animal, e.g., rodent, TMPRSS4 protein is set forth in SEQ ID NO:76.

In some embodiments, the orthologous non-human animal and human TMPRSS proteins are a non-human animal TMPRSS11 protein and a human TMPRSS11 protein, respectively. In some embodiments, the orthologous non-human animal and human TMPRSS proteins are a non-human animal TMPRSS11A protein and a human TMPRSS11A protein, respectively. In some embodiments, the orthologous non-human animal and human TMPRSS proteins are a non-human animal TMPRSS11D protein and a human TMPRSS11D protein, respectively. In some embodiments, the humanized TMPRSS11D gene encodes a recombinant TMPRSS11D protein comprising an extracellular domain substantially identical (e.g., at least 85%, 90%, 95%, 98%, 99% or 100% identical in sequence) with the extracellular domain of the human TMPRSS11D protein encoded by the human TMPRSS11D gene used in humanization. The recombinant TMPRSS11D protein contains, in some embodiments, an amino acid sequence at least 85% identical (e.g., at least 90%, 95%, 98%, 99% or 100% identical) with the amino acid sequence as set forth in SEQ ID NO:85. In some embodiments, a recombinant TMPRSS11D protein comprises an extracellular domain substantially identical (e.g., at least 85%, 90%, 95%, 98%, 99% or 100% identical) with the amino acid sequence composed of residues A42-1418 or the C-terminal 377 amino acids of a human TMPRSS11D protein as set forth in, e.g., SEQ ID NO:85. In some embodiments, the humanized TMPRSS11D gene encodes a recombinant TMPRSS11D protein further comprising a cytoplasmic and transmembrane portion that is substantially identical (e.g., at least 85%, 90%, 95%, 98%, 99% or 100% identical) with the cytoplasmic and transmembrane portion of the non-human animal TMPRSS11D protein encoded by the endogenous rodent TMPRSS11D gene being humanized. An exemplary non-human animal, e.g., rodent, TMPRSS11D protein is set forth in SEQ ID NO:83.

Also provided herein are methods of making a non-human animal, non-human animal cell, or non-human animal genome comprising humanized endogenous ACE2 and TMPRSS loci. In some embodiments, a method comprises (A) generating a genetically modified non-human embryonic stem (ES) cell comprising (I) humanized ACE2 gene encoding a recombinant ACE2 protein that comprises in operable linkage: (a) an ACE2 signal sequence of a non-human animal ACE2 protein or an ACE2 signal sequence of a human ACE2 protein, (b) an extracellular domain substantially identical to a human ACE2 protein, (c) a transmembrane domain of a non-human animal ACE2 protein or a transmembrane domain of a human ACE2 protein, and (d) a cytoplasmic domain of a non-human animal ACE2 protein or a cytoplasmic domain of a human ACE2 protein, and (II) one or more distinct and humanized TMPRSS genes, each encoding a distinct recombinant TMPRSS protein that comprises in operable likage: (a) a TMPRSS signal sequence of a non-human animal TMPRSS protein or TMPRSS signal sequence of a human TMPRSS protein, (b) an extracellular domain substantially identical to the extracellular domain of the human TMPRSS protein, and (c) a transmembrane domain of the non-human animal TMPRSS protein or a transmembrane domain of the human TMPRSS protein, and (d) a cytoplasmic domain of the non-human TMPRSS protein or a cytoplasmic domain of the human TMPRSS protein, wherein the non-human animal TMPRSS protein and the human TMPRSS protein are orthologous, (B) introducing the modified non-human animal ES cell into a host embryo of non-human animal to form a donor cell-non-human animal embryo complex; and (C) gestating the donor cell-non-human animal embryo in a surrogate non-human animal mother, wherein the surrogate non-human animal mother produces rodent progeny that express the humanized ACE2 and TMPRSS proteins. In some embodiments, generating the genetically modified non-human animal ES cell comprises: (i) obtaining a non-human ES cell that comprises the humanized ACE2 gene, and (ii) modifying the genome of the obtained non-human ES cell that comprises the humanized ACE2 gene to further comprise the one or more distinct humanized TMPRSS genes, wherein modifying the genome comprises replacing an endogenous nucleotide sequence encoding an extracellular domain of an endogenous TMPRSS protein with a nucleotide sequence encoding a transmembrane domain substantially identical (e.g., at least 85%, 90%, 95%, 98%, 99%, or 100% identical in sequence) to the extracellular domain of a human TMPRSS protein that is orthologous to the endogenous TMPRSS protein. In some embodiments, generating the genetically modified non-human animal ES cell comprises: (i) obtaining a non-human ES cell that comprises the one or more distinct humanized TMPRSS genes, and (ii) modifying the genome of the obtained non-human ES cell that comprises the one or more distinct humanized TMPRSS genes to further comprise the humanized ACE2 gene.

In some embodiments, a non-human animal cell as described herein comprises a modified ACE2 locus encoding a recombinant ACE2 protein and a modified TMPRSS locus encoding a recombinant TMPRSS protein as described herein, but does not express the recombinant ACE2 protein and/or does not express the recombinant TMPRSS protein. In some embodiments, such a cell is a non-human animal embryonic stem (ES) cell, pluripotent cell, or a germ cell. Methods for making such cells are also described, e.g., in methods of making the non-human animals.

Also described herein are a non-human animal tissue or a composition comprising the non-human animal cells described herein. In some composition embodiments, the composition further comprises a spike protein of a coronavirus, wherein the spike protein binds the recombinant ACE2 protein and/or a therapeutic agent that inhibits, prevents, or reduces the likelihood of binding of an ACE2 ligand to the recombinant ACE2 protein, optionally wherein the ACE2 ligand comprises a spike protein of a coronavirus. In some embodiments, a therapeutic agent may be an antigen-binding protein that binds the spike protein of a coronavirus.

Also described herein is use of a non-human animal as a model of coronavirus infection, wherein the non-human animal as described herein is contacted, e.g., infected with, a coronavirus comprising a spike protein that binds to a human ACE2 protein. Also provided is a method of screening drug candidates that target a ligand of a human ACE2 protein, comprising: a) introducing into a genetically modified non-human animal as described herein a ligand of a human ACE2 protein; b) contacting the non-human animal with a drug candidate of interest, wherein the drug candidate is directed against the ligand of a human ACE2 protein; and c) determining whether the drug candidate is efficacious in preventing, reducing or eliminating binding of the ligand of a human ACE2 protein to the recombinant ACE2 protein. In some embodiments, the introducing comprises infecting the non-human animal with a coronavirus, wherein the coronavirus comprises a spike protein, and wherein the spike protein comprises the ligand of a human ACE2 protein, e.g., wherein the coronavirus is SARS-COV-2.

In some embodiments, preventing, reducing or eliminating binding of SARS-CoV-2 to the recombinant ACE2 protein results in preventing, reducing, or eliminating one or more COVID-19 symptoms in the non-human animal.

Also disclosed is an ACE2-null mouse comprising an endogenous ACE2 locus comprising a deletion of a sequence encoding ACE2. In some embodiments, an ACE2-null mouse comprises an endogenous ACE2 locus as depicted in FIG. 2B, e.g., comprising a deletion of 46,894 bp of ACE2 on the X chromosome, encompassing 27 bp of ACE2 5′ untranslated region (UTR) and the entire coding sequence with intervening introns, except for 65 bp at the 3′ end of the last coding exon, optionally wherein the endogenous locus also comprises a C to T point mutation in the ACE2 3′ UTR. In some embodiments, an endogenous ACE2 locus of an ACE2-null mouse comprises a sequence set forth as SEQ ID NO: 55. In some ACE2-null embodiments, a mouse as described herein is heterozygous for the endogenous locus comprising a deletion of a sequence encoding ACE2, e.g., for the endogenous locus depicted in FIG. 2B, e.g., for an endogenous lcous comprising a deletion of 46,894 bp of ACE2 on the X chromosome, encompassing 27 bp of ACE2 5′ untranslated region (UTR) and the entire coding sequence with intervening introns, except for 65 bp at the 3′ end of the last coding exon, optionally wherein the endogenous locus also comprises a C to T point mutation in the ACE2 3′ UTR. In some embodiments, an ACE2-null mouse as described herein is homozygous for the endogenous locus comprising a deletion of a sequence encoding ACE2, e.g., for the endogenous locus depicted in FIG. 2B, e.g., for an endogenous lcous comprising a deletion of 46,894 bp of ACE2 on the X chromosome, encompassing 27 bp of A (E2 5′ untranslated region (UTR) and the entire coding sequence with intervening introns, except for 65 bp at the 3′ end of the last coding exon, optionally wherein the endogenous locus also comprises a C to T point mutation in the ACE2 3′ UTR. Also provided are nucleic acids for making an ACE2-null mouse, e.g., a nucleic acid comprising a sequence set forth as SEQ ID NO:51, SEQ ID NO:52, SEQ ID NO:53, and/or SEQ ID NO:54.

### DESCRIPTION OF THE DRAWINGS

FIG. 1A shows schematics (not-to-scale) of the human and mouse ACE2 loci. The untranslated and coding exons are represented by rectangles, coding sequences are indicated by the filled boxes, the untranslated regions (UTRs) are indicated by the unfilled boxes, and various accession numbers for non-limiting examples of ACE2 genes, along with the chromosomal locations, are indicated at the top of the figure. The asterisks indicate the locations of (A) the upstream (7878hTU) and downstream (7878hTD) primers for the gain-of-allele assay or (B) the upstream (7878mTU) and downstream (7878mTD) primers for the loss-of-allele assay. The fragment to be inserted into the mouse ACE2 locus for humanization is shown underneath the human ACE2 allele, and the fragment to be deleted from the mouse ACE2 locus is shown underneath the mouse ACE2 allele.

FIG. 1B shows a schematic (not-to-scale) of a humanized ACE2 allele (7878 allele) containing a neomycin resistance self-deleting cassette, which is depicted as the unfilled arrow. The mouse exonic sequences are indicated by the gray filled boxes, the human exonic sequences are indicated by the black filled boxes, and the mouse 5′ and 3′ untranslated regions (UTRs) are indicated by the white unfilled boxes. The sequences between the different mouse/human, human/cassette, cassette/human, and human/mouse junctions are indicated by the lines labeled “A,” “B,” “C,” and “D,” respectively. The sequences of these junctions are provided below in Table 1.

TABLE 1

| | |
|---|---|
| A. 5′ mouse/5′ human | AAGATGTCCA GCTCCTCCTG GCTCCTTCTC |
| | AGCCTTGTTG CTGTTACTAC TGCTCAGTCC/ |
| | ACCATTGAGG AACAGGCCAA GACATTTTTG |
| | GACAAGTTTA ACCACGAAGC CGAAGACCTG |
| | (SEQ ID NO: 18) |
| B. Human//XhoI//(loxP) | CATTTATACA TTTCCACACT TACAACTCAA |
| Cassette | TTTTCCAATG GAGCTGTTGA TGAACCTAAT |
| | //CTCGAG// |
| | (ATAACTTCGTATAATGTATGCTATACGAAGTTAT) |
| | ATGCATGCCA GTAGCAGCAC CCACGTCCAC |
| | CTTCTGTCTA GTAATGTCCA ACACCTCCCT |
| | (SEQ ID NO: 19) |
| C. Cassette | CATTCTCAGT ATTGTTTTGC CAAGTTCTAA |
| (loxP)/ICEUI//NheI//human | TTCCATCAGA CCTCGACCTG CAGCCCCTAG |
| | (ATAACTTCGTATAATGTATG CTATACGAAGTTAT) |
| | GCTAGG TAACTATAACGGTCCTAAGGTAGCGA |
| | /GCTAGC CTAGGTTGCA AGGCATGAAA |
| | GATGCATAAT TGTCAAAGAC TTATATCTTT |
| | AATTAGACCT |
| | (SEQ ID NO: 20) |
| D. 3′ human/3′ mouse | AGCCTAGAGT TTCTGGGGAT ACAGCCAACA |
| | CTTGGACCTC CTAACCAGCC CCCTGTTTCC/ |
| | ATATGGCTGA TTATTTTTGG TGTTGTGATG |
| | GCACTGGTAG TGGTTGGCAT CATCATCCTG |
| | (SEQ ID NO: 21) |

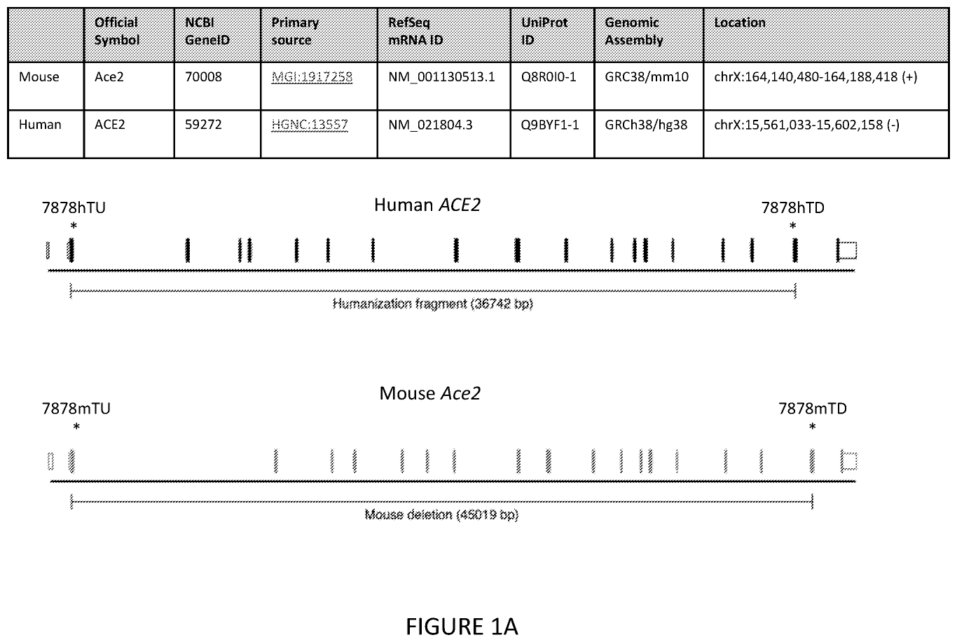

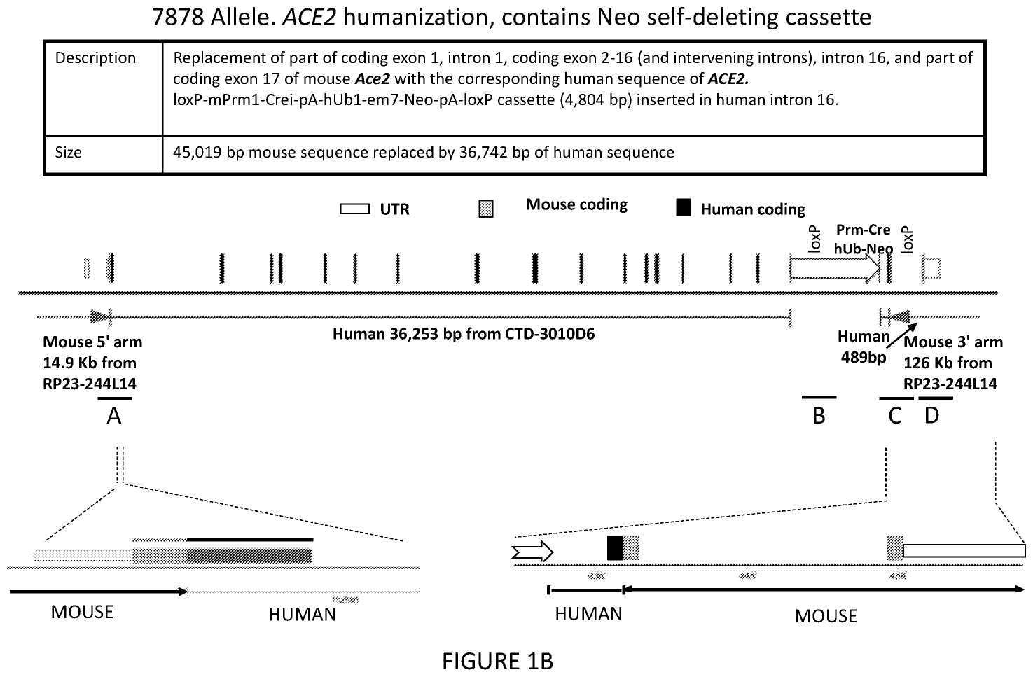

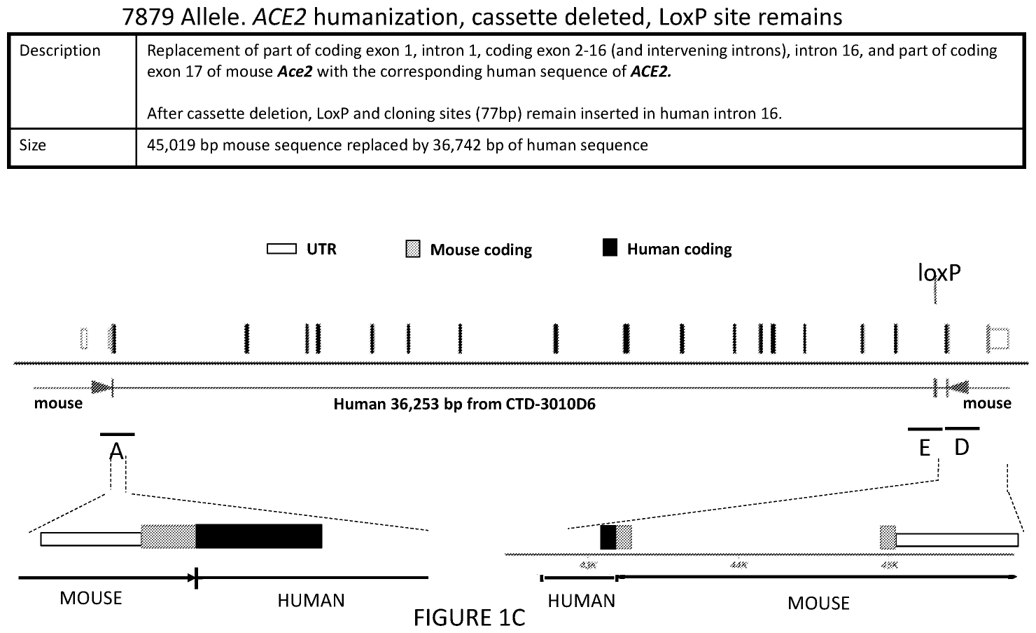

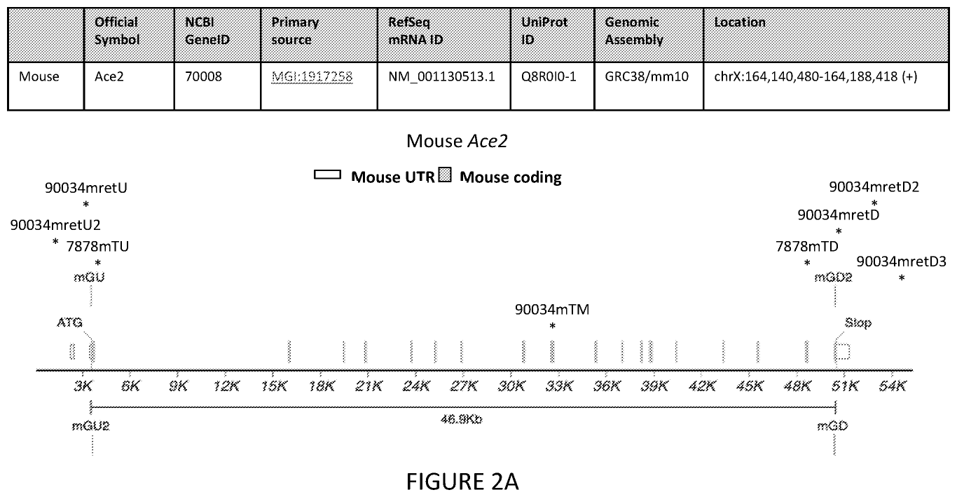

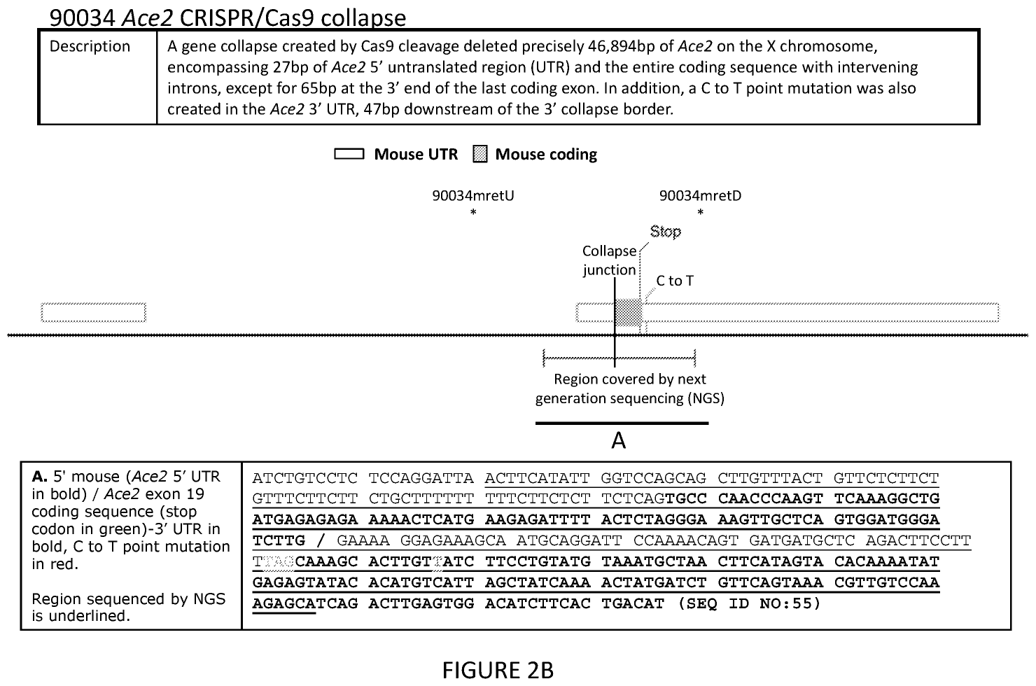

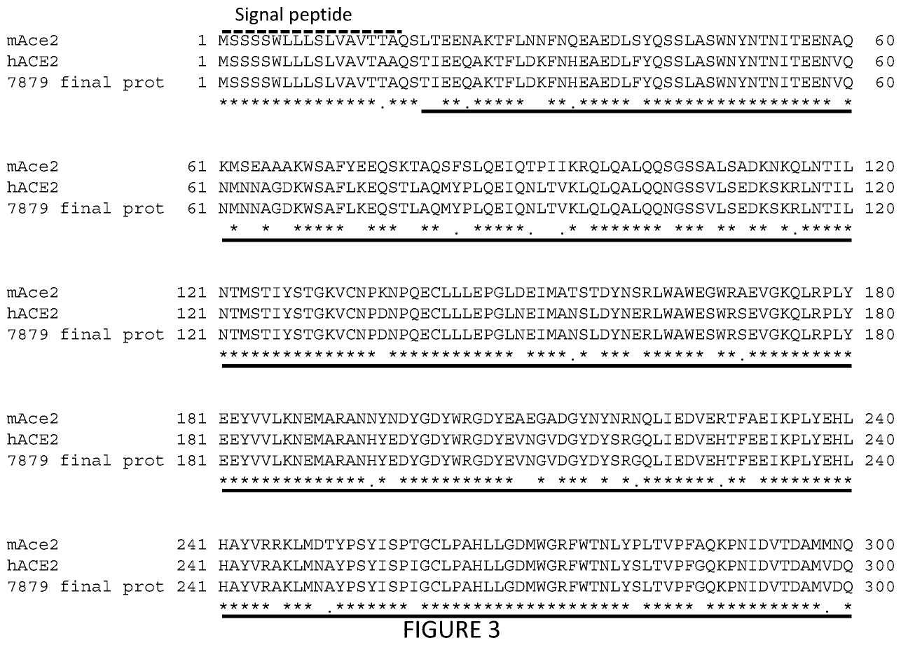

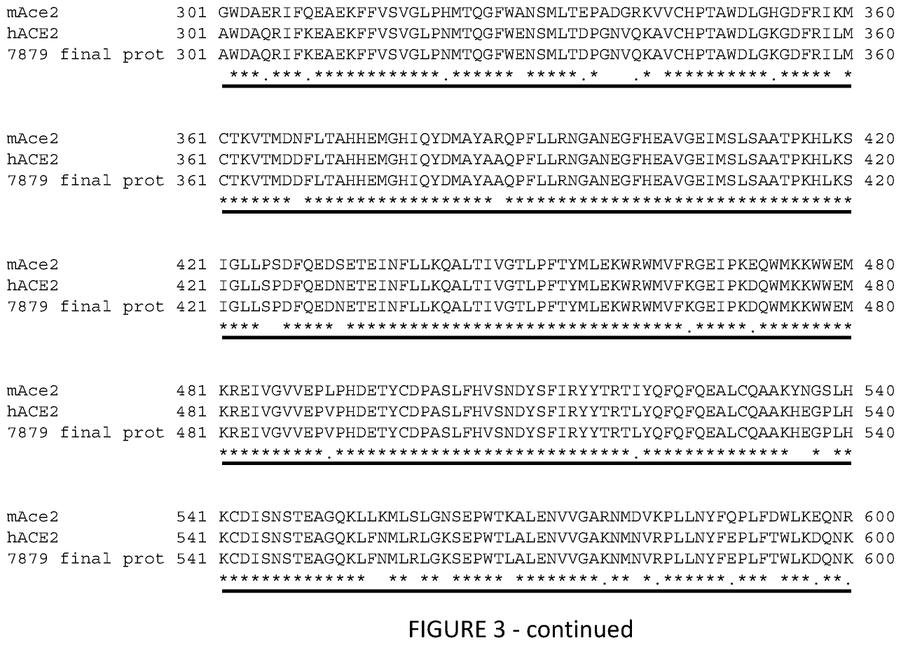

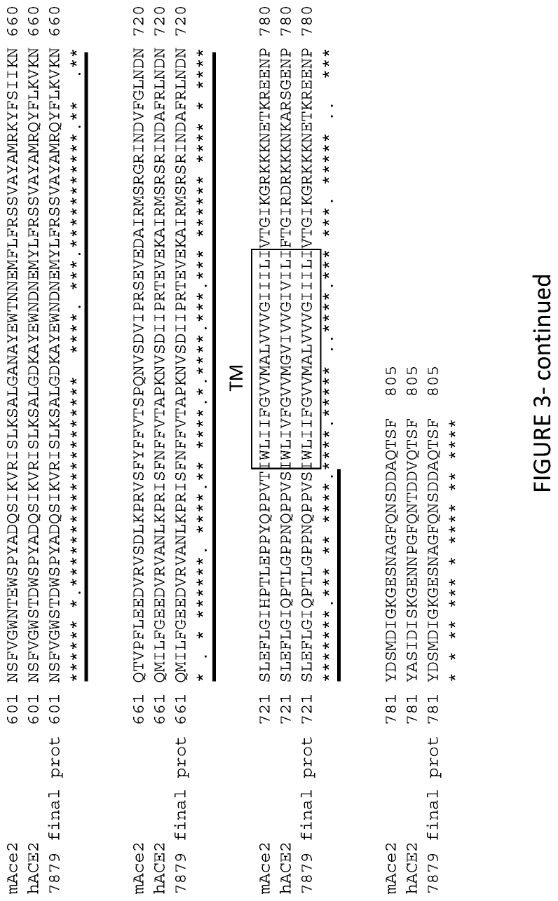

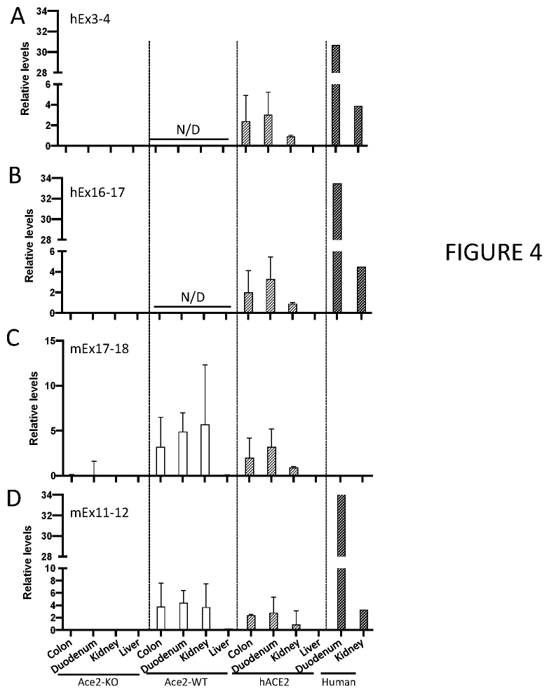

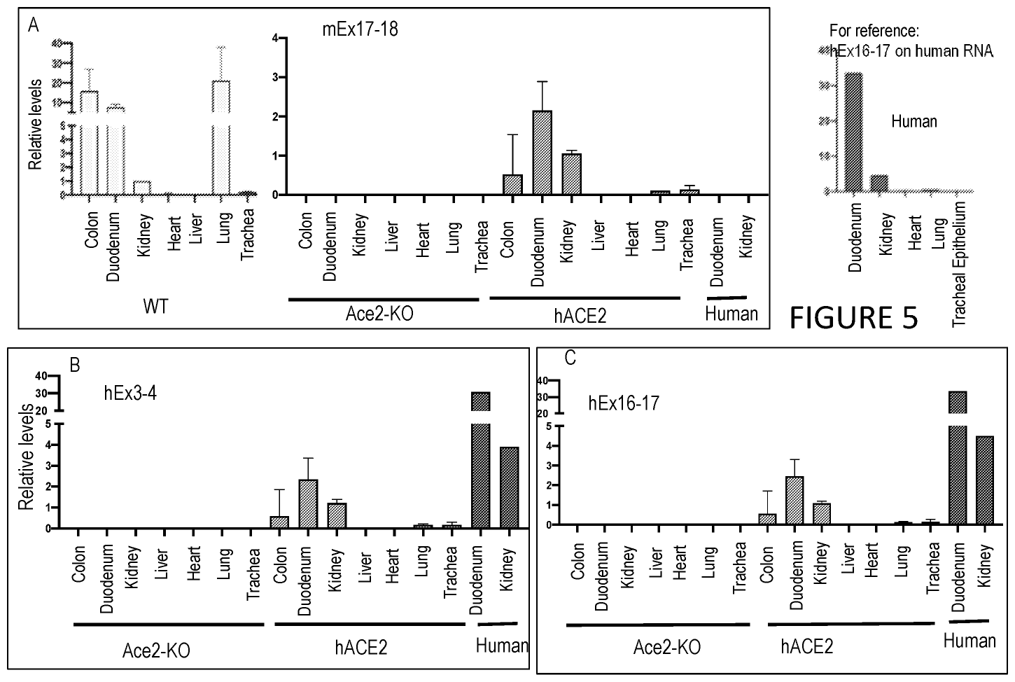

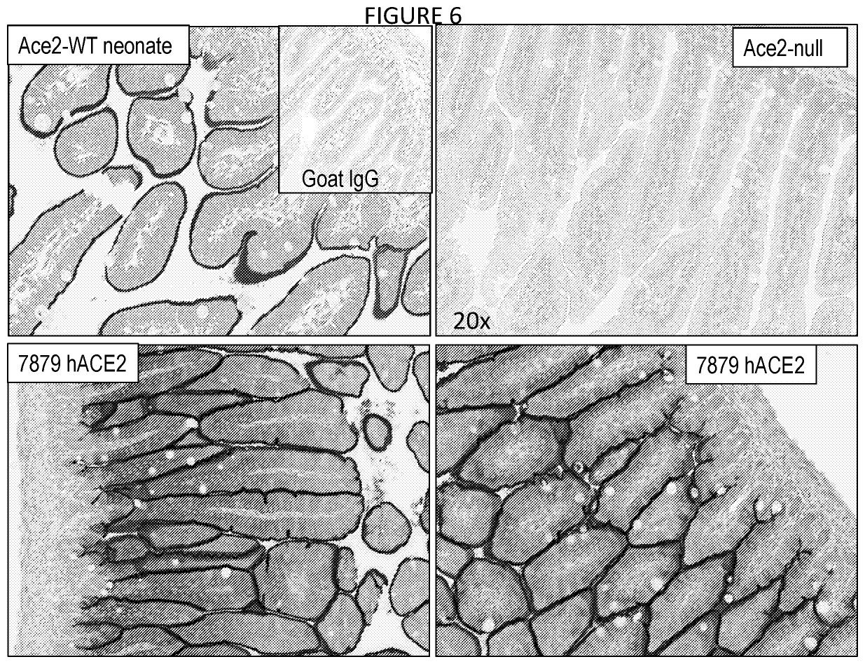

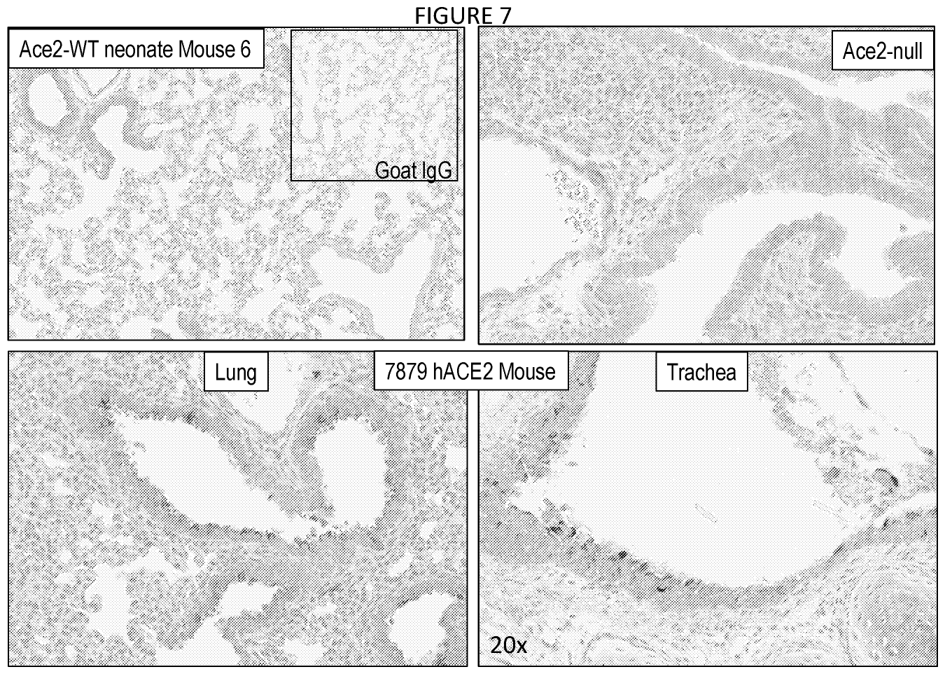

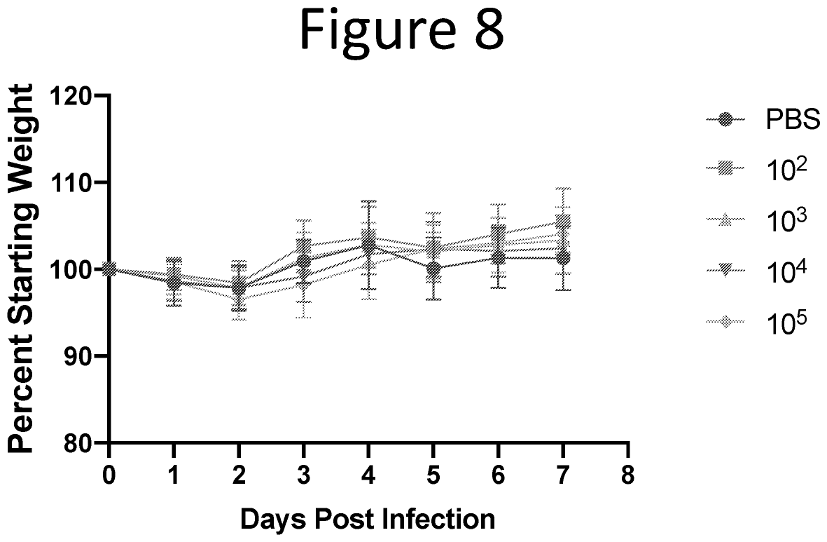

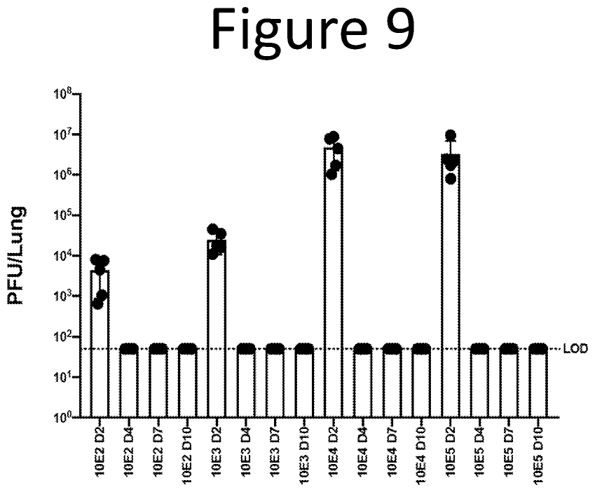

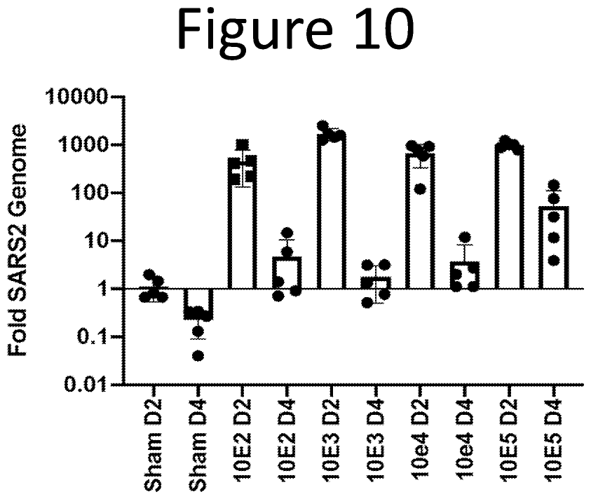

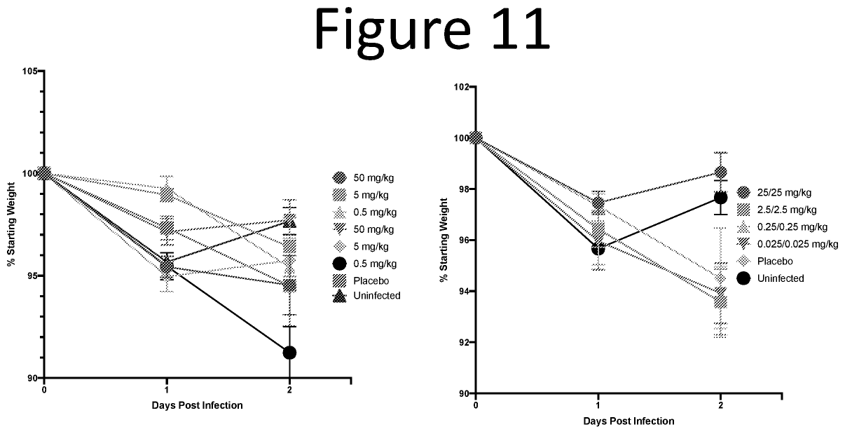

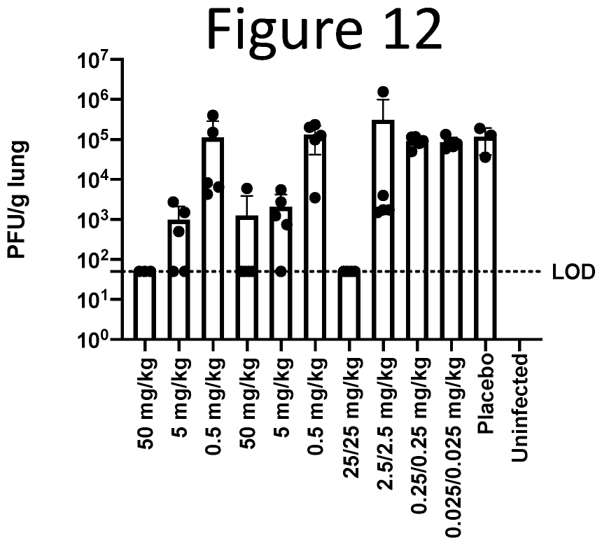

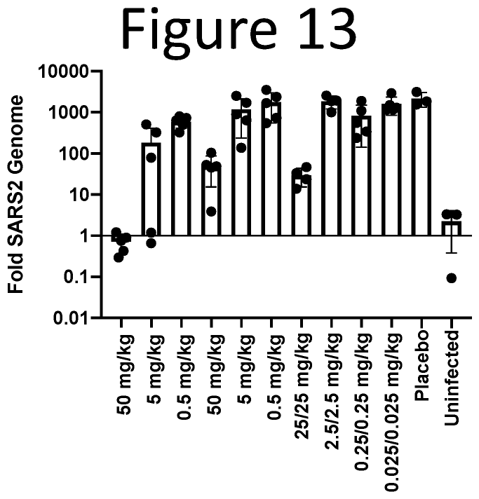

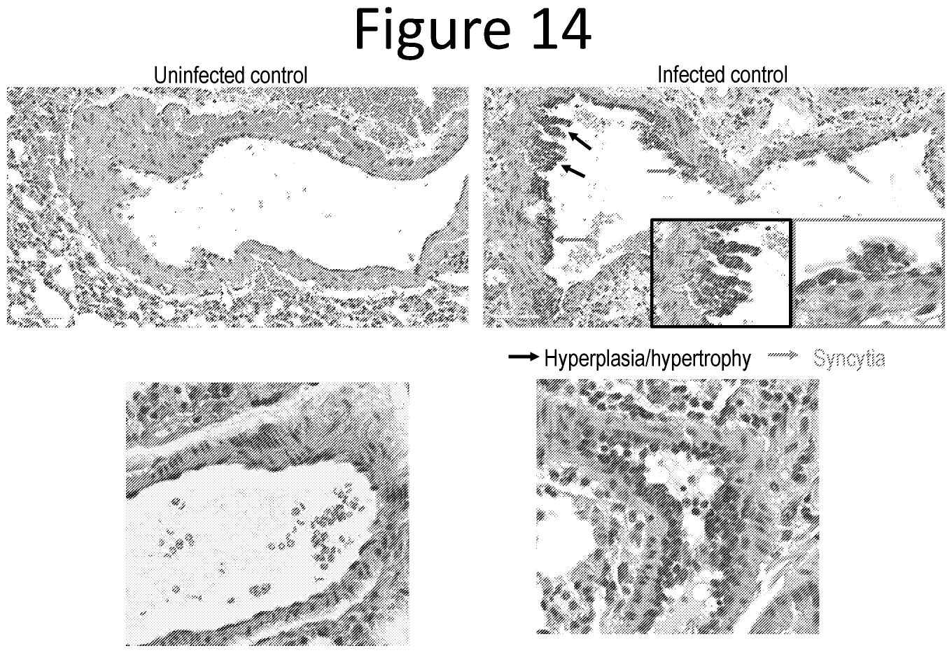

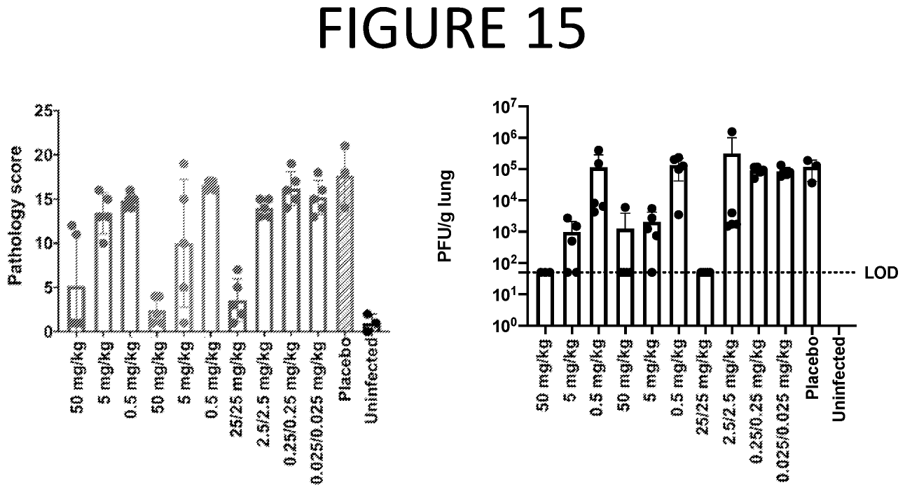

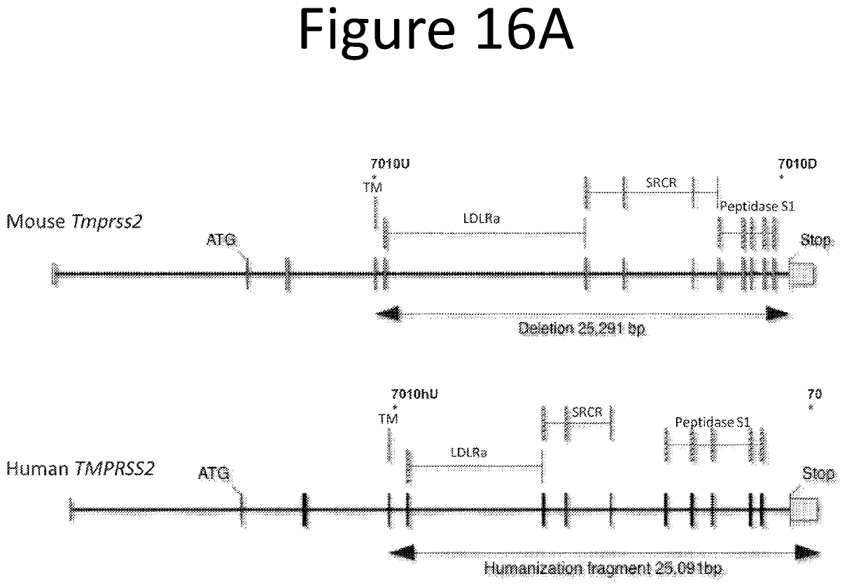

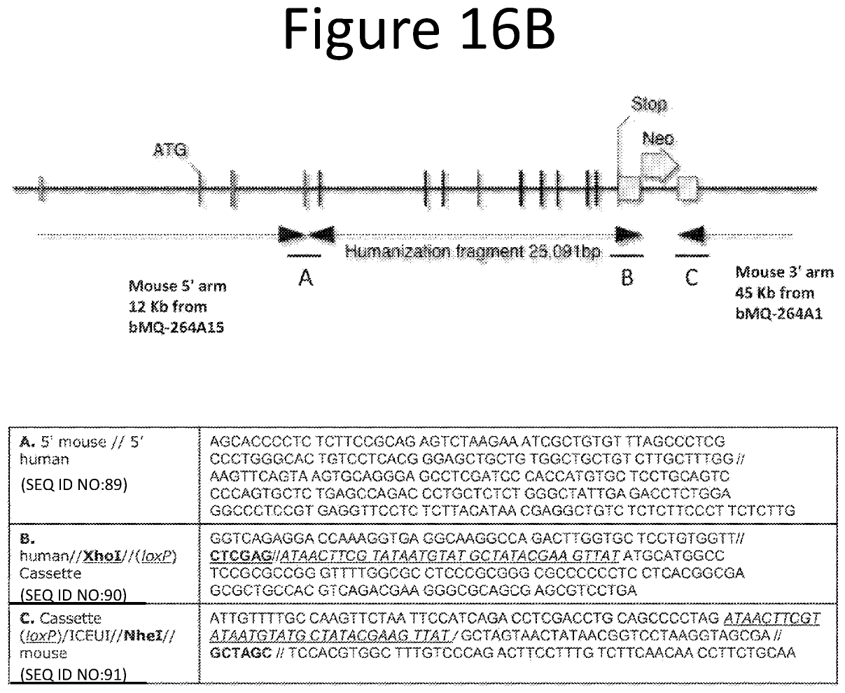

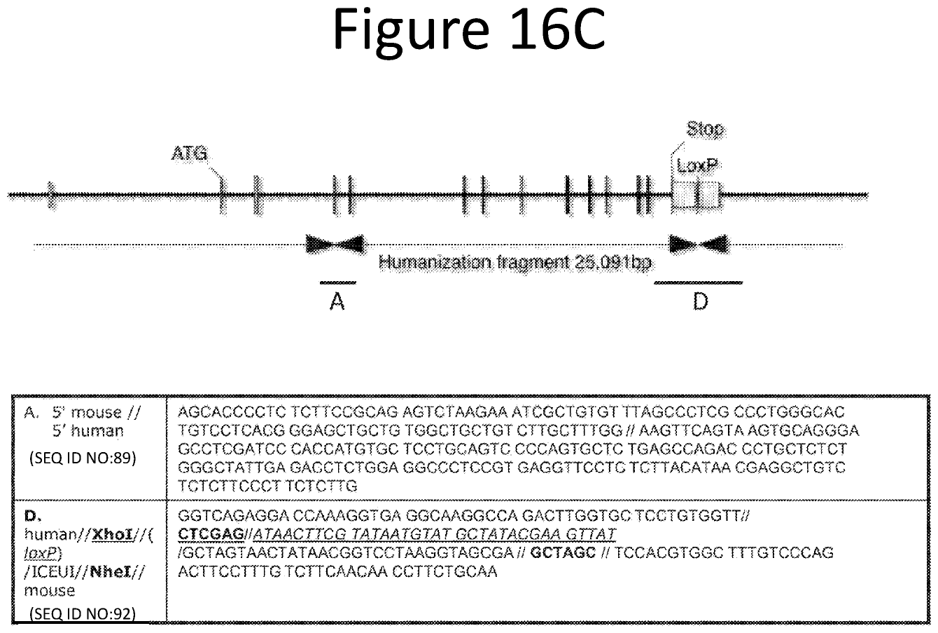

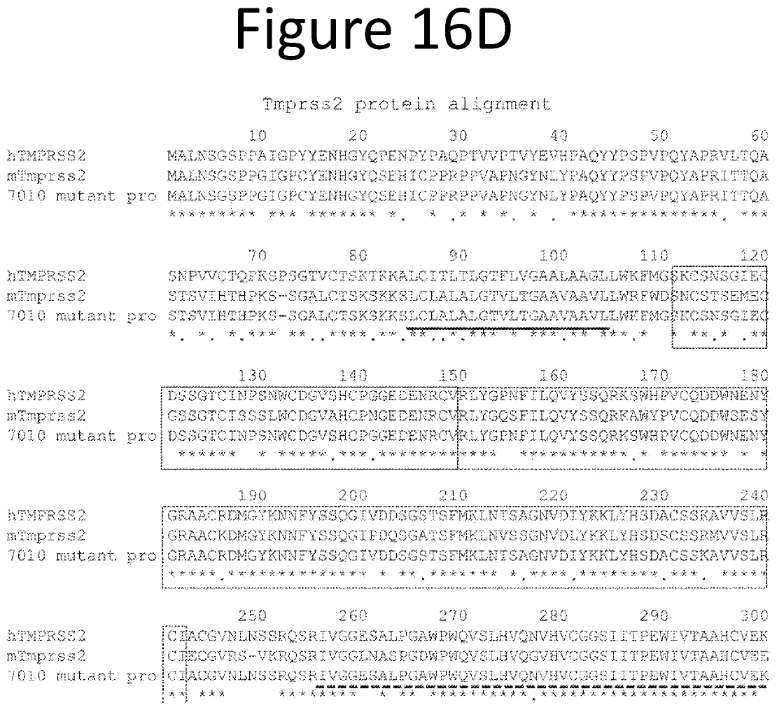

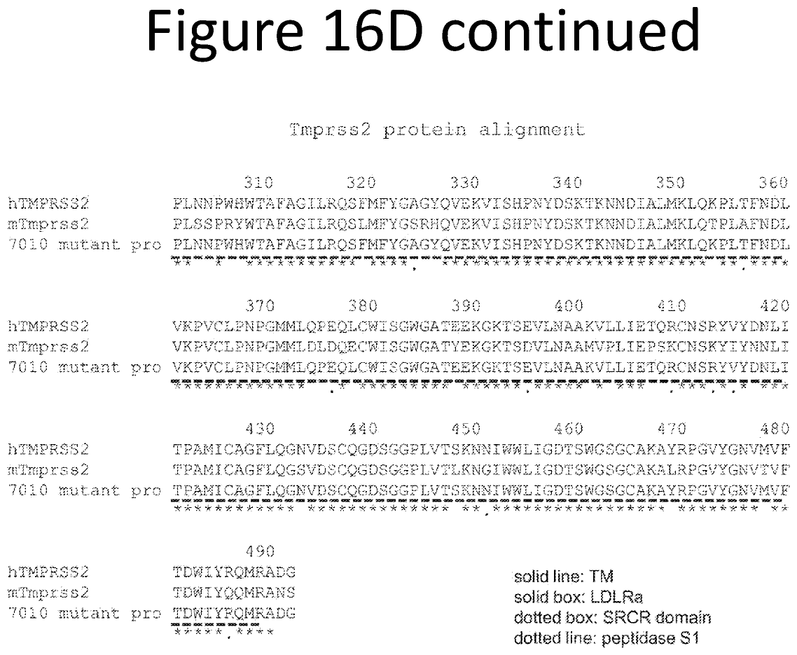

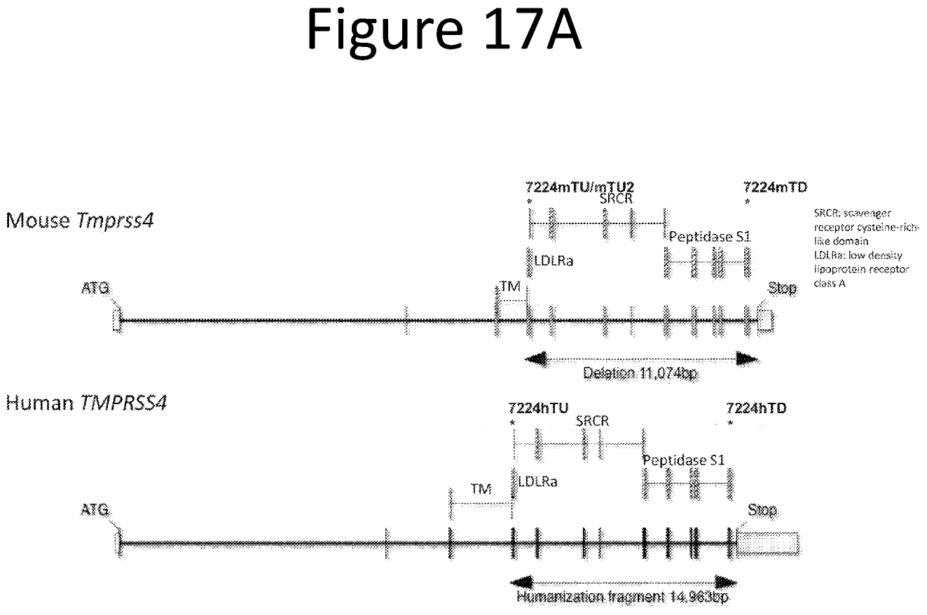

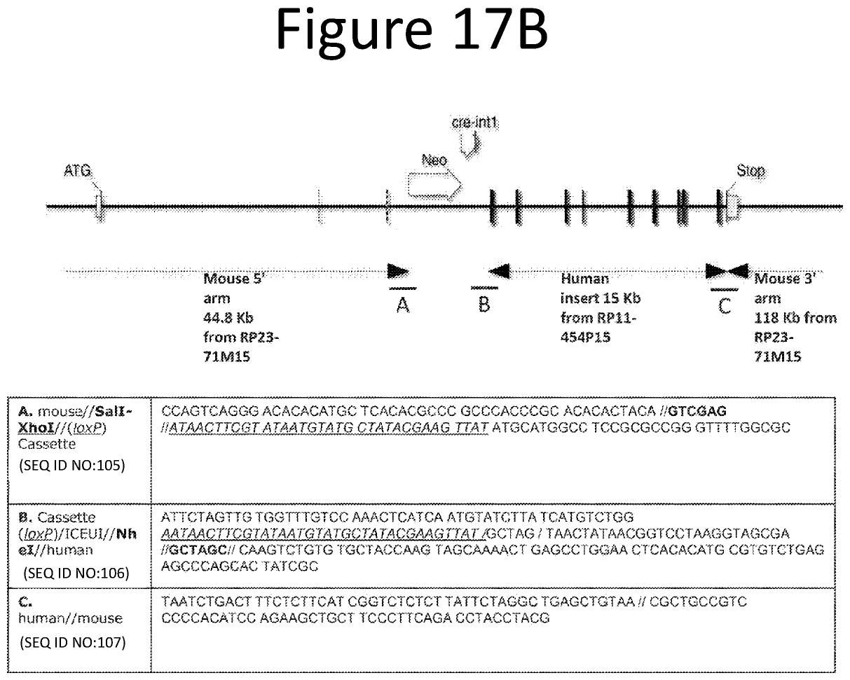

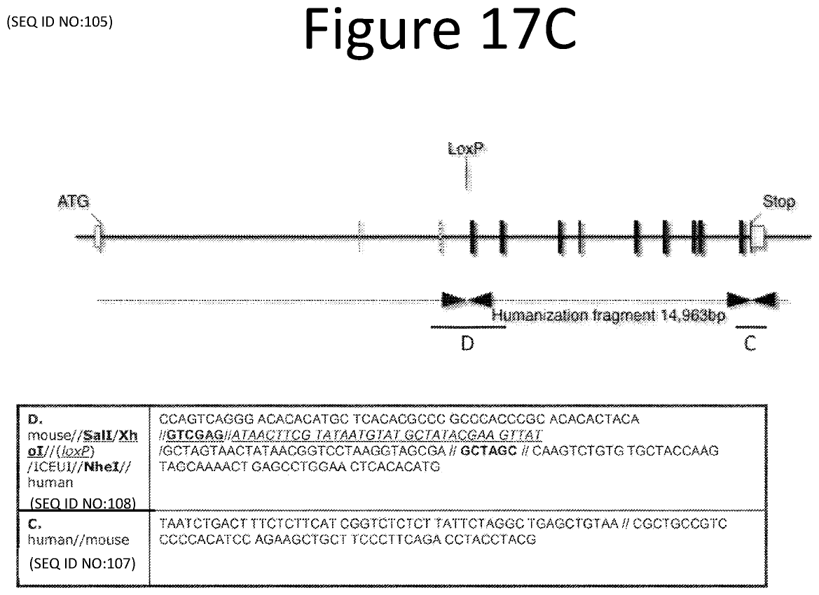

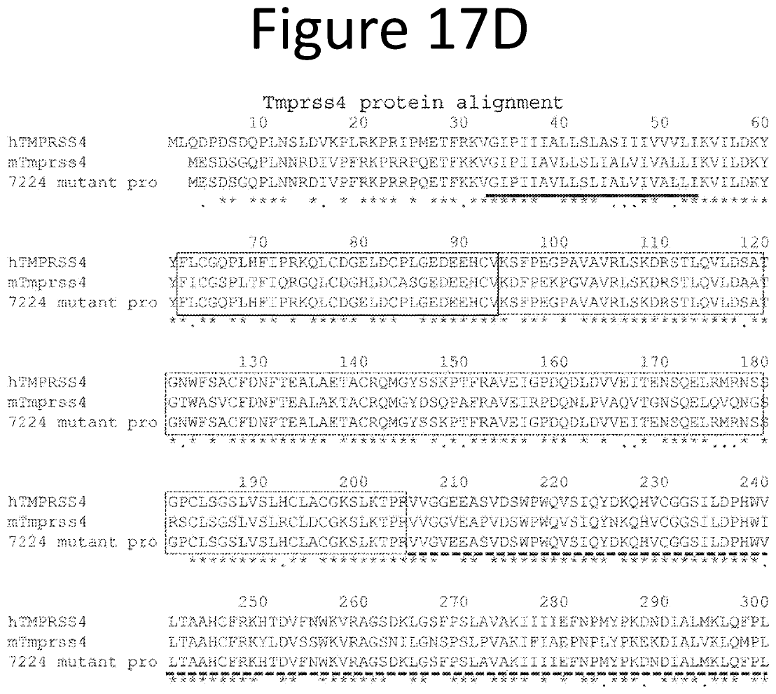

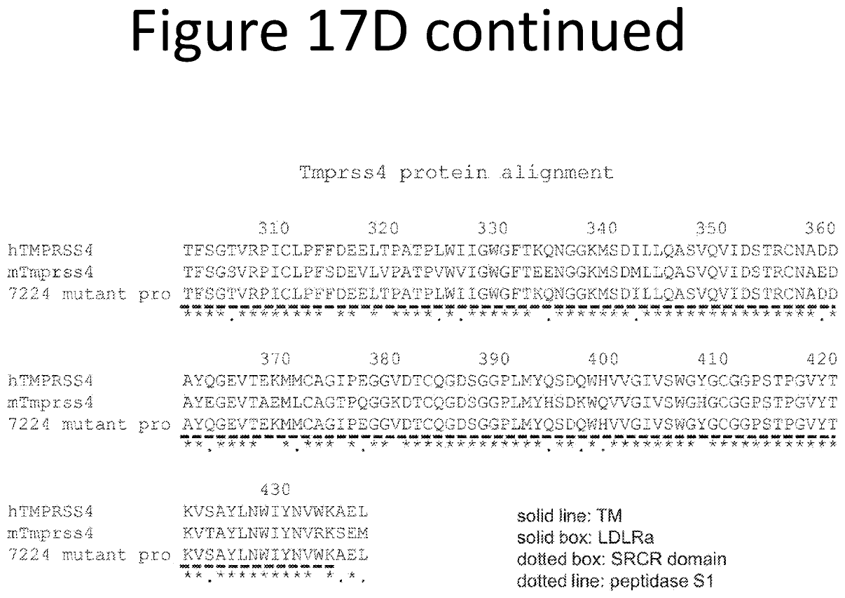

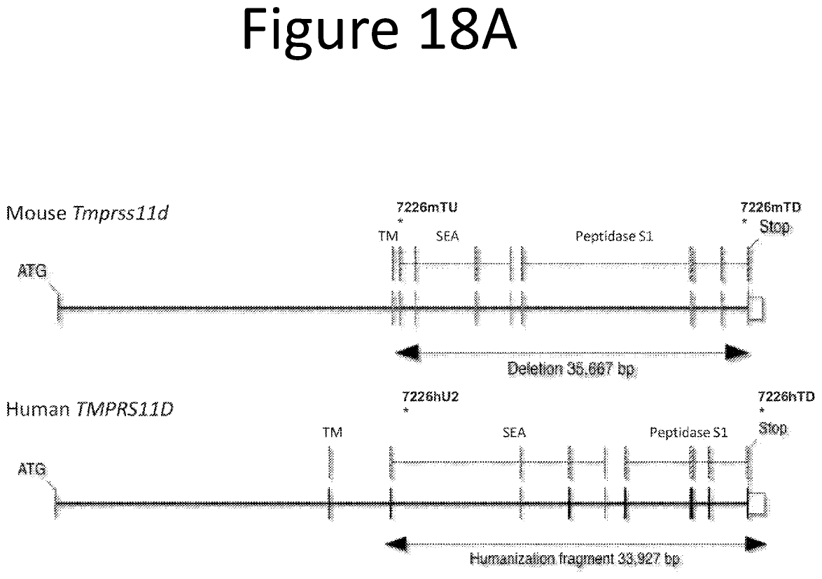

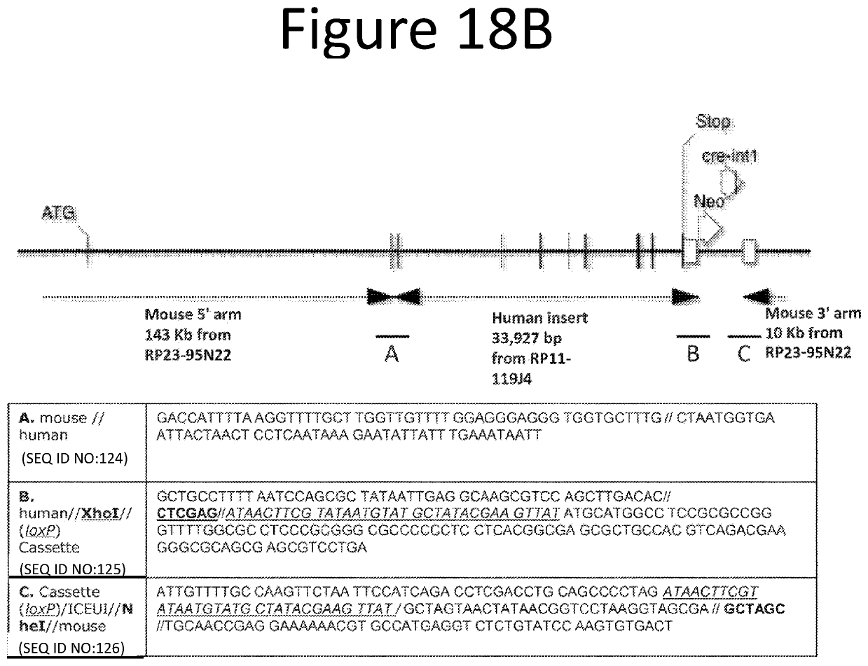

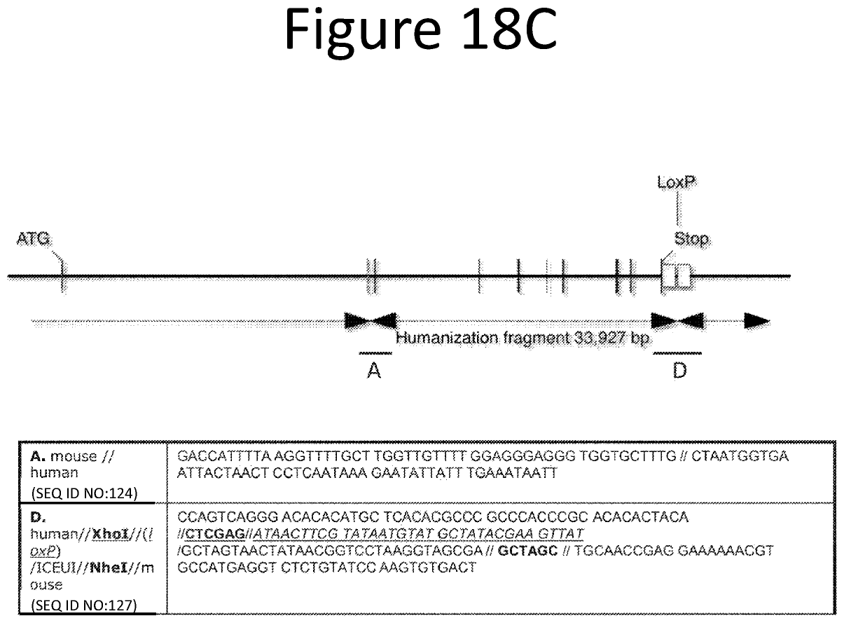

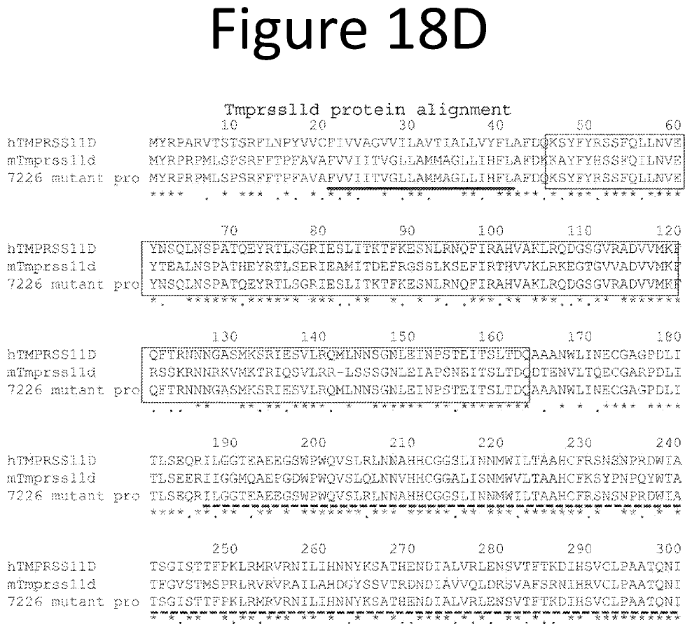

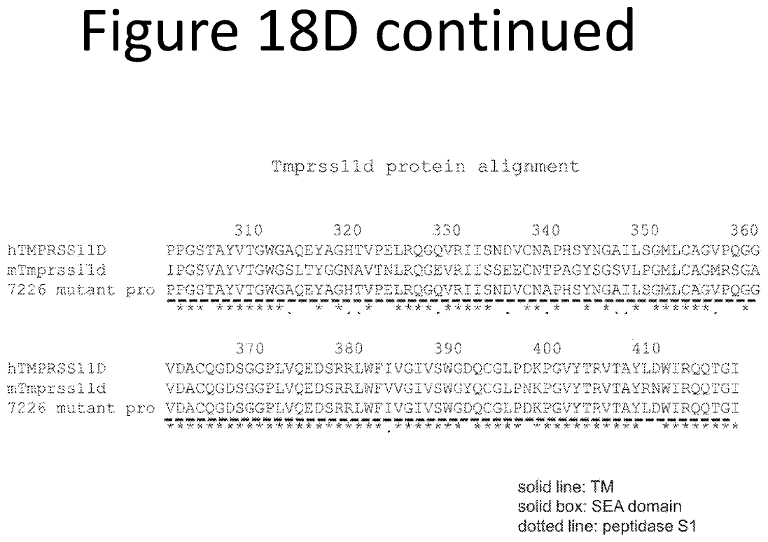

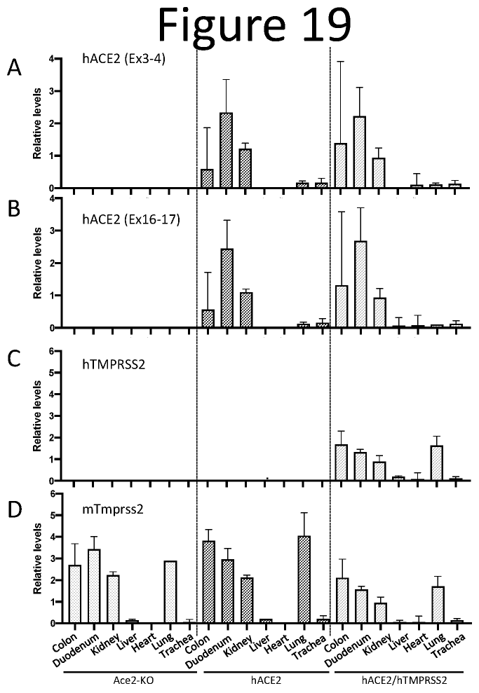

FIG. 1C shows a schematic (not-to-scale) of a cassette-deleted version of the humanized ACE2 allele in FIG. 1B. The mouse exonic sequences are indicated by the gray boxes, the human exons sequences are indicated by the black boxes, and the mouse 5′ and 3′ UTRs are indicated by the white boxes. The sequences between the different 5′ mouse/human junction, the deleted loxP and cloning site, and 3′ human/mouse junction are indicated by the lines labeled A, E, and D, respectively, at the bottom of the figure. The sequences of these junctions are provided below in Table 2.

TABLE 2

| | |
|---|---|
| A. 5′ mouse/5′ human | AAGATGTCCA GCTCCTCCTG GCTCCTTCTC |
| | AGCCTTGTTG CTGTTACTAC TGCTCAGTCC/ |
| | ACCATTGAGG AACAGGCCAA GACATTTTTG |
| | GACAAGTTTA ACCACGAAGC CGAAGACCTG |
| | (SEQ ID NO: 18) |
| E. Human//XhoI/ | CATTTATACA TTTCCACACT TACAACTCAA |
| (loxP)/ICEUI//NheI// | TTTTCCAATG GAGCTGTTGA TGAACCTAAT/ |
| Human | CTCGAG/ |
| | ATAACTTCGTATAATGTATGCTATACGAAGTTAT |
| | GCTAGG TAACTATAACGGTCCTAAGGTAGCGA |
| | GCTAGC CTAGGTTGCA AGGCATGAAA |
| | GATGCATAAT TGTCAAAGAC TTATATCTTT |
| | AATTAGACCT AT |
| | (SEQ ID NO: 23) |
| D. 3′ human/3′ mouse | AGCCTAGAGT TTCTGGGGAT ACAGCCAACA |
| | CTTGGACCTC CTAACCAGCC CCCTGTTTCC/ |
| | ATATGGCTGA TTATTTTTGG TGTTGTGATG |
| | GCACTGGTAG TGGTTGGCAT CATCATCCTG |
| | (SEQ ID NO: 21) |

FIG. 2A shows a schematic (not-to-scale) of the mouse ACE2 locus. Untranslated and coding exons are represented by rectangles, and coding sequences are indicated by the filled boxes, the untranslated regions (UTRs) are indicated by the unfilled boxes, and various accession numbers for non-limiting examples of a mouse ACE2 gene, along with the chromosomal locations, are indicated at the top of the figure. The asterisks indicate the locations of (A) upstream (7878mTU) and downstream (7878mTD) primers for the loss-of-allele assay and (B) upstream (90034metU and 90034metU2) and downstream (90034metD, 90034metD2, 90034metD3, and 90034metD4) primers for a retention assay. Also shown are the locations targeted by guide RNAs (mGU, mGU2, mGD, and mGD2) used to collapse the ACE2 gene and create a null allele (ACE2-null) shown in FIG. 2B.

FIG. 2B shows a schematic (not-to-scale) of a mouse ACE2-null allele. The remaining coding sequence is indicated by the filled box, the untranslated regions (UTRs) are indicated by the unfilled boxes and a C to T point mutation in the 3′UTR is also depicted.

FIG. 3 shows an alignment of the mouse ACE2 protein (mACE2; SEQ ID NO: 2), the human ACE2 protein (hACE2; SEQ ID NO:4), and the humanized mouse ACE2 protein (7878 final prot; SEQ ID NO:24). The dotted line above the alignment denotes the signal peptide (amino acids 1-17 of SEQ ID NO:24). The underscored residues are those encoded by the introduced human sequences (amino acids 20-740 of SEQ ID NO:24). The boxed residues constitute the transmembrane domain of the humanized mouse ACE2 protein (amino acids 741-761 of SEQ ID NO:24).

FIG. 4 provides relative levels (y-axis) of mRNA transcripts isolated from the colon, duodenum, kidney, or liver isolated from mice comprising a knockout of ACE2 (ACE2-KO), wildtype mice (ACE2-WT), mice comprising an endogenous ACE2 locus modified to encode a humanized ACE2 protein (hACE2), or isolated from a human.

FIG. 5 provides relative levels (y-axis) of mRNA transcripts isolated from colon, duodenum, kidney, liver, heart, lung, or trachea isolated from mice comprising a knockout of ACE2 (ACE2-KO), wildtype mice (ACE2-WT), mice comprising an endogenous ACE2 locus modified to encode a humanized ACE2 protein (hACE2), or isolated from a human.

FIG. 6 provides immunohistochemistry images of duodenum (i) isolated from a neonate wild-type mouse (ACE2-WT neonate mouse 6), a mouse comprising a knockout of ACE2 (ACE2-null), and two mice comprising an endogenous ACE2 locus modified to express a humanized ACE2 protein (7879 hACE2 Mouse) and (ii) stained with ACE2 antibodies that recognize mouse and human ACE2. Magnification is at 20×.

FIG. 7 provides immunohistochemistry images of lung or trachea (i) isolated from a neonate wild-type mouse (ACE2-WT neonate mouse 6), a mouse comprising a knockout of ACE2 (ACE2-null), and a mouse comprising an endogenous ACE2 locus modified to express a humanized ACE2 protein (7879 hACE2 Mouse) and (ii) stained with ACE2 antibodies that recognize mouse and human ACE2. Magnification is at 20×.

FIG. 8 provides the percent starting weight (y-axis) of mice comprising an endogenous ACE2 locus modified to express a humanized ACE2 protein and inoculated on day 0 with PBS (control), 102 PFU of SARS-COV-2 isolate, WA1, 10³ PFU of SARS-COV-2 isolate, WA1, 10⁴ PFU of SARS-COV-2 isolate, WA1, or 10⁵ PFU of SARS-COV-2 isolate, WA1 over 8 days (x-axis).

FIG. 9 provides plaque forming units (PFU) per lung isolated from mice comprising an endogenous ACE2 locus modified to express a humanized ACE2 protein and infected with varying doses of SARS-COV-2 (10E2, 10E3, 10E4, or 10E5; x-axis) 2 days (D2), 4 days (D4), or 7 days (D7) after infection.

FIG. 10 provides the level of subgenomic SARS-COV-2 expression (Fold SARS2 Genome; y-axis) found in the lungs isolated from control mice (Sham) or mice from mice comprising an endogenous ACE2 locus modified to express a humanized ACE2 protein and infected with varying doses of SARS-COV-2 (10E2, 10E3, 10E4, or 10E5; x-axis) 2 days (D2) or 4 days (D4) after infection.

FIG. 11 provides the percent starting weight (y-axis) of mice comprising an endogenous ACE2 locus modified to express a humanized ACE2 protein and prophylactially treated with a placebo, with 50 mg/kg, 5 mg/kg, or 0.5 mg/kg of a single anti-spike protein antibody (left panel) or with a combination of 25/25 mg/kg, 2.5/2.5 mg/kg, or 0.5/0.5 mg/kg of two anti-spike protein antibodies (right panel) 2 days before inoculation on day 0 with 10⁵ PFU SARS-COV-2 isolate.

FIG. 12 provides plaque forming units (PFU) per lung isolated from mice comprising an endogenous ACE2 locus modified to express a humanized ACE2 protein and prophylactially treated with a placebo, with 50 mg/kg, 5 mg/kg, or 0.5 mg/kg of a single anti-spike protein antibody or with a combination of 25/25 mg/kg, 2.5/2.5 mg/kg, or 0.5/0.5 mg/kg of two anti-spike protein antibodies 2 days before inoculation with 10⁵ PFU SARS-COV-2 isolate. Lungs were isolated 2 days after inoculation. LOD=limits of detection

FIG. 13 provides the level of subgenomic SARS-COV-2 expression (Fold SARS2 Genome; y-axis) found in the lungs isolated from mice comprising an endogenous ACE2 locus modified to express a humanized ACE2 protein and prophylactially treated with a placebo, with 50 mg/kg, 5 mg/kg, or 0.5 mg/kg of a single anti-spike protein antibody or with a combination of 25/25 mg/kg, 2.5/2.5 mg/kg, or 0.5/0.5 mg/kg of two anti-spike protein antibodies 2 days before inoculation with 10⁵ PFU SARS-COV-2 isolate. Lungs were isolated 2 days after inoculation.

FIG. 14 presents representative H&E images of blood vessel of the lungs of mice comprising an endogenous ACE2 locus modified to express a humanized ACE2 protein that were either uninfected (left) or infected with SARS-COV-2 (right). Vascular lesions in the infected lungs include endothelial hyperplasia/hypertrophy, endothelial syncytia, and endothelialitis. Magnification is at 40×.

FIG. 15 provides the pathology score (left panel) or plaque forming units (PFU) per lung (right panel) of mice comprising an endogenous ACE2 locus modified to express a humanized ACE2 protein and prophylactially treated with a placebo, with 50 mg/kg, 5 mg/kg, or 0.5 mg/kg of a single anti-spike protein antibody or with a combination of 25/25 mg/kg, 2.5/2.5 mg/kg, or 0.5/0.5 mg/kg of two anti-spike protein antibodies 2 days before inoculation with 10⁵ PFU SARS-COV-2 isolate. Lungs were isolated 2 days after inoculation.

FIG. 16A shows a diagram, not to scale, of the genomic organization of mouse TMPRSS2 and human TMPRSS2 genes. Exons are represented by thin bars placed across the genomic sequences, with the first coding exon for both genes indicated by the start codon “ATG” above the exon, and the last coding exon indicated by the “Stop” codon above the exon. A mouse genomic fragment of about 25,291 bp to be deleted and a human genomic fragment of about 25,091 bp to be inserted are indicated. Locations of probes used in an assay described in Example 4 are indicated. TM: transmembrane domain; SRCR: scavenger receptor cysteine-rich like domain; LDLRa: low density lipoprotein receptor class A.

FIG. 16B illustrates, not to scale, an exemplary modified BAC vector for humanization of an endogenous mouse TMPRSS2 gene, along with the junction sequences (SEQ ID NOS: 89, 90 and 91).

FIG. 16C illustrates, not to scale, a humanized TMPRSS2 allele after the neomycin cassette has been deleted, along with the junction sequences (SEQ ID NOS: 89 and 92).

FIG. 16D sets forth a sequence alignment of a human TMPRSS2 protein (SEQ ID NO:71), a mouse TMPRSS2 protein (SEQ ID NO:69), and a humanized TMPRSS2 protein (“7010 mutant pro”) (SEQ ID NO:74).

FIG. 17A shows a diagram, not to scale, of the genomic organization of mouse TMPRSS4 and human TMPRSS4 genes. Exons are represented by thin bars placed across the genomic sequences, with the first exon (also the first coding exon) for both genes indicated by the start codon “ATG” above the exon, and the last coding exon indicated by the “Stop” codon above the exon. The mouse genomic fragment of about 11,074 bp to be deleted and the human genomic fragment of about 14,963 bp to be inserted are indicated. Locations of probes used in an assay described in Example 5 are indicated. TM: transmembrane domain; SRCR: scavenger receptor cysteine-rich like domain; LDLRa: low density lipoprotein receptor class A.

FIG. 17B illustrates, not to scale, an exemplary modified BAC vector for humanization of an endogenous mouse TMPRSS4 gene, along with the junction sequences (SEQ ID NOS: 105, 106 and 107).

FIG. 17C illustrates, not to scale, a humanized TMPRSS4 allele after the neomycin cassette has been deleted, along with the junction sequences (SEQ ID NOS: 107 and 108).

FIG. 17D sets forth a sequence alignment of a human TMPRSS4 protein (SEQ ID NO: 78), a mouse TMPRSS4 protein (SEQ ID NO:76), and a humanized TMPRSS4 protein (“7224 mutant pro”) (SEQ ID NO:81).

FIG. 18A shows a diagram, not to scale, of the genomic organization of mouse TMPRSS11d and human TMPRSS11D genes. Exons are represented by thin bars placed across the genomic sequences, with the first exon (also the first codon exon) for both genes indicated by the start codon “ATG” above the exon, and the last coding exon indicated by the “Stop” codon above the exon. A mouse genomic fragment of about 35,667 bp to be deleted and a human genomic fragment of about 33,927 bp to be inserted are indicated. Locations of probes used in an assay described in Example 6 are indicated. TM: transmembrane domain; SEA: domain found in sea urchin sperm protein, enterokinase and agrin.

FIG. 18B illustrates, not to scale, an exemplary modified BAC vector for humanization of an endogenous mouse TMPRSS11d gene, along with the junction sequences (SEQ ID NOS: 124, 125 and 126).

FIG. 18C illustrates, not to scale, a humanized TMPRSS11 allele after the neomycin cassette has been deleted, along with the junction sequences (SEQ ID NOS: 124 and 127).

FIG. 18D sets forth a sequence alignment of a human TMPRSS11D protein (SEQ ID NO:85), a mouse TMPRSS11d protein (SEQ ID NO:83), and a humanized TMPRSS11d protein (“7226 mutant pro”) (SEQ ID NO:88).

FIG. 19 provides relative levels (y-axis) of mRNA transcripts isolated from the colon, duodenum, kidney, liver, heart, lung, or trachea isolated from mice comprising a knockout of ACE2 (ACE2-KO), mice comprising an endogenous ACE2 locus modified to encode a recombinant ACE2 protein (hACE2), or mice comprising an endogenous ACE2 locus modified to encode a recombinant ACE2 protein (hACE2) and an endogenous TMPRSS2 locus modified to encode a humanized TMPRSS2 protein (hTMPRSS2).

### DESCRIPTION

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “genomically integrated” refers to a nucleic acid that has been introduced into a cell such that the nucleotide sequence integrates into the genome of the cell and is capable of being inherited by progeny thereof. Any protocol may be used for the stable incorporation of a nucleic acid into the genome of a cell.

The term “targeting vector” refers to a recombinant nucleic acid that can be introduced by homologous recombination, non-homologous-end-joining-mediated ligation, or any other means of recombination to a target position in the genome of a cell.

The term “wild-type” includes entities having a structure and/or activity as found in a normal (as contrasted with mutant, diseased, altered, or so forth) state or context. Wild-type genes and polypeptides often exist in multiple different forms (e.g., alleles).

The term “endogenous” refers to a nucleic acid sequence that occurs naturally within a cell or non-human animal. For example, an endogenous ACE2 sequence of a non-human animal refers to a native ACE2 sequence that naturally occurs at the ACE2 locus in the non-human animal.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two portions that do not naturally occur together in the same molecule. For example, the term “heterologous,” when used with reference to portions of a nucleic acid or portions of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

The term “orthologous” when used in the context of proteins or genes encoding proteins refer to those proteins or genes that diverge after a speciation event, but the gene and protein and their respective functions are conserved. As non-limiting examples, a non-human animal (e.g., rodent, e.g., rat or mouse) TMPRSS2 gene and the encoded non-human animal (e.g., rodent, e.g., rat or mouse) TMPRSS2 protein are orthologous to a human TMPRSS2 gene and the encoded human TMPRSS2 protein, respectively. Similarly, a non-human animal (e.g., rodent, e.g., rat or mouse) TMPRSS4 gene and the encoded non-human animal (e.g., rodent, e.g., rat or mouse) TMPRSS4 protein are orthologous to a human TMPRSS4 gene and the encoded human TMPRSS4 protein, respectively. In another non-limiting example, a non-human animal (e.g., rodent, e.g., rat or mouse) TMPRSS11 gene and the encoded non-human animal (e.g., rodent, e.g., rat or mouse) TMPRSS11 protein are orthologous to a human TMPRSS11 gene and the encoded human TMPRSS11 protein, respectively

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a prokaryotic protein (i.e., a protein naturally expressed in a prokaryotic cell) can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, an “ACE2 locus” may refer to the specific location of an ACE2 gene, ACE2 DNA sequence, ACE2-encoding sequence, or ACE2 position on a chromosome of the genome of an organism that has been identified as to where such a sequence resides. An “ACE2 locus” may comprise a regulatory element of an ACE2 gene, including, for example, an enhancer, a promoter, 5′ and/or 3′ untranslated region (UTR), or a combination thereof.

The term “gene” refers to a DNA sequence in a chromosome that codes for a product (e.g., an RNA product and/or a polypeptide product) and includes the coding region interrupted with non-coding introns and sequence located adjacent to the coding region on both the 5′ and 3′ ends such that the gene corresponds to the full-length mRNA (including the 5′ and 3′ untranslated sequences). The term “gene” also includes other non-coding sequences including regulatory sequences (e.g., promoters, enhancers, and transcription factor binding sites), polyadenylation signals, internal ribosome entry sites, silencers, insulating sequence, and matrix attachment regions. These sequences may be close to the coding region of the gene (e.g., within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The term “variant” refers to a nucleotide sequence differing from the sequence most prevalent in a population (e.g., by one nucleotide) or a protein sequence different from the sequence most prevalent in a population (e.g., by one amino acid).

The term “fragment” when referring to a protein means a protein that is shorter or has fewer amino acids than the full-length protein. The term “fragment” when referring to a nucleic acid means a nucleic acid that is shorter or has fewer nucleotides than the full-length nucleic acid. A fragment can be, for example, an N-terminal fragment (i.e., removal of a portion of the C-terminal end of the protein), a C-terminal fragment (i.e., removal of a portion of the N-terminal end of the protein), or an internal fragment.

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube). The term “in vivo” includes natural environments (e.g., a cell or organism or body) and to processes or reactions that occur within a natural environment. The term “ex vivo” includes cells that have been removed from the body of an individual and to processes or reactions that occur within such cells.

The term “reporter gene” refers to a nucleic acid having a sequence encoding a gene product (typically an enzyme) that is easily and quantifiably assayed when a construct comprising the reporter gene sequence operably linked to a heterologous promoter and/or enhancer element is introduced into cells containing (or which can be made to contain) the factors necessary for the activation of the promoter and/or enhancer elements. Examples of reporter genes include, but are not limited, to genes encoding beta-galactosidase (lacZ), the bacterial chloramphenicol acetyltransferase (cat) genes, firefly luciferase genes, genes encoding beta-glucuronidase (GUS), and genes encoding fluorescent proteins. A “reporter protein” refers to a protein encoded by a reporter gene.

The term “fluorescent reporter protein” as used herein means a reporter protein that is detectable based on fluorescence wherein the fluorescence may be either from the reporter protein directly, activity of the reporter protein on a fluorogenic substrate, or a protein with affinity for binding to a fluorescent tagged compound. Examples of fluorescent proteins include green fluorescent proteins (e.g., GFP, GFP-2, tagGFP, turboGFP, eGFP, Emerald, Azami Green, Monomeric Azami Green, CopGFP, AceGFP, and ZsGreen1), yellow fluorescent proteins (e.g., YFP, eYFP, Citrine, Venus, YPet, PhiYFP, and ZsYellow1), blue fluorescent proteins (e.g., BFP, eBFP, eBFP2, Azurite, mKalamal, GFPuv, Sapphire, and T-sapphire), cyan fluorescent proteins (e.g., CFP, eCFP, Cerulean, CyPet, AmCyan1, and Midoriishi-Cyan), red fluorescent proteins (e.g., RFP, mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, and Jred), orange fluorescent proteins (e.g., mOrange, mKO, Kusabira-Orange, Monomeric Kusabira-Orange, mTangerine, and tdTomato), and any other suitable fluorescent protein whose presence in cells can be detected by flow cytometry methods.

The term “recombination” includes any process of exchange of genetic information between two polynucleotides and can occur by any mechanism. Recombination in response to double-strand breaks (DSBs) occurs principally through two conserved DNA repair pathways: non-homologous end joining (NHEJ) and homologous recombination (HR). See Kasparek & Humphrey (2011) Seminars in Cell & Dev. Biol. 22:886-897, herein incorporated by reference in its entirety for all purposes. Likewise, repair of a target nucleic acid mediated by an exogenous donor nucleic acid can include any process of exchange of genetic information between the two polynucleotides.

NHEJ includes the repair of double-strand breaks in a nucleic acid by direct ligation of the break ends to one another or to an exogenous sequence without the need for a homologous template. Ligation of non-contiguous sequences by NHEJ can often result in deletions, insertions, or translocations near the site of the double-strand break. For example, NHEJ can also result in the targeted integration of an exogenous donor nucleic acid through direct ligation of the break ends with the ends of the exogenous donor nucleic acid (i.e., NHEJ-based capture). Such NHEJ-mediated targeted integration can be preferred for insertion of an exogenous donor nucleic acid when homology directed repair (HDR) pathways are not readily usable (e.g., in non-dividing cells, primary cells, and cells which perform homology-based DNA repair poorly). In addition, in contrast to HDR, knowledge concerning large regions of sequence identity flanking the cleavage site is not needed, which can be beneficial when attempting targeted insertion into organisms that have genomes for which there is limited knowledge of the genomic sequence. The integration can proceed via ligation of blunt ends between the exogenous donor nucleic acid and the cleaved genomic sequence, or via ligation of sticky ends (i.e., having 5′ or 3′ overhangs) using an exogenous donor nucleic acid that is flanked by overhangs that are compatible with those generated by a nuclease agent in the cleaved genomic sequence. See, e.g., US 2011/020722, WO 2014/033644, WO 2014/089290, and Maresca et al. (2013) Genome Res. 23 (3): 539-546, each of which is herein incorporated by reference in its entirety for all purposes. If blunt ends are ligated, target and/or donor resection may be needed to generation regions of microhomology needed for fragment joining, which may create unwanted alterations in the target sequence.

Recombination can also occur via homology directed repair (HDR) or homologous recombination (HR). HDR or HR includes a form of nucleic acid repair that can require nucleotide sequence homology, uses a “donor” molecule as a template for repair of a “target” molecule (i.e., the one that experienced the double-strand break), and leads to transfer of genetic information from the donor to target. Without wishing to be bound by any particular theory, such transfer can involve mismatch correction of heteroduplex DNA that forms between the broken target and the donor, and/or synthesis-dependent strand annealing, in which the donor is used to resynthesize genetic information that will become part of the target, and/or related processes. In some cases, the donor polynucleotide, a portion of the donor polynucleotide, a copy of the donor polynucleotide, or a portion of a copy of the donor polynucleotide integrates into the target DNA. See Wang et al. (2013) Cell 153:910-918; Mandalos et al. (2012) PLOS ONE 7: e45768: 1-9; and Wang et al. (2013) Nat Biotechnol. 31:530-532, each of which is herein incorporated by reference in its entirety for all purposes.

The term “antigen-binding protein” includes any protein that binds to an antigen. Examples of antigen-binding proteins include an antibody, an antigen-binding fragment of an antibody, a multispecific antibody (e.g., a bi-specific antibody), an scFV, a bis-scFV, a diabody, a triabody, a tetrabody, a V-NAR, a VHH, a VL, a F (ab), a F (ab) 2, a DVD (dual variable domain antigen-binding protein), an SVD (single variable domain antigen-binding protein), a bispecific T-cell engager (BiTE), or a Davisbody (U.S. Pat. No. 8,586,713, herein incorporated by reference herein in its entirety for all purposes).

The term “multi-specific” or “bi-specific” with reference to an antigen-binding protein means that the protein recognizes different epitopes, either on the same antigen or on different antigens. A multi-specific antigen-binding protein can be a single multifunctional polypeptide, or it can be a multimeric complex of two or more polypeptides that are covalently or non-covalently associated with one another. For example, an antibody or fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity.

The term “antigen” refers to a substance, whether an entire molecule or a domain within a molecule, which is capable of eliciting production of antibodies with binding specificity to that substance. The term antigen also includes substances, which in wild-type host organisms would not elicit antibody production by virtue of self-recognition, but can elicit such a response in a host animal with appropriate genetic engineering to break immunological tolerance.

The term “epitope” refers to a site on an antigen to which an antigen-binding protein (e.g., antibody) binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996), herein incorporated by reference in its entirety for all purposes.

An “antibody paratope” as described herein generally comprises at a minimum a complementarity determining region (CDR) that specifically recognizes the heterologous epitope (e.g., a CDR3 region of a heavy and/or light chain variable domain).

The term “antibody” includes immunoglobulin molecules comprising four polypeptide chains, two heavy (H) chains and two light (L) chains inter-connected by disulfide bonds. Each heavy chain comprises a heavy chain variable domain and a heavy chain constant region (CH). The heavy chain constant region comprises three domains: CH1, CH2 and CH3. Each light chain comprises a light chain variable domain and a light chain constant region (CL). The heavy chain and light chain variable domains can be further subdivided into regions of hypervariability, termed complementarity determining regions (CDR), interspersed with regions that are more conserved, termed framework regions (FR). Each heavy and light chain variable domain comprises three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3). The term “high affinity” antibody refers to an antibody that has a KD with respect to its target epitope about of 10-9 M or lower (e.g., about 1×10-9 M, 1× 10-10 M, 1× 10-11 M, or about 1× 10-12 M). In one embodiment, KD is measured by surface plasmon resonance, e.g., BIACORE™; in another embodiment, KD is measured by ELISA.

The term “bi-specific antibody” includes an antibody capable of selectively binding two or more epitopes. Bi-specific antibodies generally comprise two different heavy chains, with each heavy chain specifically binding a different epitope-either on two different molecules (e.g., on two different antigens) or on the same molecule (e.g., on the same antigen). If a bi-specific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bi-specific antibody can be on the same or a different target (e.g., on the same or a different protein). Bi-specific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bi-specific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The term “heavy chain,” or “immunoglobulin heavy chain” includes an immunoglobulin heavy chain sequence, including immunoglobulin heavy chain constant region sequence, from any organism. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an epitope (e.g., recognizing the epitope with a KD in the micromolar, nanomolar, or picomolar range), that is capable of expressing and secreting from a cell, and that comprises at least one CDR. Heavy chain variable domains are encoded by variable region nucleotide sequences, which generally comprise VH, DH, and JH segments derived from a repertoire of VH, DH, and JH segments present in the germline. Sequences, locations and nomenclature for V, D, and J heavy chain segments for various organisms can be found in IMGT database, which is accessible via the internet on the World Wide Web (www) at the URL “imgt.org.”

The term “light chain” includes an immunoglobulin light chain sequence from any organism, and unless otherwise specified includes human kappa (κ) and lambda (λ) light chains and a VpreB, as well as surrogate light chains. Light chain variable domains typically include three light chain CDRs and four framework (FR) regions, unless otherwise specified. Generally, a full-length light chain includes, from amino terminus to carboxyl terminus, a variable domain that includes FR1-CDR1-FR2-CDR2-FR3-CDR3-FR4, and a light chain constant region amino acid sequence. Light chain variable domains are encoded by light chain variable region nucleotide sequences, which generally comprise light chain VL and light chain JL gene segments, derived from a repertoire of light chain V and J gene segments present in the germline. Sequences, locations and nomenclature for light chain V and J gene segments for various organisms can be found in IMGT database, which is accessible via the internet on the World Wide Web (www) at the URL “imgt.org.” Light chains include those, e.g., that do not selectively bind either a first or a second epitope selectively bound by the epitope-binding protein in which they appear. Light chains also include those that bind and recognize, or assist the heavy chain with binding and recognizing, one or more epitopes selectively bound by the epitope-binding protein in which they appear.

The term “complementary determining region” or “CDR,” as used herein, includes an amino acid sequence encoded by a nucleic acid sequence of an organism's immunoglobulin genes that normally (i.e., in a wild-type animal) appears between two framework regions in a variable region of a light or a heavy chain of an immunoglobulin molecule (e.g., an antibody or a T cell receptor). A CDR can be encoded by, for example, a germline sequence or a rearranged sequence, and, for example, by a naïve or a mature B cell or a T cell. A CDR can be somatically mutated (e.g., vary from a sequence encoded in an animal's germline), humanized, and/or modified with amino acid substitutions, additions, or deletions. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, e.g., as a result of splicing or connecting the sequences (e.g., V-D-J recombination to form a heavy chain CDR3.

Specific binding of an antigen-binding protein to its target antigen includes binding with an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas non-specific binding is usually the result of van der Waals forces. Specific binding does not however necessarily imply that an antigen-binding protein binds one and only one target.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which it does not.

Designation of a range of values includes all integers within or defining the range, and all subranges defined by integers within the range.

Unless otherwise apparent from the context, the term “about” encompasses values within a standard margin of error of measurement (e.g., SEM) of a stated value.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof. Statistically significant means p≤0.05.

### I. Overview

Disclosed herein are non-human animals and non-human animal cells comprising a humanized ACE2 locus and a humanized TMPRSS locus. Also described are methods of using such non-human animals and non-human animals cells. Non-human animals or non-human cells comprising a humanized ACE2 locus and a humanized TMPRSS locus respectively express a human ACE2 protein or a chimeric (e.g., humanized) ACE2 protein comprising one or more fragments of a human ACE2 protein (e.g., all or part of the human ACE2 extracellular domain) and a human TMPRSS protein or a chimeric (e.g., humanized) TMPRSS protein. Ligands binding human ACE2 often will not bind to orthologous non-human animal ACE2 proteins such as mouse ACE2 due to the sequence differences between human ACE2 and the non-human animal ACE2. For example, coronaviruses that infect cells expressing human ACE2 will often not recognize rodent ACE2. (Subbarao and Roberts (2006) TRENDS Microbiol. 14:299-303; McCray et al. (2007) J. Virol. 81:813-821; Wan (2020) J. Virol. 94:1-9; Sun et al. (2020) Cell Host & Microbe 28:1-10). Because of this, the progression of human-ACE2-mediated coronavirus infection or therapy thereof cannot be effectively assessed in wild-type non-human animals with unmodified endogenous (i.e., native) ACE2 loci.

As a binding partner for coronavirus, e.g., SARS-COV-2, non-human animals expressing human or humanized ACE2 can be utilized for studying SARS-COV-2 infection and associated diseases, e.g., COVID-19, and for determining the efficacy of therapies thereto. For example, in early 2020, efforts to identify effective measures against COVID-19 were in full swing.

### II. Non-Human Animals and Non-Human Animal Cells Comprising Humanized ACE2 and TMPRSS Loci

The cells and non-human animals disclosed herein comprise humanized ACE2 and TMPRSS, e.g., TMPRSS2, loci, and optionally express the respective humanized ACE2 and TMPRSS proteins.

### A. Angiotensin-Converting Enzyme 2 (ACE2) and Humanized ACE2 Loci

The cells and non-human animals described herein comprise a humanized ACE2 locus. Angiotensin-converting enzyme 2 (ACE2; ACEH) is encoded by the ACE2 gene. ACE2 is part of the angiotensin-converting enzyme family of dipeptidyl carboxydipeptidases and has considerable homology to human angiotensin 1 converting enzyme. ACE2 is a cell surface expressed aminopeptidase that catalyzes the cleavage of angiotensin I into angiotensin 1-9, and angiotensin II into the vasodilator angiotensin 1-7. The organ- and cell-specific expression of this gene suggests that it may play a role in the regulation of cardiovascular and renal function, as well as fertility. In addition, the encoded protein is a functional receptor for the spike glycoprotein of the human coronavirus HCoV-NL63 and the human severe acute respiratory syndrome coronaviruses, SARS-COV and SARS-COV-2 (COVID-19 virus).

Both the human and mouse ACE2 genes are located on chromosome X, and each gene comprises 19 untranslated and coding exons of which 18 exons contain coding sequences. Coding exon numbering used throughout excludes the 5′ non-coding exon. Accordingly, “coding exon 1” refers to the first exon comprising coding sequences and subsequent coding exons are numbered accordingly. As such, and in connection with coding exons, the first intron following coding exon 1 may be referred to herein as intron 1.

An exemplary coding sequence for human ACE2 is assigned NCBI Accession Number NM_021804.3. An exemplary coding sequence for mouse ACE2 is assigned NCBI Accession Number NM_001130513.1. An exemplary human ACE2 protein is assigned UniProt Accession No. Q9BYF1-1. An exemplary mouse ACE2 protein is assigned UniProt Accession No. Q8R010-1. An exemplary humanized human/mouse ACE2 protein is set forth in SEQ ID NO:24, which comprises in operable linkage a mouse ACE2 signal peptide (SEQ ID NO: 26), a human ACE2 extracellular domain (SEQ ID NO:27), a mouse ACE2 transmembrane domain (SEQ ID NO:28), and a mouse ACE2 cytoplasmic domain (SEQ ID NO: 28).

A humanized A (E2 locus can be an ACE2 locus in which the entire ACE2 gene is replaced with the corresponding orthologous human ACE2 sequence, or it can be an ACE2 locus in which only a portion of the ACE2 gene is replaced with the corresponding orthologous human ACE2 sequence (i.e., humanized). Optionally, the corresponding orthologous human ACE2 sequence is modified to be codon-optimized based on codon usage in the non-human animal. Replaced (i.e., humanized) regions can include coding regions such as an exon, non-coding regions such as an intron, an untranslated region, or a regulatory region (e.g., a promoter, an enhancer, or a transcriptional repressor-binding element), or any combination thereof. As one example, exons corresponding to 1, 2, 3, 4, 5, 6, 7, 8, 9, 10, 11, 12, 13, 14, 15, 16, 17, 18, or all 19 exons of the ACE2 gene are humanized. Likewise, introns corresponding to 1, 2, 3, 4, 5, 6, 7, 8, 9, 10, 11, 12, 13, 14, 15, 16, 16, or all 18 introns of the human ACE2 gene can be humanized. For example, the coding region of a non-human (e.g., rodent, e.g., rat or mouse) ACE2 gene could be replaced with the corresponding orthologous human ACE2 region. Alternatively, a region of ACE2 encoding an extracellular domain may be humanized. For example, at a non-human animal endogenous ACE2 locus, coding sequences starting in exon 2 (also referred to as coding exon 1) (e.g., after an endogenous ACE2 signal peptide coding region) to exon 18 (also referred to herein as coding exon 17) (e.g., up to an endogenous transmembrane domain coding region) may be replaced with corresponding human ACE2 coding sequences. Likewise, endogenous non-human ACE2 introns corresponding to coding introns 1, 2, 3, 4, 5, 6, 7, 8, 9, 10, 11, 12, 13, 14, 15, and 16 of the human ACE2 can be humanized. Flanking untranslated regions including regulatory sequences can also be humanized. For example, the 5′ untranslated region (UTR), the 3′UTR, or both the 5′ UTR and the 3′ UTR can be humanized, or the 5′ UTR, the 3′UTR, or both the 5′ UTR and the 3′ UTR can remain endogenous. In one specific example, the 3′ UTR is humanized, but the 5′ UTR remains endogenous. Depending on the extent of replacement by orthologous sequences, regulatory sequences, such as a promoter, can be endogenous or supplied by the replacing human orthologous sequence. For example, the humanized ACE2 locus can include the endogenous non-human animal ACE2 promoter.

The ACE2 protein encoded by the humanized ACE2 locus can comprise one or more domains that are from a human ACE2 protein. For example, the ACE2 protein can comprise one or more or all of a human ACE2 extracellular domain, a human ACE2 transmembrane domain, and a human ACE2 cytoplasmic domain. As one example, the ACE2 protein can comprise only a human ACE2 extracellular domain. Optionally, the ACE2 protein encoded by the humanized ACE2 locus may also comprise one or more domains that are from the endogenous (i.e., native) non-human animal ACE2 protein. As one example, the ACE2 protein encoded by the humanized ACE2 locus can comprise an extracellular domain from a human ACE2 protein, a transmembrane domain from the endogenous (i.e., native) non-human animal ACE2 protein, and an N-terminal cytoplasmic domain from the endogenous (i.e., native) non-human animal ACE2 protein. Domains from a human ACE2 protein can be encoded by a fully humanized sequence (i.e., the entire sequence encoding that domain is replaced with the orthologous human A (E2 sequence) or can be encoded by a partially humanized sequence (i.e., some of the sequence encoding that domain is replaced with the orthologous human ACE2 sequence, and the remaining endogenous (i.e., native) sequence encoding that domain encodes the same amino acids as the orthologous human ACE2 sequence such that the encoded domain is identical to that domain in the human ACE2 protein.

As one example, the ACE2 protein encoded by the humanized ACE2 locus can comprise a human ACE2 extracellular domain. Optionally, the human ACE2 extracellular domain comprises, consists essentially of, or consists of a sequence that is at least 85%, 90%, 95%, 96%, 97%, 98%, 99%, or 100% identical to SEQ ID NO:27 and the ACE2 protein retains the activity of an ACE2 protein (e.g., retains the ability to catalyze the cleavage of angiotensin I into angiotensin 1-9, and angiotensin II into the vasodilator angiotensin 1-7, permit coronavirus (e.g., SARS-COV-2) infection, etc.). The ACE2 protein encoded by the humanized ACE2 locus may comprise an endogenous non-human animal ACE2 transmembrane domain (e.g., a mouse ACE2 transmembrane domain). Optionally, the non-human animal ACE2 transmembrane domain comprises, consists essentially of, or consists of a sequence that is at least 85%, 90%, 95%, 96%, 97%, 98%, 99%, or 100% identical to SEQ ID NO: 28 and the ACE2 protein retains the activity of the native ACE2. For example, the ACE2 protein encoded by the humanized ACE2 locus can comprise, consist essentially of, or consist of a sequence that is at least 85%, 90%, 95%, 96%, 97%, 98%, 99%, or 100% identical to SEQ ID NO: 29 and the ACE2 protein retains the activity of the native ACE2.

In some embodiments, a non-human animal disclosed herein contains a humanized ACE2 gene in the genome that includes a nucleotide sequence of a human ACE2 gene and a nucleotide sequence of an endogenous ACE2 gene, wherein the nucleotide sequence of the human ACE2 gene and the nucleotide sequence of the endogenous ACE2 gene are operably linked to each other such that the humanized ACE2 gene encodes an ACE2 protein and is under control of a 5′ regulatory element(s), such as the promoter and/or enhancer(s), of the endogenous rodent ACE2 gene.

Optionally, a humanized ACE2 gene can comprise other elements. Examples of such elements can include selection cassettes, reporter genes, recombinase recognition sites, or other elements. Alternatively, the humanized ACE2 locus can lack other elements (e.g., can lack a selection marker or selection cassette). Examples of suitable reporter genes and reporter proteins are disclosed elsewhere herein. Examples of suitable selection markers include neomycin phosphotransferase (neor), hygromycin B phosphotransferase (hygr), puromycin-N-acetyltransferase (puror), blasticidin S deaminase (bsrr), xanthine/guanine phosphoribosyl transferase (gpt), and herpes simplex virus thymidine kinase (HSV-k). Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

Other elements such as reporter genes or selection cassettes can be self-deleting cassettes flanked by recombinase recognition sites. See, e.g., U.S. Pat. No. 8,697,851 and US 2013/0312129, each of which is herein incorporated by reference in its entirety for all purposes. As an example, the self-deleting cassette can comprise a Crei gene (comprises two exons encoding a Cre recombinase, which are separated by an intron) operably linked to a mouse Prm1 promoter and a neomycin resistance gene operably linked to a human ubiquitin promoter. By employing the Prm1 promoter, the self-deleting cassette can be deleted specifically in male germ cells of F0 animals. The polynucleotide encoding the selection marker can be operably linked to a promoter active in a cell being targeted. Examples of promoters are described elsewhere herein. As another specific example, a self-deleting selection cassette can comprise a hygromycin resistance gene coding sequence operably linked to one or more promoters (e.g., both human ubiquitin and EM7 promoters) followed by a polyadenylation signal, followed by a Crei coding sequence operably linked to one or more promoters (e.g., an mPrm1 promoter), followed by another polyadenylation signal, wherein the entire cassette is flanked by loxP sites.

The humanized A (E2 locus can also be a conditional allele. For example, the conditional allele can be a multifunctional allele, as described in US 2011/0104799, herein incorporated by reference in its entirety for all purposes. For example, the conditional allele can comprise: (a) an actuating sequence in sense orientation with respect to transcription of a target gene; (b) a drug selection cassette (DSC) in sense or antisense orientation; (c) a nucleotide sequence of interest (NSI) in antisense orientation; and (d) a conditional by inversion module (COIN, which utilizes an exon-splitting intron and an invertible gene-trap-like module) in reverse orientation. See, e.g., US 2011/0104799. The conditional allele can further comprise recombinable units that recombine upon exposure to a first recombinase to form a conditional allele that (i) lacks the actuating sequence and the DSC; and (ii) contains the NSI in sense orientation and the COIN in antisense orientation. See, e.g., US 2011/0104799.

One exemplary humanized A (E2 locus (e.g., a humanized mouse A (E2 locus) is one in which part of the first coding exon through part of the 17th coding exon of the endogenous ACE2 gene are replaced with the corresponding human sequence. These exons encode the extracellular domain of ACE2. Optionally, the humanized sequence can be through the stop codon and 3′ UTR, and optionally into the sequence just downstream of the 3′ UTR. Optionally, a portion o the intron upstream of coding exon 1 is also humanized.

### B. Transmembrane Serine Protease (TMPRSS) and Humanized TMPRSS Loci

Type II transmembrane serine proteases, also referred to herein as “TTSPs, “TMPRSS,” “transmembrane serine protease,” and the like, are a family of proteins characterized by an N-terminal transmembrane domain and a C-terminal extracellular serine protease domain. At least 18 members have been identified in the family, which are grouped into four subfamilies (Bugge et al. (2009) J. Biol. Chem. 284 (35): 23177-23181). All members share several common structural features that define the family, including (i) a short N-terminal cytoplasmic domain, (ii) a transmembrane domain, and (iii) an extracellular that contains a protease domain and a stem region that links the transmembrane domain with the protease domain. The stem region contains a combination of modular structural domains of six different types: a SEA (sea urchin sperm protein/enteropeptidase/agrin) domain, a group A scavenger receptor domain, a LDLA (low-density lipoprotein receptor class A) domain, a CUB (Cls/Clr urchin embryonic growth factor, bone morphogenetic protein-1) domain, a MAM (meprin/A5 antigen/receptor protein phosphatase mu) domain, and a frizzled domain. See review by Bugge et al. (2009), supra. For example, TMPRSS2 and TMPRSS4, both of which belong to the hepsin/TMPRSS subfamily, have a group A scavenger receptor domain, preceded by a single LDLA domain in the stem region. TMPRSS11D, also known as “HAT” for human airway trypsin-like protease that belongs to the HAT/DESC subfamily, has a single SEA domain. See the first figure of Bugge et al. (2009), supra.

Type II transmembrane serine proteases are produced initially as inactive proenzymes that require activation by cleavage following a basic amino acid residue in a consensus activation motif preceding the protease domain. Some of the activated proteases remain membrane bound as a result of a disulfide bond between the prodomain and the protease domain. The extracellular domains are considered to be critical for cellular localization, activation, inhibition, and/or substrate specificity of these proteases (Bugge et al. (2009), supra; Szabo et al., Int. J. Biochem. Cell Biol. 40:1297-1316 (2008)).

Various biochemical and pathophysiological information has been documented for members of the type II transmembrane serine proteases. TMPRSS2, TMPRSS4 and TMPRSS11D have been shown in vitro to cleave influenza A hemagglutinin (HA), which is the first essential step in the influenza viral life cycle. TMPRSS2 and TMPRSS4 appears to be involved in cleaving priming the S protein of coronavirus, e.g., SARS-COV-2, to initiate viral infection. Following receptor engagement, SARS-COV-2 S is processed by TMPRSS2 to allow release the viral contents into the host cell cytosol. Hoffman et al. (2020) (el/181:271-80; Matsuyama et al. (2020) Proc. Natl. Acad. Sci. USA 17:7001-3. It has also been shown that TMPRSS2 and TMPRSS4 serine proteases mediate SARS-COV-2 infection of human mature enterocytes from the apical surface by inducing cleavage of the S protein and enhancing membrane fusion. Zhang et al. recently showed that TMPRSS11A also cleaved the SARS-COV-2 protein in cell culture-derived medium, suggesting that other TTSPs may participate in SARS-COV-2 infection. Id. (2020) J. Biol. Chem.

Accordingly, to increase the efficacy of the non-human animals and non-human animal cell described herein as models to evaluate the course of human coronavirus infection and/or therapies therefor, a non-human animal as described herein may comprise multiple humanized loci. In some non-limiting embodiments, a non-human animal herein (e.g., rodent, e.g., rat or mouse) comprising a human or humanized ACE2 locus that expresses a recombinant ACE2 protein may also further comprise a humanized TMPRSS, e.g., a TMPRSS2, TMPRSS4 and/or TMPRSS11 locus. See, e.g., U.S. Pat. Nos. 10,070,631 and 10,070,632, each of which is incorporated herein by reference in its entirety.

In some embodiments, the rodent disclosed herein contains a humanized TMPRSS gene in the genome that includes a nucleotide sequence of an endogenous non-human TMPRSS gene and a nucleotide sequence of a human TMPRSS gene, wherein the nucleotide sequence of the endogenous non-human TMPRSS gene and the nucleotide sequence of the human TMPRSS gene are operably linked to each other such that the humanized TMPRSS gene encodes a TMPRSS protein and is under control of a 5′regulatory element(s), such as the promoter and/or enhancer(s), of the endogenous non-human TMPRSS gene.

The present disclosure is particularly directed to like-for-like humanization; in other words, a nucleotide sequence of an endogenous non-human TMPRSS gene is operably linked to a nucleotide sequence of an orthologous, e.g., corresponding, human TMPRSS gene to form a humanized gene. For example, in some embodiments, a nucleotide sequence of an endogenous non-human TMPRSS2 gene is operably linked to a nucleotide sequence of a human TMPRSS2 gene to form a humanized TMPRSS2 gene. In other embodiments, a nucleotide sequence of an endogenous non-human TMPRSS4 gene is operably linked to a nucleotide sequence of a human TMPRSS4 gene to form a humanized TMPRSS4 gene. In still other embodiments, a nucleotide sequence of an endogenous non-human TMPRSS11d gene is operably linked to a nucleotide sequence of a human TMPRSS11D gene to form a humanized TMPRSS11d gene.

In some embodiments, a genetically modified rodent of this invention contains a humanized TMPRSS gene in its genome, wherein the humanized TMPRSS gene encodes a recombinant TMPRSS protein that contains an extracellular portion that is substantially identical with the extracellular portion of a human TMPRSS protein. The term “extracellular” refers to the portion of a transmembrane protein that extends outside of the cell membrane, i.e., the extracellular portion of a transmembrane protein. The extracellular portion of a TMPRSS molecule includes a protease domain and a stem region that links the transmembrane domain with the protease domain. By an extracellular or polypeptide that is “substantially identical with the extracellular of a human TMPRSS protein”, it is meant in some embodiments, a polypeptide that is at least 85%, 90%, 95%, 95%, 99% or 100% identical in sequence with the extracellular portion of a human TMPRSS protein; in some embodiments, a polypeptide that differs from the extracellular of a human TMPRSS protein by not more than 10, 9, 8, 7, 6, 5, 4, 3, 2 or 1 amino acid(s); in some embodiments, a polypeptide that differs from the extracellular portion of a human TMPRSS protein only at the N- or C-terminus of the extracellular portion, e.g., by lacking amino acids or having additional amino acids at the at the N- or C-terminus of the extracellular portion; and in some embodiments, a polypeptide that is substantially the extracellular portion of a human TMPRSS protein. By “substantially the extracellular” of a human TMPRSS protein, it is meant a polypeptide that is identical with the extracellular portion, or differs from the extracellular portion by lacking 1-5 (i.e., 1, 2, 3, 4 or 5) amino acids or having additional 1-5 amino acids at the N- or C-terminus.

In some embodiments, the humanized TMPRSS gene encodes a recombinant TMPRSS protein that further contains a cytoplasmic and transmembrane portion that is substantially identical with the cytoplasmic and transmembrane portion of an endogenous non-human TMPRSS protein. By a cytoplasmic and transmembrane portion or polypeptide that is “substantially identical with the cytoplasmic and transmembrane portion of an endogenous non-human TMPRSS protein”, it is meant in some embodiments, a polypeptide that is at least 85%, 90%, 95%, 95%, 99% or 100% identical in sequence with the cytoplasmic and transmembrane portion of an endogenous non-human TMPRSS protein; in some embodiments, a polypeptide that differs from the cytoplasmic and transmembrane portion of an endogenous non-human TMPRSS protein by not more than 10, 9, 8, 7, 6, 5, 4, 3, 2 or 1 amino acid(s); in some embodiments, a polypeptide that differs from the cytoplasmic and transmembrane portion of an endogenous non-human TMPRSS protein only at the C-terminus, e.g., by lacking amino acids or having additional amino acids at the at the C-terminus of the transmembrane domain; and in some embodiments, a polypeptide composed of the cytoplasmic domain and substantially the transmembrane domain of an endogenous non-human TMPRSS protein. By “substantially the transmembrane domain” of an endogenous non-human TMPRSS protein, it is meant a polypeptide that is identical with the transmembrane domain, or differs from the transmembrane domain by lacking 1-5 amino acids or having additional 1-5 amino acids at the C-terminus.

In some embodiments, the humanized TMPRSS gene in the genome of a genetically modified rodent includes a nucleotide sequence of an endogenous non-human TMPRSS gene and a nucleotide sequence of an orthologous human TMPRSS gene, wherein the nucleotide sequence of the orthologous human TMPRSS gene encodes a polypeptide substantially identical to the extracellular of the human TMPRSS protein encoded by the human TMPRSS gene. In certain embodiments, the nucleotide sequence of an orthologous human TMPRSS gene in a humanized TMPRSS gene encodes the extracellular of the human TMPRSS protein encoded by the human TMPRSS gene.

In some embodiments, the humanized TMPRSS gene in the genome of a genetically modified rodent includes a nucleotide sequence of an endogenous non-human TMPRSS gene and a nucleotide sequence of an orthologous human TMPRSS gene, wherein the nucleotide sequence of an endogenous non-human TMPRSS gene encodes a polypeptide substantially identical to the cytoplasmic and transmembrane portion of the endogenous non-human TMPRSS protein encoded by the non-human TMPRSS gene. In specific embodiments, the nucleotide sequence of an endogenous non-human TMPRSS gene present in a humanized TMPRSS gene encodes the cytoplasmic and transmembrane domains of the endogenous non-human TMPRSS protein encoded by the endogenous non-human TMPRSS gene.

In some embodiments, a humanized TMPRSS gene results from a replacement of a nucleotide sequence of an endogenous non-human TMPRSS gene at an endogenous non-human TMPRSS locus with a nucleotide sequence of an orthologous human TMPRSS gene.

In some embodiments, a contiguous genomic sequence of a non-human TMPRSS gene at an endogenous non-human TMPRSS locus has been replaced with a contiguous genomic sequence of an orthologous human TMPRSS gene to form a humanized TMPRSS gene.

In specific embodiments, a contiguous genomic sequence of a human TMPRSS gene inserted into an endogenous non-human TMPRSS gene includes exons, in full or in part, of a human TMPRSS gene, that encode an extracellular that is substantially identical with the extracellular of the human TMPRSS protein encoded by the human TMPRSS gene.

In certain embodiments, the genomic sequence of an endogenous non-human TMPRSS gene that remains at an endogenous non-human TMPRSS locus after the humanization replacement and is operably linked to the inserted contiguous human TMPRSS genomic sequence encodes a cytoplasmic and transmembrane portion that is substantially identical with the cytoplasmic and transmembrane portion of the endogenous non-human TMPRSS protein encoded by the endogenous non-human TMPRSS gene.

In circumstances where an endogenous TMPRSS protein and a human TMPRSS protein share common amino acids near the junction between the transmembrane domain and the extracellular domain, it may not be necessary to insert a human TMPRSS genomic sequence that encodes precisely the extracellular domain of the human TMPRSS protein. It is possible to insert a slightly longer or shorter genomic sequence of a human TMPRSS gene, which encodes substantially the extracellular domain of the human TMPRSS protein, in operable linkage to a genomic sequence of an endogenous non-human TMPRSS gene that encodes the cytoplasmic domain and substantially the transmembrane domain of the endogenous non-human TMPRSS protein, such that the recombinant TMPRSS protein encoded by the resulting humanized TMPRSS gene includes an extracellular domain that is identical with the extracellular domain of the human TMPRSS protein and a transmembrane domain that is identical with the transmembrane domain of the endogenous non-human TMPRSS protein.

In some embodiments, the nucleotide sequence of a human TMPRSS gene included in a humanized TMPRSS gene also includes the 3′ untranslated region (“UTR”) of the human TMPRSS gene. In certain embodiments, in addition to the 3′ UTR of a human TMPRSS gene, a humanized TMPRSS gene also includes an additional human genomic sequence from the human TMPRSS gene locus, following the human TMPRSS 3′ UTR. The additional human genomic sequence can consist of at least 10-200 bp, e.g., 50 bp, 75 bp, 100 bp, 125 bp, 150 bp, 175 bp, 200 bp, or more, found in the human TMPRSS gene locus immediately downstream of the 3′ UTR of the human TMPRSS gene. In other embodiments, the nucleotide sequence of a human TMPRSS gene present in a humanized TMPRSS gene does not include a human 3′ UTR; instead, the 3′ UTR of an endogenous non-human TMPRSS gene is included and immediately follows the stop codon of the humanized TMPRSS gene. For example, a humanized TMPRSS gene can include a nucleotide sequence of an endogenous non-human TMPRSS gene containing exon sequences encoding the cytoplasmic and transmembrane domains of the endogenous non-human TMPRSS protein, followed by a nucleotide sequence of a human TMPRSS gene containing exon sequences encoding the extracellular through the stop codon of the human TMPRSS protein, with the 3′ UTR of the endogenous non-human TMPRSS gene following immediately after the stop codon.

In some embodiments, a humanized TMPRSS gene results in an expression of the encoded recombinant TMPRSS protein in a rodent. In some embodiments, a recombinant TMPRSS protein is expressed in a pattern comparable with, or substantially the same as, a counterpart non-human TMPRSS protein in a control rodent (e.g., a rodent without the humanized TMPRSS gene). In some embodiments, a recombinant TMPRSS protein is expressed at a level comparable with, or substantially the same as, a counterpart non-human TMPRSS protein in a control rodent (e.g., a rodent without the humanized TMPRSS gene). In certain embodiments, a recombinant TMPRSS protein is expressed and detected at the cell surface. In certain embodiments, a recombinant TMPRSS protein or a soluble form (e.g., a shed extracellular form) is expressed and detected in serum of a rodent, e.g., at a level comparable with, or substantially the same as, a counterpart non-human TMPRSS protein or a soluble form thereof in a control rodent. In the context of comparing a humanized gene or protein in a humanized rodent with an endogenous rodent gene or protein in a control rodent, the term “comparable” means that the molecules or levels being compared may not be identical to one another but are sufficiently similar to permit comparison there between so that conclusions may reasonably be drawn based on differences or similarities observed; and the term “substantially the same” in referring to expression levels means that the levels being compared are not different from one another by more than 20%, 19%, 18%, 17%, 16%, 15%, 14%, 13%, 12%, 11%, 10%, 9%, 8%, 7%, 6%, 5%, 4%, 3%, 2%, or 1%.

### C. Non-Human Cells and Non-Human Animals Comprising a Humanized ACE2 and TMPRSS Loci

Non-human animal cells and non-human animals comprising a humanized ACE2 locus and a humanized TMPRSS as described elsewhere herein are provided. The cells or non-human animals can be heterozygous or homozygous for the humanized ACE2 locus and/or for the TMRPSS locus. A diploid organism has two alleles at each genetic locus. Each pair of alleles represents the genotype of a specific genetic locus. Genotypes are described as homozygous if there are two identical alleles at a particular locus and as heterozygous if the two alleles differ.

The non-human animal cells provided herein can be, for example, any non-human cell comprising a humanized ACE2 locus or a genomic locus homologous or orthologous to the human ACE2 locus, and a humanized TMPRSS locus or a genomic locus homologous or orthologous to a human TMPRSS locus. The cells can be eukaryotic cells, which include, for example, fungal cells (e.g., yeast), plant cells, animal cells, mammalian cells, non-human mammalian cells, and human cells. The term “animal” includes mammals, fishes, and birds. A mammalian cell can be, for example, a non-human mammalian cell, a rodent cell, a rat cell, a mouse cell, or a hamster cell. Other non-human mammals include, for example, non-human primates, monkeys, apes, orangutans, cats, dogs, rabbits, horses, bulls, deer, bison, livestock (e.g., bovine species such as cows, steer, and so forth; ovine species such as sheep, goats, and so forth; and porcine species such as pigs and boars). Birds include, for example, chickens, turkeys, ostrich, geese, ducks, and so forth. Domesticated animals and agricultural animals are also included. The term “non-human” excludes humans.

The cells can also be any type of undifferentiated or differentiated state. For example, a cell can be a totipotent cell, a pluripotent cell (e.g., a human pluripotent cell or a non-human pluripotent cell such as a mouse embryonic stem (ES) cell or a rat ES cell), or a non-pluripotent cell. Totipotent cells include undifferentiated cells that can give rise to any cell type, and pluripotent cells include undifferentiated cells that possess the ability to develop into more than one differentiated cell types. Such pluripotent and/or totipotent cells can be, for example, ES cells or ES-like cells, such as an induced pluripotent stem (iPS) cells. ES cells include embryo-derived totipotent or pluripotent cells that are capable of contributing to any tissue of the developing embryo upon introduction into an embryo. ES cells can be derived from the inner cell mass of a blastocyst and are capable of differentiating into cells of any of the three vertebrate germ layers (endoderm, ectoderm, and mesoderm).

The cells provided herein can also be germ cells (e.g., sperm or oocytes). The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the cells can be liver cells, such as hepatoblasts or hepatocytes.

Suitable cells provided herein also include primary cells. Primary cells include cells or cultures of cells that have been isolated directly from an organism, organ, or tissue. Primary cells include cells that are neither transformed nor immortal. They include any cell obtained from an organism, organ, or tissue which was not previously passed in tissue culture or has been previously passed in tissue culture but is incapable of being indefinitely passed in tissue culture. Such cells can be isolated by conventional techniques and include, for example, hepatocytes.

Other suitable cells provided herein include immortalized cells. Immortalized cells include cells from a multicellular organism that would normally not proliferate indefinitely but, due to mutation or alteration, have evaded normal cellular senescence and instead can keep undergoing division. Such mutations or alterations can occur naturally or be intentionally induced. A specific example of an immortalized cell line is the HepG2 human liver cancer cell line. Numerous types of immortalized cells are well known. Immortalized or primary cells include cells that are typically used for culturing or for expressing recombinant genes or proteins.

The cells provided herein also include one-cell stage embryos (i.e., fertilized oocytes or zygotes). Such one-cell stage embryos can be from any genetic background (e.g., BALB/c, C57BL/6, 129, or a combination thereof for mice), can be fresh or frozen, and can be derived from natural breeding or in vitro fertilization.

The cells provided herein can be normal, healthy cells, or can be diseased or mutant-bearing cells.

Non-human animals comprising a humanized ACE2 locus and a humanized TMPRSS locus as described herein can be made by the methods described elsewhere herein. The term “animal” includes mammals, fishes, and birds. Non-human mammals include, for example, non-human primates, monkeys, apes, orangutans, cats, dogs, horses, bulls, deer, bison, sheep, rabbits, rodents (e.g., mice, rats, hamsters, and guinea pigs), and livestock (e.g., bovine species such as cows and steer; ovine species such as sheep and goats; and porcine species such as pigs and boars). Birds include, for example, chickens, turkeys, ostrich, geese, and ducks. Domesticated animals and agricultural animals are also included. The term “non-human animal” excludes humans. Preferred non-human animals include, for example, rodents, such as mice and rats.

The non-human animals can be from any genetic background. For example, suitable mice can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 129S1/SV, 129S1/Svlm), 129S2, 129S4, 129S5, 129S9/SvEvH, 129S6 (129/SvEvTac), 129S7, 129S8, 129T1, and 129T2. See, e.g., Festing et al. (1999) Mammalian Genome 10:836, herein incorporated by reference in its entirety for all purposes. Examples of C57BL strains include C57BL/A, C57BL/An, C57BL/GrFa, C57BL/Kal_wN, C57BL/6, C57BL/6J, C57BL/6ByJ, C57BL/6NJ, C57BL/10, C57BL/10ScSn, C57BL/10Cr, and C57BL/Ola. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 129S6 (129/SvEvTac) strain).

Similarly, rats can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rats can also be obtained from a strain derived from a mix of two or more strains recited above. For example, a suitable rat can be from a DA strain or an ACI strain. The ACI rat strain is characterized as having black agouti, with white belly and feet and an RT1^{avl} haplotype. Such strains are available from a variety of sources including Harlan Laboratories. The Dark Agouti (DA) rat strain is characterized as having an agouti coat and an RT1^{avl} haplotype. Such rats are available from a variety of sources including Charles River and Harlan Laboratories. Some suitable rats can be from an inbred rat strain. See, e.g., US 2014/0235933, herein incorporated by reference in its entirety for all purposes.

In some embodiments, a non-human animal comprising a genetically modified endogenous ACE2 locus as described herein expresses a recombinant ACE2 protein in an organ selected from the group consisting of colon, duodenum, kidney, heart, liver, lung, trachea, and any combination thereof. In some embodiments, the expression pattern of a recombinant ACE2 protein in a genetically modified non-human animal as described herein follows the expression pattern of a non-human animal ACE2 protein in a control non-human animal comprising a wildtype endogenous ACE2 locus.

In some embodiments, the recombinant ACE2 protein is expressed on epithelial cells. Accordingly, also described herein is a non-human animal cell expressing a recombinant ACE2 protein, optionally wherein the non-human animal cell (e.g., rat cell or mouse cell) is a somatic cell, optionally wherein the somatic cell is an epithelial cell. Non-limiting examples of epithetical cells that may express a recombination ACE2 protein as described herein include respiratory and/or gastrointestinal epithelial cells, e.g., an alveolar cell of the lung, an esophagus upper and stratified epithelial cell, an absorptive enterocyte from the ileum or colon, etc. In some embodiments, a non-human animal cell as described herein expresses the recombinant ACE2 protein in the epithelium of small intestine villi, surface epithelium of the large intestine (colon), the epithelium of large to small bronchioles and bronchi of the lung, respiratory epithelium of the trachea, proximal tubular epithelium of the kidney, respiratory epithelium of the nasal cavity, and/or the stratum granulosum and/or stratum spinosum of oral mucosa/tongue in the oral cavity.

### III. Methods of Using Non-Human Animals Comprising a Humanized ACE2 Locus for Assessing Coronavirus Infection and or Anti-Coronavirus Therapies

Various methods are provided for using the non-human animals comprising a human or humanized ACE2 locus and a TMPRSS locus for assessing coronavirus infection and/or the in vivo efficacy of human anti-coronavirus treatments. Because the non-human animals comprise a human or humanized ACE2 locus and a human or humanized TMPRSS locus, the non-human animals will more accurately reflect coronavirus infection mediated by human ACE2 or human anti-coronavirus therapies than non-human animals with a non-humanized A (E2 locus with or without a humanized TMPRSS locus. As one example, the methods can monitor coronavirus infection comprising infecting a non-human animal as described herein with a coronavirus that utilizes a human ACE2 and/or TMPRSS protein for infection. In some embodiments, a non-human animal as described herein may be infected by intranasal inhalation of the coronavirus, e.g., SARS-COV-2. In some embodiments, a non-human animal as described herein may be infected by intragastric injection.

In some embodiments, the method further comprises assessing the non-human animal for coronavirus related disorders and/or diseases, e.g., lung capacity, gastrointestinal disorders and/or clotting related disorders, e.g., ischemia, and/or disease progression. Disease progression may be monitored by obvious clinical signs, e.g., respiratory distress, neurological symptoms, death, etc. Disease progression may also be monitored by measuring the amount of replicating viruses of the coronavirus, e.g., SARS-COV-2, that may be isolated from organs (e.g., lungs, brain) of the infected animal, e.g., by well-known plaque assays.

In some embodiments, a non-human animal comprising a modified endogenous ACE2 locus and a modified TMPRSS locus as described herein is infected with a SARS-COV-2 strain, e.g., the non-human animal further comprises replicating SARS-COV-2. In some embodiments, a non-human animal comprising a modified endogenous ACE2 locus and a modified TMPRSS locus as described herein as described herein and replicating SARS-CoV-2 exhibits COVID-19 symptoms for at least one, at least two, at least three, at least four, or at least five days post infection. In some embodiments, the COVID-19 symptom is selected from the group consisting of viral replication in an organ, minimal to severe inflammation (perivascular, vascular, peribronchiolar, septa and alveoli), minimal to severe necrosis (vascular, bronchioles, septa and alveoli), minimal to severe syncytia (vascular endothelium, bronchiolar epithelium and alveolar epithelium), minimal to severe hypertrophy/hyperplasia (vascular endothelium, bronchiolar and alveolar epithelium), minimal to severe hemorrhage (bronchioles and alveoli), minimal to severe edema (bronchioles and alveoli), minimal to severe fibrin (alveoli) and/or minimal to severe hyaline membranes (alveoli), and any combination thereof. In some embodiments, the COVID-19 symptom is selected from the group consisting of viral replication in an organ, necrosis of epithelium, e.g., necrosis of bronchiolar epithelium, vasculitis, endothelialitis, alveolar hyperplasia and/or syncytia, bronchiolar hyperplasia and syncytia, alveolar hemorrhage, perivascular edema, and any combination thereof. In some embodiments, the amount of replicating virus isolated from an organ of an infected non-human animal comprising a genetically modified endogenous is directly correlated with the severity of at least one symptom selected from the group consisting of inflammation (perivascular, vascular, peribronchiolar, septa and alveoli), necrosis (vascular, bronchioles, septa and alveoli), syncytia (vascular endothelium, bronchiolar epithelium and alveolar epithelium), hypertrophy/hyperplasia (vascular endothelium, bronchiolar and alveolar epithelium), hemorrhage (bronchioles and alveoli), minimal to severe edema (bronchioles and alveoli), fibrin (alveoli) and/or hyaline membranes (alveoli), necrosis of epithelium, e.g., necrosis of bronchiolar epithelium, vasculitis, endothelialitis, alveolar hyperplasia and/or syncytia, bronchiolar hyperplasia and syncytia, alveolar hemorrhage, perivascular edema, and any combination thereof. In some embodiments, severity of a COVID19 symptom is scored on a scale of 0 to 4 (0-within normal limits, 1-minimal, 2-mild, 3-moderate and 4-severe).

In some embodiments, a method as described herein assesses or identifies a candidate agent capable of preventing, reducing or otherwise treating coronavirus infection and related disorders, (e.g., preventing, reducing or eliminating binding of the coronavirus (ligand of a human ACE2 protein) to the human ACE2 protein), the method comprising administering an antigen binding protein specific for a coronavirus to a non-human animal comprising a humanized ACE2 locus and a humanized TMPRSS locus and monitoring the non-human animal for coronavirus related disorders and/or diseases, e.g., lung capacity, gastrointestinal disorders and/or clotting related disorders, e.g., ischemia, wherein the non-human animal is infected with the coronavirus before, simultaneously with, or after the administration, and wherein a reduction of the coronavirus related disorders and/or diseases compared to that of a control animal identifies the candidate agent as capable of preventing, reducing or otherwise treating coronavirus infection and related disorders, e.g., identifies the candidate as capable of preventing, reducing or eliminating binding of the coronavirus (ligand of a human ACE2 protein) to human ACE2 protein.

In some embodiments, a non-human animal comprising a modified endogenous ACE2 locus and a modified TMPRSS locus as described herein is infected with a SARS-COV-2 strain, e.g., the non-human animal further comprises replicating SARS-COV-2, before, after, or simultaneously with an antigen binding protein (e.g., an antibody) specific for SARS-COV-2 (e.g., the spike protein of SARS-COV-2). Accordingly, in some embodiments, a non-human animal as described herein comprises a modified endogenous ACE2 locus, a modified endogenous TMPRSS locus, replicating SARS-COV-2, and an antigen binding protein that binds SARS-COV-2. In some embodiments, a method described herein comprises administering an antigen-binding protein that binds SARS-COV-2 and SARS-COV-2 to a non-human animal comprising a genetically modified endogenous ACE2 locus and a modified endogenous TMPRSS locus as described herein as described herein and monitoring the non-human animal for COVID-19 related symptoms, wherein the antigen-binding protein that binds SARS-COV-2 may be administered prior to, simultaneously with, or after the administration of SARS-COV-2. In some embodiments, the non-human animal is monitored within one week of administration of (infection with) SARS-COV-2. In some embodiments, the non-human animal is monitored for at least 3 days after administration of (infection with) SARS-COV-2. In some embodiments, the non-human animal is monitored for at least 3 days after administration of (infection with) SARS-COV-2. In some embodiments, the non-human animal is monitored for COVID-19 related symptoms 1 to 2 days after administration of (infection with) SARS-COV-2.

In some embodiments, the COVID-19 symptom is selected from the group consisting of viral replication in an organ, minimal to severe inflammation (perivascular, vascular, peribronchiolar, septa and alveoli), minimal to severe necrosis (vascular, bronchioles, septa and alveoli), minimal to severe syncytia (vascular endothelium, bronchiolar epithelium and alveolar epithelium), minimal to severe hypertrophy/hyperplasia (vascular endothelium, bronchiolar and alveolar epithelium), minimal to severe hemorrhage (bronchioles and alveoli), minimal to severe edema (bronchioles and alveoli), minimal to severe fibrin (alveoli) and/or minimal to severe hyaline membranes (alveoli), and any combination thereof. In some embodiments, the COVID-19 symptom is selected from the group consisting of viral replication in an organ, necrosis of epithelium, e.g., necrosis of bronchiolar epithelium, vasculitis, endothelialitis, alveolar hyperplasia and/or syncytia, bronchiolar hyperplasia and syncytia, alveolar hemorrhage, perivascular edema, and any combination thereof.

In some embodiments, the dose of the antigen-binding protein is inversely correlated with the amount of replicating virus isolated from an organ of an infected non-human animal comprising a genetically modified endogenous and with the severity of at least one symptom selected from the group consisting of inflammation (perivascular, vascular, peribronchiolar, septa and alveoli), necrosis (vascular, bronchioles, septa and alveoli), syncytia (vascular endothelium, bronchiolar epithelium and alveolar epithelium), hypertrophy/hyperplasia (vascular endothelium, bronchiolar and alveolar epithelium), hemorrhage (bronchioles and alveoli), minimal to severe edema (bronchioles and alveoli), fibrin (alveoli) and/or hyaline membranes (alveoli), necrosis of epithelium, e.g., necrosis of bronchiolar epithelium, vasculitis, endothelialitis, alveolar hyperplasia and/or syncytia, bronchiolar hyperplasia and syncytia, alveolar hemorrhage, perivascular edema, and any combination thereof. In some embodiments, severity of a COVID19 symptom is scored on a scale of 0 to 4 (0-within normal limits, 1-minimal, 2-mild, 3-moderate and 4-severe).

### IV. Methods of Making Non-Human Animals Comprising Humanized ACE2 and TMPRSS2 LOCI

Various methods are provided for making a non-human animal comprising a humanized ACE2 locus as disclosed elsewhere herein. Any convenient method or protocol for producing a genetically modified organism is suitable for producing such a genetically modified non-human animal. See, e.g., Cho et al. (2009) Current Protocols in Cell Biology 42:19.11:19.11.1-19.11.22 and Gama Sosa et al. (2010) Brain Struct. Funct. 214 (2-3): 91-109, each of which is herein incorporated by reference in its entirety for all purposes. Such genetically modified non-human animals can be generated, for example, through gene knock-in at a targeted A (E2 locus.

For example, the method of producing a non-human animal comprising a humanized ACE2 locus and a humanized TMPRSS locus can comprise first generating a pluripotent cell (e.g., a non-human animal ES cell) to comprise a humanized ACE2 locus and then further modifying the modified pluripotent cell to further comprise a humanized TMPRSS locus, and generating a non-human animal with the pluripotent cell modified to comprise both the humanized ACE2 locus and the humanized TMPRSS locus. Modifying the pluripotent cell (e.g., non-human ES cell) to comprise a humanized A (E2 locus may comprise (1) modifying the genome of a pluripotent cell to comprise the humanized ACE2 locus and (2) identifying or selecting the genetically modified pluripotent cell comprising the humanized ACE2 locus. Modifying the pluripotent cell (e.g., non-human ES cell) to comprise a humanized TMPRSS locus may comprise (1) modifying the genome of a pluripotent cell to comprise the humanized TMPRSS locus and (2) identifying or selecting the genetically modified pluripotent cell comprising the humanized TMPRSS locus. Modified ES cells may then be introduced into a non-human animal host embryo; and the host embryo implanted in a surrogate mother for gestation. Optionally, the host embryo comprising modified pluripotent cell (e.g., a non-human ES cell) can be incubated until the blastocyst stage before being implanted into and gestated in the surrogate mother to produce an F0 non-human animal. The surrogate mother can then produce an F0 generation non-human animal comprising the humanized ACE2 locus and the humanized TMPRSS locus.

The methods can further comprise identifying a cell or animal having a modified target genomic locus. Various methods can be used to identify cells and animals having a targeted genetic modification.

The screening step can comprise, for example, a quantitative assay for assessing modification of allele (MOA) of a parental chromosome. For example, the quantitative assay can be carried out via a quantitative PCR, such as a real-time PCR (qPCR). The real-time PCR can utilize a first primer set that recognizes the target locus and a second primer set that recognizes a non-targeted reference locus. The primer set can comprise a fluorescent probe that recognizes the amplified sequence.

Other examples of suitable quantitative assays include fluorescence-mediated in situ hybridization (FISH), comparative genomic hybridization, isothermic DNA amplification, quantitative hybridization to an immobilized probe(s), INVADER® Probes, TAQMAN® Molecular Beacon probes, or ECLIPSE™ probe technology (see, e.g., US 2005/0144655, incorporated herein by reference in its entirety for all purposes).

An example of a suitable pluripotent cell is an embryonic stem (ES) cell (e.g., a mouse ES cell or a rat ES cell). The modified pluripotent cell can be generated, for example, through recombination by (a) introducing into the cell, in any order, a first and second targeting vector, each comprising a distinct insert nucleic acid flanked by 5′ and 3′ homology arms corresponding to 5′ and 3′ target sites, wherein one insert nucleic acid comprises a humanized ACE2 locus and an other insert nucleic acid comprises a humanized TMPRSS locus; and (b) identifying at least one cell comprising in its genome both insert nucleic acids integrated at the target genomic locus. Alternatively, the modified pluripotent cell can be generated by (a) introducing into the cell: (i) a nuclease agent, wherein the nuclease agent induces a nick or double-strand break at a recognition site within the target genomic locus; and (ii) one or more targeting vectors comprising an insert nucleic acid flanked by 5′ and 3′ homology arms corresponding to 5′ and 3′ target sites located in sufficient proximity to the recognition site, wherein the insert nucleic acid(s) comprises the humanized ACE2 locus and/or the humanized TMPRSS locus; and (b) identifying at least one cell comprising a modification (e.g., integration of the insert nucleic acid) at the target genomic locus. Any nuclease agent that induces a nick or double-strand break into a desired recognition site can be used. Examples of suitable nucleases include a Transcription Activator-Like Effector Nuclease (TALEN), a zinc-finger nuclease (ZFN), a meganuclease, and Clustered Regularly Interspersed Short Palindromic Repeats (CRISPR)/CRISPR-associated (Cas) systems or components of such systems (e.g., CRISPR/Cas9). See, e.g., US 2013/0309670 and US 2015/0159175, each of which is herein incorporated by reference in its entirety for all purposes.

The donor cell can be introduced into a host embryo at any stage, such as the blastocyst stage or the pre-morula stage (i.e., the 4 cell stage or the 8 cell stage). Progeny that are capable of transmitting the genetic modification though the germline are generated. See, e.g., U.S. Pat. No. 7,294,754, herein incorporated by reference in its entirety for all purposes.

Alternatively, the method of producing the non-human animals described elsewhere herein can comprise: (1) modifying the genome of a one-cell stage embryo to comprise the humanized ACE2 locus using the methods described above for modifying pluripotent cells; (2) selecting the genetically modified embryo; and (3) implanting and gestating the genetically modified embryo into a surrogate mother. Progeny that are capable of transmitting the genetic modification though the germline are generated.

Nuclear transfer techniques can also be used to generate the non-human mammalian animals. Briefly, methods for nuclear transfer can include the steps of: (1) enucleating an oocyte or providing an enucleated oocyte; (2) isolating or providing a donor cell or nucleus to be combined with the enucleated oocyte; (3) inserting the cell or nucleus into the enucleated oocyte to form a reconstituted cell; (4) implanting the reconstituted cell into the womb of an animal to form an embryo; and (5) allowing the embryo to develop. In such methods, oocytes are generally retrieved from deceased animals, although they may be isolated also from either oviducts and/or ovaries of live animals. Oocytes can be matured in a variety of well-known media prior to enucleation. Enucleation of the oocyte can be performed in a number of well-known manners. Insertion of the donor cell or nucleus into the enucleated oocyte to form a reconstituted cell can be by microinjection of a donor cell under the zona pellucida prior to fusion. Fusion may be induced by application of a DC electrical pulse across the contact/fusion plane (electrofusion), by exposure of the cells to fusion-promoting chemicals, such as polyethylene glycol, or by way of an inactivated virus, such as the Sendai virus. A reconstituted cell can be activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated reconstituted cells, or embryos, can be cultured in well-known media and then transferred to the womb of an animal. See, e.g., US 2008/0092249, WO 1999/005266, US 2004/0177390, WO 2008/017234, and U.S. Pat. No. 7,612,250, each of which is herein incorporated by reference in its entirety for all purposes.

The various methods provided herein allow for the generation of a genetically modified non-human F0 animal wherein the cells of the genetically modified F0 animal comprise the humanized ACE2 locus and the humanized TMPRSS locus. It is recognized that depending on the method used to generate the F0 animal, the number of cells within the F0 animal that have the humanized ACE2 locus and the humanized TMPRSS locus will vary. The introduction of the donor ES cells into a pre-morula stage embryo from a corresponding organism (e.g., an 8-cell stage mouse embryo) via for example, the VELOCIMOUSE® method allows for a greater percentage of the cell population of the F0 animal to comprise cells having the nucleotide sequence of interest comprising the targeted genetic modification. For example, at least 50%, 60%, 65%, 70%, 75%, 85%, 86%, 87%, 87%, 88%, 89%, 90%, 91%, 92%, 93%, 94%, 95%, 96%, 97%, 98%, 99% or 100% of the cellular contribution of the non-human F0 animal can comprise a cell population having the targeted modification.

The cells of the genetically modified F0 animal can be heterozygous for the humanized ACE2 locus and/or the humanized TMPRSS locus or can be homozygous for the humanized A (E2 locus and/or the humanized TMPRSS locus.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

TABLE 3
Description of Sequences.

| SEQ | | |
|---|---|---|
| ID NO | Type | Description |
| 1 | DNA | mACE2 |
| 2 | Protein | mACE2-NP_001123985.1 |
| 3 | DNA | hACE2 |
| 4 | Protein | hACE2-NP_068576.1 |
| 5 | DNA | 7878 allele |
| 6 | DNA | 7878mTu Fwd Primer |
| 7 | DNA | 7878 mTu Probe |
| 8 | DNA | 7878 mTu Rev Primer |
| 9 | DNA | 7878mTD Fwd Primer |
| 10 | DNA | 7878mTD Probe |
| 11 | DNA | 7878mTD Rev Primer |
| 12 | DNA | 7878hTU Fwd Primer |
| 13 | DNA | 7878hTU Probe |
| 14 | DNA | 7878hTU Rev Primer |
| 15 | DNA | 7878hTD Fwd Primer |
| 16 | DNA | 7878hTD Probe |
| 17 | DNA | 7878hTD Rev Primer |
| 18 | DNA | 7878 Border A |
| 19 | DNA | 7878 Border B |
| 20 | DNA | 7878 Border C |
| 21 | DNA | 7878 Border D |
| 22 | DNA | 7879 Allele |
| 23 | DNA | 7879 Border E |
| 24 | Protein | Chimeric human/mouse ACE2 |
| 25 | DNA | Chimeric human/mouse ACE2 |
| 26 | Protein | mACE2 signal sequence |
| 27 | Protein | hACE2 extracellular domain |
| 28 | Protein | mACE2 transmembrane domain |
| 29 | Protein | mACE2 cytoplasmic domain |
| 30 | DNA | 90034mTU Fwd Primer |
| 31 | DNA | 90034mTU Probe |
| 32 | DNA | 90034mTU Rev Primer |
| 33 | DNA | 90034mretU Fwd Primer |
| 34 | DNA | 90034mretU Probe |
| 35 | DNA | 90034mretU Rev Primer |
| 36 | DNA | 90034mretU2 Fwd Primer |
| 37 | DNA | 90034mretU2 Probe |
| 38 | DNA | 90034mretU2 Rev Primer |
| 39 | DNA | 90034mretD Fwd Primer |
| 40 | DNA | 90034mretD Probe |
| 41 | DNA | 90034mretD Rev Primer |
| 42 | DNA | 90034mretD2 Fwd Primer |
| 43 | DNA | 90034mretD2 Probe |
| 44 | DNA | 90034mretD2 Rev Primer |
| 45 | DNA | 90034mretD3 Fwd Primer |
| 46 | DNA | 90034mretD3 Probe |
| 47 | DNA | 90034mretD3 Rev Primer |
| 48 | DNA | 90034mretD 4 Fwd Primer |
| 49 | DNA | 90034mretD4 Probe |
| 50 | DNA | 90034mretD4 Rev Primer |
| 51 | DNA | mGU |
| 52 | DNA | mGU2 |
| 53 | DNA | mGD |
| 54 | DNA | mGD2 |
| 55 | DNA | 90034 Border A |
| 56 | DNA | Human coding ex 3-4 Foward primer |
| 57 | DNA | Human coding ex 3-4 Reverse primer |
| 58 | DNA | Human coding ex 3-4 Probe |
| 59 | DNA | Human coding ex 16-17 Forward primer |
| 60 | DNA | Human coding ex 16-17 Reverse primer |
| 61 | DNA | Human coding ex 16-17 Probe |
| 62 | DNA | Mouse coding ex 11-12 Forward primer |
| 63 | DNA | Mouse coding ex 11-12 Reverse primer |
| 64 | DNA | Mouse coding ex 11-12 Probe |
| 65 | DNA | Mouse coding ex 17-18 Forward Primer |
| 66 | DNA | Mouse coding ex 17-18 Reverse Primer |
| 67 | DNA | Mouse coding ex 17-18 Probe |
| 68 | DNA | Mouse TMPRSS2 |
| 69 | Protein | Mouse TMPRSS2 |
| 70 | DNA | Human TMPRSS2 |
| 71 | Protein | Human TMPRSS2 |
| 72 | DNA | Portion of Modified BAC clone |
| | | bMQ-264A15 containing the human |
| | | TMPRSS2 genomic fragment and |
| | | the neomycin cassette, as |
| | | well as the upstream and downstream |
| | | insertion junctions |
| 73 | DNA | Humanization Tmprss2 genomic |
| | | fragment with cassette deleted |
| 74 | Protein | Humanized 7010 TMPRSS2 |
| 75 | DNA | Mouse TMPRSS4 |
| 76 | Protein | Mouse TMRPSS4 |
| 77 | DNA | Human TMPRSS4 |
| 78 | Protein | Human TMPRSS4 |
| 79 | DNA | Portion of modified BAC clone |
| | | RP23-71M15 containing the |
| | | neomycin cassette and the human |
| | | TMPRSS4 genomic |
| | | fragment, as well as the upstream and |
| | | downstream insertion junctions |
| 80 | DNA | Humanization Tmprss4 genomic |
| | | fragment with cassette deleted |
| 81 | Protein | Humanized “7224” TMPRSS4 |
| 82 | DNA | Mouse TMPRSS11d |
| 83 | Protein | Mouse TMRPSS11d |
| 84 | DNA | Human TMPRSS11D |
| 85 | Protein | Human TMPRSS11D |
| 86 | DNA | Portion of modified BAC clone |
| | | RP23-95N22 containing the human |
| | | TMPRSS11D genomic fragment and the |
| | | neomycin cassette, as well as the upstream |
| | | and downstream insertion junctions |
| 87 | DNA | Humanization Tmprss11d genomic |
| | | fragment with cassette deleted |
| 88 | Protein | Humanized “7226” TMPRSS11 |
| 89 | DNA | Junction A of FIG. 16B and 16C |
| 90 | DNA | Junction B of FIG. 16B |
| 91 | DNA | Junction C of FIG. 16B |
| 92 | DNA | Junction D of FIG. 16C |
| 93 | DNA | 7010U Fwd Primer |
| 94 | DNA | 7010U Probe (BHQ) |
| 95 | DNA | 7010U Rev Primer |
| 96 | DNA | 7010D Fwd Primer |
| 97 | DNA | 7010D Probe BHQ |
| 98 | DNA | 7010D Rev Primer |
| 99 | DNA | 7010hU Fwd Primer |
| 100 | DNA | 7010hU Probe (BHQ) |
| 101 | DNA | 7010hU Rev Primer |
| 102 | DNA | 7010hU Fwd Primer |
| 103 | DNA | 7010hU Probe (BHQ) |
| 104 | DNA | 7010hU Rev Primer |
| 105 | DNA | Junction A of FIG. 17B |
| 106 | DNA | Junction B of FIG. 17B |
| 107 | DNA | Junction C of FIG. 17B and 17C |
| 108 | DNA | Junction D of FIG. 17C |
| 109 | DNA | 7224mTU Fwd Primer |
| 110 | DNA | 7224mTU Probe (BHQ) |
| 111 | DNA | 7224mTU Rev Primer |
| 112 | DNA | 7224mTU2 Fwd Primer |
| 113 | DNA | 7224m TU2 Probe (BHQ) |
| 114 | DNA | 7224mTU2 Rev Primer |
| 115 | DNA | 7224mTD Fwd Primer |
| 116 | DNA | 7224mTD Probe (BHQ) |
| 117 | DNA | 7224mTD Rev Primer |
| 118 | DNA | 7224hTU Fwd Primer |
| 119 | DNA | 7224hTU Probe (BHQ) |
| 120 | DNA | 7224hTU Rev Primer |
| 121 | DNA | 7010hTD Fwd Primer |
| 122 | DNA | 7010hTD Probe (BHQ) |
| 123 | DNA | 7010hTD Rev Primer |
| 124 | DNA | junction sequence for |
| | | Tmprss11d humanization |
| 125 | DNA | junction sequence for |
| | | Tmprss11d humanization |
| 126 | DNA | junction sequence for |
| | | Tmprss11d humanization |
| 127 | DNA | |
| 128 | DNA | 7224mTU Fwd Primer |
| 129 | DNA | 7224mTU Probe (BHQ) |
| 130 | DNA | 7224mTU Rev Primer |
| 131 | DNA | 7224mTU2 Fwd Primer |
| 132 | DNA | 7224m TU2 Probe (BHQ) |
| 133 | DNA | 7224mTU2 Rev Primer |
| 134 | DNA | 7224mTD Fwd Primer |
| 135 | DNA | 7224mTD Probe (BHQ) |
| 136 | DNA | 7224mTD Rev Primer |
| 137 | DNA | 7224hTU Fwd Primer |
| 138 | DNA | 7224hTU Probe (BHQ) |
| 139 | DNA | 7224hTU Rev Primer |

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

### EXAMPLES

The following examples are provided for illustrative purposes only and are not intended to limit the scope of the invention.

### Example 1: Generation of Mice Comprising a Humanized ACE2 Locus

Described in this example is the generation of mice comprising a humanized ACE2 locus for use as a model useful in understanding coronavirus infection, particularly SARS-COV and SARS-COV-2 function, and validation of vaccination and/or treatment protocols therefor.

Illustrative (not-to-scale) diagrams of the human and mouse ACE2 gene are provided in FIG. 1A. NCBI Accession number NP-001123985.1 provides an exemplary non-limiting mouse ACE2 protein amino acid sequence, which is set forth as SEQ ID NO:2. An exemplary non-limiting example of a nucleotide sequence that encodes the exemplary non-limiting mouse ACE2 protein is set forth as SEQ ID NO:1. Nucleotides 1-51 of SEQ ID NO: 1 encodes the mouse ACE2 signal peptide (set forth as amino acids 1-17 of SEQ ID NO: 2), nucleotides 52-2220 of SEQ ID NO: 1 encode the mouse ACE2 extracellular domain set forth as amino acids 18-740 of SEQ ID NO:2), nucleotides 2221-2283 of SEQ ID NO:1 encode the mouse ACE2 transmembrane domain (set forth as amino acids 741-761 of SEQ ID NO: 2), and nucleotides 2284-2418 of SEQ ID NO: 1 encode the mouse ACE2 cytoplasmic domain (set forth as amino acids 762-805 of SEQ ID NO:2). NCBI Accession number NP-068576.1 provides an exemplary non-limiting human ACE2 protein amino acid sequence, which is set forth as SEQ ID NO:4. Amino acids 1-17 of SEQ ID NO:4 sets forth the amino acid sequence of the signal peptide of human ACE2 protein, amino acids 18-740 of SEQ ID NO: 4 sets forth the amino acid sequence of the extracellular domain of human ACE2 protein, amino acids 741-761 of SEQ ID NO:4 sets forth the amino acid sequence of the transmembrane domain of human ACE2 protein, and amino acids 762-805 of SEQ ID NO:4 sets forth the amino acid sequence of the cytoplasmic domain of human ACE2 protein. An exemplary non-limiting example of a nucleotide sequence that encodes the exemplary non-limiting mouse ACE2 protein is set forth as SEQ ID NO:3. Nucleotides 1-51 of SEQ ID NO: 3 encode a signal peptide amino acid sequence of human ACE2 protein (set forth as amino acids 1-17 of SEQ ID NO:4), nucleotides 52-2220 of SEQ ID NO:3 encode an extracellular domain amino acid sequence of human ACE2 protein (set forth as amino acids 18-740 of SEQ ID NO:4), nucleotides 2221-2283 of SEQ ID NO:3 encode a transmembrane domain amino acid sequence of human ACE2 protein (set forth as amino acids 741-761 of SEQ ID NO:4), and nucleotides 2284-2418 of SEQ ID NO:3 encode a cytoplasmic domain amino acid sequence of human ACE2 protein (set forth as amino acids 762-805 of SEQ ID NO: 4).

A large targeting vector comprising a 5′ homology arm comprising 14.9 kb from RP23-244L14 and 3′ homology arm comprising 126 kb from RP23-244L14 was generated to replace part of coding exon 1 through part of coding exon 17 (e.g., part of coding exon 1, intron 1, exons 2-16 and intervening introns, intron 16, and part of coding exon 17) of mouse ACE2 with the corresponding human sequence of ACE2. See, e.g., FIGS. 1A and 1B. The targeting vector is designed to replace 45,019 bp of the mouse sequence with 36,742 bp of the human sequence, which also is modified to comprise a self-deleting floxed neo cassette (loxP-mPrm1-Crei-pA-hUb1-em7-Neo-pA-loxP) inserted into the human intron 16. See, e.g., FIG. 1B.

Since the ACE2 locus is found on the X-chromosome, CRISPR/Cas9 components were introduced into male F1H4 mouse embryonic stem cells together with the large targeting vector described herein. Loss-of-allele assays using the primers and probes set forth in Table 4 were performed to detect loss of the endogenous mouse allele, and gain-of-allele assays using the primers and probes set forth in Table 5 were performed to detect gain of the humanized allele. Loss-of-allele and gain-of-allele assays are described, for example, in US 2014/0178879; US 2016/0145646; WO 2016/081923; and Frendewey et al. (2010) Methods Enzymol. 476:295-307, each of which is herein incorporated by reference in its entirety for all purposes.

TABLE 4
Mouse TAQMAN ® Loss-of-Allele Assays

| LOA | | |
|---|---|---|
| Assay | Primer/Probe | Sequence |
| 7878mTU | Fwd | CCCAGATGGCTAAATTCAATTGA (SEQ ID NO: 6) |
| | Probe (MBG) | TTCATCTGGAAAATTG (SEQ ID NO: 7) |
| | Rev | GGAATCTGGGCAAATAATTCATTC (SEQ ID NO: 8) |
| 7878mTD | Fwd | GGGCCGCATCAATGATGTC (SEQ ID NO: 9) |
| | Probe (BHQ) | TGGCCTGAATGATAACAGCCTGGA (SEQ ID NO: 10) |
| | Rev | GTGGCTCAAGTGTTGGGTGAATC (SEQ ID NO: 11) |

TABLE 5
Human TAQMAN ® Gain-of-Allele Assays

| GOA | | |
|---|---|---|
| Assay | Primer/Probe | Sequence |
| 7878hTU | Fwd | GTGAGGCTGGACTTGGGAAT (SEQ ID NO: 12) |
| | Probe (BHQ) | CCTTTCCTCTTTGTCACAGACCTCCA (SEQ ID NO: 13) |
| | Rev | GGAGGGCTCTGCCTGGATT (SEQ ID NO: 14) |
| 7878hTD | Fwd | GCAAAGGTCCCTTTCTTATGTGC (SEQ ID NO: 15) |
| | Probe (BHQ) | CCCAGTGCTACCTCCAAATGCCA (SEQ ID NO: 16) |
| | Rev | CAGGTCCTATGACCAAGTCTCTA (SEQ ID NO: 17) |

The resulting humanized mouse ACE2 allele comprising the self-deleting floxed neo cassette comprises a nucleotide sequence as set forth in SEQ ID NO:5 (referred to as the 7878 allele). FIG. 1B. FIG. 1B also provides the sequences at various junctions (A, B, C, D) of the 7878 allele. The nucleotide sequence of (A) the 5′ mouse/human junction is set forth as SEQ ID NO:18. FIG. 1B. The nucleotide sequence of (B) the 5′ human/cassette junction is set forth as SEQ ID NO:19. FIG. 1B. The nucleotide sequence of (C) the 3′ cassette/human junction is set forth as SEQ ID NO:20. FIG. 1B. The nucleotide sequence of (D) the 3′ human/mouse junction is set forth as SEQ ID NO:21.

F0 mice were then generated using the VELOCIMOUSE® method. See, e.g., U.S. Pat. Nos. 7,576,259; 7,659,442; 7,294,754; US 2008/007800; and Poueymirou et al. (2007) Nature Biotech. 25 (1): 91-99, each of which is herein incorporated by reference in its entirety for all purposes.

Upon removal of the self-deleting neomycin cassette with Cre recombinase, the loxP and cloning sites (77 bp) remain inserted in human intron 16. FIG. 1C. The resulting humanized mouse, cassette-deleted, ACE2 allele is set forth in SEQ ID NO:22 (referred to as the 7879 allele). FIG. 1C. FIG. 1C also provides the sequence at (E) cloning and loxp site after recombination, which sequence is set forth as SEQ ID NO:23.

The modified endogenous mouse ACE2 7879 allele encodes a chimeric human/mouse ACE2 protein under the regulatory control of endogenous mouse ACE2 promoter and other regulatory elements. The amino acid sequence of the encoded chimeric human/mouse ACE2 protein is set forth as SEQ ID NO:24. An exemplary nucleotide sequence, e.g., CDS, that encodes the chimeric human/mouse ACE protein is set forth as SEQ ID NO:25.

The chimeric human/mouse ACE2 protein comprises a mouse ACE2 signal sequence at amino acids 1-17 of SEQ ID NO:24 (the amino acid sequence of the signal sequence of the chimeric human/mouse ACE2 protein is set forth as SEQ ID NO:26 and may be encoded by nucleotides 1-51 of SEQ ID NO:25), a human ACE2 extracellular domain at amino acids 18-740 of SEQ ID NO:24 (the amino acid sequence of the extracellular domain of the chimeric human/mouse ACE2 protein is set forth as SEQ ID NO:27 and may be encoded by nucleotides 52-2220 of SEQ ID NO:25), a mouse ACE2 transmembrane domain at amino acids 741-761 of SEQ ID NO:24 (the amino acid sequence of the transmembrane domain of the chimeric human/mouse ACE2 protein is set forth as SEQ ID NO:28 and may be encoded by nucleotides 2221-2283 of SEQ ID NO:25), and a mouse ACE2 cytoplasmic domain at amino acids 762-805 of SEQ ID NO:24 (the amino acid sequence of the cytoplasmic domain of the chimeric human/mouse ACE2 protein is set forth as SEQ ID NO: 29 and may be encoded by nucleotides 2284-2418 of SEQ ID NO:25). The mouse portion(s) of the chimeric human/mouse ACE2 protein spans amino acids 1-19 and 741-805 of SEQ ID NO:24 (and may be encoded by nucleotides 1-57 and 2221-2418 of SEQ ID NO: 25, respectively). The human portion of the chimeric human/mouse ACE2 protein spans amino acids 20-740 of SEQ ID NO:24 (and may be encoded by nucleotides 58-2220 of SEQ ID NO: 25).

### Example 2: Characterization of Humanized ACE2 Mouse

Humanized ACE2 mice were compared to ACE2-null mice. ACE2-null mice lack an ACE coding sequence at an endogenous ACE2 allele (see, e.g., FIGS. 2A and 2B).

Expression Levels of Chimeric Human Mouse ACE2 Messenger RNA (mRNA)

The expression levels of the chimeric human/mouse ACE2 mRNA were analyzed by TaqMan qRT-PCR analysis as described herein. Messenger RNA levels in this Example were analyzed by Real-Time Quantitative Polymerase Chain Reaction (RT-qPCR). Total RNA from each sample was extracted and reverse transcribed using primers that amplify across intronic boundaries of mouse and human ACE2 genes. RT-qPCR was performed using probes and primers of readily available kits.

Specifically, 1 cm pieces of small intestine, colon, kidney, and liver were isolated directly into RNALater (Ambion by Life Technologies), and chilled to 4° C. Tissues were homogenized in TRIzol, and chloroform was used for phase separation. The aqueous phase, containing total RNA, was purified using the MagMAX™-96 for Microarrays Total RNA Isolation Kit (Ambion by Life Technologies) according to manufacturer's specifications. Genomic DNA was removed using RNase-Free DNase Set (Qiagen). mRNA was reverse-transcribed into cDNA using SuperScript® VILO™ Master Mix (Invitrogen by Life Technologies). cDNA was amplified with the SensiFAST Probe Lo-ROX (Meridian) using the 12K Flex System (Thermofisher). A housekeeping control gene (Gapdh) was used to normalize any cDNA input differences. Data were reported as the comparative CT method using delta delta CT.

Negative control mRNAs were extracted from mice that are deleted in the ACE2 gene (aka ACE2-null, or knockout, or KO). Positive control mRNAs were extracted from wild-type (WT) mice. Human control mRNAs from heart (Cat #1H30-50), kidney (Cat #1H50-50), lung (Cat #1H40-50), small intestine (Cat #1H24-50), and adult human tracheal epithelial cells (Cat #504-R25a) were purchased from Cell Applications Inc. (San Diego, CA). The sequences of the primers and probes used in the analysis are provided in Table 6 below. Note that exons denoted in Table 6 are numbered counting coding exons only.

TABLE 6

| | | | |
|---|---|---|---|
| | Forward | Reverse | |
| | Primer | Primer | Probe |
| | SEQ ID | SEQ ID | SEQ ID |
| Assay | NO | NO | NO |

ACE2

| | | | |
|---|---|---|---|
| Human coding ex 3-4 | 56 | 57 | 58 |
| Human coding ex 16-17 | 59 | 60 | 61 |
| Mouse coding ex 11-12 | 62 | 63 | 64 |
| Mouse coding ex 17-18 | 65 | 66 | 67 |

FIG. 4 shows that post natal (P4-P7) mice comprising an endogenous ACE2 locus modified to encode a humanized ACE2 protein expresses levels of the humanized ACE2 similar to the levels of wild-type ACE2 expressed by wild-type mice. Panel A and B provide data from the two human-specific assays (hEx3-4 and hEx16-17), which amplify only humanized ACE2 allele and normal human RNA. No amplification was detected in ACE2-KO tissues. Zooming into relative levels shows that humanized ACE2 allele is expressed at lower, but detectable, levels compared with human tissues. The mEx17-18 assay is shown in panel C. This assay amplifies both mouse and humanized ACE2 allele, but does not amplify the human samples. KO mice are negative controls. The mEx11-12 assay amplifies both human and mouse genes due to identity of the base pairs in this region between these species. Here it fails to amplify ACE2-KO tissues. The levels of humanized ACE2 mRNA in aged mice (P40) is shown in FIG. 5. As shown in FIG. 5, as mice age, the levels of humanized ACE2 mRNA in animal genetically expressing a humanized endogenous ACE2 locus remained low when compared to the levels of murine ACE2 mRNA in wildtype mice. Notably, the levels of murine ACE2 mRNA in wildtype mice increase in the colon, duodenum, and lungs as the mice age (FIGS. 4 and 5).

### Expression Levels of Chimeric Human Mouse ACE2 Protein in the Respiratory and Gastrointestinal Tracts

Pieces of tissues (lung, trachea, duodenum) from the same mouse cohort as qPCR experiment were harvested directly into chilled 4% paraformaldehyde and post-fixed overnight with mild rocking. Tissues were rinsed 3× with PBS, then serially dehydrated in 70%, 85%, 95%, and 100% ethanol, defatted with xylene, and embedded in paraffin.

Automated staining was performed on a Ventana Ultra autostainer (Roche Diagnostics) using Universal protocol. Four micrometer paraffin sections of different tissues were used.

Slides were deparaffinized with EZPrep (Roche Diagnostics) at 69° C. for 24 minutes; antigen retrieval was performed with CC1 buffer (Roche Diagnostics) at 100° C. for 56 minutes, followed by blocking with 10% normal horse serum (Vector Labs) for 32 minutes.

Anti-ACE2 antibody recognizing both mouse and human ectodomain (AF933, R&D Systems) or anti-ACE2 antibody recognizing only mouse ectodomain (AF3437 R&D Systems) were added at concentration of 0.5 ug/ml followed by 5-hour incubation at room temperature. Secondary horse anti-goat IgG antibodies (Vector Labs) at 1:200 dilution were incubated for 1 hour at room temperature, followed by detection with DABMap kit (Roche diagnostics) according to the preset protocol parameters. Slides were counterstained with Hematoxylin (Roche Diagnostics) for 32 minutes followed by incubation with Bluing Reagent (Roche Diagnostics) for 8 minutes, washed with soap and tap water, dehydrated in increasing concentrations of ethyl alcohol and xylenes and mounted with Cytoseal-60 (Thermo Scientific). All images were obtained at 20× magnification.

FIGS. 6 and 7 are representative images that show that a mouse comprising an endogenous ACE2 locus modified to encode a ACE2 protein expresses the ACE2 protein on respiratory and gastrointestinal epithelial cells. Provided in Table 7 is a summary of ACE2 expression as observed by immunohistochemistry in various organs of an endogenous ACE2 locus modified to encode a humanized ACE2 protein (hACE2), and newborn (P7) ACE2 knockout (KO) mice.

TABLE 7

| | 5 Wk WT | | P7 hACE2 | | P7 ACE2 KO | |
|---|---|---|---|---|---|---|
| | AF933 | AF3437 | AF933 | AF3437 | AF933 | AF3437 |
| Small intestine | ++ | +++ | +++ | − | − | − |
| | Staining in the epithelium of the villi | | | | | |
| Large intestine | ++ | +++ | ++ | − | − | − |
| (colon) | Staining in the surface epithelium | | | | | |
| Lungs | + | + | + | − | − | − |
| | Staining in the epithelium of large to small bronchioles and bronchi | | | | | |
| Trachea | + | + | + | − | − | − |
| | Staining in the respiratory epithelium | | | | | |
| Liver | + | + | | | | |
| | Staining in the sinusoidal endothelium | | | | | |
| Kidney | ++ | ++ | ++ | + | − | − |
| | Staining in the proximal tubular epithelium | | | | | |
| Heart | + | + | − | − | − | − |
| | Staining in the muscular junctions | | | | | |
| Nasal cavity | N/A | N/A | −/++ | − | − | − |
| | Staining in the respiratory epithelium | | | | | |
| Oral Cavity | N/A | N/A | +++ | − | − | − |

Staining in the Str. granulosum and
Str. spinosum of oral mucosa/tongue

The expression of hACE2 in hACE2 mice was similar to ACE2 expression in WT mice and humans with few exceptions. Staining of sinusoidal staining and muscular junctions in the heart observed in WT mice was absent in hACE2 mice. Staining in the nasal mucosa, oral mucosa and tongue was present in hACE2 mice but was absent in WT mice. These exceptions were likely due to human ACE2 gene expression in hACE2 mice.

### Example 3: Humanized ACE2 Mice as Models of SARS-COV-2 Infection and Use in

### Assessing Anti-SARS-COV-2 Therapies

Humanized ACE2 mice allow for the study of not only non-mouse adapted SARS-COV-2 infection, but also the efficacy of antibodies for improving SARS-COV-2-mediated pathologies.

Generally, mice expressing humanized ACE2 as described in Example 1 are treated with antibodies 1 day prior to infection with SARS-COV-2. Mice are then intranasally inoculated with SARS-COV-2 and monitored daily for weight loss and signs of clinical disease. At 2 days post-infection, mice are euthanized and lungs were harvested for lung pathology by histological analysis and qRT-PCR for viral titers.

### SARS-COV-2 Model

Humanized ACE2 mice as described in Example 1 were infected with a human SARS-COV-2 isolate, WA1 at doses of 102 (e.g., 10E2) PFU, 10³ (e.g., 10E3) PFU, 10+ (e.g., 10E4) PFU, or 10⁵ (e.g., 10E5) PFU. Mice were monitored and weighed daily for 7 days. No significant weight loss was observed among mice infected with SARS-COV-2 and control mice exposed to PBS only (FIG. 8).

Both plaque assay and qPCR for the presence of the N gene were performed on lung homogenates collected on days 2, 4. 7 and 10 post-infection. As shown in FIG. 9, replicating virus could be isolated at day 2 at all inoculums, however, all groups cleared the virus to below the limit of detection by day 4 post-infection. This trend is confirmed by qPCR. At day 2, mice at all inoculums had detectable virus which exhibited a dose dependent pattern. At day 4 post-infection, only those mice in the 10⁵ group had appreciable expression of the nucleocapsid gene of SARS-COV-2, FIG. 10.

H&E stained lung sections were examined for pathological changes and providing with a pathology score. Lung sections showed low levels of inflammatory infiltrate and damage (data not shown), demonstrating that the humanized ACE2 mice as described in Example 1 are feasible for use in antibody testing experiments and use of 1×10⁵ PFU of SARS-COV-2 WA1 is an appropriate inoculum.

### Antibody Prophylaxis Against SARS-Cov-2 in SARS-Cov-2 Model

Humanized ACE2 mice were prophylactically treated at 2 days prior to infection via intraperitoneal injection with either one of two single monoclonal antibody specific for SARS-COV-2 spike protein at 50 mg/kg, 5 mg/kg or 0.5 mg/kg or a combination of both antibodies, each at 25 mg/kg, 2.5 mg/kg, 0.25 mg/kg or 0.25 mg/kg diluted in PBS. On day 0, mice were anesthetized and intranasally infected with 1×10⁵ PFU of WA1 SARS-COV-2.

FIG. 11 shows that 50 μg of either a single anti-spike protein antibody or 25 ug each of a combination of two anti-spike antibodies were able to reduce weight loss of mice infected with SARS-COV-2 over the 2 days of observation.

On day 2 post-infection, mice were sacrificed, and lungs were harvested for analysis of lung pathology and viral titer. Both by plaque assay and by qPCR for the presence of the N gene, a reduction in viral titer was observed in a dose dependent fashion for both the single monoclonal antibodies as well as the combination. (FIG. 12 and FIG. 13). Lung sections of all infected control animals that received placebo showed SARS-COV-2 induced inflammation characterized by minimal to mild infiltration of lymphocytes, macrophages, and neutrophils, most commonly in the peribronchiolar and perivascular areas, and less commonly in the alveolar septa and vascular wall. Necrosis was most prominently observed in the bronchiolar epithelium. Inflammation in the vascular walls (vasculitis/endothelitis) was accompanied by endothelial hypertrophy/hyperplasia, endothelial syncytia, and vascular necrosis (FIG. 14). Other changes include alveolar and bronchiolar hyperplasia and syncytia, alveolar hemorrhage, and perivascular edema. Histopathological evaluation show that the monotherapies and combination therapy reduced the lung pathology and total pathology score in a dose-dependent manner; significant reduction was observed only at 50 mg/kg. (FIG. 15). This was correlated well with virus load data, which also showed a greatest reduction at 50 mg/kg (FIG. 15).

Experimental Materials and Methodologies for Examples 1-3 Viruses and Cells

SARS-COV-2 WA-1 was obtained from the CDC following isolation from a patient in Washington State (WA-1 strain-BEI #NR-52281). All virus stocks were stored at −80° C. until ready to use. VeroE6 cells from ATCC (catalog #CRL-1586) (Manassas, VA) were used for growing SAR-COV-2 virus as well as in plaque assays. VeroE6 cells were grown in DMEM (Invitrogen, Carlsbad, CA) with 10% FBS (Atlanta Biologicals, Lawerenceville, GA), 1% penicillin/streptomycin (Gemini Bioproducts, West Sacramento, CA) and 1% L-glutamine (Gibco).

### In Vivo Mouse Infections

All infections were performed in an animal biosafety level 3 facility using appropriate practices including HEPA filtered BCON caging system, HEPA filtered PAPR respirators and Tyvek suiting. Animals were anesthetized using a mixture of xylazine (0.38 mg/mouse) and ketamine (1.3 mg/mouse) in 50 μL total volume by intraperitoneal injection. Mice were inoculated intranasally with 50 μL of either PBS or 1×10⁵ PFU of WA1 SARS-CoV-2 after which all animals were monitored daily for weight loss. Mice were euthanized at day 2, 4 and 7 post-infection and lung tissue was harvested for further analysis. All animals were housed and used in accordance with appropriate Institutional Animal Care and Use Committee guidelines.

### Antibody Treatments

Mice were given monoclonal antibodies via intraperitoneal injection at 2 days prior to infection with SARS-COV-2. Mice received 50 mg/kg, 5 mg/kg or 0.5 mg/kg of a single monoclonal antibody or a combination of 2 monoclonal antibodies each at 25 mg/kg, 2.5 mg/kg or 0.25 mg/kg (totaling 50 mg/kg, 25 mg/kg and 0.5 mg/kg combined) diluted in sterile PBS (Quality Biological) to a total volume of 100 μL.

### Histology:

Lung sections were fixed in 4% paraformaldehyde (PFA) in phosphate-buffered saline (PBS) for a minimum of 48 h, after which they were sent to the Histology Core at the University of Maryland, Baltimore, for paraffin embedding and sectioning. Five-micrometer sections were prepared and used for hematoxylin and eosin (H&E) staining by the Histology Core Services. Sections were imaged at 10× magnification and figures were put together using Adobe Photoshop and Illustrator software.

Histopathological evaluation was done by a board-certified veterinary pathologist. The following parameters were evaluated: inflammation (perivascular, vascular, peribronchiolar, septa and alveoli), necrosis (vascular, bronchioles, septa and alveoli), syncytia (vascular endothelium, bronchiolar epithelium and alveolar epithelium), hypertrophy/hyperplasia (vascular endothelium, bronchiolar and alveolar epithelium), hemorrhage (bronchioles and alveoli), edema (bronchioles and alveoli), fibrin (alveoli) and hyaline membranes (alveoli). A 0-4 severity scoring scale was used (0-within normal limits, 1-minimal, 2-mild, 3-moderate and 4-severe) to score the above 21 parameters. A total pathology score was calculated for each mouse by adding the individual histopathological feature scores, and a maximum pathology score of 84 was possible for an individual animal. Statistical analysis was performed using one-way analysis of variance followed by the Tukey's HSD test and a P value of <0.05 was considered significant.

### Plaque Assay on Lung Homogenate

SARS-COV-2 lung titers were quantified by homogenizing mouse lungs in 1 mL phosphate buffered saline (PBS; Quality Biological Inc.) using 1.0 mm glass beads (Sigma Aldrich) and a Beadruptor (Omni International Inc). VeroE6 cells are plated in 6 well plates with 1×10⁵ cells per well. SARS-COV-2 virus titer in plaque forming units was determined by plaque assay. In the plaque assay, 25 μl of the lung homogenate is added to 225 ul of PBS and diluted 10 fold across a 6 point dilution curve with 200 μl of diluent added to each well. After 1 hour, a 3 ml agar overlay containing DMEM is added to each well. Plates are incubated for 3 days at 37° C. (5% CO2) before plaques are counted.

### Example 4: Humanization of an Endogenous TMPRSS2 Gene

This example illustrates exemplary methods of humanizing an endogenous gene encoding TMPRSS2 in a rodent (e.g., a mouse). The methods described in this example can be employed to humanize an endogenous TMPRSS2 gene of a rodent using any human sequence, or combination of human sequences (or sequence fragments) as desired.

A targeting vector for humanization of an endogenous TMPRSS2 gene was constructed using bacterial artificial chromosome (BAC) clones and VELOCIGENE® technology (see, e.g., U.S. Pat. No. 6,586,251 and Valenzuela et al. (2003) High-throughput engineering of the mouse genome coupled with high-resolution expression analysis, Nature Biotech. 21 (6): 652-659; incorporated herein by reference).

Briefly, mouse bacterial artificial chromosome (BAC) clone bMQ-264A15 containing a mouse TMPRSS2 gene was used and modified as follows. A DNA fragment was generated to include a 5′ mouse homology nucleotide sequence, a human TMPRSS2 genomic DNA of about 25,091 bp (containing the last 7 bp of coding exon 3, intron 3, and coding exon 4 through coding exon 13 (including the 3′ UTR which is part of coding exon 13), of a human TMPRSS2 gene), a self-deleting neomycin cassette of about 2,691 bp, and a 3′ mouse homology sequence. This DNA fragment was used to modify BAC clone bMQ-264A15 through homologous recombination in bacterial cells. As a result, an extracellular-encoding mouse TMPRSS2 genomic fragment (of about 25,291 bp) in the BAC clone was replaced with the human TMPRSS2 genomic fragment of about 25,091 bp, followed by a self-deleting neomycin cassette of about 2691 bp. Specifically, the mouse TMPRSS2 genomic fragment that was replaced included the last 7 bp of coding exon 3, intron 3, and coding exon 4 through the stop codon in coding exon 13 of the mouse TMPRSS2 gene (FIGS. 16A-16B). The human TMPRSS2 genomic fragment that was inserted included the last 7 bp of coding exon 3, intron 3, and coding exon 4 through coding exon 13 of a human TMPRSS2 gene (including the 3′ UTR of human TMPRSS2), and a human 3′ genomic sequence of 131 bp downstream of the 3′ UTR of human TMPRSS2 (FIGS. 16A-16B). The resulting modified BAC clone included, from 5′ to 3′, (i) a 5′mouse homology arm containing about 12 kb of mouse genomic DNA including a mouse TMPRSS2 5′ UTR, mouse TMPRSS2 exon 1 (non-coding), coding exons 1-3 (except the last 7 bp of coding exon 3); (ii) a human TMPRSS2 genomic fragment of about 25,091 bp including the last 7 bp of human coding exon 3, intron 3, human coding exons 4 through 13 (including the 3′ UTR of human TMPRSS2), and a human 3′ genomic sequence; (iii) a self-deleting neomycin cassette of about 2691 bp, followed by (iv) a 3′ mouse homology arm of 45 kb containing the mouse TMPRSS2 3′UTR and the remaining mouse genomic DNA in the original BAC clone. See FIGS. 16A-16B. The junction sequences are also set forth at the bottom of FIG. 16B. The part of the modified BAC clone containing the human TMPRSS2 genomic fragment and the neomycin cassette, as well as the upstream and downstream insertion junctions, is set forth in SEQ ID NO:72. The amino acid sequence of the protein encoded by the humanized TMPRSS2 gene is set forth in SEQ ID NO:74. An alignment of this humanized TMPRSS2 protein (“7010 mutant protein”), a mouse TMPRSS2 protein (SEQ ID NO:69), and a human TMPRSS2 protein (SEQ ID NO: 71), is provided in FIG. 16D.

The modified BAC clone containing the humanized TMPRSS2 gene, as described above, was used to electroporate mouse embryonic stem (ES) cells to create modified ES cells comprising a humanized TMPRSS2 gene. Positively targeted ES cells containing a humanized TMPRSS2 gene were identified by an assay (Valenzuela et al., supra) that detected the presence of the human TMPRSS2 sequences (e.g., coding exons 4-13 of human TMPRSS2) and confirmed the loss and/or retention of mouse TMPRSS2 sequences (e.g., loss of coding exons 4-13 of mouse TMPRSS2). Table 8 sets forth the primers and probes that were used to confirm humanization of an endogenous TMPRSS2 gene as described above (FIGS. 16A-16B). Once a correctly targeted ES cell clone has been selected, the neomycin selection cassette can be excised by introducing a Cre recombinase, e.g., via electroporation. Alternatively, the neomycin selection cassette can be removed by crossing the progeny generated from the ES clone with a deletor rodent strain that expresses a Cre recombinase. The humanized TMPRSS2 locus after the deletion of the cassette is depicted in FIG. 16C, with the junction sequences shown at the bottom of FIG. 16C.

Selected ES cell clones (with or without the cassette) were used to implant female mice using the VELOCIMOUSE®. method (see, e.g., U.S. Pat. No. 7,294,754 and Poueymirou et al., F0 generation mice that are essentially fully derived from the donor gene-targeted ES cells allowing immediate phenotypic analyses, 2007, Nature Biotech. 25 (1): 91-99) to generate a litter of pups containing a humanized TMPRSS2 allele in the genome. Mice bearing a humanized TMPRSS2 allele can be again confirmed and identified by genotyping of DNA isolated from tail snips using a modification of allele assay (Valenzuela et al., supra) that detects the presence of the human TMPRSS2 gene sequences. Pups are genotyped and cohorts of animals heterozygous for the humanized TMPRSS2 locus are selected for characterization. Animals homozygous for the humanized TMPRSS2 locus are made by crossing heterozygous animals.

TABLE 8

| Assay | Primer/Probe | Sequence |
|---|---|---|
| 7010U | Fwd | GCCGTGACTGTGACCTTCTC (SEQ ID NO: 93) |
| | Probe (BHQ) | TGGAGGAGCCACCTGATGCCTC (SEQ ID NO: 94) |
| | Rev | GCCTTGCCCTCAATGGAAAC (SEQ ID NO: 95) |
| 7010D | Fwd | GGTTGCACAGCAAGGAAGAAG (SEQ ID NO: 96) |
| | Probe (BHQ) | CCAGGAGTTCCTGTGAGCCTACCC (SEQ ID NO: 97) |
| | Rev | TGGAATGGAAGGAGCTGGAG (SEQ ID NO: 98) |
| 7010hU | Fwd | GTCCCACCTCCTGCAACTG (SEQ ID NO: 99) |
| | Probe (BHQ) | TGAGCCTTCCCATCAGCCTGGG (SEQ ID NO: 100) |
| | Rev | CCACAATGGCACATGGGTCTG (SEQ ID NO: 101) |
| 7010hTD | Fwd | GGTGCTTGCTCCCCAAGA (SEQ ID NO: 102) |
| | Probe (BHQ) | CCTAAAAGGTGTTGTAATGG (SEQ ID NO: 103) |
| | Rev | GGCAATAAAGAAGGAAGACGTTTT (SEQ ID NO: 104) |

### Example 5. Humanization of an Endogenous TMPRSS4 Gene

This example illustrates exemplary methods of humanizing an endogenous gene encoding TMPRSS4 in a rodent (e.g., a mouse). The methods described in this example can be employed to humanize an endogenous TMPRSS4 gene of a rodent using any human sequence, or combination of human sequences (or sequence fragments) as desired.

A targeting vector for humanization of an endogenous TMPRSS4 gene was constructed using bacterial artificial chromosome (BAC) clones and VELOCIGENE® technology (see, e.g., U.S. Pat. No. 6,586,251 and Valenzuela et al. (2003), supra).

Briefly, mouse bacterial artificial chromosome (BAC) clone RP23-71M15 containing a mouse TMPRSS4 gene was used and modified as follows. A DNA fragment was generated to include a 5′ mouse homology nucleotide sequence, a self-deleting neomycin cassette of about 4,996 bp, a human genomic DNA of about 14,963 bp (containing coding exon 4 through the stop codon in coding exon 13 of a human TMPRSS4 gene), and a 3′ mouse homology sequence. This DNA fragment was used to modify BAC clone RP23-71M15 through homologous recombination in bacterial cells. As a result, an extracellular-encoding mouse genomic fragment (of about 11,074 bp) in the BAC clone was replaced with a self-deleting neomycin cassette of about 4,996 bp, followed by the human genomic DNA of about 14,963 bp. Specifically, the mouse genomic fragment that was deleted and replaced included the 3′ 130 bp of mouse intron 3, coding exon 4 through the stop codon in coding exon 13 of the mouse TMPRSS4 gene (FIGS. 17A-17B). The human genomic fragment that was inserted included a 3′ portion of human TMPRSS4 intron 3 of about 150 bp, and human TMPRSS4 coding exon 4 through the stop codon in coding exon 13 (FIGS. 17A-17B). The resulting modified BAC clone included, from 5′ to 3′, a 5′ mouse homology arm containing about 44.8 kb of mouse genomic DNA (including a mouse TMPRSS4 5′ UTR, mouse TMPRSS4 coding exons 1 through 3, mouse TMPRSS4 intron 3 in part (without the 3′ 130 bp), a self-deleting neomycin cassette of about 4996 bp, a 3′ portion of human TMPRSS4 intron 3 of about 150 bp, human TMPRSS4 coding exons 4 through the stop codon in coding exon 13, followed directly by the mouse TMPRSS4 3′ UTR and the remaining mouse genomic DNA in the original BAC clone (a 3′ mouse homology arm of about 118 kb in total). See FIGS. 17A-17B. The junction sequences are also set forth at the bottom of FIG. 17B. The part of the modified BAC clone containing the neomycin cassette and the human TMPRSS4 genomic fragment, as well as the upstream and downstream insertion junctions, is set forth in SEQ ID NO:79. The amino acid sequence of the protein encoded by the humanized TMPRSS4 gene is set forth in SEQ ID NO:81. An alignment of this humanized TMPRSS4 protein (“7224 mutant pro”), a mouse TMPRSS4 protein (SEQ ID NO:76), and a human TMPRSS4 protein (SEQ ID NO:78), is provided in FIG. 17D.

The modified BAC clone containing the humanized TMPRSS4 gene, as described above, was used to electroporate mouse embryonic stem (ES) cells to create modified ES cells comprising a humanized TMPRSS4 gene. Positively targeted ES cells containing a humanized TMPRSS4 gene were identified by an assay (Valenzuela et al., supra) that detected the presence of the human TMPRSS4 sequences (e.g., coding exons 4-13 of human TMPRSS4) and confirmed the loss and/or retention of mouse TMPRSS4 sequences (e.g., loss of coding exons 4-13 of mouse TMPRSS4). Table 9 sets forth the primers and probes that were used to confirm humanization of an endogenous TMPRSS4 gene as described above (FIGS. 17A-17B). Once a correctly targeted ES cell clone has been selected, the neomycin selection cassette can be excised by introducing a Cre recombinase, e.g., via electroporation. Alternatively, the neomycin selection cassette can be removed by crossing the progeny generated from the ES clone with a deletor rodent strain that expresses a Cre recombinase. The humanized TMPRSS4 locus after the deletion of the cassette is depicted in FIG. 17C, with the junction sequences shown at the bottom of FIG. 17C.

Selected ES cell clones (with or without the cassette) were used to implant female mice using the VELOCIMOUSE® method (see, e.g., U.S. Pat. No. 7,294,754 and Poueymirou et al. (2007), supra) to generate a litter of pups containing a humanized TMPRSS4 allele in the genome. Mice bearing a humanized TMPRSS4 allele were again confirmed and identified by genotyping of DNA isolated from tail snips using a modification of allele assay (Valenzuela et al., supra) that detected the presence of the human TMPRSS4 gene sequences. Pups were genotyped and cohorts of animals heterozygous for the humanized TMPRSS4 locus were selected for characterization. Animals homozygous for the humanized TMPRSS4 locus were made by crossing heterozygous animals.

TABLE 9

| Assay | Primer/Probe | Sequence |
|---|---|---|
| 7224mU | Fwd | GAGCAGGGCCATGACACAT (SEQ ID NO: 109) |
| | Probe (BHQ) | ACCATTAGATCCCAGCACTGGACA (SEQ ID NO: 110) |
| | Rev | AAACCCTTCCCGAGAGAGAA (SEQ ID NO: 111) |
| 7224mTU2 | Fwd | GAGGAACACTGTGTCAAGGACTT (SEQ ID NO: 112) |
| | Probe (BHQ) | CCTGAAAAGCCCGGAGTGGCAG (SEQ ID NO: 113) |
| | Rev | GGGCAGAGACCACATCTGA (SEQ ID NO: 114) |
| 7224mTD | Fwd | GGAAGCCCTCTCTCGATACTTG (SEQ ID NO: 115) |
| | Probe (BHQ) | TTCTACCCTGAGGGCATGCAGC (SEQ ID NO: 116) |
| | Rev | TGGGATGTAGAAGGTTGTCAGA (SEQ ID NO: 117) |
| 7224hTU | Fwd | CTGAGCCTGGAACTCACACATG (SEQ ID NO: 118) |
| | Probe (BHQ) | TCTGAGAGCCCAGCACTATCGCC (SEQ ID NO: 119) |
| | Rev | GCTGAGGGTCAGGCTTGAG (SEQ ID NO: 120) |
| 7224hTD | Fwd | TCTGCAGGGTAGGGAGAGAAG (SEQ ID NO: 121) |
| | Probe (BHQ) | TGTTTCAGAAAAGGAAGACTCACGT (SEQ ID |
| | | NO: 122) |
| | Rev | GAGACCGATGAAGAGAAAGTCAGA (SEQ ID |
| | | NO: 123) |

### Example 6. Humanization of an Endogenous TMPRSS11d Gene

This example illustrates exemplary methods of humanizing an endogenous gene encoding TMPRSS11d in a rodent (e.g., a mouse). The methods described in this example can be employed to humanize an endogenous TMPRSS11d gene of a rodent using any human sequence, or combination of human sequences (or sequence fragments) as desired.

A targeting vector for humanization of an endogenous TMPRSS11d gene was constructed using bacterial artificial chromosome (BAC) clones and VELOCIGENE® technology (see, e.g., U.S. Pat. No. 6,586,251 and Valenzuela et al. (2003), supra).

Briefly, mouse bacterial artificial chromosome (BAC) clone RP23-95N22 containing a mouse TMPRSS11d gene was used and modified as follows. A DNA fragment was generated to include a 5′ mouse homology nucleotide sequence, a human TMPRSS11D genomic DNA of about 33,927 bp (containing 444 bp at the 3′ end of intron 2, and coding exon 3 through coding exon 10 (including the 3′ UTR which is part of coding exon 10), of a human TMPRSS11D gene), a self-deleting neomycin cassette of about 4,996 bp, and a 3′ mouse homology sequence. This DNA fragment was used to modify BAC clone RP23-95N22 through homologous recombination in bacterial cells. As a result, an extracellular-encoding mouse TMPRSS11d genomic fragment (of about 35,667 bp) in the BAC clone was replaced with the human TMPRSS11D genomic fragment of about 33,927 bp, followed by a self-deleting neomycin cassette of about 4,996 bp. Specifically, the mouse TMPRSS11d genomic fragment that was replaced included a 3′ portion of intron 2, and coding exon 3 through the stop codon in coding exon 10 of the mouse TMPRSS11d gene (FIGS. 18A-18B). The human TMPRSS11D genomic fragment that was inserted included 444 bp at the 3′ end of intron 2, and coding exon 3 through coding exon 10 of a human TMPRSS11D gene (including the 3′ UTR of human TMPRSS11D), and a human 3′ genomic sequence of about 172 bp downstream of the 3′ UTR of human TMPRSS11D (FIGS. 18A-18B). The resulting modified BAC clone included, from 5′ to 3′, (i) a 5′ mouse homology arm containing about 143 kb of mouse genomic DNA including a mouse TMPRSS11d 5′ UTR, mouse TMPRSS11d coding exons 1-2 and a 5′ portion of intron 2; (ii) a human TMPRSS11D genomic fragment including a 3′ portion of intron 2 and coding exons 3 through 10 (including the 3′ UTR) of human TMPRSS11D, and a human 3′ genomic sequence; (iii) a self-deleting neomycin cassette of about 4,996 bp, followed by (iv) a 3′ mouse homology arm of 10 kb containing the mouse TMPRSS11d 3′UTR and the remaining mouse genomic DNA in the original BAC clone. See FIGS. 18A-18B. The junction sequences are also set forth at the bottom of FIG. 18B. The part of the modified BAC clone containing the human TMPRSS11D genomic fragment and the neomycin cassette, as well as the upstream and downstream insertion junctions, is set forth in SEQ ID NO:86. The amino acid sequence of the protein encoded by the humanized TMPRSS11d gene is set forth in SEQ ID NO:88. An alignment of this humanized TMPRSS11d protein (“7226 mutant pro”), a mouse TMPRSS11d protein (SEQ ID NO:83), and a human TMPRSS11D protein (SEQ ID NO:85), is provided in FIG. 18D.

The modified BAC clone containing the humanized TMPRSS11d gene, as described above, is used to electroporate mouse embryonic stem (ES) cells to create modified ES cells comprising a humanized TMPRSS11d gene. Positively targeted ES cells containing a humanized TMPRSS11d gene are identified by an assay (Valenzuela et al., supra) that detects the presence of the human TMPRSS11D sequences (e.g., coding exons 3-10 of human TMPRSS11D) and confirms the loss and/or retention of mouse TMPRSS11d sequences (e.g., loss of coding exons 3-10 of mouse TMPRSS11d). Table 10 sets forth the primers and probes that were used to confirm humanization of an endogenous TMPRSS11d gene as described above (FIGS. 18A-18B). Once a correctly targeted ES cell clone has been selected, the neomycin selection cassette can be excised by introducing a Cre recombinase, e.g., via electroporation. Alternatively, the neomycin selection cassette can be removed by crossing the progeny generated from the ES clone with a deletor rodent strain that expresses a Cre recombinase. The humanized TMPRSS11d locus after the deletion of the cassette is depicted in FIG. 18C, with the junction sequences shown at the bottom of FIG. 18C.

Selected ES cell clones (with or without the cassette) are used to implant female mice using the VELOCIMOUSE® method (see, e.g., U.S. Pat. No. 7,294,754 and Poueymirou et al. (2007), supra) to generate a litter of pups containing a humanized TMPRSS11d allele in the genome. Mice bearing a humanized TMPRSS11d allele are again confirmed and identified by genotyping of DNA isolated from tail snips using a modification of allele assay (Valenzuela et al., supra) that detects the presence of the human TMPRSS11D gene sequences. Pups are genotyped and cohorts of animals heterozygous for the humanized TMPRSS11d locus are selected for characterization. Animals homozygous for the humanized TMPRSS11d locus are made by crossing heterozygous animals.

TABLE 10

| Assay | Primer/Probe | Sequence |
|---|---|---|
| 7226mTU | Fwd | TCCTCTCCAGACAAGAAAGCT (SEQ ID NO: 128) |
| | Probe (BHQ) | TCATAGCAGCTTTCAAATCCTAAACGTTGA (SEQ ID |
| | | NO: 129) |
| | Rev | TCGTGTGTAGCTGGTGAGTT (SEQ ID NO: 130) |
| 7010D | Fwd | CATGCGATCACAGGAGGAGATC (SEQ ID NO: 131) |
| | Probe (BHQ) | AATTGGGCCCGAAGCCAGATGC (SEQ ID NO: 132) |
| | Rev | CGGAAGGCTTCTGTGACTTC (SEQ ID NO: 133) |
| 7010hU | Fwd | GTCTCCCACTTCTGACATAATGAAC (SEQ ID NO: 134) |
| | Probe (BHQ) | CCCAGTGTTAACCCTACATCTGGTTCC (SEQ ID |
| | | NO: 135) |
| | Rev | TGGGAAGAGACTCTTGGACA (SEQ ID NO: 136) |
| 7010hTD | Fwd | ATGAGCTCCTAGTACAGCTAAAGTT (SEQ ID |
| | | NO: 137) |
| | Probe (BHQ) | ATGCATGATCATCTATGCGTCAGAGC (SEQ ID |
| | | NO: 138) |
| | Rev | TGCCCAGATGCAGGGAGTTAG (SEQ ID NO: 139) |

### Example 6. Characterization of Humanized ACE2 and TMPRSS2 Mouse

Mice comprising a humanized ACE2 gene at an endogenous ACE2 locus were generated as described above. Briefly, in the ACE2 mouse, a nucleotide sequence comprising part of coding exon 1 through part of coding exon 17 of an endogenous ACE2 gene was replaced with the correspondence sequence of a human ACE2 gene such that the human ACE2 nucleotide sequence was operably inked to endogenous nucleotide sequences encoding endogenous ACE2 transmembrane and cytoplasmic domains.

Mice comprising a genetically engineered TMPRSS2 gene were generated as described above. Briefly, in the TMPRSS2 mouse, a human TMPRSS2 genomic DNA of about 25,091 bp (containing the last 7 bp of coding exon 3, intron 3, and coding exon 4 through coding exon 13 (including the 3′ UTR which is part of coding exon 13), of a human TMPRSS2 gene replaced the orthologous sequence at an endogenous TMPRSS2 locus.

Homozygous ACE2 mice described above were bred to homozygous TMPRSS2 mice to produce a mouse heterozygous for the ACE2 allele and the TMPRSS2 allele. F1 heterozygous mice generated from this cross were bred to each other to obtain mice homozygous for each allele. Such mice express chimeric human ACE2 and TMPRSS.

The presence of the genetically modified alleles in the endogenous ACE2 and TMPRSS2 loci was confirmed by TAQMAN™ screening and karyotyping using specific probes and primers described above.

Alternatively, to generate mice comprising both ACE2 allele and TMPRSS2 alleles, ES cells harboring an ACE2 modification or ES cells harboring a TMPRSS2 modification are targeted with the TMPRSS2 or ACE2 targeting vector, respectively. Mice are generated from ES cells harboring both modifications by introducing ES cells into an 8 stage mouse embryo by VELOCIMMUNE® method and screening as described above. F1 heterozygous mice are bred to obtain homozygous mice.

All mice were housed and bred in specific pathogen-free conditions. Five week old mice comprising a humanized ACE2 locus alone or both a humanized ACE2 locus and a humanized TMPRSS2 locus were compared to ACE2-null mice. ACE2-null mice lack an ACE coding sequence at an endogenous ACE2 allele (see, e.g., FIG. 1C).

Expression Levels of Human ACE2 and Mouse or Human TMPRSS2 Messenger RNA (mRNA)

The expression levels of human ACE2, mouse TMPRSS2, or human TMPRSS2 mRNA were analyzed by TaqMan qRT-PCR analysis as described herein. Messenger RNA levels in this Example were analyzed by Real-Time Quantitative Polymerase Chain Reaction (RT-qPCR). Total RNA from each sample was extracted and reverse transcribed using primers that amplify human ACE2, mouse TMPRSS2, and human TMPRSS2 genes. RT-qPCR was performed using probes and primers of readily available kits.

Specifically, 1 cm pieces of duodenum, colon, liver, kidney, heart, lung, and trachea were isolated directly into RNALater (Ambion by Life Technologies), and chilled to 4′C. Tissues were homogenized in TRIzol, and chloroform was used for phase separation. The aqueous phase, containing total RNA, was purified using the MagMAX™-96 for Microarrays Total RNA Isolation Kit (Ambion by Life Technologies) according to manufacturer's specifications. Genomic DNA was removed using RNase-Free DNase Set (Qiagen). mRNA was reverse-transcribed into cDNA using SuperScript® VILO™ Master Mix (Invitrogen by Life Technologies). cDNA was amplified with the SensiFAST Probe Lo-ROX (Meridian) using the 12K Flex System (Thermofisher). A housekeeping control gene (Gapdh) was used to normalize any cDNA input differences and relative levels were plotted against kidney levels in the ACE2/TMPRSS2 mouse. Negative control mRNAs were extracted from mice that are deleted in the ACE2 gene (aka ACE2-null, or knockout, or KO). Positive control mRNAs were extracted from wild-type (WT) mice.

As shown in FIGS. 19A and 19B, two human-specific assays (hEx3-4 and hEx16-17) amplified only the humanized Ace2 allele. No amplification was detected in Ace2-KO) tissues. Human-specific TMPRSS2 assay detected variable levels of human mRNA in double ACE2/TMPRSS2 mice (FIG. 19C). KO and hACE2 mice are negative controls. The Mouse Tmprss2 assay amplified at variable levels in all mice, since only one allele of the gene is humanized in the ACE2/TMPRSS2 mice tested.

The sequences of the primers and probes used in the analysis are provided in Table 11 below.

| Assay | | |
|---|---|---|
| name | Primer | Sequence |
| hTMPRSS2 | Forward | CACGGACTGGATTTATCGACAA |
| | Reverse | AAGGACGAAGAGGATGTGGATT |
| | Probe | TGAGGGCAGAGGGG |
| mTmprss2 | Forward | Life Technologies assay ID |
| | Reverse | Mm00443687_m1, ThermoFisher |
| | Probe | |

What is claimed is:

1. A non-human animal, non-human animal cell, or non-human animal genome comprising:
(I) a humanized ACE2 gene encoding a recombinant ACE2 protein, wherein the recombinant ACE2 protein comprises in operable linkage:
(a) an ACE2 signal sequence of a non-human animal ACE2 protein or an ACE2 signal sequence of a human ACE2 protein,
(b) an extracellular domain substantially identical to an extracellular domain of a human ACE2 protein,
(c) a transmembrane domain of anon-human animal ACE2 protein or a transmembrane domain of a human ACE2 protein, and
(d) a cytoplasmic domain of a non-human animal ACE2 protein or a cytoplasmic domain of a human ACE2 protein, and
(II) one or more distinct and humanized TMPRSS genes, each respectively encoding one or more distinct recombinant TMPRSS proteins that comprises in operable linkage:
(a) a TMPRSS signal sequence of a non-human animal TMPRSS protein or TMPRSS signal sequence of a human TMPRSS protein,
(b) an extracellular domain substantially identical to the extracellular domain of the human TMPRSS protein, and
(c) a transmembrane domain of the non-human animal TMPRSS protein or a transmembrane domain of the human TMPRSS protein, and
(d) a cytoplasmic domain of the non-human TMPRSS protein or a cytoplasmic domain of the human TMPRSS protein,
wherein the non-human animal TMPRSS protein and the human TMPRSS protein are orthologous.

2. The non-human animal, non-human animal cell, or non-human animal genome of claim 1, wherein there combinant ACE2 protein comprises in operable linkage:
(a) an ACE2 signal sequence of an endogenous non-human animal ACE2 protein,
(b) an extracellular domain substantially identical to the extracellular domain of a human ACE2 protein,
(c) a transmembrane domain of an endogenous non-human animal ACE2 protein, and
(d) a cytoplasmic domain of an endogenous non-human animal ACE2 protein, and
(II) wherein the recombinant TMPRSS protein comprises in operable likage:
(a) a TMPRSS signal sequence of the non-human animal TMPRSS protein
(b) an extracellular domain substantially identical to the extracellular domain of a human TMPRSS protein, and
(c) a transmembrane domain of the non-human TMPRSS protein, and
(d) a cytoplasmic domain of the non-human TMPRSS protein,
wherein the non-human animal TMPRSS protein and the human TMPRSS protein are orthologous.

3. The non-human animal, non-human animal cell, or non-human animal genome of claim 1 or claim 2, wherein
(I) the humanized ACE2 gene comprises a nucleotide sequence encoding an extracellular domain substantially identical to the extracellular domain of a human ACE2 protein operably linked to an endogenous nucleotide sequence encoding (iii) the transmembrane domain of an endogenous non-human animal ACE2 protein, and (iv) the cytoplasmic domain of an endogenous non-human animal ACE2 protein; and/or
(II) each of the one or more distinct and humanized TMPRSS gene comprises a nucleotide sequence encoding an extracellular domain substantially identical to the extracellular domain of a human TMPRSS protein operably linked to an endogenous nucleotide sequence encoding (iii) the transmembrane domain of an endogenous and cognate animal TMPRSS protein and (iv) the cytoplasmic domain of an endogenous and non-human animal TMPRSS protein,
wherein the non-human animal TMPRSS protein and the human TMPRSS protein are orthologous.

4. The non-human animal, non-human animal cell, or non-human animal genome of any one of the preceding claims, wherein
(I) an endogenous ACE2 locus comprises the humanized ACE2 gene,
wherein the endogenous ACE2 locus comprises a replacement of the nucleotide sequence encoding the extracellular domain of the endogenous ACE2 protein with the nucleotide sequence encoding the extracellular domain of a human ACE2 protein such that the nucleotide sequence encoding the extracellular domain of a human ACE2 protein is operably linked to an endogenous nucleotide sequence encoding (iii) the transmembrane domain of an endogenous non-human animal ACE2 protein, and (iv) the cytoplasmic domain of an endogenous non-human animal ACE2 protein and/or
(II) an endogenous TMPRSS locus comprises one of the one or more distinct and humanized TMPRSS genes,
wherein the endogenous TMPRSS locus comprises a replacement of the nucleotide sequence encoding the extracellular domain of the endogenous TMPRSS protein with the nucleotide sequence encoding the extracellular domain of a human TMPRSS protein such that the nucleotide sequence encoding the extracellular domain of the human TMPRSS protein is operably linked to an endogenous nucleotide sequence encoding (iii) the transmembrane domain of an endogenous TMPRSS protein at the endogenous TMPRSS locus, and (iv) the cytoplasmic domain of the endogenous TMPRSS protein,
wherein the non-human animal TMPRSS protein and the human TMPRSS protein are orthologous.

5. The non-human animal, non-human animal cell, or non-human animal genome of any one of the preceding claims, wherein
(I) the humanized ACE2 gene encodes a recombinant ACE2 protein comprising in operable linkage:
(i) an ACE2 signal sequence of an endogenous ACE2 protein,
(ii) an extracellular domain of a human ACE2 protein,
(iii) a transmembrane domain of an endogenous ACE2 protein, and
(iv) a cytoplasmic domain of an endogenous mouse ACE2 protein, optionally
wherein the extracellular domain of a human ACE2 protein is encoded by part of the coding sequence of coding exon 1, all of the coding sequences of coding exon 2 to coding exon 16, inclusive, and part of coding exon 17 of a human ACE2 gene and/or wherein the amino acid sequence of the recombinant ACE2 protein is set forth in SEQ ID NO:20, and
(II) one of the one or more distinct and humanized TMPRSS genes is:
(A) a humanized TMPRSS2 gene, optionally wherein the humanized TMPRSS2 gene:
(i) is at an endogenous TMPRSS2 locus, optionally under control of the endogenous TMPRSS2 promoter at the endogenous TMPRSS2 locus,
(ii) is formed by a replacement of an endogenous nucleotide sequence of an endogenous TMPRSS2 gene, or portion thereof, with an orthologous nucleotide sequence of a human TMPRSS2 gene,
(iii) comprises coding exon 4 through the stop codon in coding exon 13 of the human TMPRSS2 gene, and optionally further comprises the 3′ UTR of the human TMPRSS2 gene,
(iv) comprises coding exons 1-2 of the endogenous TMPRSS2 gene and coding exon 4 through coding exon 13 of the human TMPRSS2 gene, and optionally the 3′ UTR of the human TMPRSS2 gene
(v) comprises an exon 3 that comprises a 5′ portion of coding exon 3 of the endogenous TMPRSS2 gene and a 3′ portion of coding exon 3 of the human TMPRSS2 gene and/or
(vi) encodes a humanized TMPRSS2 protein that comprises (i) an extracellular domain of the human TMPRSS2 protein encoded by a human TMPRSS2 gene, and (ii) a cytoplasmic and transmembrane portion encoded by the endogenous TMPRSS2 gene, optionally wherein the extracellular domain of the human TMPRSS2 protein comprises the residues W 106 to G492 of SEQ ID NO:71;
(B) a humanized TMPRSS4, optionally wherein the humanized TMPRSS4 gene:
(i) is at an endogenous TMPRSS4 locus, optionally under control of the endogenous TMPRSS4 promoter at the endogenous TMPRSS4 locus,
(ii) is formed by a replacement of an endogenous nucleotide sequence of an endogenous TMPRSS4 gene, or portion thereof, with an orthologous nucleotide sequence of a human TMPRSS4 gene,
(iii) comprises coding exon 4 through the stop codon in coding exon 13 of the human TMPRSS4 gene, and optionally further comprises the 3′UTR of the human TMPRSS2 gene,
(iv) comprises coding exons 1-3 of the endogenous TMPRSS2 gene and coding exon 4 through coding exon 13 of the human TMPRSS4 gene, and optionally the 3′ UTR of the human TMPRSS4 gene and/or
(v) encodes a humanized TMPRSS4 protein that comprises (i) an extracellular domain of the human TMPRSS4 protein encoded by a human TMPRSS4 gene, and (ii) a cytoplasmic and transmembrane portion encoded by the endogenous TMPRSS4 gene, optionally wherein the extracellular domain of the human TMPRSS4 protein comprises the residues K54-L437 of SEQ ID NO:78; and/or
(C) a humanized TMPRSS11, optionally wherein the humanized TMPRSS11 gene:
(i) is at an endogenous TMPRSS11 locus, optionally under control of the endogenous TMPRSS11 promoter at the endogenous TMPRSS11 locus,
(ii) is formed by a replacement of an endogenous nucleotide sequence of an endogenous TMPRSS11 gene, or portion thereof, with an orthologous nucleotide sequence of a human TMPRSS11 gene,
(iii) comprises coding exon 3 through the stop codon in coding exon 10 of the human TMPRSS11 gene, and optionally further comprises the 3′ UTR of the human TMPRSS11 gene,
(iv) comprises coding exons 1-2 of the endogenous TMPRSS11 gene and coding exon 3 through coding exon 10 of the human TMPRSS11 gene, and optionally the 3′ UTR of the human TMPRSS11 gene and/or
(v) encodes a humanized TMPRSS11 protein that comprises (i) an extracellular domain of the human TMPRSS11 protein encoded by a human TMPRSS11 gene, and (ii) a cytoplasmic and transmembrane portion encoded by the endogenous TMPRSS11 gene, optionally wherein the extracellular domain of the human TMPRSS11 protein comprises the residues A42 to 1418 of SEQ ID NO:85.

6. The non-human animal, non-human animal cell, or non-human animal genome of any one of the preceding claims, wherein the non-human animal, non-human animal cell, or non-human animal genome comprises at least two humanized TMPRSS genes, each at orthologous endogenous TMPRSS gene loci.

7. The non-human animal, non-human animal cell, or non-human animal genome of any one of the preceding claims wherein
(I) the humanized ACE2 gene is under control of the promoter of the endogenous ACE2 gene and/or
(II) each of the one or more distinct and human TMPRSS genes is under the control of the promoter of the endogenous and orthologous TMPRESS gene.

8. The non-human animal, non-human animal cell, or non-human animal genome of any one of the preceding claims, wherein the non-human animal is heterozygous for the humanized ACE2 and/or humanized TMPRSS gene(s).

9. The non-human animal, non-human animal cell, or non-human animal genome of any one of claim 108 wherein the non-human animal is homozygous for the humanized ACE2 and/or humanized TMPRSS gene(s).

10. The non-human animal, non-human animal cell, or non-human animal genome of any one of the preceding claims, wherein
the non-human animal is a rodent,
the non-human animal cell is a rodent cell, or
the non-human animal genome is a rodent genome.

11. The non-human animal, non-human animal cell, or non-human animal genome of any one of the preceding claims, wherein
the non-human animal is a rat,
the non-human animal cell is a rat cell, or
the non-human animal genome is a rat genome.

12. The non-human animal, non-human animal cell, or non-human animal genome of any one of claims 1-10, wherein
the non-human animal is a mouse,
the non-human animal cell is a mouse cell, or
the non-human animal genome is a mouse genome.

13. The non-human animal, non-human animal cell, or non-human animal genome of any one of the preceding claims, wherein the non-human animal, non-human animal cell, or non-human animal genome expresses the recombinant ACE2 protein and/or the one or more distinct recombinant TMPRSS proteins.

14. A non-human animal cell of any one of claims 1-12, wherein the cell is an embryonic stem (ES) cell.

15. A non-human animal tissue comprising the non-human animal cell of any one of the preceding claims.

16. A method for making a non-human animal, non-human animal cell, or non-human animal genome comprising a humanized ACE2 and a humanized TMPRSS gene, the method comprising:
(A) generating a genetically modified non-human embryonic stem (ES) cell comprising:
(I) humanized ACE2 gene encoding a recombinant ACE2 protein, wherein the recombinant ACE2 protein comprises in operable linkage:
(a) an ACE2 signal sequence of a non-human animal ACE2 protein or an ACE2 signal sequence of a human ACE2 protein,
(b) an extracellular domain substantially identical to an extracellular domain of a human ACE2 protein,
(c) a transmembrane domain of a non-human animal ACE2 protein or a transmembrane domain of a human ACE2 protein, and
(d) a cytoplasmic domain of a non-human animal ACE2 protein or a cytoplasmic domain of a human ACE2 protein, and
(II) one or more distinct and humanized TMPRSS genes, each encoding a distinct recombinant TMPRSS protein that comprises in operable likage:
(a) a TMPRSS signal sequence of a non-human animal TMPRSS protein or TMPRSS signal sequence of a human TMPRSS protein,
(b) an extracellular domain substantially identical to the extracellular domain of the human TMPRSS protein, and
(c) a transmembrane domain of the non-human animal TMPRSS protein or a transmembrane domain of the human TMPRSS protein, and
(d) a cytoplasmic domain of the non-human TMPRSS protein or a cytoplasmic domain of the human TMPRSS protein,
wherein the non-human animal TMPRSS protein and the human TMPRSS protein are orthologous
(B) introducing the modified non-human animal ES cell into a host embryo of non-human animal to form a donor cell-non-human animal embryo complex; and
(C) gestating the donor cell-non-human animal embryo in a surrogate non-human animal mother, wherein the surrogate non-human animal mother produces rodent progeny that express the humanized ACE2 and TMPRSS proteins.

17. The method of claim 16, wherein generating the genetically modified non-human animal ES cell comprises:
(i) obtaining a non-human ES cell that comprises the humanized ACE2 gene, and
(ii) modifying the genome of the obtained non-human ES cell that comprises the humanized ACE2 gene to further comprise the one or more distinct humanized TMPRSS genes,
wherein modifying the genome comprises replacing an endogenous nucleotide sequence encoding an extracellular domain of an endogenous TMPRSS protein with a nucleotide sequence encoding a transmembrane domain substantially identical (e.g., at least 85%, 90%, 95%, 98%, 99%, or 100% identical in sequence) to the extracellular domain of a human TMPRSS protein that is orthologous to the endogenous TMPRSS protein.

18. The method of claim 16, wherein generating the genetically modified non-human animal ES cell comprises:
(i) obtaining a non-human ES cell that comprises the one or more distinct humanized TMPRSS genes, and
(ii) modifying the genome of the obtained non-human ES cell that comprises the tone or more distinct humanized TMPRSS genes to further comprise the humanized ACE2 gene,
wherein modifying the genome comprises replacing an endogenous nucleotide sequence encoding an extracellular domain of an endogenous ACE2 protein with a nucleotide sequence encoding an extracellular domain substantially identical (e.g., at least 85%, 90%, 95%, 98%, 99%, or 100% identical in sequence) to the extracellular domain of a human ACE2 protein.

19. A non-human animal, non-human animal cell, or non-human animal genome made according to the method of any one of claims claim 16-18.

20. A composition comprising the non-human animal cell or tissue of any one of claims 1-15 and 19.

21. The composition of claim 20, further comprising a spike protein of a coronavirus, wherein the spike protein binds the human ACE2 protein.

22. The composition of claim 20 or 21, further comprising a therapeutic agent that inhibits or prevents binding of an ACE2 ligand to the recombinant ACE2 protein, optionally wherein the ACE2 ligand comprises a spike protein of a coronavirus.

23. The composition of claim 22, wherein the therapeutic agent is an antigen-binding protein that binds the spike protein of a coronavirus.

24. A non-human animal model of coronavirus infection comprising
(a) a non-human animal or non-human animal cell according to any one of claims 1-14 and 19 or made according to any one of claims 16-18, wherein the non-human animal or non-human animal cell expresses the recombinant ACE2 protein and/or the one or more distinct recombinant TMPRSS proteins, and
(b) a coronavirus comprising a spike protein that binds to a human ACE2 protein.

25. A method of screening drug candidates that target a ligand of a human ACE2 protein, comprising:
a. introducing the ligand of a human ACE2 protein into a genetically modified non-human animal according to any one of claims 1-14 and 19 or made according to a method of any one of claims 16-18, wherein the non-human animal or non-human animal cell expresses the recombinant ACE2 protein and/or the one or more distinct recombinant TMPRSS proteins,
b. contacting the non-human animal with a drug candidate of interest, wherein the drug candidate is directed against the ligand of a human ACE2 protein, and
c. determining if the drug candidate is efficacious in preventing, reducing or eliminating binding of the ligand of a human ACE2 protein to the recombinant ACE2 protein.

26. The method of claim 25, wherein the step of introducing comprises infecting the non-human animal with a coronavirus, wherein the coronavirus comprises a spike protein, and wherein the spike protein comprises the ligand of a human ACE2 protein.
